(19) United States
(12) Patent Application Publication
(10) Pub. No.: US 2025/0101003 A1
(43) Pub. Date: Mar. 27, 2025

(54) SPIROCYCLIC CAV2.3 ANTAGONISTS

(71) Applicant: LARIO THERAPEUTICS LIMITED, Edinburgh (GB)

(72) Inventors: Henning STEINHAGEN, Schwalbach (DE); Paolo PEVARELLO, Pavia (IT); Jean-Francois ROLLAND, Bari (IT); Maria Pia CATALANI, Verona (IT)

(21) Appl. No.: 18/713,002

(22) PCT Filed: Nov. 25, 2022
(86) PCT No.: PCT/GB2022/052997

§ 371 Date: May 23, 2024

(30) Foreign Application Priority Data
Nov. 26, 2021 (GB) 2117126.9

(51) Int. Cl. C07D 403/12; A61K 31/403; A61K 31/4178; A61K 31/4184; A61K 31/422; A61K 31/423; A61K 31/427; A61K 31/428; A61K 31/433; A61K 31/438; A61K 31/4439; A61K 31/4709; A61K 31/4725; A61K 31/497; A61K 31/506; C07D 209/54; C07D 401/12; C07D 405/12; C07D 409/12; C07D 413/12; C07D 413/14; C07D 417/12

(52) U.S. Cl. CPC C07D 403/12; A61K 31/403; A61K 31/4178; A61K 31/4184; A61K 31/422; A61K 31/423; A61K 31/427; A61K 31/428; A61K 31/433; A61K 31/438; A61K 31/4439; A61K 31/4709; A61K 31/4725; A61K 31/497; A61K 31/506; C07D 209/54; C07D 401/12; C07D 405/12; C07D 409/12; C07D 413/12; C07D 413/14; C07D 417/12

(57) ABSTRACT

Disclosed are compounds of the formula (I) and pharmaceutically acceptable salts thereof wherein X¹, R³, R⁴, a, b, p, q, r, L, and Ring A are as defined herein. The compounds are antagonists of the resistant (R-type) voltage-gated calcium ion channel Cav 2.3. Also disclosed are pharmaceutical compositions comprising the compounds; and the compounds for use in the treatment of diseases modulated Cav 2.3, including neurodegenerative conditions such as Parkinson's disease, focal, drug-resistant forms of epilepsy, and other neurological disorders such as developmental and epileptic encephalopathies and Fragile X syndrome.

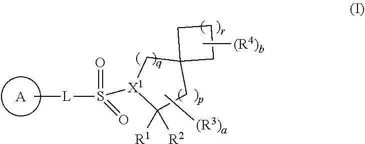

This invention relates to compounds that are antagonists of the resistant (R-type) voltage-gated calcium ion channel Cav 2.3, and the use of the compounds in the treatment and prevention of diseases and conditions associated with Cav2.3, for example neurodegenerative conditions such as Parkinson's disease, focal, drug-resistant forms of epilepsy, and other neurological disorders such as developmental and epileptic encephalopathies and Fragile X syndrome.

### BACKGROUND

Voltage-dependent calcium channels are multi-subunit complexes consisting of alpha-1, alpha-2, beta, and delta subunits in a 1:1:1:1 ratio. Cav2.3 channels belong to the so called pharmaco-resistant or “residual” (R-type) membrane-bound voltage-gated calcium channels and are responsible for calcium ion influx into cells that express them. These channels are structurally only partially characterised. Nevertheless, it is well accepted that most of them are encoded by the CACNA1E gene (Gene ID 777) and are expressed as different Cav2.3 splice variants (variant Cav2.3a to Cav2.3e or f) as the ion conducting subunit (Schneider et al., Pflügers Arch. 2020; 472(7): 811-816).

Cav2.3 is highly expressed in neuronal and endocrine tissues and has also been detected in heart, kidney, sperm, spleen, and retina, and is associated with numerous physiologic and pathophysiologic processes in the central nervous system, vascular system and in endocrine systems (Schneider et al., Pharmaceuticals 2013, 6(6), 759-776, Schneider et al., Pflügers Arch. 2020; 472(7): 811-816).

Parkinson disease is the second-most common neurodegenerative disorder that affects 2-3% of the population ≥65 years of age. The primary motor symptoms of Parkinson's are caused by the progressive degeneration of dopaminergic midbrain neurons, particularly those within the substantia nigra (SN) neurones (Giguere et al. 2018, Front. Neurol. 9, 455). This leads to a striatal dopamine deficiency, and intracellular inclusions containing aggregates of α-synuclein are the neuropathological hallmarks of Parkinson disease (Poewe et al, Nat Rev Dis Primers. 2017 Mar. 23; 3:17013). Currently there are no curative therapies available for Parkinson's disease (Bloem et al, Lancet. 2021 Jun. 12; 397(10291):2284-2303). Parkinson's disease is a multifactorial disease, and besides genetic risk-factors for Parkinson's disease like PARK-gene mutations, numerous Parkinson's disease-stressors have been identified, including inflammation, viral infections, trauma, gut bacteria, or environmental toxins. Most of these factors lead to mitochondria, proteasomal, and/or lysosomal dysfunction, and elevated metabolic stress, key pathophysiological events in Parkinson's disease. As PARK mutations and also most external factors are global Parkinson's disease-stressors, additional cell-specific features must also contribute to the Parkinson's disease-pathophysiology, and in particular to the differential neuronal vulnerability.

Dopaminergic midbrain neurons display pacemaker activity, which is important for dopamine release and e.g., voluntary movement control. In SN dopaminergic neurons (and other highly vulnerable neurons), this activity generates oscillatory increases in free cytosolic Ca²⁺ levels, which are associated with oscillatory elevated levels of metabolic stress (Guzman et al, Nature. 2010 Dec. 2; 468(7324):696-700; Liss & Striessnig, Annu. Rev. Pharmacol Toxicol. 2019 Jan. 6; 59:263-289; Ortner, Front. Synaptic Neurosci. 2021 Feb. 26; 13:636103; Zampese & Surmeier, Cells 2020 Sep. 8; 9(9):2045). These increased stresses are thought to render SN neurons more vulnerable to degeneration by Parkinson's disease stressors. Cav2.3 is highly expressed in adult SN dopaminergic neurons and accounts for ˜50% of somatic Ca2+ oscillations in SN DA neurons (Benkert et al., 2019, Nat. Commun. 10, 5094).

In patch clamp electrophysiology experiments on brain slices from Cav2.3 knockout mice, the amplitude of the activity-related Ca²⁺ oscillations were significantly reduced by ˜50% in somata of SN neurones compared to wild-type mice. Ca²⁺-dependent action potential after-hyperpolarizations (AHPs), were also significantly reduced in SN dopaminergic neurons of Cav2.3 knockout mice, consistent with the reduction in the Ca²⁺ signals. Similar effects were also observed when Cav2.3 channels were partially blocked using low concentrations of the non-selective peptide antagonist SNX-482, blocking Cav2.3 but also A-type Kv4 potassium channels with protective effects in SN dopaminergic (DA) neurons (Kimm et al, 2014, 34 (28) 9182-9189). Cav 2.3 has also been shown to be implicated the preferential degeneration of these SN DA neurons in an in-vivo model of Parkinson's disease (Benkert et al., 2019, Nat. Commun. 10, 5094).

In a mouse Cav2.3 knockout model in which mice were subjected to low-dose MPTP/probenecid (neurotoxin) knockout of Cav2.3 was shown to provide a significant 100% neuroprotective effect on SN dopaminergic neurons compared to wild-type mice. These data identify Cav2.3 as mediator of SN dopaminergic neuron vulnerability to a degenerative stressor and suggest that Cav2.3 antagonists would be useful in the treatment of Parkinson's disease, for example by providing a neuroprotective treatment of the disease that prevents or inhibits disease progression (Benkert et al., 2019, Nat. Commun. 10, 5094).

In addition to neurodegenerative diseases such as Parkinson's disease Cav2.3 channels are also associated with other diseases and medical disorders, for example Fragile X syndrome (Gray et al., J Neurosci. 2019 Sep. 18; 39(38):7453-7464), monogenic developmental and epileptic encephalopathies (DEE) (Carvill, Epilepsy Curr. May-June 2019; 19(3):199-201; Helbig et al., Am J Hum Genet. 2019 Mar. 7; 104(3): 562; Ortiz Cabrera, Mol Syndromol. 2021 March; 12(1):25-32), focal, drug-resistant forms of epilepsy (Weiergraber et al., Epilepsia, 2006, 47:839-50; Weiergraber et al., J. Neurophysiol., 2007, 97:3660-69; Zaman et al., Neuron, 2011, 70:95-108), neurodevelopmental disorders, endocrine disorders such as diabetes (e.g., glucose-induced insulin release, glucose-mediated glucagon suppression, or glucose-mediated somatostatin-release) (Jing et al, 2005, The Journal of clinical investigation 115: 146-154, Rorsman et al, 2018, Physiological reviews 98: 117-214), the treatment of vasospasm following cerebral aneurism or subarachnoidal haemorrhage (Wang et al., 2010, Journal of Neurotrauma, vol. 27, no. 9, pp. 1723-1732), and pain (for example, chronic pain, inflammatory pain, neuropathic pain (e.g. peripheral neuropathic pain (Shan et al., ACS Chem. Neurosci. 2019, 10, 6, 2939-2955) or central neuropathic pain), or nociceptive pain) (Schneider et al.; Ishiguro et al, Circ. Res. 2005, 96, 419-426, Patel et al, British Journal of Pharmacology 2018, 175, 2173-2184; Wormuth et al., Open Neurol J. 2016; 10:99-126).

WO2018/228692 discloses that Cav2.3 antagonists are beneficial in the neuroprotective treatment of Parkinson's disease and other neurodegenerative diseases.

SNX-482 is a peptide antagonist of Cav2.3 derived from the venom of the tarantula Hysterocratis gigas. SNX-482 has an IC₅₀ of 15-30 nM against Cav2.3, however at higher concentrations SNX-482 also inhibits N-type Ca²⁺ currents Newcomb et al., Biochemistry 1998, 37, 15353-15362); while at similar low nM concentration it inhibits A-Type Kv4 Potassium Currents (Kim et al., J Neurosci. 2014 Jul. 9; 34(28):9182-9). The off-target effects of SNX-482 and its general toxicity renders it unsuitable as a neuroprotective treatment for a therapeutic treatment of humans with neurodegenerative conditions such as Parkinson's disease.

Accordingly, there remains a need for Cav2.3 antagonists. Particularly desirable would be Cav2.3 antagonists that are also brain permeable.

### BRIEF SUMMARY OF THE DISCLOSURE

In accordance with the present inventions there is provided a compound of the formula (I), or a pharmaceutically acceptable salt thereof:

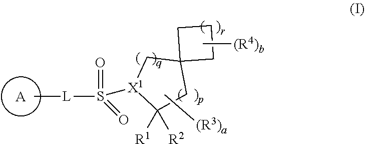

wherein:

### X¹ is CH or N;

R¹ is selected from: halo, C₁₋₆ alkyl, C₁₋₆ haloalkyl, C₃₋₆ cycloalkyl, and C₃₋₆ cycloalkyl-C₁₋₆ alkyl-,
wherein said C₁₋₆ alkyl, C₃₋₆ cycloalkyl and C₃₋₆ cycloalkyl-C₁₋₆ alkyl-is each optionally substituted by one or more substituents independently selected from: halo, C₁₋₄ alkyl, ═O, —CN, —OR^{1A}, —S(O)XR^{1A} and —NR^{1A}R^{1B};
   R² is selected from: H, halo, C₁₋₆ alkyl and C₁₋₆ haloalkyl; or

R¹ and R² together with the carbon atom to which they are attached form a C₃₋₆ cycloalkyl,

wherein said C₃₋₆ cycloalkyl is optionally substituted by one or more substituent selected from: halo, C₁₋₄ alkyl, ═O, —CN, —OR^{2A}, —S(O)ₓR^{2A} and —NR^{2A}R^{2B};

each R³ and R⁴ is independently selected from: halo, C₁₋₆ alkyl and C₁₋₆ haloalkyl;
L is independently selected from: a bond, and C₁₋₃ alkylene;
Ring A is selected from 5- to 12-membered heteroaryl and C₆₋₁₀ aryl; wherein Ring A is optionally substituted by one or more R⁵;
each R⁵ is independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷, wherein said C₁₋₆ alkyl, C₂₋₆ alkenyl and C₂₋₆ alkynyl is optionally substituted by one or more R⁸;
R⁶ and R⁷ are each independently selected from: H, C₁₋₆ alkyl, C₁₋₆ haloalkyl and Q¹,

wherein said C₁₋₆ alkyl is optionally substituted by one or more R⁹;

each R⁸ and R⁹ is independently selected from: halo, —CN, —OR^{8A}, —S(O)ₓR^{8A}, —NR^{8A}R^{8B} and Q²;
each Q¹ and Q² is independently selected from: C₃₋₆ cycloalkyl, 4- to 7-membered heterocyclyl, phenyl and 5- or 6-membered heteroaryl,

wherein said C₃₋₆ cycloalkyl and 4- to 7-membered heterocyclyl is optionally substituted by one or more R¹⁰,

wherein said phenyl and 5- or 6-membered heteroaryl is optionally substituted by one or more R¹¹:

each R¹⁰ is independently selected from: halo, ═O, —CN, —NO₂, C₁₋₄ alkyl, C₁₋₄ haloalkyl, —OR^{10A}, —S(O)₂R^{10A}, —NR^{10A}R^{10B}, —C(O)R^{10A}, —OC(O)R^{10A}, —C(O)OR^{10A}, —NR^{10B}C(O)R^{10A}, —C(O)NR^{10A}R^{10B}, —NR^{10B}C(O)OR^{10A}, —OC(O)NR^{10A}R^{10B}, —NR^{10B}SO₂R^{10A} and —SO₂NR^{10A}R^{10B},

wherein said C₁₋₄alkyl is optionally substituted by 1 or 2 substituents selected from: halo, —CN, —OR^{10C}, —NR^{10C}R^{10D} and —SO₂R^{10C};

each R¹¹ is independently selected from: halo, —CN, —NO₂, C₁₋₄ alkyl, C₁₋₄ haloalkyl, —OR^{7A}, —S(O)₂R^{11A}, —NR^{11A}R^{11B}, —C(O)R^{11A}, —OC(O)R^{11A}, —C(O)OR^{11A}, —NR^{11B}C(O)R^{11A}, —C(O)NR^{11A}R^{11B}, —NR^{11B}C(O)OR^{11A}, —OC(O)NR^{11A}R^{11B}, —NR^{11B}SO₂R^{11A} and —SO₂NR^{11A}R^{11B},

wherein said C₁₋₄alkyl is optionally substituted by 1 or 2 substituents selected from: halo, —CN, —OR^{11C}, —NR^{11C}R^{11D} and —SO₂R^{11C};

R^{1A}, R^{1B}, R^{2A}, R^{2B}, R^{8A}, R^{8B}, R^{10A}, R^{10B}, R^{10C}, R^{10D}, R^{11A}, R^{11B}, R^{11C}, R^{11D} are at each occurrence independently selected from: H, C₁₋₄ alkyl and C₁₋₄ haloalkyl;

and wherein any —NR^{1A}R^{1B}, —NR^{2A}R^{2B}, —NR⁶R⁷, —NR^{8A}R^{8B}, —NR^{10A}R^{10B}, —NR^{10C}R^{10D}, —NR^{11A}R^{11B}, and —NR^{11A}R^{11B} within a substituent may form a 4- to 6-membered heterocyclyl, wherein said 4- to 6-membered heterocyclyl is optionally substituted by one or more substituents selected from: halo, ═O, C₁₋₄ alkyl and C₁₋₄ haloalkyl;

a and b are each independently an integer from 0 to 4;
p is 1 or 2;
q is 1 or 2;
r is 0, 1 or 2; and
each x is independently 0, 1, or 2;
with the proviso that the compounds in List 1 are excluded:

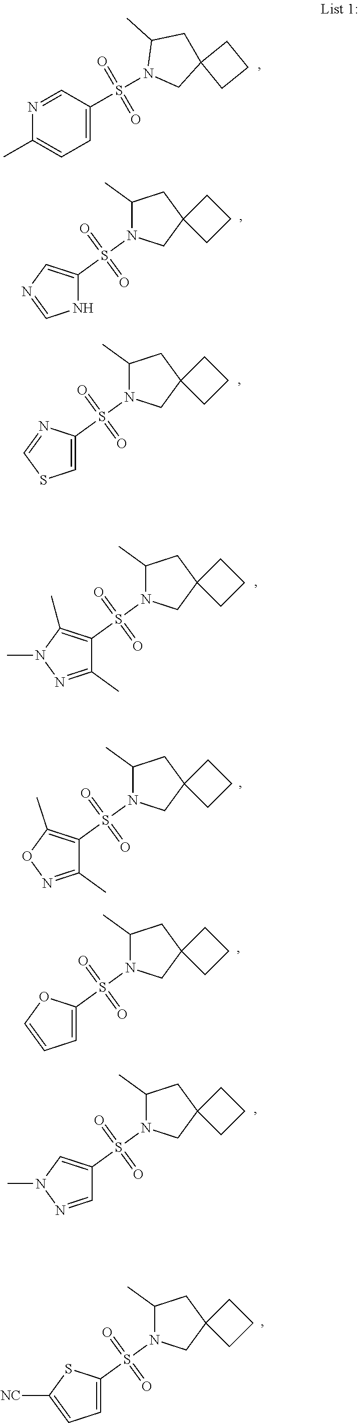

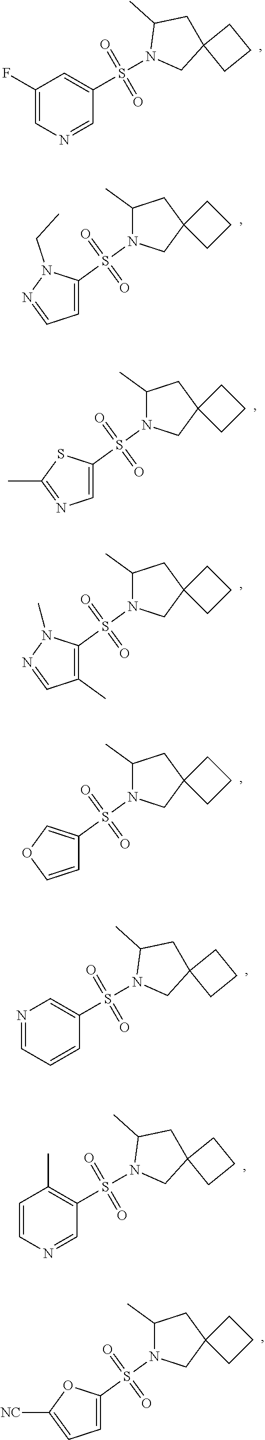

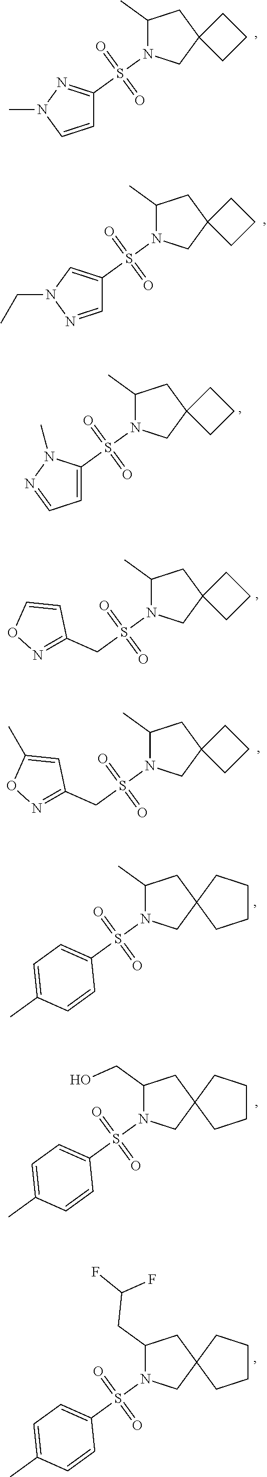

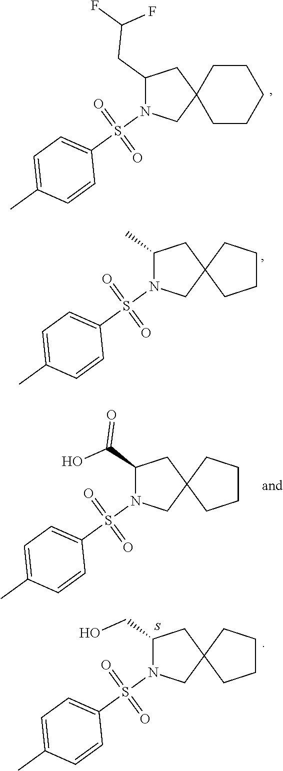

Also provided is a pharmaceutical composition comprising a compound of the invention, except the compounds from List 1 are not excluded, or a pharmaceutically acceptable salt thereof, and a pharmaceutically acceptable excipient.

Also provided is a compound of the invention, except the compounds from List 1 are not excluded, or a pharmaceutically acceptable salt thereof, for use as a medicament.

Also provided is a compound of the invention, or a pharmaceutically acceptable salt thereof, except the compounds from List 1 are not excluded, for use in the treatment of a disease or medical disorder mediated by Cav2.3.

Also provided is the use of a compound of the invention, except the compounds from List 1 are not excluded, or a pharmaceutically acceptable salt thereof, for the manufacture of a medicament for the treatment of a disease or medical disorder mediated by Cav2.3.

Also provided is a method of treating a disease or medical disorder mediated by Cav2.3 in a subject in need thereof, the method comprising administering to the subject an effective amount of a compound of the invention, except the compounds from List 1 are not excluded, or a pharmaceutically acceptable salt thereof.

In certain embodiments there is provided a compound of the invention, or a pharmaceutically acceptable salt thereof, for use in the prevention or treatment of a disease or medical disorder selected from: a neurodegenerative disease, a neurodevelopmental disorder, epilepsy, an endocrine disorder, cerebral vasospasm, and pain. In certain embodiments there is provided a compound of the invention, or a pharmaceutically acceptable salt thereof for use in the treatment of a neurodegenerative disease, for example Parkinson's disease, Alzheimer's disease, Huntington's disease, dystonia, amyotrophic lateral sclerosis (ALS), and age-related neurodegeneration. Further therapeutic uses of the compounds of the invention are set out in the Detailed Description.

### DETAILED DESCRIPTION

### Definitions

Unless otherwise stated, the following terms used in the specification and claims have the following meanings set out below.

Reference herein to a “compound of the invention” is a reference to any of the compounds disclosed herein including compounds of the formulae (I) to (XXIV), a compound selected from Compound List 1, or a compound described in any of the Examples, or a pharmaceutically acceptable salt, solvate, or salt of a solvate of any thereof.

The term “antagonist” for example “Cav2.3 antagonist” refers to any molecule that is capable of blocking or decreasing the amount of ions, particularly calcium ions through Cav2.3 channels. An antagonist may prevent of inhibit opening of the channel, or otherwise disrupt the normal operation of the channel. The antagonist may act directly on the channel or indirectly, for example by binding to an allosteric site on the channel.

As used herein, the term “selective antagonist” refers to an antagonist having greater affinity for its target than for one or more related receptors. For example, a “Cav2.3-selective antagonist” has greater affinity for Cav2.3 than for one or more similar calcium-ion channels (e.g., other Cav2, L-type, or N-type family members. The greater affinity of its Cav2.3 target may be, for example, at least: 1.1-fold, 1.2-fold, 1.3-fold, 1.4-fold, 1.5-fold, 2-fold, 3-fold, 4-fold, 5-fold, 10-fold, 20-fold, 50-fold, 100-fold, 1000-fold, 10000-fold, etc. The selectivity of a compound of the invention for Cav2.3 over other ion channels (e.g., one or more other Cav channels selected from Cav1.2, Cav1.2, Cav1.3, Cav1.4, Cav2.1, and Cav 2.2) can be assessed using methods analogous to the Cav2.3 channel calcium-influx assay described herein, using cells which express the channels of interest and comparing the Ic50 values.

The terms “treating”, or “treatment” refer to any beneficial effect in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; modifying the progression of a disease or condition, making the final point of degeneration less debilitating; improving a patient's physical or mental well-being. The treatment or amelioration of symptoms can be based on objective or subjective parameters; including the results of a physical examination, neuropsychiatric examinations, and/or a psychiatric evaluation. The term “treating” and conjugations thereof, includes prevention of an injury, pathology, condition, or disease (i.e., prophylaxis or prevention). For example, the term “treating” and conjugations thereof, include prevention of a pathology, condition, or disease associated with Cav2.3 (e.g., reducing or preventing symptoms or effects of the disease or condition or preventing or inhibiting progression of the disease or condition. For example, a compound of the invention may be for use in preventing, or reducing neurodegeneration in a neurodegenerative disease (e.g. Parkinson's disease), or delaying the onset of symptoms, or delaying the progression of a neurodegenerative disease.

The term “associated” or “associated with”, “involving” or “mediated by” in the context of a Cav2.3 associated with a disease means that the disease is caused by (in whole or in part), or a symptom of the disease is caused by (in whole or in part) by Cav2.3 channels, or channel activity or function. For example, a symptom of a disease or condition associated with Cav2.3 activity may be a symptom that results (entirely or partially) from an increase in the level of activity of Cav2.3 channels and or increased expression of Cav2.3 channels. A disease or medical disorder associated with a Cav2.3 activity or expression, may be treated with a compound of the invention effective for decreasing the level of activity of Cav2.3 channels, for example by blocking or partially blocking the channel, inhibiting the function of the channel, preventing or inhibiting the expression of the channel and/or degrading the channel.

An “effective amount” is an amount sufficient to accomplish a stated purpose. For example, an amount sufficient to achieve the effect for which it is administered, treat a disease, reduce enzyme activity, increase enzyme activity, reduce receptor signalling, increase receptor signalling, reduce one or more symptoms of a disease or condition, or to provide a disease modifying effect (i.e., alter the underlying pathophysiology of the disease). An example of an “effective amount” is an amount sufficient to contribute to the treatment, prevention, or reduction of a symptom or symptoms of a disease, or modify the progression of a disease, which could also be referred to as a “therapeutically effective amount.” A “reduction” of a symptom or symptoms means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology, or condition, or reducing the likelihood of the onset (or reoccurrence) of an injury, disease, pathology, or condition, or their symptoms. The full prophylactic effect does not necessarily occur by administration of one dose and may occur only after administration of a series of doses. Thus, a prophylactically effective amount may be administered in one or more administrations. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The therapeutically effective amount of a compound of the invention can be initially estimated from cell culture assays. Target concentrations will be those concentrations of active compound(s) that are capable of achieving the therapeutic effect described herein, as measured using the methods described herein or known in the art.

Therapeutically effective amounts for use in humans can also be determined from animal models using known methods. For example, a dose for humans can be formulated to achieve a concentration that has been found to be effective in animals. The dosage in humans can be adjusted by monitoring compound effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated, or in response to a biomarker or other correlate or surrogate end-point of the disease. This will provide a therapeutic regimen that is commensurate with the severity of the individual's disease state.

A prophylactic or therapeutic treatment regimen is suitably one that does not cause substantial toxicity and yet is effective to treat the clinical symptoms demonstrated by the particular patient. This determination of a dosage regimen is generally based upon an assessment of the active compound by considering factors such as compound potency, relative bioavailability, patient body weight, presence and severity of adverse side effects, preferred mode of administration and the toxicity profile of the selected agent.

The term “halo” or “halogen” refers to one of the halogens, group 17 of the periodic table. In particular, the term refers to fluorine, chlorine, bromine and iodine. Preferably, the term refers to fluorine or chlorine.

The term Cₘ₋ₙ refers to a group with m to n carbon atoms.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, iso-butyl, sec-butyl, tert-butyl, n-pentyl and n-hexyl. “C₁₋₄ alkyl” similarly refers to such groups containing up to 4 carbon atoms. Alkylene groups are divalent alkyl groups and may likewise be linear or branched and have two points of attachment to the remainder of the molecule. Furthermore, an alkylene group may, for example, correspond to one of those alkyl groups listed in this paragraph. For example, C₁₋₆ alkylene may be —CH₂—, —CH₂CH₂—, —CH₂CH(CH₃)—, —CH₂CH₂CH₂— or —CH₂CH(CH₃)CH₂—. The alkyl and alkylene groups may be unsubstituted or substituted by one or more substituents. Possible substituents are described herein. For example, substituents for an alkyl or alkylene group may be halogen, e.g. fluorine, chlorine, bromine and iodine, OH, C₁-C₄alkoxy, —NR′R″ amino, wherein R′ and R″ are independently H or alkyl. Other substituents for the alkyl group may alternatively be used.

The term “C₁₋₆ haloalkyl”, e.g., “C₁₋₄ haloalkyl”, refers to a hydrocarbon chain substituted with at least one halogen atom independently chosen at each occurrence, for example fluorine, chlorine, bromine, and iodine. The halogen atom may be present at any position on the hydrocarbon chain. For example, C₁₋₆ haloalkyl may refer to chloromethyl, fluoromethyl, trifluoromethyl, chloroethyl e.g., 1-chloromethyl and 2-chloroethyl, trichloroethyl e.g., 1,2,2-trichloroethyl, 2,2,2-trichloroethyl, fluoroethyl e.g., 1-fluoromethyl and 2-fluoroethyl, trifluoroethyl e.g., 1,2,2-trifluoroethyl and 2,2,2-trifluoroethyl, chloropropyl, trichloropropyl, fluoropropyl, trifluoropropyl. A haloalkyl group may be, for example, —CX₃, —CHX₂, —CH₂CX₃, —CH₂CHX₂ or —CX(CH₃)CH₃ wherein X is a halo (e.g., F, Cl, Br, or I). A fluoroalkyl group, i.e., a hydrocarbon chain substituted with at least one fluorine atom (e.g., —CF₃, —CHF₂, —CH₂CF₃ or —CH₂CHF₂).

The term “C₂₋₆ alkenyl” includes a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl. Alkenylene groups are divalent alkenyl groups and may likewise be linear or branched and have two points of attachment to the remainder of the molecule. Furthermore, an alkenylene group may, for example, correspond to one of those alkenyl groups listed in this paragraph. For example, alkenylene may be —CH═CH—, —CH₂CH═CH—, —CH(CH₃)CH═CH— or —CH₂CH═CH—. Alkenyl and alkenylene groups may unsubstituted or substituted by one or more substituents. Possible substituents are described herein. For example, substituents may be those described above as substituents for alkyl groups.

The term “C₂₋₆ alkynyl” includes a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl. Alkynylene groups are divalent alkynyl groups and may likewise be linear or branched and have two points of attachment to the remainder of the molecule. Furthermore, an alkynylene group may, for example, correspond to one of those alkynyl groups listed in this paragraph. For example alkynylene may be —C≡C—, —CH₂C≡C—, —CH₂C≡CCH₂—, —CH(CH₃)CH≡C— or —CH₂C≡CCH₃. Alkynyl and alkynylene groups may unsubstituted or substituted by one or more substituents. Possible substituents are described herein. For example, substituents may be those described above as substituents for alkyl groups.

The term “C₃₋₆ cycloalkyl” includes a saturated hydrocarbon ring system containing 3, 4, 5 or 6 carbon atoms. For example, the “C₃-C₆ cycloalkyl” may be cyclopropyl, cyclobutyl, cyclopentyl, cyclohexyl, bicyclo[2.1.1]hexane or bicyclo[1.1.1]pentane. Suitably the “C₃-C₆ cycloalkyl” may be cyclopropyl, cyclobutyl, cyclopentyl or cyclohexyl.

The term “heterocyclyl”, “heterocyclic” or “heterocycle” includes a non-aromatic saturated or partially saturated monocyclic or fused, bridged, or spiro bicyclic heterocyclic ring system. Monocyclic heterocyclic rings may contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles may contain from 7 to 12-member atoms in the ring. Bicyclic heterocyclic(s) rings may be fused, spiro, or bridged ring systems. The heterocyclyl group may be a 3-12, for example, a 3- to 9- (e.g., a 3- to 7-) membered non-aromatic monocyclic or bicyclic saturated or partially saturated group comprising 1, 2 or 3 heteroatoms independently selected from O, S and N in the ring system (in other words 1, 2 or 3 of the atoms forming the ring system are selected from O, S and N). By partially saturated it is meant that the ring may comprise one or two double bonds. This applies particularly to monocyclic rings with from 5 to 7 members. The double bond will typically be between two carbon atoms but may be between a carbon atom and a nitrogen atom. Bicyclic systems may be spiro-fused, i.e., where the rings are linked to each other through a single carbon atom; vicinally fused, i.e., where the rings are linked to each other through two adjacent carbon and/or nitrogen atoms; or they may be share a bridgehead, i.e. the rings are linked to each other through two non-adjacent carbon or nitrogen atoms (a bridged ring system). Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyrazolyl, tetrahydropyridinyl, homopiperidinyl, homopiperazinyl, 2,5-diaza-bicyclo[2.2.1]heptanyl and the like. Typical sulfur containing heterocycles include tetrahydrothienyl, dihydro-1,3-dithiol, tetrahydro-2H-thiopyran, and hexahydrothiepine. Other heterocycles include dihydro oxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (=O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person will appreciate, any heterocycle may be linked to another group via any suitable atom, such as via a carbon or nitrogen atom. For example, the term “piperidino” or “morpholino” refers to a piperidin-1-yl or morpholin-4-yl ring that is linked via the ring nitrogen.

The term “bridged ring systems” includes ring systems in which two rings share more than two atoms, see for example Advanced Organic Chemistry, by Jerry March, 4th Edition, Wiley Interscience, pages 131-133, 1992. Suitably the bridge is formed between two non-adjacent carbon or nitrogen atoms in the ring system. The bridge connecting the bridgehead atoms may be a bond or comprise one or more atoms. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane, and quinuclidine.

The term “spiro bi-cyclic ring systems” includes ring systems in which two ring systems share one common spiro carbon atom, i.e., the heterocyclic ring is linked to a further carbocyclic or heterocyclic ring through a single common spiro carbon atom. Examples of spiro ring systems include 3,8-diaza-bicyclo[3.2.1]octane, 2,5-diaza-bicyclo[2.2.1]heptane, 6-azaspiro[3.4]octane, 2-oxa-6-azaspiro[3.4]octane, 2-azaspiro[3.3]heptane, 2-oxa-6-azaspiro[3.3]heptane, 6-oxa-2-azaspiro[3.4]octane, 2,7-diaza-spiro[4.4]nonane, 2-azaspiro[3.5]nonane, 2-oxa-7-azaspiro[3.5]nonane and 2-oxa-6-azaspiro[3.5]nonane.

“Heterocyclyl-Cₘ₋ₙ alkyl” includes a heterocyclyl group covalently attached to a Cₘ₋ₙ alkylene group, both of which are defined herein; and wherein the Heterocyclyl-Cₘ₋ₙ alkyl group is linked to the remainder of the molecule via a carbon atom in the alkylene group. The groups “aryl-Cₘ₋ₙ alkyl”, “heteroaryl-Cₘ₋ₙ alkyl” and “cycloalkyl-Cₘ₋ₙ alkyl” are defined in the same way.

“—Cₘ₋ₙ alkyl substituted by —NRR” and “Cₘ₋ₙ alkyl substituted by —OR” similarly refer to an —NRR″ or —OR″ group covalently attached to a Cₘ₋ₙ alkylene group and wherein the group is linked to the remainder of the molecule via a carbon atom in the alkylene group.

The term “aromatic” when applied to a substituent as a whole includes a single ring or polycyclic ring system with 4n+2 electrons in a conjugated π system within the ring or ring system where all atoms contributing to the conjugated π system are in the same plane.

The term “aryl” includes an aromatic hydrocarbon ring system. The ring system has 4n+2 electrons in a conjugated π system within a ring where all atoms contributing to the conjugated π system are in the same plane. An aryl may be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. For example, the “aryl” may be a C₆₋₁₂ aryl, suitably phenyl or naphthyl. The aryl system itself may be substituted with other groups. The term “aryl” also covers partially aromatic bi- or polycyclic ring systems wherein at least one ring is an aromatic ring and one or more of the other ring(s) is a non-aromatic, saturated or partially saturated ring.

The term “heteroaryl” includes an aromatic mono- or bicyclic ring incorporating one or more (for example 1-4, particularly 1, 2 or 3) heteroatoms selected from nitrogen, oxygen or sulfur. The ring or ring system has 4n+2 electrons in a conjugated π system where all atoms contributing to the conjugated π system are in the same plane.

Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Bicyclic heteroaryl groups can be vicinally fused, i.e., where the rings are linked to each other through two adjacent carbon and/or nitrogen atoms. Each ring may contain up to about four heteroatoms typically selected from nitrogen, sulfur and oxygen. Typically, the heteroaryl ring will contain up to 4, for example up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 1H-pyrazolo[4,3-d]-oxazolyl, 4H-imidazo[4,5-d]thiazolyl, pyrazino[2,3-d]pyridazinyl, imidazo[2,1-b]thiazolyl and imidazo[1,2-b][1,2,4]triazinyl. Examples of heteroaryl groups comprising at least one nitrogen in a ring position include pyrrolyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, indolyl, isoindolyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl and pteridinyl.

“Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Partially aromatic heteroaryl bicyclic ring systems can be vicinally fused, i.e., where the rings are linked to each other through two adjacent carbon and/or nitrogen atoms. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five-membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six-membered heteroaryl groups include but are not limited to pyridyl, pyrazinyl, pyridazinyl, pyrimidinyl and triazinyl.

Particular examples of bicyclic heteroaryl groups containing a six-membered ring fused to a five-membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “oxo,” or “=O” as used herein, means an oxygen that is double bonded to a carbon atom.

The term “optionally substituted” includes either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups, which may be the same or different. For example, “one or more optional substituents” may refer to 1 or 2 or 3 substituents (e.g., 1 substituent or 2 substituents).

Where a moiety is substituted, it may be substituted at any point on the moiety where chemically possible and consistent with atomic valency requirements. The moiety may be substituted by one or more substituents, e.g., 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible, and which are not. For example, it will be recognised that when Ring A is pyridyl the ring nitrogen is not substituted and p is 0 to 4, similarly when Ring A is pyrimidyl, p is 0 to 3.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”:

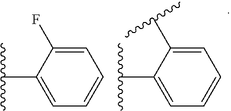

“Meta” substitution is a substitution pattern where two substituents are on carbons one carbon removed from each other, i.e., with a single carbon atom between the substituted carbons. In other words, there is a substituent on the second atom away from the atom with another substituent. For example, the groups below are meta substituted:

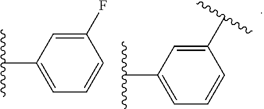

“Para” substitution is a substitution pattern where two substituents are on carbons two carbons removed from each other, i.e., with two carbon atoms between the substituted carbons. In other words, there is a substituent on the third atom away from the atom with another substituent. For example, the groups below are para substituted:

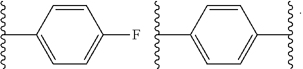

Where Ring A comprises an NH group, such as in formulae (VII), (VIII), (IX), (X), (XI) and (XII), the NH group may be substituted by R^{5b} to give NR^{5b}. When X¹ in formulae (I), (II), (Ill), (IV), (VII), (VIII), (IX), (X), (XI), (XII), (XIII), (XIV), (XV), (XVI), (XIX), (XX), (XXI), (XXII), (XXIII) and (XXIV) is CH and a is 1, 2, or 4, R³ may substitute the carbon atom represented by X¹. Thus, X¹ may be CH or CR³ when a is 1, 2, 3, or 4. As will be recognised when X¹ is N, the nitrogen atom is not substituted by R³ and a is 0, 1, 2, or 3.

Reference to a —NRR′ group forming a 4 to 6 membered heterocyclyl refers to R and R′ together with the nitrogen atom to which they are attached forming a 4 to 6 membered heterocyclyl group. For example, a —NR^{1A}R^{1B}, —NR^{2A}R^{2B}, —NR⁶R⁷, —NR^{8A}R^{8B}, —NR^{10A}R^{10B}, —NR^{10C}R^{10D}, —NR^{11A}R^{11B}, and —NR^{11A}R^{11B} group may form:

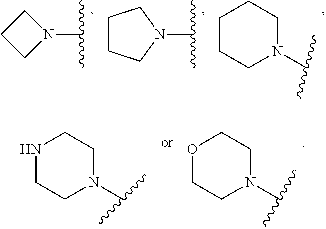

Similarly, an —NRR′ group within a substituent may form a carbonyl-linked 4 to 6 membered heterocyclyl, for example a —C(O)NRR′ group may form:

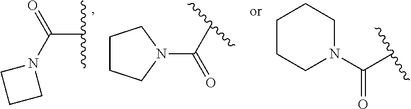

—NRR′ groups within substituents such as —OC(O)NRR′, —SO₂NRR′, or —NRC(O)NRR′, may similarly form a 4 to 6 membered heterocyclyl within such substituents.

A bond terminating in a “ [symbol] ”, i.e.

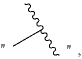

represents that the bond is connected to another atom that is not shown in the structure. A bond terminating inside a cyclic structure and not terminating at an atom of the ring structure represents that the bond may be connected to any of the atoms in the ring structure where allowed by valency.

The various functional groups and substituents making up the compounds of the present invention are typically chosen such that the molecular weight of the compound does not exceed 1000. More usually, the molecular weight of the compound will be less than 750, for example less than 700, or less than 650, or less than 600, or less than 550.

Suitable or preferred features of any compounds of the present invention may also be suitable features of any other aspect.

The invention contemplates pharmaceutically acceptable salts of the compounds of the invention. These may include the acid addition and base salts of the compounds. These may be acid addition and base salts of the compounds.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of the invention may be prepared by for example, one or more of the following methods:
(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

These methods are typically carried out in solution. The resulting salt may precipitate out and be collected by filtration or may be recovered by evaporation of the solvent. The degree of ionisation in the resulting salt may vary from completely ionised to almost non-ionised.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereo centres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer, the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%, for example at least 90%, at least 95% or at least 99%.

The compounds of this invention may possess one or more asymmetric centres; such compounds can therefore be produced as individual (R) or (S)stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E and Z isomers). It is to be understood that the present invention encompasses all optical, diastereoisomers and geometric isomers and mixtures thereof

Z/E (e.g., cis/trans) isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high-pressure liquid chromatography (HPLC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g., 0.1% diethylamine. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include ²H (also written as “D” for deuterium), ³H (also written as “T” for tritium), ¹¹C, ¹³C, ¹⁴C, ¹⁵O, ¹⁷O, ¹⁸O, ¹³N, ¹⁵N, ¹⁸F, ³⁶Cl, ¹²³I, ²⁵I, ³²P, ³⁵S and the like. The radionuclide that is used will depend on the specific application of that radio-labelled derivative. For example, for in vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

Isotopically-labelled compounds can generally be prepared by conventional techniques known to those skilled in the art or by processes analogous to those described using an appropriate isotopically-labelled reagent in place of the non-labelled reagent previously employed.

The selective replacement of hydrogen with deuterium in a compound may modulate the metabolism of the compound, the PK/PD properties of the compound and/or the toxicity of the compound. For example, deuteration may increase the half-life or reduce the clearance of the compound in vivo. Deuteration may also inhibit the formation of toxic metabolites, thereby improving safety and tolerability. It is to be understood that the invention encompasses deuterated derivatives of compounds of formula (I). As used herein, the term deuterated derivative refers to compounds of the invention where in a particular position at least one hydrogen atom is replaced by deuterium. Accordingly, in a compound of the invention one or more hydrogen atom is optionally replaced by deuterium. For example, one or more hydrogen atoms in a C₁₋₄-alkyl group may be replaced by deuterium to form a deuterated C₁₋₄-alkyl group. By way of example, if R⁴ is methyl the invention also encompasses —CD₃, —CHD₂ and —CH₂D.

Certain compounds of the invention may exist in solvated as well as unsolvated forms such as, for example, hydrated forms. It is to be understood that the invention encompasses all such solvated forms.

It is also to be understood that certain compounds of the invention may exhibit polymorphism, and that the invention encompasses all such forms.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

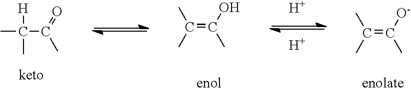

It is to be understood that when X¹ is CH and a is 1, 2, 3, or 4, the R³ substituent may be present on the carbon atom represented by X¹ (i.e., X¹ may be CH or CR³ when a is 1, 2 3, or 4).

The in vivo effects of a compound of the invention may be exerted in part by one or more metabolites that are formed within the human or animal body after administration of a compound of the invention.

It is further to be understood that a suitable pharmaceutically-acceptable pro-drug of a compound of the formula (I) also forms an aspect of the present invention. Accordingly, the compounds of the invention encompass pro-drug forms of the compounds and the compounds of the invention may be administered in the form of a pro-drug (i.e., a compound that is broken down in the human or animal body to release a compound of the invention). A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo-cleavable ester derivatives that may be formed at a carboxy group or a hydroxy group in a compound of the invention and in vivo-cleavable amide derivatives that may be formed at a carboxy group or an amino group in a compound of the invention.

Accordingly, the present invention includes those compounds of the invention as defined herein when made available by organic synthesis and when made available within the human or animal body by way of cleavage of a pro-drug thereof. Accordingly, the present invention includes those compounds of the formula (I) that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically-acceptable pro-drug of a compound of the invention is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988); f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the formula (I) that possesses a carboxy group is, for example, an in vivo-cleavable ester thereof. An in vivo-cleavable ester of a compound of the invention containing a carboxy group is, for example, a pharmaceutically-acceptable ester which is cleaved in the human or animal body to produce the parent acid. Suitable pharmaceutically-acceptable esters for carboxy include C₁₋₆ alkyl esters such as methyl, ethyl and tert-butyl, C₁₋₆ alkoxymethyl esters such as methoxymethyl esters, C₁₋₆ alkanoyloxymethyl esters such as pivaloyloxymethyl esters, 3-phthalidyl esters, C₃₋₈ cycloalkylcarbonyloxy-C₁₋₆ alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆ alkoxycarbonyloxy-C₁₋₆ alkyl esters such as methoxycarbonyloxymethyl and 1-methoxycarbonyloxyethyl esters.

A suitable pharmaceutically-acceptable pro-drug of a compound of the invention that possesses a hydroxy group is, for example, an in vivo-cleavable ester or ether thereof. An in vivo-cleavable ester or ether of a compound of the invention containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include C₁₋₁₀ alkanoyl groups such as acetyl, benzoyl, phenylacetyl and substituted benzoyl and phenylacetyl groups, C₁₋₁₀ alkoxycarbonyl groups such as ethoxycarbonyl, N,N—(C₁₋₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl)piperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the invention that possesses a carboxy group is, for example, an in vivo-cleavable amide thereof, for example an amide formed with an amine such as ammonia, a C₁₋₄ alkylamine such as methylamine, a (C₁₋₄ alkyl)₂amine such as dimethylamine, N-ethyl-N-methylamine or diethylamine, a C₁₋₄ alkoxy-C₂₋₄ alkylamine such as 2-methoxyethylamine, a phenyl-C₁₋₄ alkylamine such as benzylamine and amino acids such as glycine or an ester thereof.

A suitable pharmaceutically-acceptable pro-drug of a compound of the invention that possesses an amino group is, for example, an in vivo-cleavable amide or carbamate derivative thereof. Suitable pharmaceutically-acceptable amides from an amino group include, for example an amide formed with C₁₋₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl)piperazin-1-ylmethyl. Suitable pharmaceutically-acceptable carbamates from an amino group include, for example acyloxyalkoxycarbonyl and benzyloxycarbonyl groups.

Throughout the description and claims of this specification, the words “comprise” and “contain” and variations of them mean “including but not limited to”, and they are not intended to (and do not) exclude other moieties, additives, components, integers, or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties, or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Compounds

The following paragraphs are applicable to the compounds of the invention.

In certain embodiments the compound of the formula (I) is a compound of the formula (II), or a pharmaceutically acceptable salt thereof:

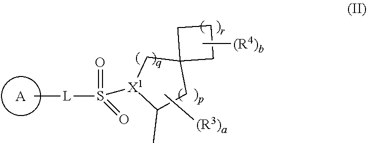

wherein X¹, R³, R⁴, a, b, p, q, r, L, and Ring A are as defined for formula (I).

In certain embodiments the compound of the formula (I) is a compound of the formula (III), or a pharmaceutically acceptable salt thereof:

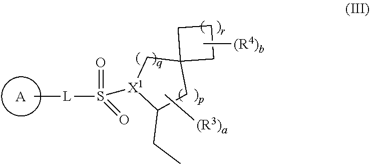

wherein X¹, R³, R⁴, a, b, p, q, r, L, and Ring A are as defined for formula (I).

In certain embodiments the compound of the formula (I) is a compound of the formula (IV), or a pharmaceutically acceptable salt thereof:

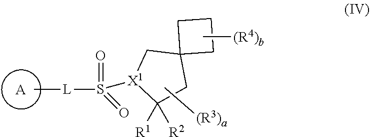

wherein X¹, R¹, R², R³, R⁴, a, b, L, and Ring A are as defined for formula (I).

In certain embodiments the compound of the formula (I) is a compound of the formula (V), or a pharmaceutically acceptable salt thereof:

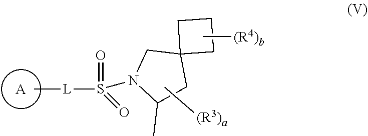

wherein R³, R⁴, a, b, L, and Ring A are as defined for formula (I).

In certain embodiments the compound of the formula (I) is a compound of the formula (VI), or a pharmaceutically acceptable salt thereof:

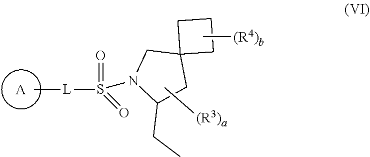

wherein R³, R⁴, a, b, L, and Ring A are as defined for formula (I).

In certain embodiments the compound of the formula (I) is a compound of the formula (VII), or a pharmaceutically acceptable salt thereof:

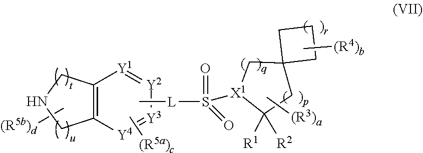

wherein:
Y¹, Y², Y³ and Y⁴ are each independently CH or N, provided no more than two of Y¹, Y², Y³ and Y⁴ are N;
t and u are each independently 0, 1, 2, or 3;
R^{5a} and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,

wherein said C₁₋₆ alkyl, C₂₋₆ alkenyl and C₂₋₆ alkynyl is optionally substituted by one or more R⁸;

c is an integer from 0 to 3;
d is an integer from 0 to 5; and
X¹, R¹, R², R³, R⁴, R⁶, R⁷, R⁸, a, b, p, q, r, and L are as defined for formula (I).

In certain embodiments the compound of the formula (I) is a compound of the formula (VIII), or a pharmaceutically acceptable salt thereof:

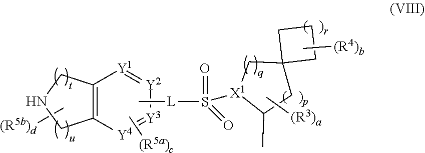

wherein:
Y¹, Y², Y³ and Y⁴ are each independently CH or N, provided no more than two of Y¹, Y², Y³ and Y⁴ are N;
t and u are each independently 0, 1, 2, or 3;
R^{5a} and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,

wherein said C₁₋₆ alkyl, C₂₋₆ alkenyl and C₂₋₆ alkynyl is optionally substituted by one or more R⁸

c is an integer from 0 to 3;
d is an integer from 0 to 5; and
X¹, R³, R⁴, R⁶, R⁷, R⁸, a, b, p, q, r, and L are as defined for formula (I).

In certain embodiments the compound of the formula (I) is a compound of the formula (IX), or a pharmaceutically acceptable salt thereof:

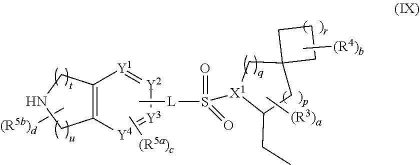

wherein:
Y¹, Y², Y³ and Y⁴ are each independently CH or N, provided no more than two of Y¹, Y², Y³ and Y⁴ are N;
t and u are each independently 0, 1, 2, or 3;
R^{5a} and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,

wherein said C₁₋₆ alkyl, C₂₋₆ alkenyl and C₂₋₆ alkynyl is optionally substituted by one or more R⁸

c is an integer from 0 to 3;
d is an integer from 0 to 5; and
X¹, R³, R⁴, R⁶, R⁷, R⁸, a, b, p, q, r, and L are as defined for formula (I).

In certain embodiments the compound of the formula (I) is a compound of the formula (X), or a pharmaceutically acceptable salt thereof:

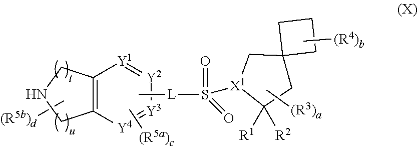

wherein:
Y¹, Y², Y³ and Y⁴ are each independently CH or N, provided no more than two of Y¹, Y², Y³ and Y⁴ are N;
t and u are each independently 0, 1, 2, or 3;
R^{5a} and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,

wherein said C₁₋₆ alkyl, C₂₋₆ alkenyl and C₂₋₆ alkynyl is optionally substituted by one or more R⁸;

c is an integer from 0 to 3;
d is an integer from 0 to 5; and
X¹, R¹, R², R³, R⁴, R⁶, R⁷, a, b, and L are as defined for formula (I).

In certain embodiments the compound of the formula (I) is a compound of the formula (XI), or a pharmaceutically acceptable salt thereof:

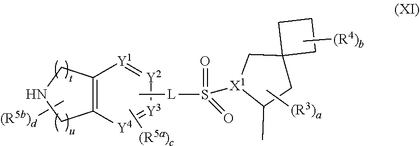

wherein:
Y¹, Y², Y³ and Y⁴ are each independently CH or N, provided no more than two of Y¹, Y², Y³ and Y⁴ are N;
t and u are each independently 0, 1, 2, or 3;
R^{5a} and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,

wherein said C₁₋₆ alkyl, C₂₋₆ alkenyl and C₂₋₆ alkynyl is optionally substituted by one or more R⁸

c is an integer from 0 to 3;
d is an integer from 0 to 5; and
X¹, R³, R⁴, R⁶, R⁷, R⁸, a, b, and L are as defined for formula (I).

In certain embodiments the compound of the formula (I) is a compound of the formula (XII), or a pharmaceutically acceptable salt thereof:

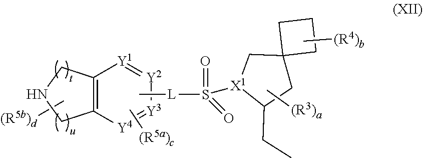

wherein:
Y¹, Y², Y³ and Y⁴ are each independently CH or N, provided no more than two of Y¹, Y², Y³ and Y⁴ are N;
t and u are each independently 0, 1, 2, or 3;
R^{5a} and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,

wherein said C₁₋₆ alkyl, C₂₋₆ alkenyl and C₂₋₆ alkynyl is optionally substituted by one or more R⁸;

c is an integer from 0 to 3;
d is an integer from 0 to 5; and
X¹, R³, R⁴, R⁶, R⁷, R⁸, a, b, and L are as defined for formula (I).

In certain embodiments the compound of the formula (I) is a compound according to formula (XIII), or a pharmaceutically acceptable salt thereof:

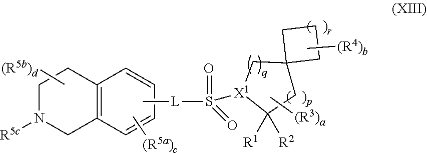

wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,

wherein said C₁₋₆ alkyl, C₂₋₆ alkenyl and C₂₋₆ alkynyl is optionally substituted by one or more R⁸

c is an integer from 0 to 3;
d is an integer from 0 to 4; and
X¹, R¹, R², R³, R⁴, R⁶, R⁷, R⁸, a, b, p, q, r, and L are as defined for formula (I).

In certain embodiments the compound of the formula (I) is a compound according to formula (XIV), or a pharmaceutically acceptable salt thereof:

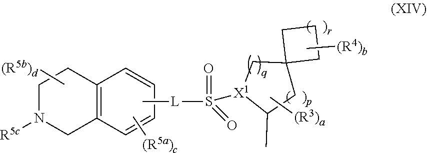

wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,

wherein said C₁₋₆ alkyl, C₂₋₆ alkenyl and C₂₋₆ alkynyl is optionally substituted by one or more R⁸

c is an integer from 0 to 3;
d is an integer from 0 to 4; and
X¹, R³, R⁴, R⁶, R⁷, R⁸, a, b, p, q, r, and L are as defined for formula (I).

In certain embodiments the compound of the formula (I) is a compound according to formula (XV), or a pharmaceutically acceptable salt thereof:

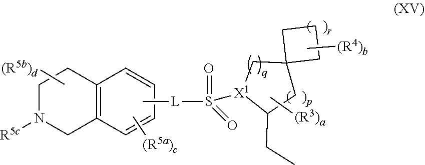

wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,

wherein said C₁₋₆ alkyl, C₂₋₆ alkenyl and C₂₋₆ alkynyl is optionally substituted by one or more R⁸

c is an integer from 0 to 3;
d is an integer from 0 to 4; and
X¹, R³, R⁴, R⁶, R⁷, R⁸, a, b, p, q, r, and L are as defined for formula (I).

In certain embodiments the compound of the formula (I) is a compound according to formula (XVI), or a pharmaceutically acceptable salt thereof:

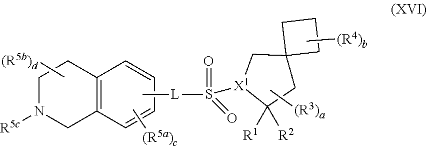

wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,

wherein said C₁₋₆ alkyl, C₂₋₆ alkenyl and C₂₋₆ alkynyl is optionally substituted by one or more R⁸

c is an integer from 0 to 3;
d is an integer from 0 to 4; and
X¹, R¹, R², R³, R⁴, R⁶, R⁷, R⁸, a, b, and L are as defined for formula (I).

In certain embodiments the compound of the formula (I) is a compound according to formula (XVII), or a pharmaceutically acceptable salt thereof:

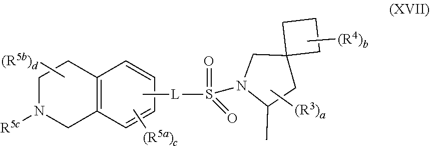

wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,

wherein said C₁₋₆ alkyl, C₂₋₆ alkenyl and C₂₋₆ alkynyl is optionally substituted by one or more R⁸

c is an integer from 0 to 3;
d is an integer from 0 to 4; and
R³, R⁴, R⁶, R⁷, R⁸, a, b, and L are as defined for formula (I).

In certain embodiments the compound of the formula (I) is a compound according to formula (XVIII), or a pharmaceutically acceptable salt thereof:

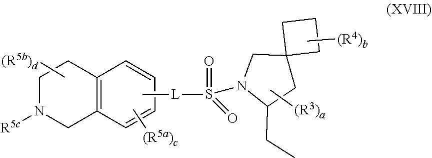

wherein:
R^{5a} and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,

wherein said C₁₋₆ alkyl, C₂₋₆ alkenyl and C₂₋₆ alkynyl is optionally substituted by one or more R⁸;

c is an integer from 0 to 3;
d is an integer from 0 to 4; and
R³, R⁴, R⁶, R⁷, R⁸, a, b, and L are as defined for formula (I).

In certain embodiments the compound of the formula (I) is a compound according to formula (XIX), or a pharmaceutically acceptable salt thereof:

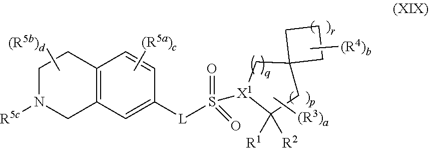

wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,

wherein said C₁₋₆ alkyl, C₂₋₆ alkenyl and C₂₋₆ alkynyl is optionally substituted by one or more R⁸;

c is an integer from 0 to 3;
d is an integer from 0 to 4; and
X¹, R¹, R², R³, R⁴, R⁶, R⁷, R⁸, a, b, p, q, r, and L are as defined for formula (I).

In certain embodiments the compound of the formula (I) is a compound according to formula (XX), or a pharmaceutically acceptable salt thereof:

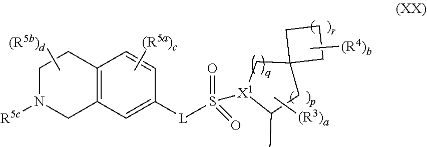

wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,

wherein said C₁₋₆ alkyl, C₂₋₆ alkenyl and C₂₋₆ alkynyl is optionally substituted by one or more R⁸

c is an integer from 0 to 3;
d is an integer from 0 to 4; and
X¹, R³, R⁴, R⁶, R⁷, R⁸, a, b, p, q, r, and L are as defined for formula (I).

In certain embodiments the compound of the formula (I) is a compound according to formula (XXI), or a pharmaceutically acceptable salt thereof:

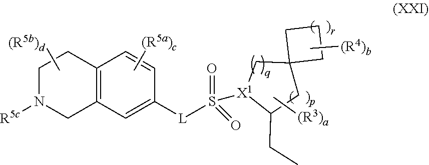

wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,

wherein said C₁₋₆ alkyl, C₂₋₆ alkenyl and C₂₋₆ alkynyl is optionally substituted by one or more R⁸;

c is an integer from 0 to 3;
d is an integer from 0 to 4; and
X¹, R³, R⁴, R⁶, R⁷, R⁸, a, b, p, q, r, and L are as defined for formula (I).

In certain embodiments the compound of the formula (I) is a compound according to formula (XXII), or a pharmaceutically acceptable salt thereof:

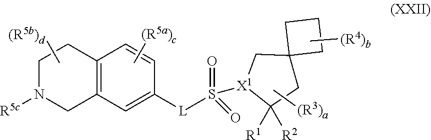

wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁸, —OC(O)R⁸, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,

wherein said C₁₋₆ alkyl, C₂₋₆ alkenyl and C₂₋₆ alkynyl is optionally substituted by one or more R⁸;

c is an integer from 0 to 3;
d is an integer from 0 to 4; and
X¹, R¹, R², R³, R⁴, R⁶, R⁷, R⁸, a, b, and L are as defined for formula (I).

In certain embodiments the compound of the formula (I) is a compound according to formula (XXII), or a pharmaceutically acceptable salt thereof:

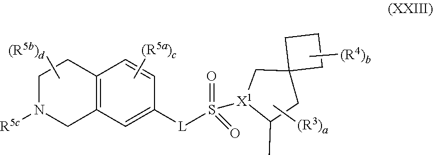

wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,

wherein said C₁₋₆ alkyl, C₂₋₆ alkenyl and C₂₋₆ alkynyl is optionally substituted by one or more R⁸;

c is an integer from 0 to 3;
d is an integer from 0 to 4; and
X¹, R³, R⁴, R⁶, R⁷, R⁸, a, b, and L are as defined for formula (I).

In certain embodiments the compound of the formula (I) is a compound according to formula (XXIV), or a pharmaceutically acceptable salt thereof:

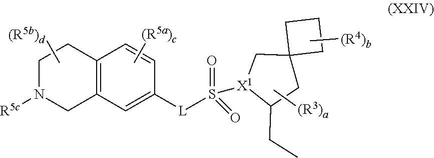

wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: H C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,

wherein said C₁₋₆ alkyl, C₂₋₆ alkenyl and C₂₋₆ alkynyl is optionally substituted by one or more R⁸;

c is an integer from 0 to 3;
d is an integer from 0 to 4; and
X¹, R³, R⁴, R⁶, R⁷, R⁸, a, b, and L are as defined for formula (I).

In certain embodiments compounds of the invention include, for example, compounds of formulae (I) to (XXIV), or a pharmaceutically acceptable salt thereof, wherein, unless otherwise stated, each of Ring A, X¹, Y¹, Y², Y³, Y⁴, R¹, R², R³, R⁴, R⁵, R^{5a}, R^{5b}, R^{5c}, R⁶, R⁷, R⁸, R^{1A}, R^{1B}, R^{2A}, R^{2B}, R^{8A}, R^{8B}, R^{10A}, R^{10B}, R^{10C}, R^{10D}, R^{11A}, R^{11B}, R^{11C}, R^{11D}, Q¹, Q², L, a, b, c, d, p, q, r, t, u, x, and y has any of the meanings defined hereinbefore or in any of the following statements in the numbered paragraphs 1 to 136 hereinafter. These statements are independent and interchangeable. In other words, any of the features described in any one of the following statements may (where chemically allowable) be combined with the features described in one or more other statements below. In particular, where a compound is exemplified or illustrated in this specification, any two or more of the statements below which describe a feature of that compound, expressed at any level of generality, may be combined so as to represent subject matter which is contemplated as forming part of the disclosure of this invention in this specification.
1. X¹ is CH.
2. X¹ is N.
3. R¹ is selected from: C₁₋₆ alkyl, C₁₋₆ haloalkyl, C₃₋₆ cycloalkyl, and C₃₋₆ cycloalkyl-C₁₋₆ alkyl-.
4. R¹ is selected from: C₁₋₆ alkyl, C₁₋₆ haloalkyl, and C₃₋₆ cycloalkyl.
5. R¹ is selected from: C₁₋₆ alkyl and C₃₋₆ cycloalkyl.
6. R¹ is C₁₋₆ alkyl.
7. R¹ is C₁₋₃ alkyl.
8. R¹ is selected from ethyl and methyl.
9. R¹ is ethyl.
10. R¹ is methyl.
11. Where R¹ is an C₁₋₆ alkyl, cycloalkyl or cycloalkyl-alkyl-group, each said alkyl, cycloalkyl and cycloalkyl-alkyl-is optionally substituted by one or more substituents independently selected from: =O, —CN, —OR^{1A}, and —NR^{1A}R^{1B}. Thus, each said alkyl, cycloalkyl and cycloalkyl-alkyl-in any one of 3 to 10 may be substituted by one or more substituents independently selected from: =O, —CN, —OR^{1A}, and —NR^{1A}R^{1B}.
12. Where R¹ is an alkyl, cycloalkyl or cycloalkyl-alkyl-group, each said alkyl, cycloalkyl and cycloalkyl-alkyl- may be substituted by —OR^{1A}. Thus, each said alkyl, cycloalkyl and cycloalkyl-alkyl- in any one of 3 to 10 may be substituted by —OR^{1A}.
13. Where R¹ is an alkyl, cycloalkyl or cycloalkyl-alkyl-group, each said alkyl, cycloalkyl and cycloalkyl-alkyl- may be substituted by —OH or —OMe. Thus, each said alkyl, cycloalkyl and cycloalkyl-alkyl- in any one of 3 to 10 may be substituted by —OH or —OMe.
14. R¹ is selected from methyl, ethyl, cyclohexyl, —CH₂OH, and CH₂OMe.
15. R² is selected from: H, C₁₋₆ alkyl and C₁₋₆ haloalkyl.
16. R² is selected from: H, and C₁₋₆ alkyl.
17. R² is selected from: H, and C₁₋₃ alkyl.
18. R² is selected from: H, and methyl.
19. R² is H.
20. R¹ and R² together with the carbon atom to which they are attached form a C₃ cycloalkyl, i.e. cyclopropyl.
21. R¹ is as defined in any of 3 to 14 and R² is H.
22. X¹ is CH and R¹ is halo.
23. X¹ is CH and R² is halo.
24. X¹ is CH and R¹ and R² are halo.
25. Said halo in any of 22 to 24 is fluoro.
26. R³ and R⁴ are each independently selected from: halo, C₁₋₃ alkyl and C₁₋₃ haloalkyl.
27. L is selected from a bond and C₁₋₂ alkylene.
28. L is selected from a bond, —CH₂—, and —CH₂CH₂—.
29. L is selected from a bond, —CH₂—, and —CH₂CH₂—. Thus, it may be that L is selected from a bond and —CH₂—.
30. L is —CH₂—.
31. L is a bond.
32. Ring A is 5- to 12-membered heteroaryl; optionally substituted with one or more R⁵.
33. Ring A is 5- to 10-membered heteroaryl; optionally substituted with one or more R⁵.
34. Ring A is 5-membered heteroaryl; optionally substituted with one or more R⁵.
35. Ring A is 6-membered heteroaryl; optionally substituted with one or more R⁵.
36. Ring A is 9-membered heteroaryl; optionally substituted with one or more R⁵.
37. Ring A is 10-membered heteroaryl; optionally substituted with one or more R⁵.
38. Ring A is phenyl; optionally substituted with one or more R⁵.
39. Ring A is furanyl, thienyl, imidazolyl, pyrazolyl, oxazolyl, isoxazolyl, thiazolyl, isothiazolyl, thiadiazolyl, phenyl, pyridyl, pyrimidinyl, pyrazinyl, or a compound of the structure:

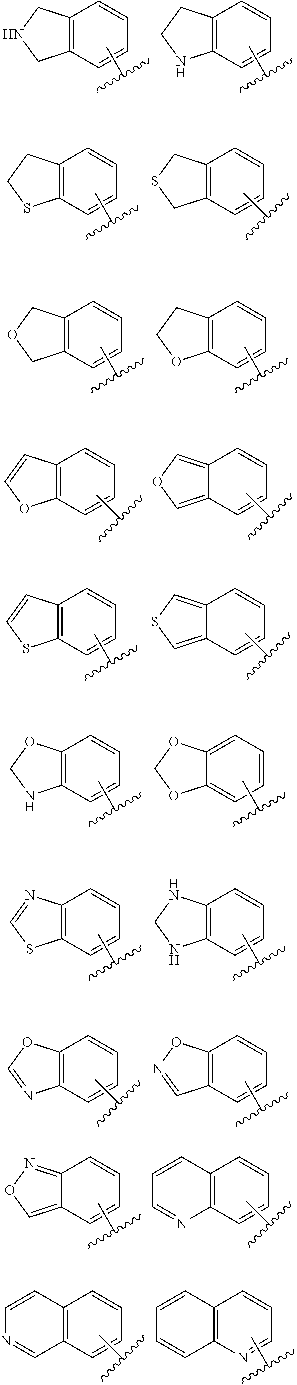

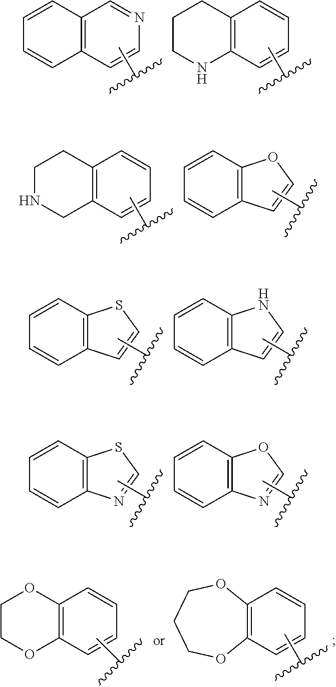

wherein Ring A is optionally substituted with one or more R⁵.
40. Ring A is

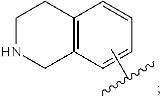

wherein Ring A is optionally substituted with one or more R⁵.
41. Ring A is selected from:

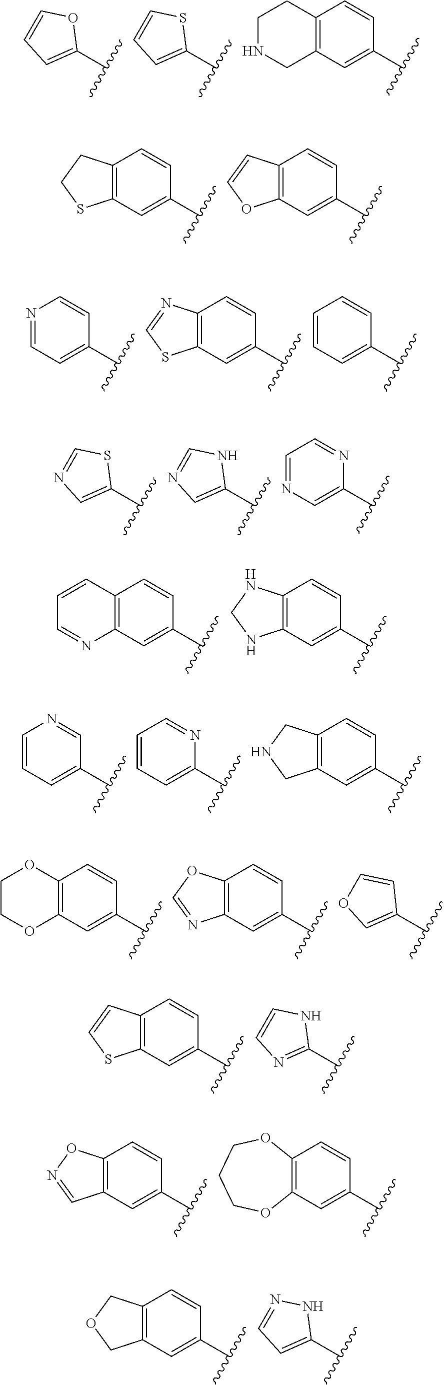

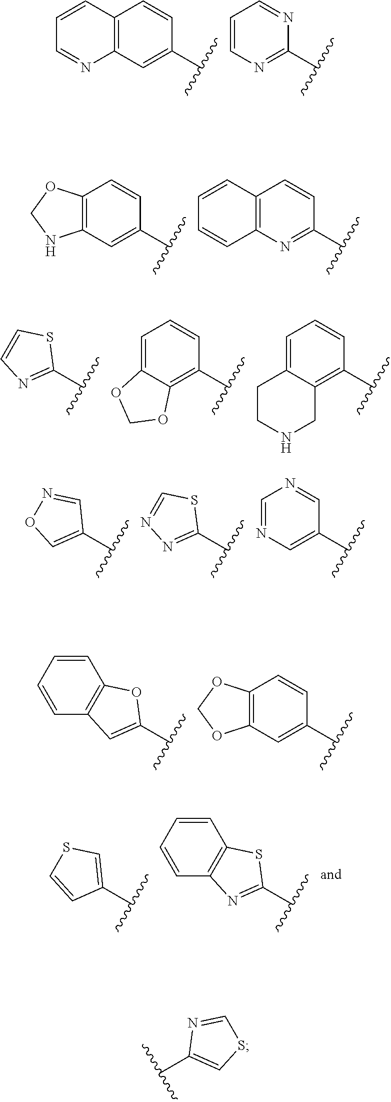

wherein Ring A is optionally substituted with one or more R⁵.
42. Ring A is selected from:

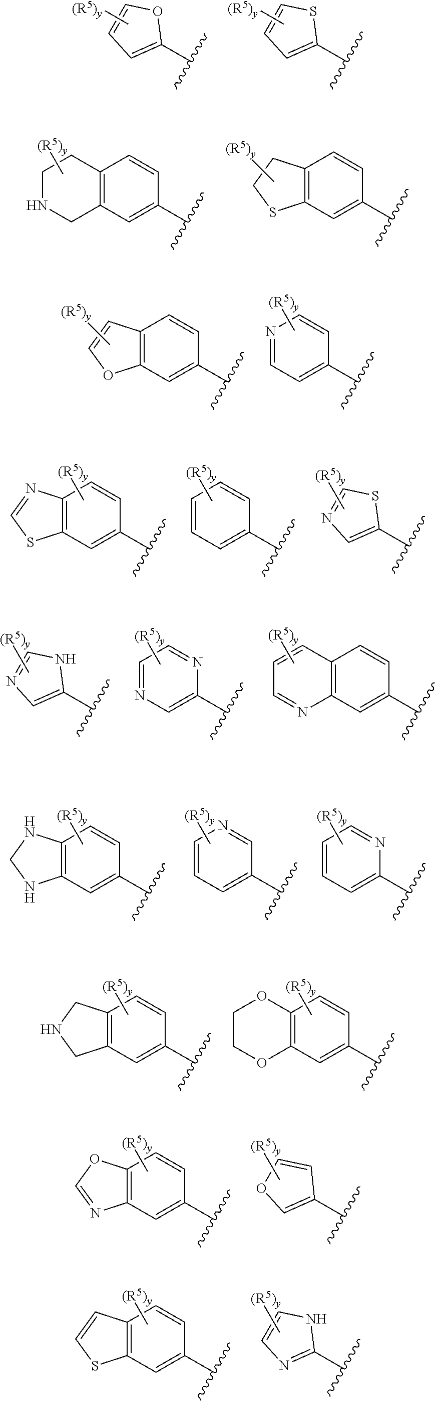

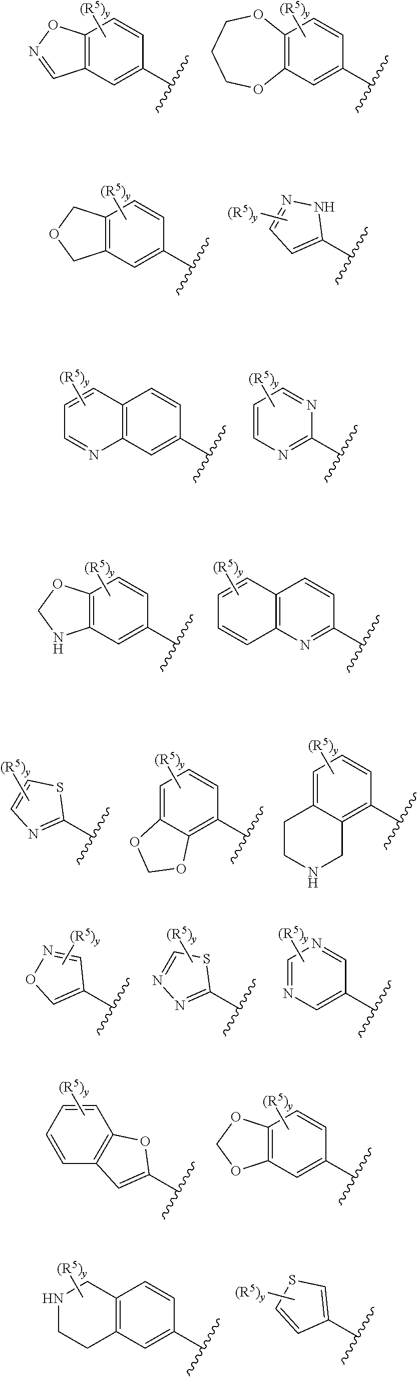

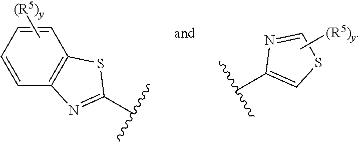

43. Ring A is selected from:

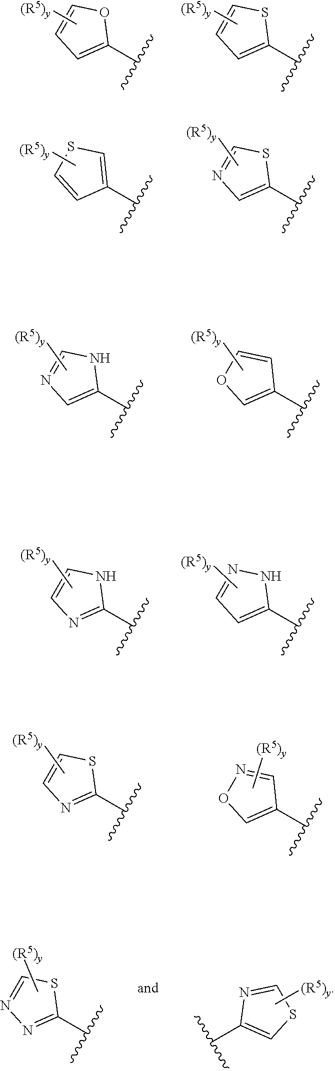

44. Ring A is selected from:

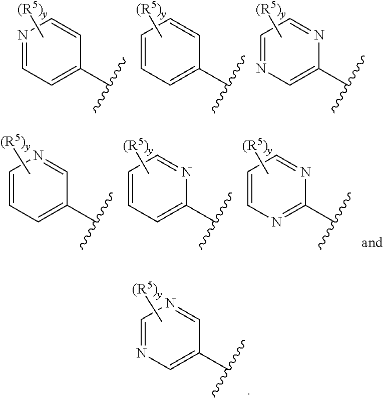

45. Ring A is selected from:

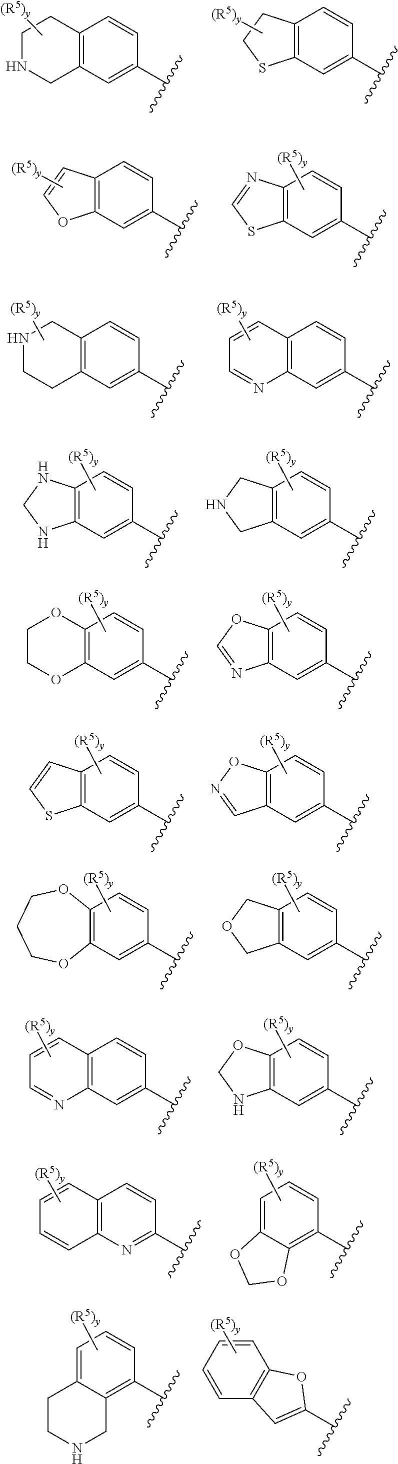

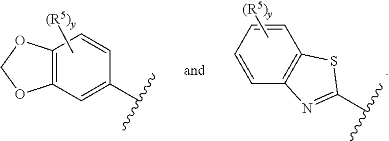

46. Ring A is as defined in any of 42 to 45, wherein y is an integer from 0 to 10, where chemically possible.
47. Ring A is as defined in any of 42 to 45, wherein y is an integer from 0 to 5, where chemically possible.
48. Ring A is as defined in any of 42 to 45, wherein y is an integer from 0 to 3, where chemically possible.
49. Ring A is as defined in any of 42 to 45, wherein y is 0.
50. Ring A is as defined in any of 42 to 45, wherein y is 1.
51. Ring A is as defined in any of 42 to 45, wherein y is 2.
52. Ring A is as defined in 39 and is substituted by one or more R⁵.
53. R⁵ is independently selected from: halo, —ON, —NO₂, ═O, C₁₋₆ alkyl, C₁₋₆ haloalkyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶SO₂R⁷, and —SO₂NR⁶R⁷.
54. R⁵ is independently selected from: halo, —CN, ═O, C₁₋₆ alkyl, Q¹, —OR⁶, —NR⁶R⁷, —C(O)R⁶, and —NR⁶C(O)R⁷.
55. R⁵ is independently selected from: halo and C₁₋₄ alkyl.
56. R⁵ is as defined in any one of 53 to 55, wherein said C₁₋₆ alkyl is substituted by one or more R⁸.
57. R⁶ and R⁷ are each independently selected from: H, C₁₋₆ alkyl, and Q¹.
58. R⁶ and R⁷ are each independently selected from: H, C₁₋₃ alkyl, and Q¹.
59. R⁶ and R⁷ are as defined in 57 to 58, wherein said alkyl is substituted by one or more R⁹.
60. R⁸ and R⁹ are each independently selected from: halo, —CN, —OR^{8A}, —NR^{8A}R^{8B} and Q².
61. R⁸ and R⁹ are each independently selected from: halo, —OR^{8A}, and Q².
62. Q¹ and Q² are each independently selected from: C₃₋₆ cycloalkyl, 4- to 7-membered heterocyclyl.
63. Q¹ and Q² are as defined in 62, wherein said C₃₋₆ cycloalkyl and 4- to 7-membered heterocyclyl is optionally substituted by one or more R¹⁰.
64. Q¹ and Q² are each independently selected from: phenyl and 5- or 6-membered heteroaryl.
65. Q¹ and Q² are as defined in 64, wherein said phenyl and 5- or 6-membered heteroaryl is substituted by one or more R¹¹.
66. Each R¹⁰ is independently selected from: halo, ═O, —CN, —NO₂, C₁₋₄ alkyl, C₁₋₄ haloalkyl, —OR^{10A}, and —NR^{10A}R^{10B}.
67. R¹⁰ is as defined in 66, wherein said C₁₋₄ alkyl is substituted by 1 or 2 substituents selected from: halo, —CN, —OR^{10C}, —NR^{10C}R^{10D} and —SO₂R^{10C}.
68. Each R¹¹ is independently selected from: halo, —CN, —NO₂, C₁₋₄ alkyl, C₁₋₄ haloalkyl, —OR^{7A}, and —NR^{11A}R^{11B}.
69. R¹¹ is as defined in 68, wherein said C₁₋₄ alkyl is substituted by 1 or 2 substituents selected from: halo, —CN, —OR^{11C}, —NR^{11C}R^{11D} and —SO₂R^{11C}.
70. R^{1A}, R^{1B}, R^{2A}, R^{2B}, R^{8A}, R^{8B}, R^{10A}, R^{10B}, R^{10C}, R^{10D}, R^{11A}, R^{11B}, R^{11C}, R^{11D} are at each occurrence independently selected from: H, and C₁₋₄ alkyl.
71. R^{1A}, R^{1B}, R^{2A}, R^{2B}, R^{8A}, R^{8B}, R^{10A}, R^{10B}, R^{10C}, R^{10D}, R^{11A}, R^{11B}, R^{11C}, R^{11D} are at each occurrence independently selected from: H, methyl, and ethyl.
72. Each R⁵ is independently selected from:

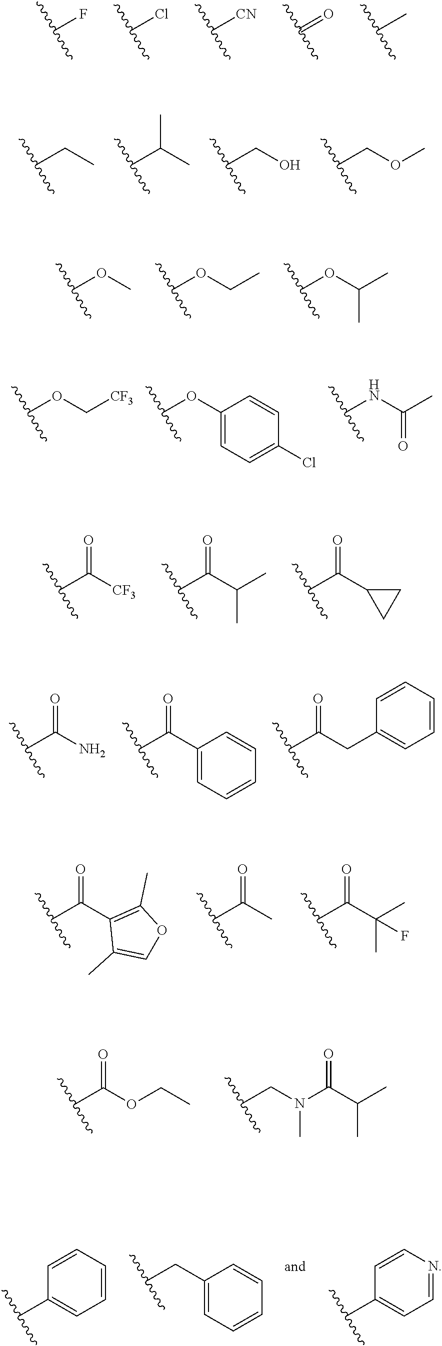

73. Ring A is selected from:

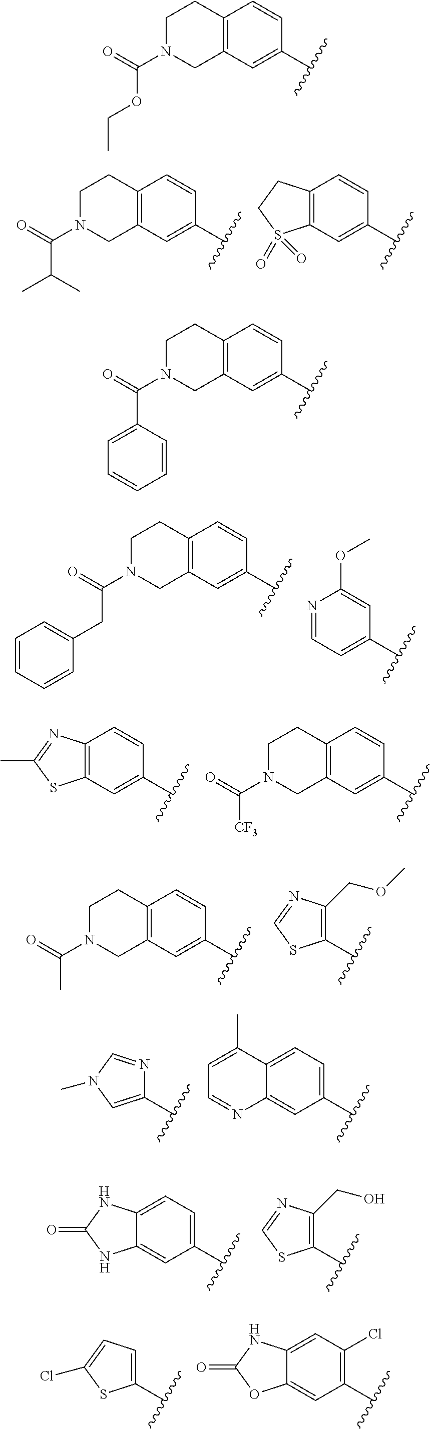

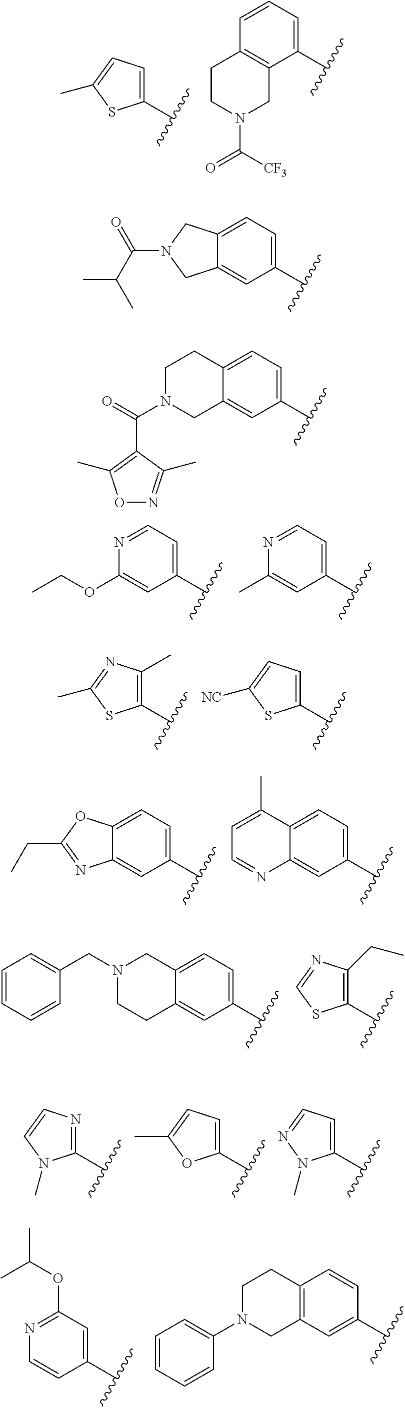

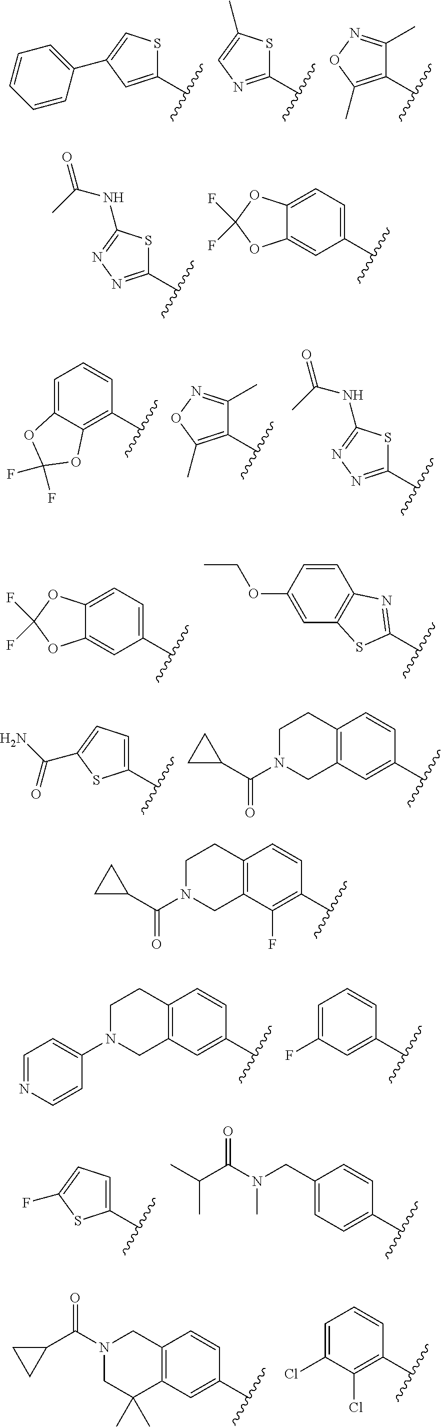

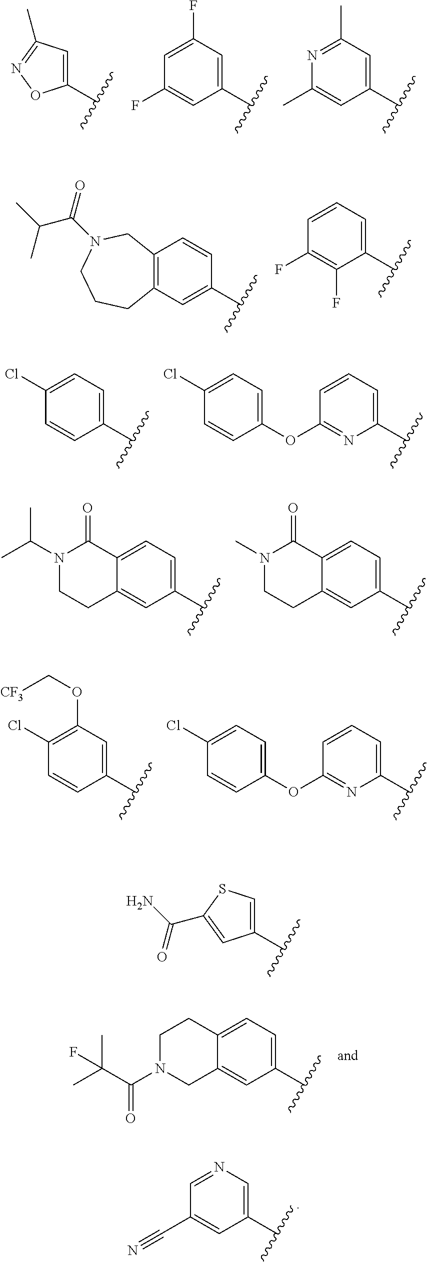

74. p is 1.
75. p is 2.
76. g is 1.
77. q is 2.
78. r is 0.
79. r is 1.
80. r is 2.
81. x is 0.
82. x is 1.
83. x is 2.
84. Y¹, Y², Y³ and Y⁴ are each CH.
85. t and u are each independently 1.
86. t and u are each independently 2.
87. t and u are each independently 3.
88. At least one of t and u is 0.
89. t and u are not both 0.
90. t is 2 and u is 1.
91. t is 1 and u is 2.
92. t is 1 and u is 3.
93. t is 3 and u is 1.
94. The group of the formula

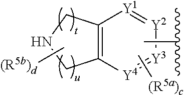

in any of formulae (VII), (VIII), (IX), (X), (XI), (XII) is selected from:

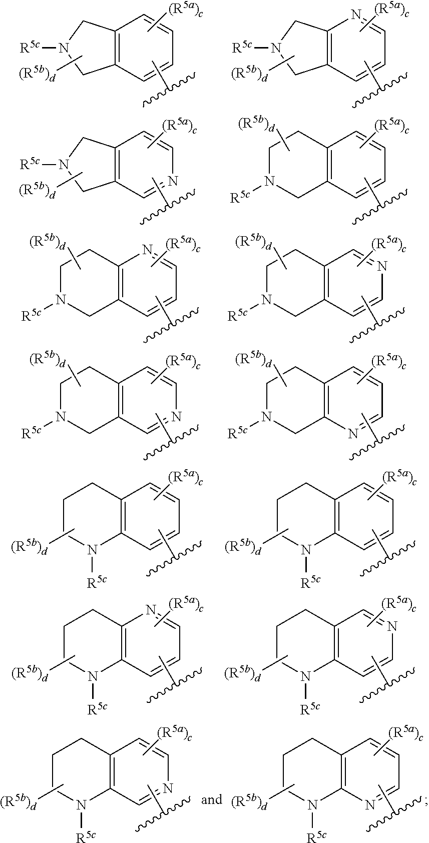

wherein R^{5c} has any of the values defined herein. Thus it may be that R^{5c} is selected from R^{5c} is selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷, wherein said C₁₋₆ alkyl, C₂₋₆ alkenyl and C₂₋₆ alkynyl is optionally substituted by one or more R⁸. It may be that R^{5c} is as defined in any of 106 to 120.
95. The group of the formula

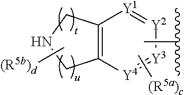

in any of formulae (VII), (VIII), (IX), (X), (XI), (XII) is selected from is selected from:

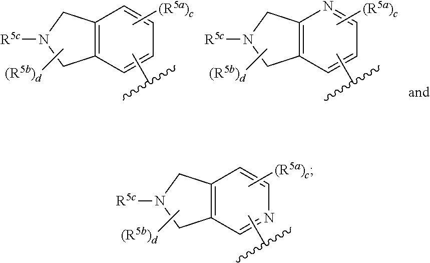

wherein R^{5c} has any of the values defined herein. Thus it may be that R^{5c} is selected from R^{5c} is selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷, wherein said C₁₋₆ alkyl, C₂₋₆ alkenyl and C₂₋₆ alkynyl is optionally substituted by one or more R⁸. It may be that R^{5c} is as defined in any of 106 to 120.
96. The group of the formula

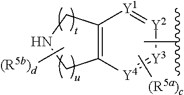

in any of formulae (VII), (VIII), (IX), (X), (XI), (XII) is selected from:

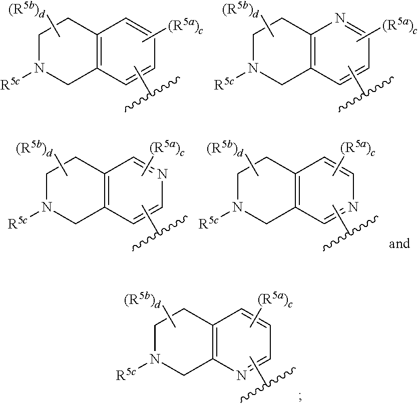

wherein R^{5c} has any of the values defined herein. Thus, it may be that R^{5c} is selected from R^{5c} is selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷, wherein said C₁₋₆ alkyl, C₂₋₆ alkenyl and C₂₋₆ alkynyl is optionally substituted by one or more R⁸. It may be that R^{5c} is as defined in any of 106 to 120.
97. The group of the formula

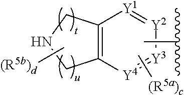

in any of formulae (VII), (VIII), (IX), (X), (XI), (XII) is selected from:

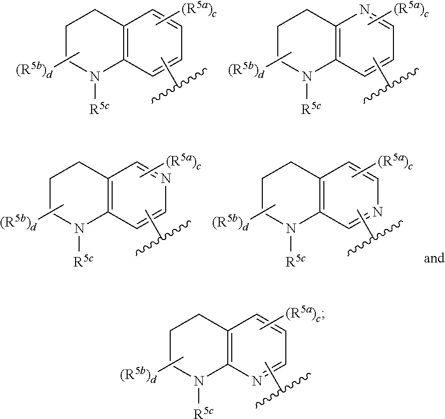

wherein R^{5c} has any of the values defined herein. Thus, it may be that R^{5c} is selected from R^{5c} is selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷, wherein said C₁₋₆ alkyl, C₂₋₆ alkenyl and C₂₋₆ alkynyl is optionally substituted by one or more R⁸. It may be that R^{5c} is as defined in any of 106 to 120.
98. The group of the formula

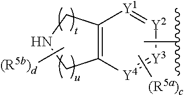

in any of formulae (VII), (VIII), (IX), (X), (XI), (XII) is selected from:

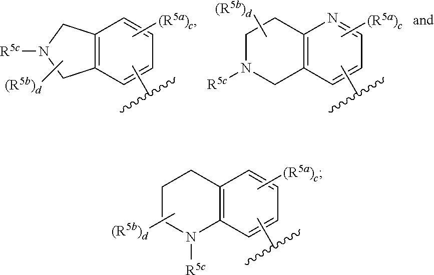

wherein R^{5c} has any of the values defined herein. Thus, it may be that R^{5c} is selected from R^{5c} is selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷, wherein said C₁₋₆ alkyl, C₂₋₆ alkenyl and C₂₋₆ alkynyl is optionally substituted by one or more R⁸. It may be that R^{5c} is as defined in any of 106 to 120.
99. The group of the formula

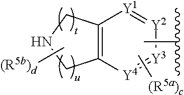

in any of formulae (VII), (VIII), (IX), (X), (XI), (XII) is selected from:

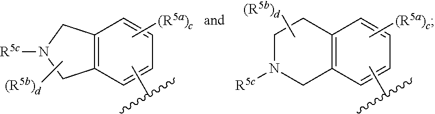

wherein R^{5c} has any of the values defined herein. Thus it may be that R^{5c} is selected from R^{5c} is selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷, wherein said C₁₋₆ alkyl, C₂₋₆ alkenyl and C₂₋₆ alkynyl is optionally substituted by one or more R⁸. It may be that R^{5c} is as defined in any of 106 to 120.
100. The group of the formula

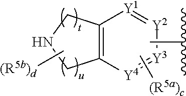

in any of formulae (VII), (VIII), (IX), (X), (XI), (XII) is selected from:

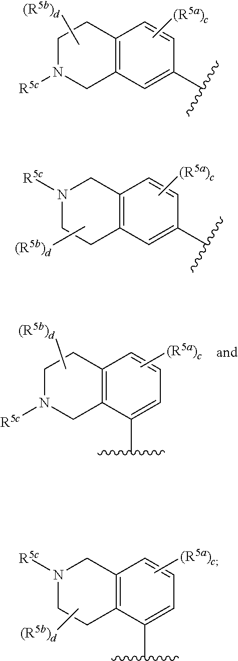

wherein R^{5c} has any of the values defined herein. Thus it may be that R^{5c} is selected from R^{5c} is selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷, wherein said C₁₋₆ alkyl, C₂₋₆ alkenyl and C₂₋₆ alkynyl is optionally substituted by one or more R⁸. It may be that R^{5c} is as defined in any of 106 to 120.
101. The group of the formula

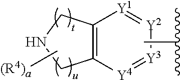

is selected from:

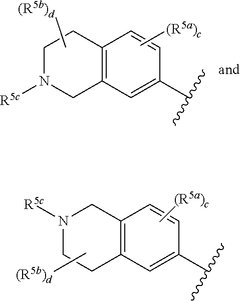

wherein R^{5c} has any of the values defined herein. Thus, it may be that R^{5c} is selected from R^{5c} is selected from: H, C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷, wherein said C₁₋₆ alkyl, C₂₋₆ alkenyl and C₂₋₆ alkynyl is optionally substituted by one or more R⁸. It may be that R^{5c} is as defined in any of 106 to 121.
102. R^{5a} and R^{5b} are each independently selected from: halo, —CN, —NO₂, ═O, C₁₋₆ alkyl, C₁₋₆ haloalkyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶SO₂R⁷, and —SO₂NR⁶R⁷.
103. R^{5a} and R^{5b} are each independently selected from: halo, —CN, ═O, C₁₋₆ alkyl, Q¹, —OR⁶, —NR⁶R⁷, —C(O)R⁶, and —NR⁶C(O)R⁷.
104. R^{5a}, and R^{5b} are each as defined in any one of 102 to 103, wherein said C₁₋₆ alkyl is substituted by one or more R⁸.
105. R^{5a} and R^{5b} are each independently selected from: halo and C₁₋₄ alkyl.
106. R^{5c} is independently selected from: C₁₋₆ alkyl, C₁₋₆ haloalkyl, C₂₋₆ alkenyl, C₂₋₆ alkynyl, Q¹, —S(O)ₓR^{B}, —C(O)R⁸, —C(O)OR^{B}, —C(O)NR⁶R⁷, and —SO₂NR⁶R⁷, wherein said C₁₋₆ alkyl, C₂₋₆ alkenyl and C₂₋₆ alkynyl is optionally substituted by one or more R⁸.
107. R^{5c} is independently selected from: H, C₁₋₆ alkyl, C₁₋₆ haloalkyl, C₂₋₆ alkenyl, C₂₋₆ alkynyl, Q¹, —C(O)R⁸, —C(O)OR^{B}, —C(O)NR⁶R⁷, wherein said C₁₋₆ alkyl, C₂₋₆ alkenyl and C₂₋₆ alkynyl is optionally substituted by one or more R⁸.
108. R^{5c} is independently selected from: C₁₋₆ alkyl, C₁₋₆ haloalkyl, C₂₋₆ alkenyl, C₂₋₆ alkynyl, Q¹, —C(O)R⁶, —C(O)OR⁶, —C(O)NR⁶R⁷, wherein said C₁₋₆ alkyl, C₂₋₆ alkenyl and C₂₋₆ alkynyl is optionally substituted by one or more R⁸.
109. R^{5c} is independently selected from: C₁₋₆ alkyl, C₁₋₆ haloalkyl, Q¹, —C(O)R⁶, and —C(O)OR⁶, wherein said C₁₋₆ alkyl is optionally substituted by one or more R⁸.
110. R^{5c} is independently selected from: C₁₋₆ alkyl, Q¹, —C(O)R⁶, and —C(O)OR⁶, wherein said C₁₋₆ alkyl is optionally substituted by one or more R⁸.
111. R^{5c} is independently selected from: —C(O)R⁶, and —C(O)OR⁶.
112. R^{5c} is as defined in any of 106 to 111, wherein Q¹ is independently selected from: 4- to 7-membered heterocyclyl, phenyl and 5- or 6-membered heteroaryl.
113. R^{5c} is as defined in any of 106 to 111, wherein Q¹ is independently selected from: phenyl and 5- or 6-membered heteroaryl.
114. R^{5c} is as defined in any of 106 to 111, wherein R⁸ is independently selected from: halo, and Q².
115. R^{5c} is as defined in any of 106 to 111, wherein R⁸ is Q².
116. R^{5c} is as defined in any of 106 to 111, wherein R⁸ is fluoro.
117. R^{5c} is as defined in any of 114 or 115, wherein Q² is independently selected from: 4- to 7-membered heterocyclyl, phenyl and 5- or 6-membered heteroaryl.
118. R^{5c} is as defined in any of 114 or 115, wherein Q² is independently selected from: phenyl and 5- or 6-membered heteroaryl.
119. R^{5c} is selected from: C₁₋₄ alkyl, C₁₋₄ haloalkyl, C₃₋₆ cycloalkyl, C₃₋₆ cycloalkyl-C₁₋₂ alkyl, phenyl, benzyl, —C(O)R⁶, —C(O)OR⁶ and —C(O)NR⁶R⁷; R⁶ is selected from; H, C₁₋₄ alkyl, C₁₋₄ haloalkyl, C₃₋₆ cycloalkyl, C₃₋₆ cycloalkyl-C₁₋₂ alkyl, phenyl and benzyl; and R⁷ is selected from: H, C₁₋₄ alkyl and C₁₋₄ haloalkyl
120. R^{5c} is selected from: C₁₋₄ alkyl, C₁₋₄ haloalkyl, C₃₋₄ cycloalkyl, C₃₋₄ cycloalkyl-methyl-, phenyl, benzyl, —C(O)R⁶ and —C(O)OR⁶; and R⁶ is selected from: H, C₁₋₄ alkyl, C₁₋₄ haloalkyl, C₃₋₄ cycloalkyl, C₃₋₄ cycloalkyl-methyl-, phenyl and benzyl.
121. R^{5c} is selected from:

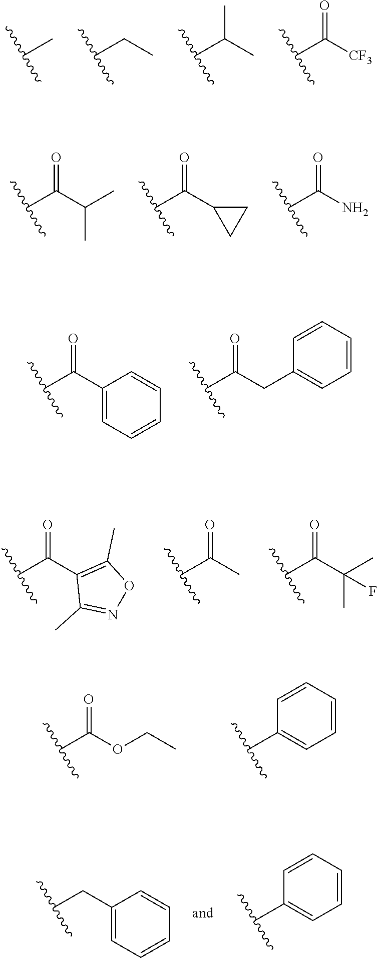

122. R^{5a} and R^{5b} are each independently selected from: halo, C₁₋₄ alkyl and C₁₋₄ haloalkyl; and R^{5c} is as defined in any of 106 to 121. Thus, it may be that c and d are each independently 0 or 1. It may be that c and d are both 0.
123. The group of the formula

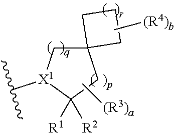

is

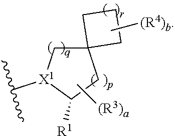

124. The group of the formula

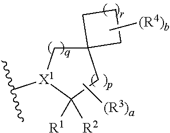

is

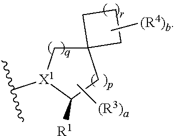

125. The group of the formula

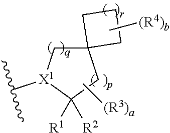

is selected from:

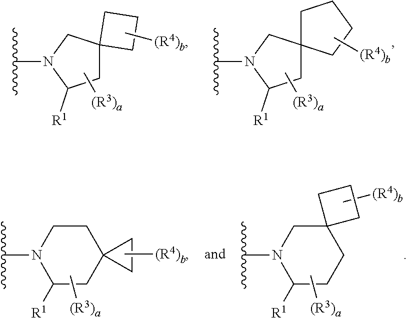

126. The group of the formula

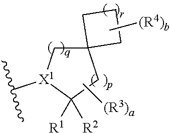

is selected from:

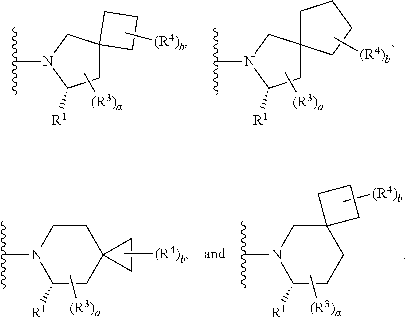

127. The group of the formula

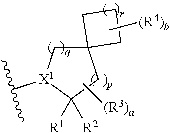

is selected from:

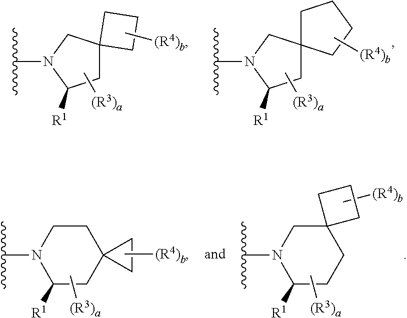

128. The group of the formula

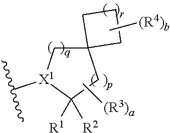

is selected from:

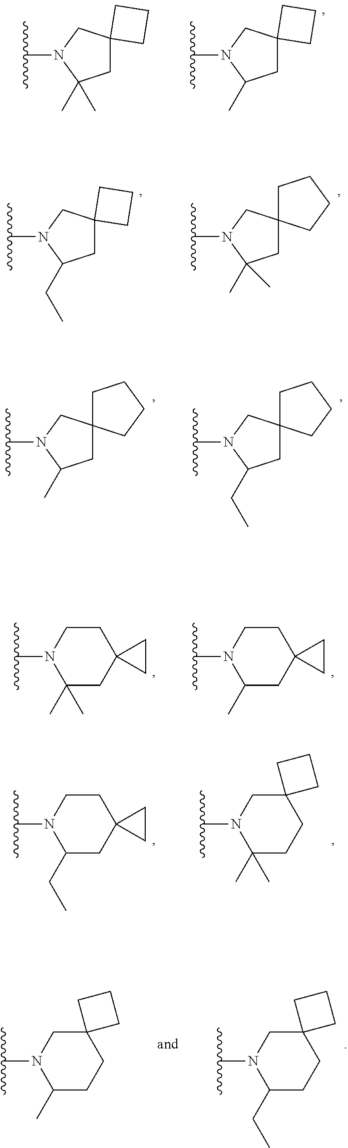

129. The group of the formula

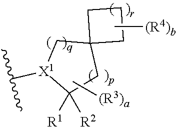

is selected from:

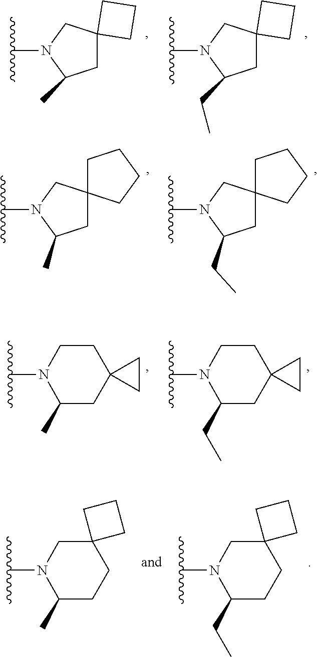

130. The group of the formula

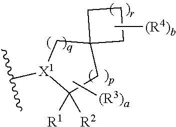

is selected from:

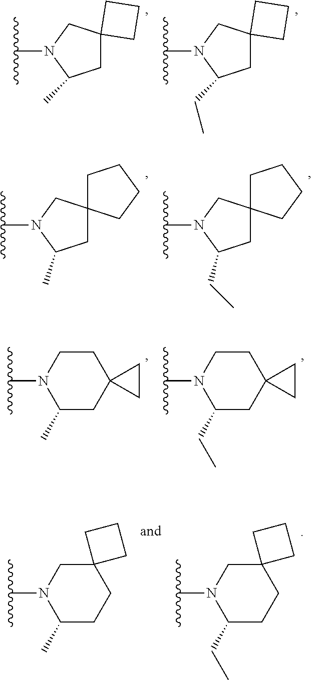

131. The group of the formula

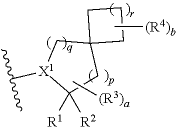

is:

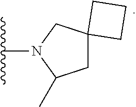 [SMILES: *N1CC2(CCC2)CC1C]

It may be that the group is

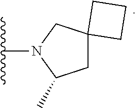 [SMILES: *N1CC2(CCC2)C[C@@H]1C]

It may be that the group is

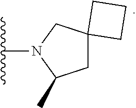 [SMILES: *N1CC2(CCC2)C[C@H]1C]

132. c is 0.
133. c is 1.
134. d is 0.
135. d is 1.
136. d is 2.

In an embodiment, the compound of formula (I) is a compound of any of formulae (I) to (XXIV), wherein L is a bond.

In an embodiment, the compound of formula (I) is a compound of any of formulae (I) to (XXIV), wherein X¹ is N and L is a bond.

In an embodiment, the compound of formula (I) is a compound of any of formulae (I) to (XXIV), wherein L is —CH₂—.

In an embodiment, the compound of formula (I) is a compound of any of formulae (I) to (XXIV), wherein X¹ is N and L is —CH₂—.

In an embodiment, the compound of formula (I) is a compound according to any of formulae (XIII) to (XXIV), wherein R^{5c} is according to any of paragraphs 106 to 121.

In an embodiment, the compound of formula (I) is a compound according to any of formulae (XIII) to (XXIV), wherein:
X¹ is N, and
R^{5c} is according to any of paragraphs 106 to 121.

In an embodiment, the compound of formula (I) is a compound according to any of formulae (XIII) to (XXIV), wherein:
X¹ is N,
L is a bond, and
R^{5c} is according to any of paragraphs 106 to 121.

In an embodiment, the compound of formula (I) is a compound according to any of formulae (XIII) to (XXIV), wherein:
X¹ is N,
Lis a bond,
R^{5c} is according to any of paragraphs 106 to 121, and
c and d are each 0.

In an embodiment, the compound of formula (I) is a compound according to any of formulae (VII) to (XII), wherein:
X¹ is N,
L is a bond,
Y¹, Y², Y³, Y⁴, are each CH, optionally substituted with R^{5a},
t and u are each 1,
each R^{5b} is independently according to any of paragraphs 102 to 105.

In an embodiment, the compound of formula (I) is a compound according to any of formulae (VII) to (XII), wherein:
X¹ is N,
L is a bond to Y² or Y³,
Y¹, Y², Y³, Y⁴, are each CH, optionally substituted with R^{5a},
t and u are each 1,
each R^{5b} is independently according to any of paragraphs 102 to 105

In an embodiment, the compound of formula (I) is a compound according to any of formulae (I), (VII), (X), (XIII), (XIX) and (XXII) wherein:

L is a bond or —CH₂—; and
the group of the formula

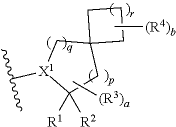

is as defined in any one of paragraphs 123 to 131.

Thus it may be in this embodiment that L is a bond.

In certain embodiments in the group of the formula

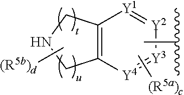

described herein (for example in compounds according to formula (VII) to (XII)), t and u are not both 0. For example in certain embodiments t is 1, 2 or 3; and u is 0, 1, 2 or 3. In certain embodiments t is 0, 1, 2 or 3; and u is 1, 2 or 3. In certain embodiments t is 1 or 2; and u is 0, 1 or 2. In certain embodiments t is 0, 1 or 2; and u is 1 or 2.

In certain embodiments, the compound of formula (I) is a compound according to any of formulae (XIII) to (XXIV), wherein c is an integer from 0 to 2. In certain embodiments, the compound of formula (I) is a compound according to any of formulae (XIII) to (XXIV), wherein d is an integer from 0 to 2. In certain embodiments, the compound of formula (I) is a compound according to any of formulae (XIII) to (XV), wherein c is an integer from 0 to 2, and d is an integer from 0 to 2. In certain embodiments, the compound of formula (I) is a compound according to any of formulae (XIII) to (XXIV), wherein c and d are 0.

In another embodiment there is provided a compound selected from Compound List A, or a pharmaceutically acceptable salt thereof:

### Compound List A:

| Example | Structure | IUPAC Name |
|---|---|---|
| 1 | 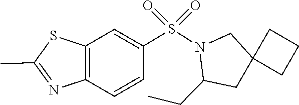 [SMILES: CCC1CC2(CCC2)CN1S(=O)(=O)c1ccc2nc(C)sc2c1] | 5-(2-chloro-6-fluorobenzyl)-4-((4,4- difluorocyclohexyl)methyl)-2- methyl-2,4-dihydro-3H-1,2,4- triazol-3-one |
| 2 | 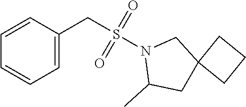 [SMILES: CC1CC2(CCC2)CN1S(=O)(=O)Cc1ccccc1] | 6-(Benzylsulfonyl)-7-methyl-6- azaspiro[3.4]octane |
| 3 | 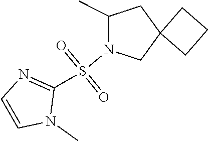 [SMILES: CC1CC2(CCC2)CN1S(=O)(=O)c1nccn1C] | 7-Methyl-6-((1-methyl-1H-imidazol- 2-yl)sulfonyl)-6- azaspiro[3.4]octane |
| 4 | 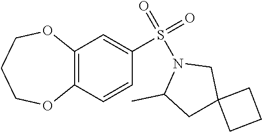 [SMILES: CC1CC2(CCC2)CN1S(=O)(=O)c1ccc2c(c1)OCCCO2] | 6-((3,4-Dihydro-2H- benzo[b][1,4]dioxepin-7- yl)sulfonyl)-7-methyl-6- azaspiro[3.4]octane |
| 5 | 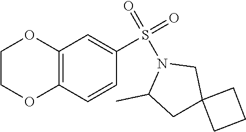 [SMILES: CC1CC2(CCC2)CN1S(=O)(=O)c1ccc2c(c1)OCCO2] | 6-((2,3-Dihydrobenzo[b][1,4]dioxin- 6-yl)sulfonyl)-7-methyl-6- azaspiro[3.4]octane |
| 6 | 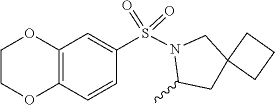 [SMILES: CC1CC2(CCC2)CN1S(=O)(=O)c1ccc2c(c1)OCCO2] | 6-((2,3-Dihydrobenzo[b][1,4]dioxin- 6-yl)sulfonyl)-7-methyl-6- azaspiro[3.4]octane (enantiomer 1) |
| 7 | 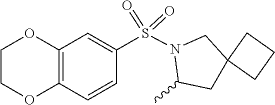 [SMILES: CC1CC2(CCC2)CN1S(=O)(=O)c1ccc2c(c1)OCCO2] | 6-((2,3-dihydrobenzo[b][1,4]dioxin- 6-yl)sulfonyl)-7-methyl-6- azaspiro[3.4]octane (enantiomer 2) |
| 8 | 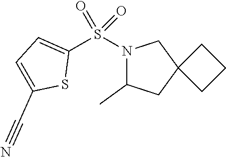 [SMILES: CC1CC2(CCC2)CN1S(=O)(=O)c1ccc(C#N)s1] | 5-((7-Methyl-6-azaspiro[3.4]octan- 6-yl)sulfonyl)thiophene-2- carbonitrile |
| 9 | 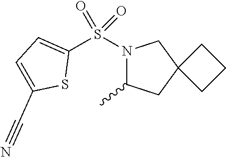 [SMILES: CC1CC2(CCC2)CN1S(=O)(=O)c1ccc(C#N)s1] | 5-((7-Methyl-6-azaspiro[3.4]octan- 6-yl)sulfonyl) thiophene-2- carbonitrile (enantiomer 1) |
| 10 | 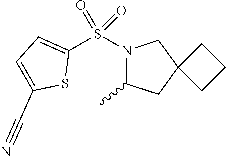 [SMILES: CC1CC2(CCC2)CN1S(=O)(=O)c1ccc(C#N)s1] | 5-((7-methyl-6-azaspiro[3.4]octan- 6-yl)sulfonyl)thiophene-2- carbonitrile (enantiomer 2) |
| 11 | 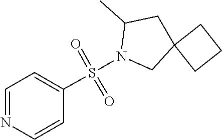 [SMILES: CC1CC2(CCC2)CN1S(=O)(=O)c1ccncc1] | 7-Methyl-6-(pyridin-4-ylsulfonyl)-6- azaspiro[3.4]octane |
| 12 | 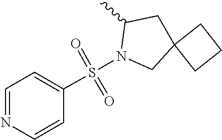 [SMILES: CC1CC2(CCC2)CN1S(=O)(=O)c1ccncc1] | 7-Methyl-6-(pyridin-4-ylsulfonyl)-6- azaspiro[3.4]octane (enantiomer 1) |
| 13 | 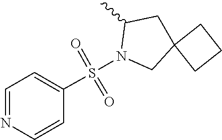 [SMILES: CC1CC2(CCC2)CN1S(=O)(=O)c1ccncc1] | 7-methyl-6-(pyridin-4-ylsulfonyl)-6- azaspiro[3.4]octane (enantiomer 2) |
| 14 | 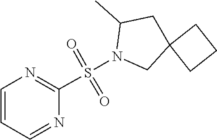 [SMILES: CC1CC2(CCC2)CN1S(=O)(=O)c1ncccn1] | 7-Methyl-6-(pyrimidin-2-ylsulfonyl)- 6-azaspiro[3.4]octane |
| 15 | 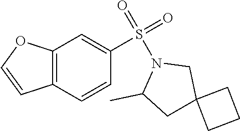 [SMILES: CC1CC2(CCC2)CN1S(=O)(=O)c1ccc2ccoc2c1] | 6-(Benzofuran-6-ylsulfonyl)-7- methyl-6-azaspiro[3.4]octane |
| 16 | 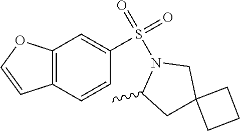 [SMILES: CC1CC2(CCC2)CN1S(=O)(=O)c1ccc2ccoc2c1] | 6-(Benzofuran-6-ylsulfonyl)-7- methyl-6-azaspiro[3.4]octane (enantiomer 1) |
| 17 | 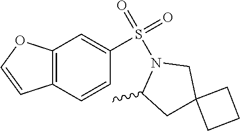 [SMILES: CC1CC2(CCC2)CN1S(=O)(=O)c1ccc2ccoc2c1] | 6-(benzofuran-6-ylsulfonyl)-7- methyl-6-azaspiro[3.4]octane (enantiomer 2) |
| 18 | 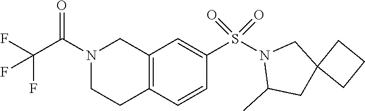 [SMILES: CC1CC2(CCC2)CN1S(=O)(=O)c1ccc2c(c1)CN(C(=O)C(F)(F)F)CC2] | 2,2,2-Trifluoro-1-(7-((7-methyl-6- azaspiro[3.4]octan-6-yl)sulfonyl)- 3,4-dihydroisoquinolin-2(1H)- yl)ethan-1-one |
| 19 | 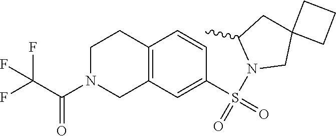 [SMILES: CC1CC2(CCC2)CN1S(=O)(=O)c1ccc2c(c1)CN(C(=O)C(F)(F)F)CC2] | 2,2,2-Trifluoro-1-(7-((7-methyl-6- azaspiro[3.4]octan-6-yl)sulfonyl)- 3,4-dihydroisoquinolin-2(1H)- yl)ethan-1-one (enantiomer 1) |
| 20 | 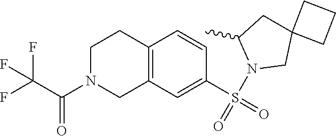 [SMILES: CC1CC2(CCC2)CN1S(=O)(=O)c1ccc2c(c1)CN(C(=O)C(F)(F)F)CC2] | 2,2,2-trifluoro-1-(7-((7-methyl-6- azaspiro[3.4]octan-6-yl)sulfonyl)- 3,4-dihydroisoquinolin-2(1H)- yl)ethan-1-one (enantiomer 2) |
| 21 | 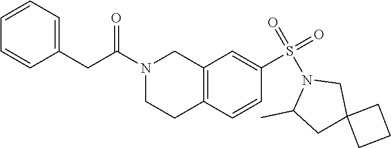 [SMILES: CC1CC2(CCC2)CN1S(=O)(=O)c1ccc2c(c1)CN(C(=O)Cc1ccccc1)CC2] | 1-(7-((7-Methyl-6- azaspiro[3.4]octan-6-yl)sulfonyl)- 3,4-dihydroisoquinolin-2(1H)-yl)-2- phenylethan-1-one |
| 22 | 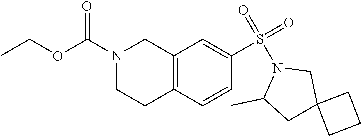 [SMILES: CCOC(=O)N1CCc2ccc(S(=O)(=O)N3CC4(CCC4)CC3C)cc2C1] | Ethyl 7-((7-methyl-6- azaspiro[3.4]octan-6-yl)sulfonyl)- 3,4-dihydroisoquinoline-2(1H)- carboxylate |
| 23 | 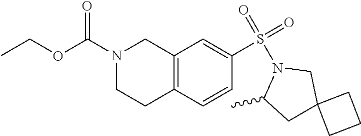 [SMILES: CCOC(=O)N1CCc2ccc(S(=O)(=O)N3CC4(CCC4)CC3C)cc2C1] | Ethyl 7-((7-methyl-6- azaspiro[3.4]octan-6-yl)sulfonyl)- 3,4-dihydroisoquinoline-2(1H)- carboxylate (enantiomer 1) |
| 24 | 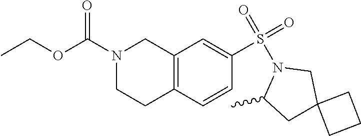 [SMILES: CCOC(=O)N1CCc2ccc(S(=O)(=O)N3CC4(CCC4)CC3C)cc2C1] | Ethyl 7-((7-methyl-6- azaspiro[3.4]octan-6-yl)sulfonyl)- 3,4-dihydroisoquinoline-2(1H)- carboxylate (enantiomer 2) |
| 25 | 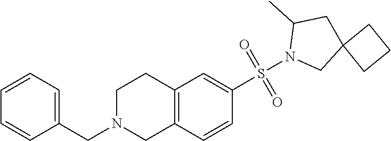 [SMILES: CC1CC2(CCC2)CN1S(=O)(=O)c1ccc2c(c1)CCN(Cc1ccccc1)C2] | 2-Benzyl-6-((7-methyl-6- azaspiro[3.4]octan-6-yl)sulfonyl)- 1,2,3,4-tetrahydroisoquinoline |
| 26 | 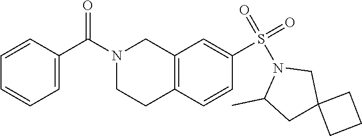 [SMILES: CC1CC2(CCC2)CN1S(=O)(=O)c1ccc2c(c1)CN(C(=O)c1ccccc1)CC2] | (7-((7-Methyl-6-azaspiro[3.4]octan- 6-yl)sulfonyl)-3,4- dihydroisoquinolin-2(1H)- yl)(phenyl)methanone |
| 27 | 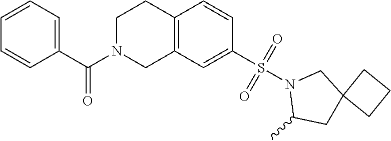 [SMILES: CC1CC2(CCC2)CN1S(=O)(=O)c1ccc2c(c1)CN(C(=O)c1ccccc1)CC2] | (7-((7-Methyl-6-azaspiro[3.4]octan- 6-yl)sulfonyl)-3,4- dihydroisoquinolin-2(1H)- yl)(phenyl)methanone (enantiomer 1) |
| 28 | 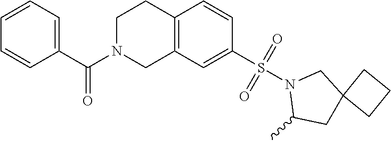 [SMILES: CC1CC2(CCC2)CN1S(=O)(=O)c1ccc2c(c1)CN(C(=O)c1ccccc1)CC2] | (7-((7-methyl-6-azaspiro[3.4]octan- 6-yl)sulfonyl)-3,4- dihydroisoquinolin-2(1H)- yl)(phenyl)methanone (enantiomer 2) |
| 29 | 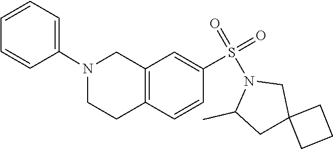 [SMILES: CC1CC2(CCC2)CN1S(=O)(=O)c1ccc2c(c1)CN(c1ccccc1)CC2] | 7-((7-Methyl-6-azaspiro[3.4]octan- 6-yl)sulfonyl)-2-phenyl-1,2,3,4- tetrahydroisoquinoline |
| 30 | 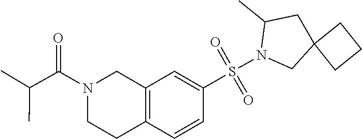 [SMILES: CC(C)C(=O)N1CCc2ccc(S(=O)(=O)N3CC4(CCC4)CC3C)cc2C1] | 2-Methyl-1-(7-((7-methyl-6- azaspiro[3.4]octan-6-yl)sulfonyl)- 3,4-dihydroisoquinolin-2(1H)- yl)propan-1-one |
| 31 | 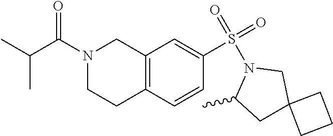 [SMILES: CC(C)C(=O)N1CCc2ccc(S(=O)(=O)N3CC4(CCC4)CC3C)cc2C1] | 2-Methyl-1-(7-((7-methyl-6- azaspiro[3.4]octan-6-yl)sulfonyl)- 3,4-dihydroisoquinolin-2(1H)- yl)propan-1-one (enantiomer 1) |
| 32 | 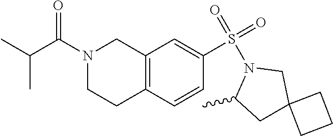 [SMILES: CC(C)C(=O)N1CCc2ccc(S(=O)(=O)N3CC4(CCC4)CC3C)cc2C1] | 2-methyl-1-(7-((7-methyl-6- azaspiro[3.4]octan-6-yl)sulfonyl)- 3,4-dihydroisoquinolin-2(1H)- yl)propan-1-one (enantiomer 2) |
| 33 | 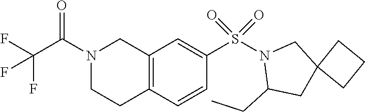 [SMILES: CCC1CC2(CCC2)CN1S(=O)(=O)c1ccc2c(c1)CN(C(=O)C(F)(F)F)CC2] | 1-(7-((7-Ethyl-6- azaspiro[3.4]octan-6-yl)sulfonyl)- 3,4-dihydroisoquinolin-2(1H)-yl)- 2,2,2-trifluoroethan-1-one |
| 34 | 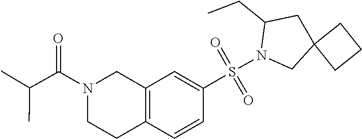 [SMILES: CCC1CC2(CCC2)CN1S(=O)(=O)c1ccc2c(c1)CN(C(=O)C(C)C)CC2] | 1-(7-((7-ethyl-6-azaspiro[3.4]octan- 6-yl)sulfonyl)-3,4- dihydroisoquinolin-2(1H)-yl)-2- methylpropan-1-one |
| 35 | 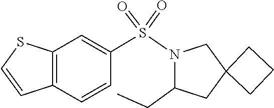 [SMILES: CCC1CC2(CCC2)CN1S(=O)(=O)c1ccc2ccsc2c1] | 6-(Benzo[b]thiophen-6-ylsulfonyl)- 7-ethyl-6-azaspiro[3.4]octane |
| 36 | 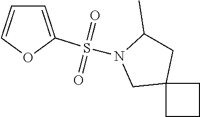 [SMILES: CC1CC2(CCC2)CN1S(=O)(=O)c1ccco1] | 6-(Furan-2-ylsulfonyl)-7-methyl-6- azaspiro[3.4]octane |
| 37 | 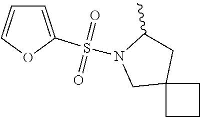 [SMILES: CC1CC2(CCC2)CN1S(=O)(=O)c1ccco1] | 6-(Furan-2-ylsulfonyl)-7-methyl-6- azaspiro[3.4]octane (enantiomer 1) |
| 38 | 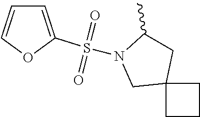 [SMILES: CC1CC2(CCC2)CN1S(=O)(=O)c1ccco1] | 6-(furan-2-ylsulfonyl)-7-methyl-6- azaspiro[3.4]octane (enantiomer 2) |
| 39 | 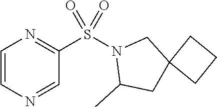 [SMILES: CC1CC2(CCC2)CN1S(=O)(=O)c1cnccn1] | 7-Methyl-6-(pyrazin-2-ylsulfonyl)-6- azaspiro[3.4]octane |
| 40 | 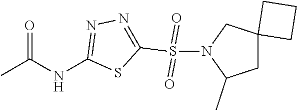 [SMILES: CC(=O)Nc1nnc(S(=O)(=O)N2CC3(CCC3)CC2C)s1] | N-(5-((7-methyl-6- azaspiro[3.4]octan-6-yl)sulfonyl)- 1,3,4-thiadiazol-2-yl)acetamide |
| 41 | 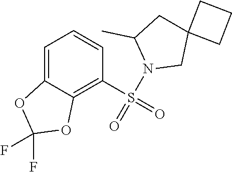 [SMILES: CC1CC2(CCC2)CN1S(=O)(=O)c1cccc2c1OC(F)(F)O2] | 6-((2,2-Difluorobenzo[d][1,3]dioxol- 4-yl)sulfonyl)-7-methyl-6- azaspiro[3.4]octane |
| 42 | 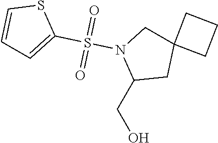 [SMILES: O=S(=O)(c1cccs1)N1CC2(CCC2)CC1CO] | (6-(Thiophen-2-ylsulfonyl)-6- azaspiro[3.4]octan-7-yl)methanol |
| 43 | 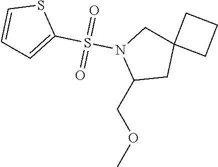 [SMILES: COCC1CC2(CCC2)CN1S(=O)(=O)c1cccs1] | 7-(methoxymethyl)-6-(thiophen-2- ylsulfonyl)-6-azaspiro[3.4]octane |
| 44 | 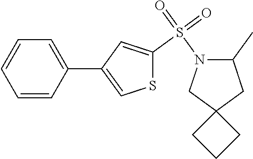 [SMILES: CC1CC2(CCC2)CN1S(=O)(=O)c1cc(-c2ccccc2)cs1] | 7-Methyl-6-((4-phenylthiophen-2- yl)sulfonyl)-6-azaspiro[3.4]octane |
| 45 | 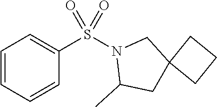 [SMILES: CC1CC2(CCC2)CN1S(=O)(=O)c1ccccc1] | 7-Methyl-6-(phenylsulfonyl)-6- azaspiro[3.4]octane |
| 46 | 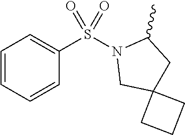 [SMILES: CC1CC2(CCC2)CN1S(=O)(=O)c1ccccc1] | 7-Methyl-6-(phenylsulfonyl)-6- azaspiro[3.4]octane (enantiomer 1) |
| 47 | 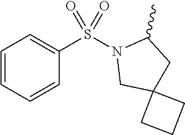 [SMILES: CC1CC2(CCC2)CN1S(=O)(=O)c1ccccc1] | 7-methyl-6-(phenylsulfonyl)-6- azaspiro[3.4]octane (enantiomer 2) |
| 48 | 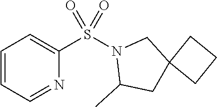 [SMILES: CC1CC2(CCC2)CN1S(=O)(=O)c1ccccn1] | 7-Methyl-6-(pyridin-2-ylsulfonyl)-6- azaspiro[3.4]octane |
| 49 | 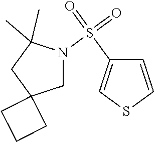 [SMILES: CC1(C)CC2(CCC2)CN1S(=O)(=O)c1ccsc1] | 7,7-Dimethyl-6-(thiophen-3- ylsulfonyl)-6-azaspiro[3.4]octane |
| 50 | 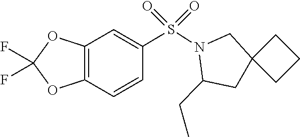 [SMILES: CCC1CC2(CCC2)CN1S(=O)(=O)c1ccc2c(c1)OC(F)(F)O2] | 6-((2,2-Difluorobenzo[d][1,3]dioxol- 5-yl)sulfonyl)-7-ethyl-6- azaspiro[3.4]octane |
| 51 | 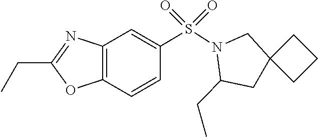 [SMILES: CCc1nc2cc(S(=O)(=O)N3CC4(CCC4)CC3CC)ccc2o1] | 2-Ethyl-5-((7-ethyl-6- azaspiro[3.4]octan-6- yl)sulfonyl)benzo[d]oxazole |
| 52 | 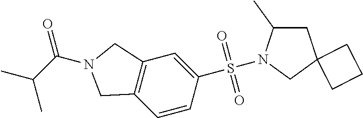 [SMILES: CC(C)C(=O)N1Cc2ccc(S(=O)(=O)N3CC4(CCC4)CC3C)cc2C1] | 2-Methyl-1-(5-((7-methyl-6- azaspiro[3.4]octan-6- yl)sulfonyl)isoindolin-2-yl)propan- 1-one |
| 53 | 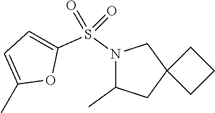 [SMILES: Cc1ccc(S(=O)(=O)N2CC3(CCC3)CC2C)o1] | 7-Methyl-6-((5-methylfuran-2- yl)sulfonyl)-6-azaspiro[3.4]octane |
| 54 | 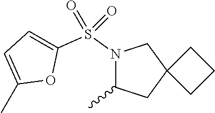 [SMILES: Cc1ccc(S(=O)(=O)N2CC3(CCC3)CC2C)o1] | 7-Methyl-6-((5-methylfuran-2- yl)sulfonyl)-6-azaspiro[3.4]octane (enantiomer 1) |
| 55 | 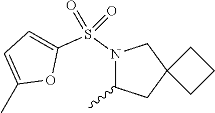 [SMILES: Cc1ccc(S(=O)(=O)N2CC3(CCC3)CC2C)o1] | 7-methyl-6-((5-methylfuran-2- yl)sulfonyl)-6-azaspiro[3.4]octane (enantiomer 2) |
| 56 | 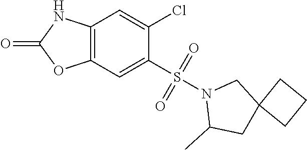 [SMILES: CC1CC2(CCC2)CN1S(=O)(=O)c1cc2oc(=O)[nH]c2cc1Cl] | 5-Chloro-6-((7-methyl-6- azaspiro[3.4]octan-6- yl)sulfonyl)benzo[d]oxazol-2(3H)- one |
| 57 | 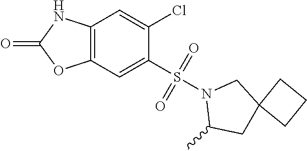 [SMILES: CC1CC2(CCC2)CN1S(=O)(=O)c1cc2oc(=O)[nH]c2cc1Cl] | 5-Chloro-6-((7-methyl-6- azaspiro[3.4]octan-6- yl)sulfonyl)benzo[d]oxazol-2(3H)- one (enantiomer 1) |
| 58 | 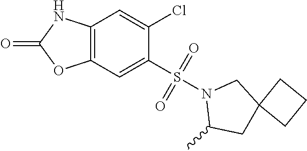 [SMILES: CC1CC2(CCC2)CN1S(=O)(=O)c1cc2oc(=O)[nH]c2cc1Cl] | 5-chloro-6-((7-methyl-6- azaspiro[3.4]octan-6- yl)sulfonyl)benzo[d]oxazol-2(3H)- one (enantiomer 2) |
| 59 | 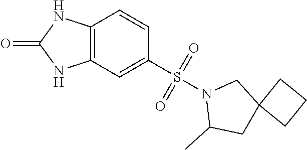 [SMILES: CC1CC2(CCC2)CN1S(=O)(=O)c1ccc2[nH]c(=O)[nH]c2c1] | 5-((7-Methyl-6-azaspiro[3.4]octan- 6-yl)sulfonyl)-1,3-dihydro-2H- benzo[d]imidazol-2-one |
| 60 | 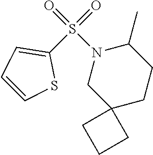 [SMILES: CC1CCC2(CCC2)CN1S(=O)(=O)c1cccs1] | 7-Methyl-6-(thiophen-2-ylsulfonyl)- 6-azaspiro[3.5]nonane |
| 61 | 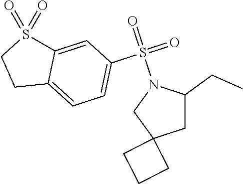 [SMILES: CCC1CC2(CCC2)CN1S(=O)(=O)c1ccc2c(c1)S(=O)(=O)CC2] | 6-((7-Ethyl-6-azaspiro[3.4]octan-6- yl)sulfonyl)-2,3- dihydrobenzo[b]thiophene 1,1- dioxide |
| 62 | 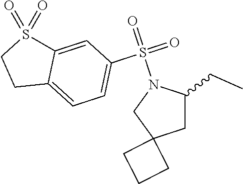 [SMILES: CCC1CC2(CCC2)CN1S(=O)(=O)c1ccc2c(c1)S(=O)(=O)CC2] | 6-((7-Ethyl-6-azaspiro[3.4]octan-6- yl)sulfonyl)-2,3- dihydrobenzo[b]thiophene 1,1- dioxide (enantiomer 1) |
| 63 | 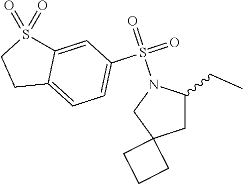 [SMILES: CCC1CC2(CCC2)CN1S(=O)(=O)c1ccc2c(c1)S(=O)(=O)CC2] | 6-((7-ethyl-6-azaspiro[3.4]octan-6- yl)sulfonyl)-2,3- dihydrobenzo[b]thiophene 1,1- dioxide (enantiomer 2) |
| 64 | 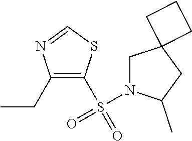 [SMILES: CCc1ncsc1S(=O)(=O)N1CC2(CCC2)CC1C] | 4-Ethyl-5-((7-methyl-6- azaspiro[3.4]octan-6- yl)sulfonyl)thiazole |
| 65 | 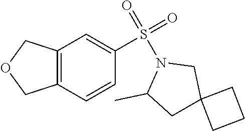 [SMILES: CC1CC2(CCC2)CN1S(=O)(=O)c1ccc2c(c1)COC2] | 6-((1,3-Dihydroisobenzofuran-5- yl)sulfonyl)-7-methyl-6- azaspiro[3.4]octane |
| 66 | 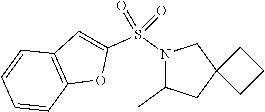 [SMILES: CC1CC2(CCC2)CN1S(=O)(=O)c1cc2ccccc2o1] | 6-(Benzofuran-2-ylsulfonyl)-7- methyl-6-azaspiro[3.4]octane |
| 67 | 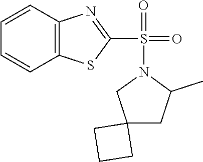 [SMILES: CC1CC2(CCC2)CN1S(=O)(=O)c1nc2ccccc2s1] | 2-((7-Methyl-6-azaspiro[3.4]octan- 6-yl)sulfonyl)benzo[d]thiazole |
| 68 | 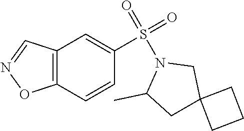 [SMILES: CC1CC2(CCC2)CN1S(=O)(=O)c1ccc2oncc2c1] | 5-((7-Methyl-6-azaspiro[3.4]octan- 6-yl)sulfonyl)benzo[d]isoxazole |
| 69 | 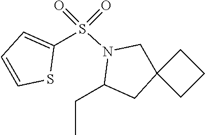 [SMILES: CCC1CC2(CCC2)CN1S(=O)(=O)c1cccs1] | 7-Ethyl-6-(thiophen-2-ylsulfonyl)-6- azaspiro[3.4]octane |
| 70 | 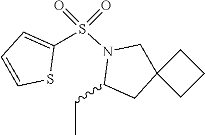 [SMILES: CCC1CC2(CCC2)CN1S(=O)(=O)c1cccs1] | 7-Ethyl-6-(thiophen-2-ylsulfonyl)-6- azaspiro[3.4]octane (enantiomer 1) |
| 71 | 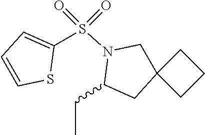 [SMILES: CCC1CC2(CCC2)CN1S(=O)(=O)c1cccs1] | 7-ethyl-6-(thiophen-2-ylsulfonyl)-6- azaspiro[3.4]octane (enantiomer 2) |
| 72 | 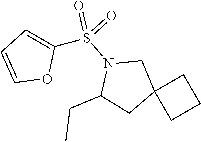 [SMILES: CCC1CC2(CCC2)CN1S(=O)(=O)c1ccco1] | 7-Ethyl-6-(furan-2-ylsulfonyl)-6- azaspiro[3.4]octane |
| 73 | 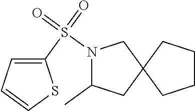 [SMILES: CC1CC2(CCCC2)CN1S(=O)(=O)c1cccs1] | 3-Methyl-2-(thiophen-2-ylsulfonyl)- 2-azaspiro[4.4]nonane |
| 74 | 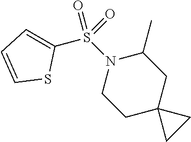 [SMILES: CC1CC2(CCN1S(=O)(=O)c1cccs1)CC2] | 5-Methyl-6-(thiophen-2-ylsulfonyl)- 6-azaspiro[2.5]octane |
| 75 | 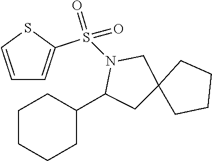 [SMILES: O=S(=O)(c1cccs1)N1CC2(CCCC2)CC1C1CCCCC1] | 3-Cyclohexyl-2-(thiophen-2- ylsulfonyl)-2-azaspiro[4.4]nonane |
| 76 | 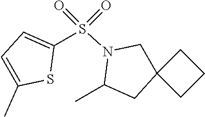 [SMILES: Cc1ccc(S(=O)(=O)N2CC3(CCC3)CC2C)s1] | 7-Methyl-6-((5-methylthiophen-2- yl) sulfonyl)-6-azaspiro[3.4]octane |
| 77 | 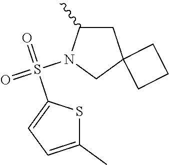 [SMILES: Cc1ccc(S(=O)(=O)N2CC3(CCC3)CC2C)s1] | 7-Methyl-6-((5-methylthiophen-2- yl)sulfonyl)-6-azaspiro[3.4]octane (enantiomer 1) |
| 78 | 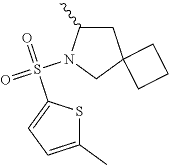 [SMILES: Cc1ccc(S(=O)(=O)N2CC3(CCC3)CC2C)s1] | 7-methyl-6-((5-methylthiophen-2- yl)sulfonyl)-6-azaspiro[3.4]octane (enantiomer 2) |
| 79 | 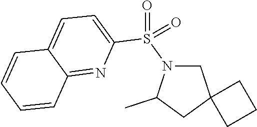 [SMILES: CC1CC2(CCC2)CN1S(=O)(=O)c1ccc2ccccc2n1] | 2-((7-Methyl-6-azaspiro[3.4]octan- 6-yl)sulfonyl)quinoline |
| 80 | 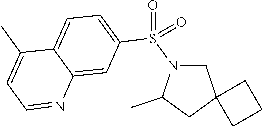 [SMILES: Cc1ccnc2cc(S(=O)(=O)N3CC4(CCC4)CC3C)ccc12] | 4-Methyl-7-((7-methyl-6- azaspiro[3.4]octan-6- yl)sulfonyl)quinoline |
| 81 | 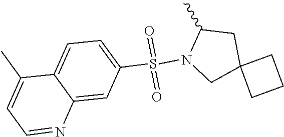 [SMILES: Cc1ccnc2cc(S(=O)(=O)N3CC4(CCC4)CC3C)ccc12] | 4-Methyl-7-((7-methyl-6- azaspiro[3.4]octan-6- yl)sulfonyl)quinoline (enantiomer 1) |
| 82 | 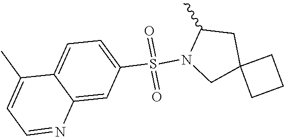 [SMILES: Cc1ccnc2cc(S(=O)(=O)N3CC4(CCC4)CC3C)ccc12] | 4-methyl-7-((7-methyl-6- azaspiro[3.4]octan-6- yl)sulfonyl)quinoline (enantiomer 2) |
| 83 | 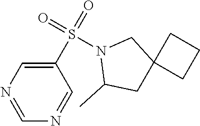 [SMILES: CC1CC2(CCC2)CN1S(=O)(=O)c1cncnc1] | 7-Methyl-6-(pyrimidin-5-ylsulfonyl)- 6-azaspiro[3.4]octane |
| 84 | 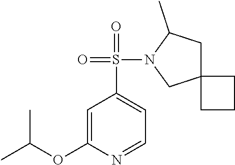 [SMILES: CC(C)Oc1cc(S(=O)(=O)N2CC3(CCC3)CC2C)ccn1] | 6-((2-Isopropoxypyridin-4- yl)sulfonyl)-7-methyl-6- azaspiro[3.4]octane |
| 85 | 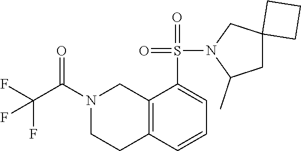 [SMILES: CC1CC2(CCC2)CN1S(=O)(=O)c1cccc2c1CN(C(=O)C(F)(F)F)CC2] | 2,2,2-Trifluoro-1-(8-((7-methyl-6- azaspiro[3.4]octan-6-yl)sulfonyl)- 3,4-dihydroisoquinolin-2(1H)- yl)ethan-1-one |
| 86 | 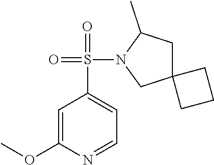 [SMILES: COc1cc(S(=O)(=O)N2CC3(CCC3)CC2C)ccn1] | 6-((2-Methoxypyridin-4-yl)sulfonyl)- 7-methyl-6-azaspiro[3.4]octane |
| 87 | 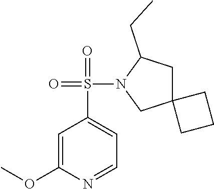 [SMILES: CCC1CC2(CCC2)CN1S(=O)(=O)c1ccnc(OC)c1] | 7-Ethyl-6-((2-methoxypyridin-4- yl)sulfonyl)-6-azaspiro[3.4]octane |
| 88 | 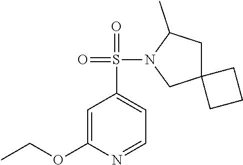 [SMILES: CCOc1cc(S(=O)(=O)N2CC3(CCC3)CC2C)ccn1] | 6-((2-Ethoxypyridin-4-yl)sulfonyl)- 7-methyl-6-azaspiro[3.4]octane |
| 89 | 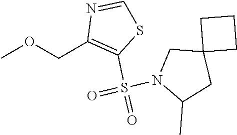 [SMILES: COCc1ncsc1S(=O)(=O)N1CC2(CCC2)CC1C] | 4-(Methoxymethyl)-5-((7-methyl-6- azaspiro[3.4]octan-6- yl)sulfonyl)thiazole |
| 90 | 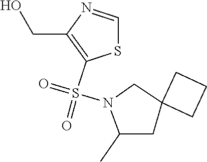 [SMILES: CC1CC2(CCC2)CN1S(=O)(=O)c1scnc1CO] | (5-((7-Methyl-6-azaspiro[3.4]octan- 6-yl)sulfonyl)thiazol-4-yl)methanol |
| 91 | 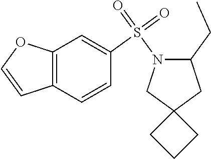 [SMILES: CCC1CC2(CCC2)CN1S(=O)(=O)c1ccc2ccoc2c1] | 6-(Benzofuran-6-ylsulfonyl)-7- ethyl-6-azaspiro[3.4]octane |
| 92 | 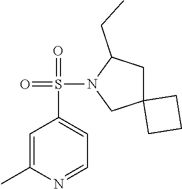 [SMILES: CCC1CC2(CCC2)CN1S(=O)(=O)c1ccnc(C)c1] | 7-Ethyl-6-((2-methylpyridin-4- yl)sulfonyl)-6-azaspiro[3.4]octane |
| 93 | 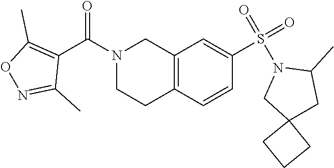 [SMILES: Cc1noc(C)c1C(=O)N1CCc2ccc(S(=O)(=O)N3CC4(CCC4)CC3C)cc2C1] | (3,5-Dimethylisoxazol-4-yl)(7-((7- methyl-6-azaspiro[3.4]octan-6- yl)sulfonyl)-3,4-dihydroisoquinolin- 2(1H)-yl)methanone |
| 94 | 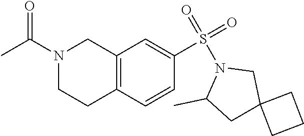 [SMILES: CC(=O)N1CCc2ccc(S(=O)(=O)N3CC4(CCC4)CC3C)cc2C1] | 1-(7-((7-Methyl-6- azaspiro[3.4]octan-6-yl)sulfonyl)- 3,4-dihydroisoquinolin-2(1H)- yl)ethan-1-one |
| 95 | 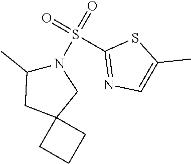 | 5-Methyl-2-((7-methyl-6- azaspiro[3.4]octan-6- yl)sulfonyl)thiazole |
| 96 | 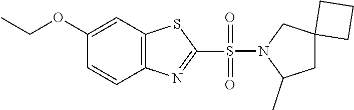 [SMILES: CCOc1ccc2nc(S(=O)(=O)N3CC4(CCC4)CC3C)sc2c1] | 6-Ethoxy-2-((7-methyl-6- azaspiro[3.4]octan-6- yl)sulfonyl)benzo[d]thiazole |
| 97 | 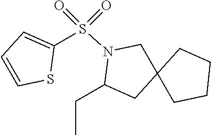 [SMILES: CCC1CC2(CCCC2)CN1S(=O)(=O)c1cccs1] | 3-Ethyl-2-(thiophen-2-ylsulfonyl)-2- azaspiro[4.4]nonane |
| 98 | 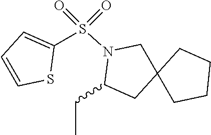 [SMILES: CCC1CC2(CCCC2)CN1S(=O)(=O)c1cccs1] | 3-Ethyl-2-(thiophen-2-ylsulfonyl)-2- azaspiro[4.4]nonane (enantiomer 1) |
| 99 | 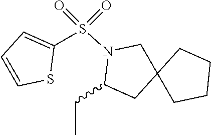 [SMILES: CCC1CC2(CCCC2)CN1S(=O)(=O)c1cccs1] | 3-ethyl-2-(thiophen-2-ylsulfonyl)-2- azaspiro[4.4]nonane (enantiomer 2) |
| 100 | 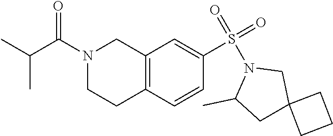 [SMILES: CC(C)C(=O)N1CCc2ccc(S(=O)(=O)N3CC4(CCC4)CC3C)cc2C1] | 2-Methyl-1-(7-((3-methyl-2- azaspiro[4.4]nonan-2-yl)sulfonyl)- 3,4-dihydroisoquinolin-2(1H)- yl)propan-1-one |
| 101 | 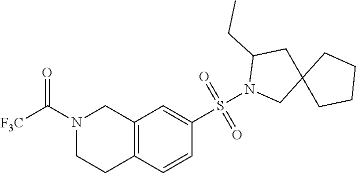 [SMILES: CCC1CC2(CCCC2)CN1S(=O)(=O)c1ccc2c(c1)CN(C(=O)C(F)(F)F)CC2] | 1-(7-((3-Ethyl-2- azaspiro[4.4]nonan-2-yl)sulfonyl)- 3,4-dihydroisoquinolin-2(1H)-yl)- 2,2,2-trifluoroethan-1-one |
| 102 | 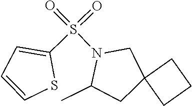 [SMILES: CC1CC2(CCC2)CN1S(=O)(=O)c1cccs1] | 7-Methyl-6-(thiophen-2-ylsulfonyl)- 6-azaspiro[3.4]octane |
| 103 | 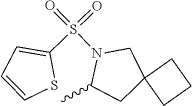 [SMILES: CC1CC2(CCC2)CN1S(=O)(=O)c1cccs1] | 7-Methyl-6-(thiophen-2-ylsulfonyl)- 6-azaspiro[3.4]octane (enantiomer 1) |
| 104 | 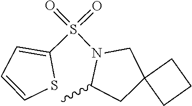 [SMILES: CC1CC2(CCC2)CN1S(=O)(=O)c1cccs1] | 7-methyl-6-(thiophen-2-ylsulfonyl)- 6-azaspiro[3.4]octane (enantiomer 2) |
| 105 | 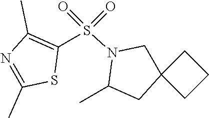 [SMILES: Cc1nc(C)c(S(=O)(=O)N2CC3(CCC3)CC2C)s1] | 2,4-Dimethyl-5-((7-methyl-6- azaspiro[3.4]octan-6- yl)sulfonyl)thiazole |
| 106 | 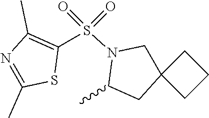 [SMILES: Cc1nc(C)c(S(=O)(=O)N2CC3(CCC3)CC2C)s1] | 2,4-Dimethyl-5-((7-methyl-6- azaspiro[3.4]octan-6- yl)sulfonyl)thiazole (enantiomer 1) |
| 107 | 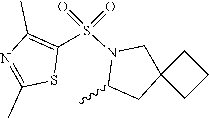 [SMILES: Cc1nc(C)c(S(=O)(=O)N2CC3(CCC3)CC2C)s1] | 2,4-dimethyl-5-((7-methyl-6- azaspiro[3.4]octan-6- yl)sulfonyl)thiazole (enantiomer 2) |
| 108 | 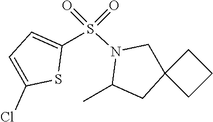 [SMILES: CC1CC2(CCC2)CN1S(=O)(=O)c1ccc(Cl)s1] | 6-((5-Chlorothiophen-2- yl)sulfonyl)-7-methyl-6- azaspiro[3.4]octane |
| 109 | 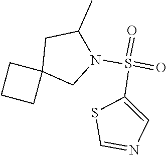 [SMILES: CC1CC2(CCC2)CN1S(=O)(=O)c1cncs1] | 5-((7-Methyl-6-azaspiro[3.4]octan- 6-yl)sulfonyl)thiazole |
| 110 | 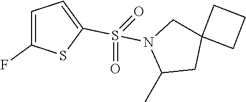 [SMILES: CC1CC2(CCC2)CN1S(=O)(=O)c1ccc(F)s1] | 6-((5-fluorothiophen-2-yl)sulfonyl)- 7-methyl-6-azaspiro[3.4]octane |
| 111 | 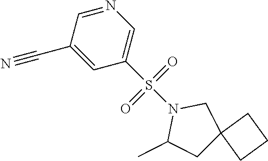 [SMILES: CC1CC2(CCC2)CN1S(=O)(=O)c1cncc(C#N)c1] | 5-((7-methyl-6-azaspiro[3.4]octan- 6-yl)sulfonyl)nicotinonitrile |

In another embodiment there is provided a compound selected from any one of the Examples herein, or a pharmaceutically acceptable salt thereof.

Particular compounds of the invention are those that have an PIC₅₀ of greater than 5.5, preferably those with a pIC₅₀ of 6 or more when measured in the Human Cav2.3 channel calcium-influx assay described in the Examples.

### Pharmaceutical Compositions

In accordance with another aspect, the present invention provides a pharmaceutical composition comprising a compound of the invention, except the compounds specified in List 1 are not excluded, or a pharmaceutically acceptable salt thereof, and a pharmaceutically acceptable excipient.

It may be that the pharmaceutical composition comprises a compound selected from a compound according to any of formulae (I) to (XXIV), and List 1, or a pharmaceutically acceptable salt thereof.

It may be that the pharmaceutical composition comprises a compound selected from a compound according to any of formulae (I) to (XXIV), or a pharmaceutically acceptable salt thereof, with the proviso that the compounds specified in List 1 are excluded.

It may be that the pharmaceutical composition comprises a compound selected from List 1.

Conventional procedures for the selection and preparation of suitable pharmaceutical compositions are described in, for example, “Pharmaceuticals—The Science of Dosage Form Designs”, M. E. Aulton, Churchill Livingstone, 1988.

The compositions of the invention may be in a form suitable for oral use (for example as tablets, lozenges, hard or soft capsules, aqueous or oily suspensions, emulsions, dispersible powders or granules, syrups or elixirs), for sublingual use, for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intraperitoneal dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of a condition is an amount sufficient to symptomatically relieve in a warm-blooded animal, particularly a human the symptoms of the condition or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from 0.1 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, a daily dose selected from 0.1 mg/kg to 100 mg/kg, 1 mg/kg to 75 mg/kg, 1 mg/kg to 50 mg/kg, 1 mg/kg to 20 mg/kg or 5 mg/kg to 10 mg/kg body weight is received, given if required in divided doses. In general, lower doses will be administered when a parenteral route is employed. Thus, for example, for intravenous, subcutaneous, intramuscular or intraperitoneal administration, a dose in the range, for example, 0.1 mg/kg to 30 mg/kg body weight may be suitable. Similarly, for administration by inhalation, a dose in the range, for example, 0.05 mg/kg to 25 mg/kg body weight may be suitable. When administered orally a total daily dose of a compound of the invention may be, for example, selected from: 1 mg to 1000 mg, 5 mg to 1000 mg, 10 mg to 750 mg or 25 mg to 500 mg. Typically, unit dosage forms will contain about 0.5 mg to 0.5 g of a compound of the invention. In a particular embodiment the compound of the invention is administered parenterally, for example by intravenous administration. In another particular embodiment the compound of the invention is administered orally.

### Therapeutic Uses and Applications

In this section describing therapeutic uses, applications and methods of treatment reference to “a compound of the invention” includes compounds according to any to any of formulae (I) to (XXIV), or a pharmaceutically acceptable salt thereof, except that the compounds in List 1 are not excluded. Thus “a compound of the invention” in this section may be a compound according to any to any of formulae (I) to (XXIV) and List 1, or a pharmaceutically acceptable salt thereof.

However, it is to be understood that in some embodiments in this section the compound of the invention may be a compound according to any to any of formulae (I) to (XXIV), or a pharmaceutically acceptable salt thereof, with the proviso that the compounds in List 1 are excluded. In other embodiments in this section the compound of the invention may be a compound selected from List 1, or a pharmaceutically acceptable salt thereof.

In accordance with another aspect, the present invention provides a compound of the invention, for use as a medicament.

A further aspect of the invention provides a compound of the invention, or a pharmaceutically acceptable salt thereof, for use in the prevention or treatment of a disease or medical disorder mediated by Cav2.3.

Also provided is a method of preventing or treating a disease or medical disorder mediated by Cav2.3 in a subject, the method comprising administering to the subject an effective amount of a compound of the invention, or a pharmaceutically acceptable salt thereof.

Also provided is the use of a compound of the invention, or a pharmaceutically acceptable salt thereof, for the manufacture of a medicament for the prevention or treatment of a disease or medical disorder mediated by Cav2.3.

In the following sections of the application reference is made to a compound of the invention, or a pharmaceutically acceptable salt thereof for use in the treatment of certain diseases or medical disorders. It is to be understood that any reference herein to a compound for a particular use is also intended to be a reference to (i) the use of the compound of the invention, or pharmaceutically acceptable salt thereof, in the manufacture of a medicament for the treatment of that disease or disorder; and (ii) a method for the treatment of the disease or disorder in a subject, the method comprising administering to the subject a therapeutically effective amount of the compound of the invention, or pharmaceutically acceptable salt thereof.

In certain embodiments the disease or medical disorder mediated by Cav2.3 is selected from: a neurodegenerative disease, a neurodevelopmental disorder, epilepsy, an endocrine disorder, cerebral vasospasm, and pain.

In certain embodiments there is provided a compound of the invention, or a pharmaceutically acceptable salt thereof, for use in the treatment of a disease or medical disorder selected from: a neurodegenerative disease, a neurodevelopmental disorder, epilepsy, an endocrine disorder, cerebral vasospasm, and pain.

### Neurodegenerative Diseases

In some embodiments the disease or medical disorder is a neurodegenerative disease. For example, the disease or medical disorder is selected from: Parkinson's disease, Alzheimer's disease, Huntington's disease, dystonia, amyotrophic lateral sclerosis (ALS), multiple sclerosis, and age-related neurodegeneration. In a particular embodiment a compound of the invention is for use in the treatment of Parkinson's disease.

It may be that a compound of the invention provides a neuroprotective effect in subjects with a neurodegenerative disease. Accordingly, a compound of the invention may be for use in the neuroprotective treatment of a neurodegenerative disease (e.g., Parkinson's disease). In some embodiments, a compound of the invention may be for use in preventing or delaying the onset of symptoms associated with a neurodegenerative disease. Thus, compound of the invention may be for use in preventing or reducing neurodegeneration associated with a neurodegenerative disease.

In certain embodiments a compound of the invention is for use in preventing or inhibiting degeneration of dopaminergic neurons in a subject with a neurodegenerative disease (e.g. Parkinson's disease). Accordingly, it may be that a compound of the invention is for use in the prevention or inhibition of degeneration of dopaminergic substantia nigra (SN) neurones in a subject with Parkinson's disease.

In certain embodiments a compound of the invention is for use in the treatment or prevention of one or more symptoms of a neurodegenerative disease. For example, a compound may be for use in the treatment or prevention of one or more symptoms of Parkinson's disease selected from: tremor, bradykinesia, dystonia, stiffness, balance, coordination, cognitive impairment, and speech impairment.

### Neurodevelopmental Disorders

In certain embodiments a compound of the invention is for use in the treatment of a neurodevelopmental disorder. In certain embodiments the neurodevelopmental disorder is selected from: CACNA1E Gain-of-function Syndrome (DEE69), CDKL5 Deficiency (DEE2), Fragile X syndrome, Down syndrome, Rett syndrome, Angelman syndrome, autism, motor disorders (e.g., developmental coordination disorder, stereotypic movement disorder and tic disorders), and attention deficit hyperactivity disorder (ADHD).

As discussed in the introduction, Cav2.3 channels are associated with developmental and epileptic encephalopathies (DEEs). The term “DEE” refers to a group a heterogeneous group of rare neurodevelopmental disorders, characterised by (a) early-onset seizures that are often intractable, (b) electroencephalographic abnormalities, (c) developmental delay or regression and (d) in some cases, early death. DEE is classified by the 2017 International League Against Epilepsy (ILAE) Classification of the Epilepsies as an epilepsy associated with developmental impairment that may be due to both the underlying etiology (developmental encephalopathy) and superimposed epileptic activity (epileptic encephalopathy) (Scheffer et al. ILAE classification of the epilepsies: position paper of the ILAE commission for classification and terminology. Epilepsia. 2017; 58:512-21).

In certain embodiments a compound of the invention is for use in the prevention or treatment of a developmental and epileptic encephalopathy. In certain embodiments a compound of the invention is for use in the prevention or treatment of a monogenic developmental and epileptic encephalopathy In certain embodiments a compound of the invention is for use in the treatment or prevention of CACNA1E Gain-of-function Syndrome (DEE69), CDKL5 Deficiency (DEE2), DEE9 (caused by mutation in the PCDH19 gene), DEE11 (SCN2A gain of function), DEE13 (SCN8A gain of function), Dravet syndrome (DEE6A) or a DEE caused by or associated with a loss of function of GABAa receptors (e.g. DEE19, DEE43, DEE45, DEE59, DEE74, DEE78, DEE79 or DEE92).

In one embodiment a compound of the invention is for use in the treatment or prevention of DEE is Dravet syndrome (DEE6A). In a particular embodiment a compound of the invention is for use in the treatment or prevention of CACNA1E Gain-of-function Syndrome (DEE69) or CDKL5 Deficiency (DEE2).

In another embodiment a compound of the invention is for use in the treatment of a DEE caused by or associated with a loss of function of GABAa receptors. For example a compound of the invention is for use in the treatment of a DEE selected from: DEE19, DEE43, DEE45, DEE59, DEE74, DEE78, DEE79 and DEE92.

The genetic phenotypes and clinical features of the DEEs described herein are set out in entry #30008 in the Online Mendelian Inheritance in Man@(OMIM) database (https://www.omim.org/about).

### Epilepsy

In certain embodiments a compound of the invention is for use in the treatment of epilepsy.

Epilepsy is a chronic brain disease in which unprovoked epileptic seizures are the predominant feature. Epileptic seizures can vary from brief and nearly undetectable to long periods of vigorous shaking. Epilepsy and its related syndromes may be classified according to whether seizures are partial or generalized, and whether the aetiology is idiopathic or symptomatic or cryptogenic. The term “epilepsy” comprises both generalized and focal forms, with generalized epilepsy affecting both hemispheres while focal epilepsy includes unifocal and multifocal disorders as well as seizures involving one hemisphere.

In certain embodiments a compound of the invention is for use in the treatment of an epilepsy selected from: idiopathic epilepsy, cryptogenic epilepsy and symptomatic epilepsy. Idiopathic epilepsy is epilepsy with no apparent cause. Cryptogenic epilepsy occurs when the cause of epilepsy in a subject has not been identified despite investigation. Symptomatic epilepsy is epilepsy with a known cause. Causes of symptomatic epilepsy include, for example, brain injury, a bacterial or viral infection (e.g. meningitis), stroke or a tumour.

In some embodiments a compound of the invention is for use in the treatment of an epilepsy syndrome. For example a compound of the invention may be for use in the treatment of an epilepsy syndrome selected from: childhood absence epilepsy, benign Rolandic epilepsy, Doose syndrome, Dravet syndrome, early myoclonic encephalopathy, epilepsy in infancy with migrating focal seizures, Jeavons syndrome, epilepsy with myoclonic absences, epilepsy with generalised tonic-clonic seizures, epileptic encephalopathy with continuous spike and wave during sleep, febrile illness-related epilepsy syndrome, genetic epilepsy with febrile seizures plus, West syndrome, juvenile absence epilepsy, juvenile myoclonic epilepsy, Landau-Kleffner syndrome, Lennox-Gastaut syndrome, myoclonic epilepsy of infancy, Ohtahara syndrome, Panayiotopoulos syndrome, progressive myoclonic epilepsies, reflex epilepsies, self-limited familial and non-familial neonatal-infantile seizures, Gastaut syndrome, sleep-related hypermotor epilepsy, and temporal lobe epilepsy.

In some embodiments a compound of the invention is for use in the treatment or prevention of drug-resistant epilepsy. Drug-resistant epilepsy (also known as “uncontrolled,” “intractable” or “refractory” epilepsy) refers to epilepsy that fails to respond to, or relapses following treatment with an anti-epileptic therapy. Accordingly, in subjects with drug-resistant epilepsy seizures persist despite treatment with one or more anti-epileptic therapies. For example, a subject may not respond to, or relapses after treatment with one or more anti-epileptic therapy (for example the subject does not respond to, or relapses after treatment with at least two anti-epileptic therapies). In certain embodiments the subject fails to respond to, or relapses after treatment with one or more anti-epileptic drug (AED), for example one or more of the AEDs listed herein in relation to combination therapies. The drug-resistant epilepsy may be any of the forms of epilepsy described herein that is, or has become resistant to treatment with one or more (e.g. at least two) anti-epileptic therapy. In some embodiments the drug-resistant epilepsy is a drug-resistant focal epilepsy.

In certain embodiments a compound of the invention is for use in preventing or treating seizures. Thus, in certain embodiments a compound of the invention is for use in preventing or treating an epileptic seizure. For example, a compound of the invention may reduce the occurrence of epileptic seizures, reduce the severity and/or duration of epileptic seizures, or reduce the frequency of seizures. In some embodiments a compound of the invention is for use in the prevention or treatment of partial, generalized, convulsive and non-convulsive seizures. In some embodiments a compound of the invention is for use in preventing or treating a seizure selected from: tonic-clonic, tonic, clonic, myoclonic, absence, and atonic seizures.

### Endocrine Disorders

In certain embodiments a compound of the invention is for use in the treatment of an endocrine disorder. For example, a compound of the invention may be for use in the treatment of an endocrine disorder selected from: diabetes (e.g., treating glucose-induced insulin release, glucose-mediated glucagon suppression, or glucose-mediated somatostatin-release), acromegaly, Addison's disease, Cushing's syndrome, Graves' disease, Hashimoto's thyroiditis, hyperthyroidism, hypothyroidism (underactive thyroid), and prolactinoma.

### Pain

In certain embodiments a compound of the invention is for use in the treatment or prevention of pain. In some embodiments a compound of the invention is for use in the treatment of chronic pain, inflammatory pain, neuropathic pain (e.g., peripheral neuropathic pain or central neuropathic pain), or nociceptive pain.

### Cerebral Vasospasm

Subjects which suffer a cerebral aneurism or aneurysmal subarachnoid haemorrhage (bleeding on the surface of the brain) often survive the initial trauma. However, often within a few days to two weeks subjects experience cerebral vasospasm, a constriction, or tightening, of arteries in the brain. Cerebral vasospasm restricts blood flow to the brain and may subsequently lead to the death of blood-starved brain tissue resulting in cerebral infarction. Expression of Cav2.3 may be increased following a cerebral aneurism or aneurysmal subarachnoid haemorrhage and may be implicated in cerebral vasospasm (Wang et al., supra). Accordingly, in some embodiments a compound of the invention is for use in the treatment or prevention of cerebral vasospasm. For example, a compound of the invention is for use in the treatment or prevention of cerebral vasospasm in a subject who has suffered a cerebral aneurism or aneurysmal subarachnoid haemorrhage. In some embodiments a compound of the invention is for use in the treatment or prevention of cerebral infarction.

### Selectivity

Without wishing to be bound by theory it is expected that the selective modulation of Cav2.3 will provide compounds with a desirable therapeutic effect whilst avoiding or minimising the side effects associated with a non-selective Cav2.3 antagonist.

In certain embodiments such selective compounds may be used in the treatment or prevention of any of the diseases or medical disorders described herein.

### Combination Therapies

The compounds of the invention may be used alone to provide a therapeutic effect. The compounds of the invention may also be used in combination with one or more additional therapeutic agents.

In some embodiments the additional therapeutic agent is selected from one or more of:
an anti-epileptic drug (AED), for example acetazolamide, benzodiazepine, cannabadiols, carbamazepine, clobazam, clonazepam, diazepam, eslicarbazepine acetate, ethosuximide, ethotoin, felbamate, fenfluramine, fosphenytoin, gabapentin, ganaxolone, huperzine A, lacosamide, lamotrigine, levetiracetam, nitrazepam, oxcarbazepine, perampanel, piracetam, phenobarbital, phenytoin, potassium bromide, pregabalin, primidone, retigabine, rufinamide, valproic acid, sodium valproate, soticlestat, stiripentol, tiagabine, topiramate, vigabatrin, or zonisamide.
a drug for the treatment of Parkinson's disease, for example a dopamine mimetic (substances which regulate/modulate the dopamine metabolism, e.g., levodopa or carbidopa); a dopamine receptor agonist (e.g. pramipexole, ropinirole, rotigotine or apomorphine); a monaminoxidase inhibitor, for example an MAO B inhibitor (e.g. selegiline, rasagiline or safinamide); a catechol O-methyltransferase (COMT) inhibitor (e.g. entacapone, opicapone or tolcapone); an anticholinergic (e.g. benztropine or trihexyphenidyl); adamantane; or an adenosine A2A receptor antagonist (e.g. istradefylline).

Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In some embodiments in which a combination treatment is used, the amount of the compound of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of the invention and an approved or otherwise published dosage range(s) of the other pharmaceutically active compound(s).

### Biological Assays

The effect of a compound of the invention on inhibiting calcium ion influx into cells via human Cav2.3 channels can be assessed using the human Cav2.3 channel calcium-influx assay described in the examples section. The effects of compounds of the invention inhibiting the function of Cav2.3 ion channels in-vitro can be assessed by, for example using whole cell patch clamp methods such as that described in the example section.

The effects of the compounds blocking R-type calcium current with whole cell patch clamp electrophysiology in substantia nigra dopamine neurons in an ex vivo brain slice can be assessed using the methods described in Siller et al., Elife, 11:e67464 (2022) http://doi_org/10.7554/eLife. 67464.

Effects of the compounds on diseases or medical disorders mediated by Cav2.3 may be assessed using suitable in-vitro and in-vivo models for such diseases and medical disorders. For example, the effects of a compound of the invention on Parkinson's disease may be assessed using the methods and models described in WO2018/228692. Other suitable models for Parkinson's disease include, for example, the MitoPark mouse model described in Galter et al. (Genes Brain Behav. 2010 Mar. 1; 9(2): 173-181); and the SNCA-OVX transgenic mouse model described in Janezic et al. (Proceedings of the National Academy of Sciences, 2013, September, 201309143 DOI: 10.1073/pnas. 1309143110).

Suitable models for testing a compound of the invention for the treatment of seizures or epilepsy include, for example, one or more of the models described in Löscher (Seizure, 2011, (20), 359-368). Alternatively a compound of the invention may be tested in the Maximal Electroshock Stimulation (MES) model described in Kehne et al, Neurochemistry Research 42:1894-1903 (2017); https://doi.org/10.1007/s11064-017-2275-z.

### Synthesis

In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl or trifluoroacetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃·OEt₂. A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, or sodium hydroxide, or ammonia. Alternatively, an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

### General Synthetic Routes

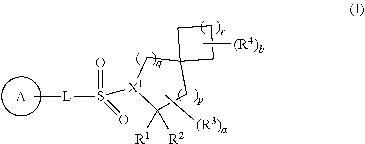

Compounds of formula (I) can generally be prepared by reacting a compound of formula (Ia)

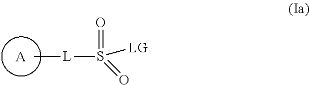

wherein ring A and L are as defined above for any of formulae (I) to (XXIV), and LG is a leaving group, with a compound of formula (Ib):

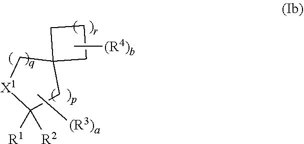

wherein, X¹, R¹, R², R³, R⁴, a, b, p, q, and r are as defined above for any of formulae (I) to (XXIV), except that any functional group is protected if necessary;

and optionally thereafter carrying out one or more of the following procedures:

converting a compound of formula (I) into another compound of formula (I); removing any protecting groups; forming a pharmaceutically acceptable salt; and/or preparing stereochemically isomeric forms thereof.

Alternatively, compounds of formula (I) can generally be prepared by reacting a compound of formula (Ia) with a compound of formula (Ic):

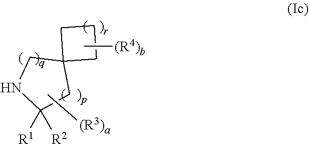

wherein, the meanings of X¹, R¹, R², R³ and R⁴, a, b, p, q, and r are as defined above for any of formulae (I) to (XXIV), except that any functional group is protected if necessary;

and optionally thereafter carrying out one or more of the following procedures:

converting a compound of formula (I) into another compound of formula (I); removing any protecting groups; forming a pharmaceutically acceptable salt; and/or preparing stereochemically isomeric forms thereof.

The reaction of a compound of formula (Ia) with a compound of formula (Ic), may be carried out in a at least one reaction-inert solvent, for example, DCM, DCE, THF, 1,4-dioxane, acetonitrile and the like and optionally in the presence of at least one suitable base thereof. Non-limiting examples of such reaction promoters include DIPEA, TEA, pyridine, NMM, 2,6-lutidine, DMAP or a functional derivative thereof.

LG in the compound of formula (Ia) is an appropriate leaving group, for example, halo, such as fluoro, chloro, bromo and the like.

The reaction of a compound of formula (Ia) with a compound of formula (Ic), may be performed in a reaction-inert solvent, for example, DCM, THF, acetonitrile, pyridine and optionally in the presence of a suitable base such as, sodium carbonate, potassium carbonate or trimethylamine, DIPEA, pyridine. Stirring may enhance the rate of the reaction. The reaction may conveniently be carried out at a temperature ranging between water freezing temperature (0° C.) and the reflux temperature of the reaction mixture.

Compounds of formula (Ia) can be prepared according to the following scheme (General Scheme 1):

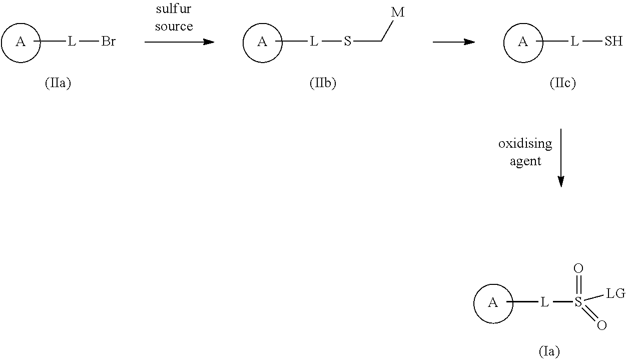

Sulfide compound (IIb) can be obtained by reaction of the respective bromo derivatives (IIa) in a sulfur-carbon bond forming reaction in an inert atmosphere in the presence of a metal catalyst such as Pd catalyst, for example, Pd(OAc)₂, PdCl₂(MeCN)₂, PdCl₂(PPh₃)₂, BnPdCl(PPh₃)₂, etc. Examples of such reactions include reaction of aromatic bromo, chloro or iodo compound with an appropriate sulfur compound as an example, but not limited to, phenylmethanethiol, (4-methoxyphenyl)methanethiol and 2-ethylhexyl 3-mercaptopropanoate. The reaction may be performed in a suitable solvent, such as, 1,4-dioxane, toluene, benzene, DMF, DME, DMA solution preferably at temperatures between rt and 150° C.

Sulfide compound (IIc) can be obtained by the reaction of the respective sulfide derivatives (IIb) in a sulfur-carbon bond cleavage reaction in an inert atmosphere in the presence of a base. Suitable bases for this type of conversion used include, for example, NaOEt, NaOMe, K₂CO₃, Na₂CO₃, tBuONa and tBuOK. Suitable solvents for this type of conversion include, for example, EtOH, MeOH, THF, DCE, DCM, MeCN preferably at temperatures between −78° C. and RT.

Sulfonyl chloride (Ia) can be obtained from the respective thiol derivatives (IIc) via a sulfur-oxygen and sulfur-chlorine bond forming reaction. Non-limiting examples of such reaction include reaction with:
halogen or a chlorine source in the presence of a such as chlorine gas, NaOCl, NCS, NBS, 1,3-Dibromo-5,5-Dimethylhydantoin, oxone, isocyanuric chloride or a derivative thereof;
an oxygen source such as ammonium nitrate, an aqueous solution of AcOH, HCl and HBr and oxygen as a terminal oxidant was developed in that process.
   The reaction may be performed in a suitable solvent, such as, DCM, tetrahydrofuran, acetic acid, diethyl ether, toluene preferably at temperatures between −20° C. and RT.

Compounds of formula (Ib) and (Ic) are known in the art or can be prepared following the processes reported in the examples.

Accordingly, compounds of formula (Ic), wherein, X¹, R¹, R², R³, R⁴, a, b, p, q, and r are as defined above for any of formulae (I) to (XXIV), can be prepared through four steps procedure as mentioned in the following scheme (General Scheme 2):

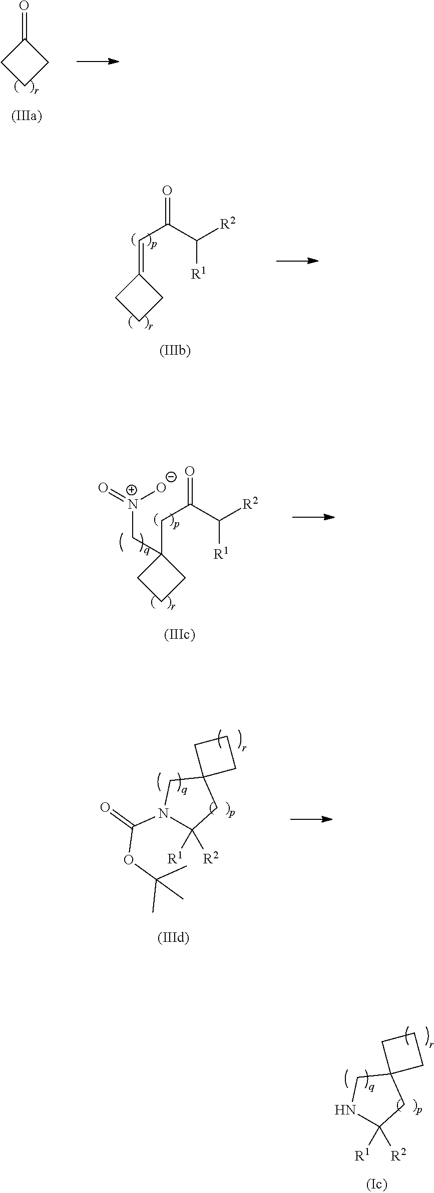

Reaction of compounds of formula (IIIa) may be carried out with a Wittig reagent, preferably with or without base under thermal condition preferably at temperature between 50° C. and 150° C. to give the compounds of formula (IIIb). The base for this type of conversion is, for example, K₂CO₃, tBuONa, nBuLi, NaOH or NaH. The solvent may be, for example, THF, silicon oil and other aprotic solvent.

Compounds of the formula (IIIc) can be obtained by reaction of compounds of formula (IIIb) with a nitroalkanes reagent, preferably with or without base under thermal condition and under inert atmosphere preferably at temperatures between 50° C. and 110° C. Base source of this type of conversion generally used are TBAF, TEA and DIPEA in the presence of a solvent such as THF.

Cyclic N—BOC protected compound (IIId) can be obtained by a two-step procedure in one pot, i.e., reduction of nitro compound to a primary amine followed by reductive amination in the presence of (BOC)₂O. Thus, primary amines can be obtained by reduction of the respective nitro derivatives (IIIc) in a nitrogen-hydrogen bond forming reaction. Non-limiting examples of such reaction include reduction with:
hydrogen or a hydrogen source in the presence of a metal such as platinum, nickel or palladium or a derivative thereof such as Raney-Ni, platinum oxide, palladium oxide, palladium on carbon, as catalyst.
   The reaction may be performed in a suitable solvent, such as methanol, tetrahydrofuran, or methanolic ammonia solution preferably at temperatures between 0° C. and RT.

Reductive amination may be carried out in the presence of a hydride source such as, lithium borohydride, sodium cyanoborohydride, sodium triacetoxyborohydride, boron hydride or a functional derivative thereof. The reaction may be performed in a suitable solvent, such as methanol, tetrahydrofuran, acetic acid, toluene preferably at temperatures between 0° C. and RT and in the presence of a protecting group, such as, BOC₂O to obtain Boc-protected spiro amines of formula (IIId).

Deprotection of compounds of formula (IIId) with a suitable acid, preferably 4M HCl in 1,4-dioxane, can provide compounds of formula (Ic).

### EXAMPLES

### Abbreviations

Ac—acetyl
BINAP—2,2′-bis(diphenylphosphino)-1,1′-binaphthyl
Bn—benzyl
Boc—tert-butoxycarbonyl
CBz—benzyloxycarbonyl
CPME—cyclopentyl methyl ether
dba—dibenzylideneacetone
DCM—dichloromethane
DIEA—N,N-diisopropylethylamine
DIPA—diisopropylamine
DMAc—dimethylacetamide
DMF—N,N-dimethylformamide
DMSO—dimethylsulfoxide
EDCI—1-ethyl-3-(3-dimethylaminopropyl)carbodiimide hydrochloride salt
ee—enantiomeric excess
eq.—equivalents
Ghosez's Reagent—1-chloro-N,N-2-trimethyl-1-propenylamine
HATU—1-[bis(dimethylamino)methylene]-1H-1,2,3-triazolo[4,5-b]pyridinium 3-oxide hexafluorophosphate
HOAt—1-hydroxy-7-azabenzotriazole
HPLC—high performance liquid chromatography
IPA—isopropanol
KHMDS—potassium bis(trimethylsilyl)amide
LC-MS—liquid chromatograph-mass spectrometer
LDA—lithium diisopropylamide
mCPBA—3-chloroperbenzoic acid
MeCN—acetonitrile
MS—mass spectrometry
Ms—mesyl
MTBE—methyl tert-butyl ether
MW—microwave
NBS—N-bromosuccinimide
NMM—N-methylmorpholine
NMP—N-methyl-2-pyrrolidone
NMR—nuclear magnetic resonance
o/n—overnight
Pd/C—palladium-on-carbon
Piv—pivaloyl
Prep—preparative
pTSA—p-toluene sulfonic acid
Py—pyridine
rt—retention time
RT—room temperature
RM—reaction mass
SFC—supercritical fluid chromatography
SEM—trimethylsilylethoxymethyl
SPE—solid phase extraction
Su—succinimide
TBAB—tetrabutylammonium bromide
TBAF—tetrabutylammonium fluoride
TEA—triethylamine
TFA—trifluoroacetic acid
TFAA—trifluoroacetic anhydride
THF—tetrahydrofuran
TLC—Thin-layer chromatography

### Reagents and Conditions

Unless syntheses are given, reagents and starting materials were obtained from commercial sources. All reactions, unless otherwise stated, were carried out under an inert atmosphere of either nitrogen or argon.

### Compound Names

New compounds were named using ChemDraw Ultra 14.0 from CambridgeSoft. Other compounds, particularly commercial reagents, either use names generated by ChemDraw Ultra 14.0 or names commonly found in online databases and catalogues.

### Analytical Methods

Method 1: (K84-3 min):

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C8 column (1.7 μm, 50×2.1 mm) with a flow rate of 0.800 ml/min. Two mobile phases were used, mobile phase A: 0.05% HCOOH in water; mobile phase B: 0.05% HCOOH in ACN:Water (90:10)], and they were employed to run a gradient condition from 10% B for 0.75 minutes, from 10% to 50% in 0.25 minutes, and from 50% to 98% in 1.00 minutes, 98% B for 0.25 minutes and then 10% B in 0.35 minutes and hold these conditions for 0.40 minutes in order to re-equilibrate the column (Total Run Time 3.00 minutes). An injection volume of 0.5 μl was used.

Method 2: (K70/71/55/63 3 min):

The HPLC measurement was performed using Waters Acquity UPLC comprising a binary pump with degasser, a sample manager, a column oven (set at 50° C.), a diode-array detector DAD and a column as specified in the respective methods below. Flow from the column was split to a MS spectrometer. The MS detector (Waters ZQ SQD) was configured with an electrospray ionization source. Mass spectra were acquired by scanning from 100 to 1000 in 0.40 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.1 Software. Reversed phase HPLC was carried out on a Waters YMC Triart C18 column (3 μm, 33×2.1 mm) with a flow rate of 1.00 ml/min. Two mobile phases were used, mobile phase A: 0.05% HCOOH in water; mobile phase B: 0.05% HCOOH in ACN:Water (90:10)], and they were employed to run a gradient condition from 2% B for 0.75 minutes, from 2% to 10% in 0.25 minutes, and from 10% to 98% in 1.00 minutes, 98% B for 0.50 minutes and then 2% B in 0.40 minutes and hold these conditions for 0.10 minutes in order to re-equilibrate the column (Total Run Time 3.00 minutes). An injection volume of 0.5 to 3 μl was used (Depending on the sample concentration).

Method 3: (K03/04/05/06/07/08/72/78 5 min):

The HPLC measurement was performed using Shimadzu HPLC comprising a binary pump with degasser, a sample manager a dual channel UV detector and a column as specified in the respective methods below. Flow from the column was split to a MS spectrometer. The MS detector (Applied Biosystems AP12000/2000 Trap) was configured with an electrospray ionization source. Mass spectra were acquired by scanning from 100 to 800 in 0.40 second. The ion spray voltage 5500 V in positive and 4500 V in negative ionization mode and the source temperature was maintained at 300° C. and Declusturing Potential 8-50 V depending on compound. Data acquisition was performed with Analyst 1.6.3 Software. Reversed phase HPLC was carried out on a Waters Xbridge C18/Agilent Zorbax C18 column (5 μm, 50×4.6 mm) with a flow rate of 1.20 ml/min. Two mobile phases were used, mobile phase A: 10 mm Ammonium Acetate in water; mobile phase B: ACN, and they were employed to run a gradient condition from 10% B to 30% B in 1.50 minutes, and from 30% to 90% in 1.50 minutes, 90% B for 1.00 minutes and 10% B in 1.00 minutes and hold these conditions for 0.10 minutes. Pre run Equilibration Time 0.50 min (Total Run Time 5.10 minutes). An injection volume of 1 μl to 3 μl was used (Depending on the sample concentration).

Method 4: (K70/71/55/63 12 min):

The HPLC measurement was performed using Waters Acquity UPLC comprising a binary pump with degasser, a sample manager, a column oven (set at 50° C.), a diode-array detector DAD and a column as specified in the respective methods below. Flow from the column was split to a MS spectrometer. The MS detector (Waters ZQ SQD) was configured with an electrospray ionization source. Mass spectra were acquired by scanning from 100 to 1000 in 0.40 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.1 Software. Reversed phase HPLC was carried out on a Waters YMC Triart C18 column (3 μm, 33×2.1 mm) with a flow rate of 1.00 ml/min. Two mobile phases were used, mobile phase A: 0.05% HCOOH in water; mobile phase B: 0.05% HCOOH in ACN:Water (90:10)], and they were employed to run a gradient condition from 5% B for 1.00 minutes, from 5% to 50% in 4.00 minutes, and from 50% to 90% in 3.00 minutes, 90% B for 2.00 minutes and then 5% B in 1.50 minutes and hold these conditions for 0.50 minutes in order to re-equilibrate the column (Total Run Time 12.00 minutes). An injection volume of 0.5 to 3 μl was used (Depending on the sample concentration).

### Method 5: (K83 3 Min):

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Xbridge C18 column (3.5 μm, 50×3 mm) with a flow rate of 1.20 ml/min. Two mobile phases were used, mobile phase A: 5 Mm NH4oAc in water; mobile phase B: 5 Mm NH4oAc in ACN:Water (90:10)], and they were employed to run a gradient condition from 5% B for 0.75 minutes, from 5% to 30% in 0.25 minutes, and from 30% to 98% in 1.00 minutes, 98% B for 0.25 minutes and 5% B in 0.50 minutes and hold these conditions for 0.25 minutes in order to re-equilibrate the column (Total Run Time 3.00 minutes). An injection volume of 0.5 μl was used.

### Method 6: (K84 12 Min):

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C8 column (1.7 μm, 50×2.1 mm) with a flow rate of 0.800 ml/min. Two mobile phases were used, mobile phase A: 0.05% HCOOH in water; mobile phase B: 0.05% HCOOH in ACN:Water (90:10)], and they were employed to run a gradient condition from 5% B for 1.00 minutes, from 5% to 50% in 4.00 minutes, and from 50% to 90% in 3.00 minutes, 90% B for 2.00 minutes and then 5% B in 1.50 minutes and hold these conditions for 0.50 minutes in order to re-equilibrate the column (Total Run Time 12.00 minutes). An injection volume of 0.5 μl was used.

### Method 7: (K79 3 Min):

The HPLC measurement was performed using Agilent 1260 Infinity II UPLC comprising a quaternary pump with degasser, a sample manager, a column oven (set at 50° C.), a diode-array detector DAD and a column as specified in the respective methods below. Flow from the column was split to a MS spectrometer. The MS detector (Agilent SQD) was configured with an electrospray ionization source. Mass spectra were acquired by scanning from 100 to 1200 in 0.20 second. The capillary needle voltage was 4.00 kV in positive and negative ionization mode and the source temperature was maintained at 350° C. Nitrogen was used as the desolvation gas, the flow was 12 L/Min. Data acquisition was performed with Open Lab CDS. Reversed phase HPLC was carried out on a YMC Triart C18 column (3 μm, 33×2.1 mm) with a flow rate of 1.00 ml/min. Two mobile phases were used, mobile phase A: 0.1% HCOOH in water; mobile phase B: 0.1% HCOOH in ACN, and they were employed to run a gradient condition from 2% B for 0.50 minutes, from 2% to 30% in 0.50 minutes, and from 30% to 98% in 1.00 minutes, 98% B for 0.25 minutes and then 2% B in 0.75 minutes (Total Run Time 3.00 minutes). An injection volume of 0.5 μl was used.

### Method 8: (K83 3 Min-BEH):

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C18 column (1.7 μm, 30×2.1 mm) with a flow rate of 1.20 ml/min. Two mobile phases were used, mobile phase A: 5 Mm NH4oAc in water; mobile phase B: 5 Mm NH4oAc in ACN:Water (90:10)], and they were employed to run a gradient condition from 2% B for 0.50 minutes, from 2% to 98% in 1.00 minutes, 98% B for 1.00 minutes and 2% B in 0.25 minutes and hold these conditions for 0.25 minutes in order to re-equilibrate the column (Total Run Time 3.00 minutes). An injection volume of 0.5 μl was used.

### Method 9: (K83 5 Min)-OLD:

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Xbridge C18 column (5 μm, 50×4.6 mm) with a flow rate of 1.50 ml/min. Two mobile phases were used, mobile phase A: 5 Mm NH4oAc in water; mobile phase B: 5 Mm NH4oAc in ACN:Water (90:10)], and they were employed to run a gradient condition from 2% B for 0.75 minutes, from 2% to 15% in 0.50 minutes, from 15% to 70% in 1.25 minutes and from 70% to 98% in 1.25 minutes, 98% B for 0.75 minutes and 2% B in 0.50 minutes and hold these conditions for 0.10 minutes in order to re-equilibrate the column (Total Run Time 5.10 minutes). An injection volume of 0.5 μl was used.

### Method 10: (K83 12 Min)-OLD:

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Xbridge C18 column (5 μm, 50×4.6 mm) with a flow rate of 1.20 ml/min. Two mobile phases were used, mobile phase A: 5 Mm NH4oAc in water; mobile phase B: 5 Mm NH4oAc in ACN:Water (90:10)], and they were employed to run a gradient condition from 2% B for 1.00 minutes, from 2% to 50% in 4.00 minutes, from 50% to 90% in 3.00 minutes and 90% B for 2.00 minutes and 2% B in 2.00 minutes and hold these conditions for 0.10 minutes in order to re-equilibrate the column (Total Run Time 12.10 minutes). An injection volume of 0.5 μl was used.

### Method 11:

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Luna Omega Polar C18 column (3 μm, 100×4.6 mm) with a flow rate of 1.00 ml/min. Two mobile phases were used, mobile phase A: 0.05% HCOOH in water; mobile phase B: 0.05% HCOOH in ACN:Water (90:10)], and they were employed to run a gradient condition from 2% B for 1.00 minutes, from 2% to 50% in 4.00 minutes, and from 50% to 95% in 4.00 minutes, 95% B for 3.00 minutes and then 5% B in 0.50 minutes. (Total Run Time 12.50 minutes). An injection volume of 0.5 μl was used.

### Method 12:

The HPLC measurement was performed using Shimadzu HPLC comprising a binary pump with degasser, a sample manager a dual channel UV detector and a column as specified in the respective methods below. Flow from the column was split to a MS spectrometer. The MS detector (Applied Biosystems AP12000/2000 Trap) was configured with an electrospray ionization source. Mass spectra were acquired by scanning from 100 to 800 in 0.40 second. The ion spray voltage 5500 V in positive and 4500 V in negative ionization mode and the source temperature was maintained at 300° C. and Declusturing Potential 8-50 V depending on compound. Data acquisition was performed with Analyst 1.6.3 Software. Reversed phase HPLC was carried out on a Waters Xbridge C18/Agilent Zorbax Ext C18 column (5 μm, 100×4.6 mm) with a flow rate of 1.00 ml/min. Two mobile phases were used, mobile phase A: 10 mm Ammonium Acetate in water); mobile phase B: ACN, and they were employed to run a gradient condition from 50% B for 2.00 min, from 50% B to 95% B in 6.00 minutes, and 95% B for 3.00 minutes and 50% B in 3.00 minutes and hold these conditions for 4.00 minutes. Pre run Equilibration Time 4.00 min (Total Run Time 18.00 minutes). An injection volume of 1 μl to 3 μl was used (Depending on the sample concentration).

### Method 13 (K84 3 Min)-OLD

The HPLC measurement was performed using Waters Acquity H Class UPLC comprising a quaternary pump with degasser, a sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a YMC Triart C18 column (3 μm, 33×2.1 mm) with a flow rate of 1.00 ml/min. Two mobile phases were used, mobile phase A: 0.05% HCOOH in water; mobile phase B: 0.05% HCOOH in ACN:Water (90:10)], and they were employed to run a gradient conditions from 2% B for 0.75 minutes, from 2% to 10% in 0.25 minutes, and from 10% to 98% in 1.00 minutes, 98% B for 0.50 minutes and then 2% B in 0.40 minutes and hold these conditions for 0.10 minutes in order to re-equilibrate the column (Total Run Time 3.00 minutes). An injection volume of 0.5 μl was used.

### Method 14 (K84 3 Min)-OLD BEH POLAR

The HPLC measurement was performed using Waters Acquity H Class UPLC comprising a quaternary pump with degasser, an sample manager, a column oven (set at 50° C.), a diode-array detector DAD and a column as specified in the respective methods below. Flow from the column was split to a MS spectrometer. The MS detector (Waters SQ Detector 2) was configured with an electrospray ionization source. Mass spectra were acquired by scanning from 160 to 1200 in 0.20 second. The capillary needle voltage was 3.50 kV in positive and negative ionization mode and the source temperature was maintained at 150° C. Nitrogen was used as the desolvation gas, the flow was 750 L/Hour. Data acquisition was performed with Mass Lynx 4.2 Software. Reversed phase HPLC was carried out on a Waters Acquity BEH C8 column (1.7 μm, 50×2.1 mm) with a flow rate of 1.00 ml/min. Two mobile phases were used, mobile phase A: 0.05% HCOOH in water; mobile phase B: 0.05% HCOOH in ACN:Water (90:10)], and they were employed to run a gradient conditions from 2% B for 0.75 minutes, from 2% to 10% in 0.25 minutes, and from 10% to 98% in 1.00 minutes, 98% B for 0.50 minutes and then 2% B in 0.40 minutes and hold these conditions for 0.10 minutes in order to re-equilibrate the column (Total Run Time 3.00 minutes). An injection volume of 0.5 μl was used.

### NMR

All NMR spectra were obtained using Bruker Avance 400 MHz spectrometers running Topspin Software.

### GCMS

### GCMS-Method-1:

GC-MS was taken on Agilent 6890 and 5973 N MSD series instrument.

Column: HP-5MS (30×250 μm×0.25 μm)

### Carrier Gas:—Helium

### Inlet Temperature: 250° C.

Split ratio: 5:1
Carrier Gas flow: 1.0 ml/min

### Solvent Delay: 3 min

Mass range: 50 to 550 amu
Injection volume: 1 ul

### Ramp Profile:

Oven temperature initial from 100° C. held for 2 min then, 310° C. increasing at the rate of 35° C. held for 6 min. Total run time is 14 min.

### Gcms-Method-2:

GC-MS was taken on Agilent 7890B and 5977B MSD series instrument.

Column: HP-5MS (30×250 μm×0.25 μm)

### Carrier Gas:—Helium

### Inlet Temperature: 250° C.

Split ratio: 20:1
Carrier Gas flow: 1.0 ml/min

### Ramp Profile:

Oven temperature initial from 60° C. held for 2 min then, 100° C. increasing at the rate of 20° C. held for 2 min, 310° C. increasing at the rate of 40° C. held for 4 min. Total run time is 15.25 min SFC

Supercritical fluid chromatography (SFC) analysis was performed on a WATERS SFC-analytical instrument. Column: Chiralpak IG 250×4.6 mm, particle size 5 μm. Method: mobile phase: A: carbon dioxide, mobile phase B: Hexane/IPA/Methanol 2/1/1 (0.3% Isopropyl amine), with isocratic flow 4.0 mL/min; 20% of B; wavelength: 240 nm.

### Synthesis of Intermediate 1.6 [Spiro amine-1]

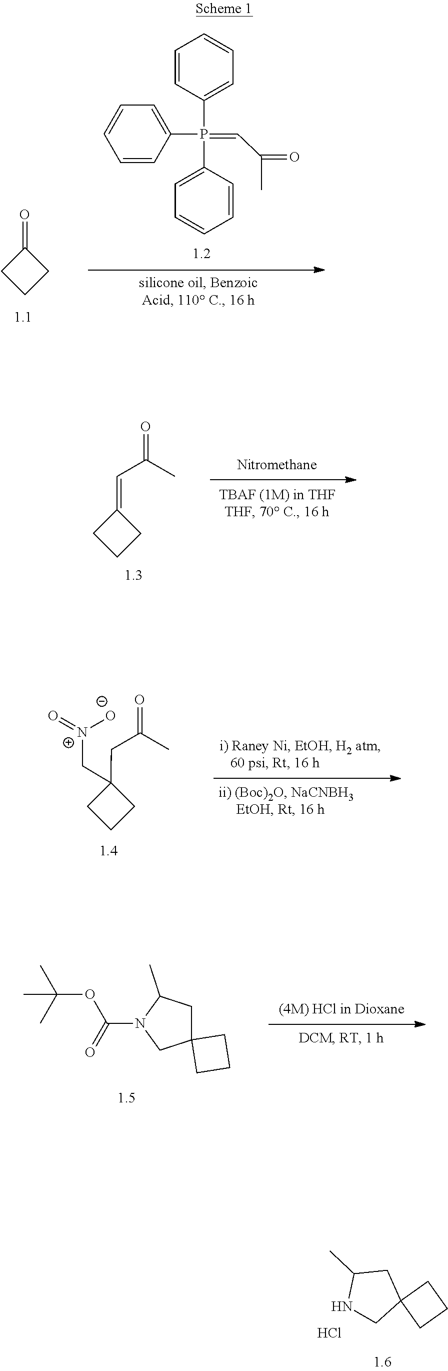

### 1-Cyclobutylidenepropan-2-one 1.3

The suspension of acetylmethylene-triphenylphosphorane 1.2 (27.2 g, 85.5 mmol) in silicone oil (60 mL) was stirred at 110° C. Benzoic acid (1.09 g, 0.86 mmol) was added to the suspension followed by cyclobutanone 1.1 (5 g, 81.4 mmol). The reaction mixture was heated at 110° C. for 16 h. Intermediate 1.3 was then distilled out as colourless oil (6.2 g, 69% yield). ¹H NMR (400 MHz, DMSO-d₆): δ 5.91-5.84 (m, 1H), 3.08 (t, 2H), 2.83 (t, 2H), 2.09 (s, 3H), 2.08-2.00 (m, 2H).

### 1-(1-(Nitromethyl)cyclobutyl)propan-2-one 1.4

To a stirred solution of intermediate 1.3 (3.0 g) in THF (6 mL) nitromethane (3 mL) was added, followed by TBAF solution (1M in THF) (32.7 mL) and the reaction mixture was stirred at 70° C. for 16 h under inert atmosphere. The volatiles were evaporated under reduced pressure. The reaction mixture was diluted with water and extracted with diethyl ether (3×25 mL). The combined organic part was washed with cold water (2×10 mL), dried over anhydrous Na₂SO₄ and concentrated under reduced pressure. The crude was purified by column chromatography over silica gel using 15% ethyl acetate in hexane provided 1.4 (2.9 g, 62% yield). ¹H NMR (400 MHz, DMSO-d₆): δ 4.73 (s, 2H), 2.84 (s, 2H), 2.07 (s, 3H), 2.07-1.96 (m, 2H), 1.96-1.80 (m, 4H).

### tert-Butyl 7-methyl-6-azaspiro[3.4]octane-6-carboxylate 1.5

Intermediate 1.4 (3.0 g) was dissolved in EtOH (70 mL) and Raney Ni was added to it. It was then stirred under hydrogen atm. at 60 psi pressure at RT for 16 h. The RM was filtered through celite bed. To the filtrate Na BH₃CN (1.38 g, 29.2 mmol) was added and the reaction mixture was stirred at RT for 16 h. The volatiles were evaporated under reduced pressure and the crude was diluted with water. It was then extracted with ethyl acetate and the organic part was washed with water and brine. The solution was dried over Na₂SO₄ and evaporated under reduced pressure. The crude was further purified by column chromatography over silica gel using 15% ethyl acetate in hexane provided title compound 1.5 as a light-yellow oil (1.5 g, 57% yield).

### 7-Methyl-6-azaspiro[3.4]octane hydrochloride 1.6

Intermediate 1.5 (1 g, 4.439 mmol) was dissolved in DCM (5 mL) and 4M HCl in dioxane (1 mL) was added at 5° C. The mixture was then stirred at RT for 1 h. The volatiles were evaporated under reduced pressure to provide title compound 1.6 as brown solid (1.1 g, quantitative yield). This hydrochloride salt was used for the following reactions. ¹H NMR (400 MHz, DMSO-d₆): δ 3.60-3.43 (m, 1H), 3.21-3.10 (m, 2H), 2.21 (dd, 1H), 2.17-1.86 (m, 4H), 1.88-1.75 (m, 2H), 1.58 (dd, 1H), 1.47 (s, 2H), 1.29 (d, 3H).

### Example-1

### 6-((7-Ethyl-6-azaspiro[3.4]octan-6-yl)sulfonyl)-2-methylbenzo[d]thiazole

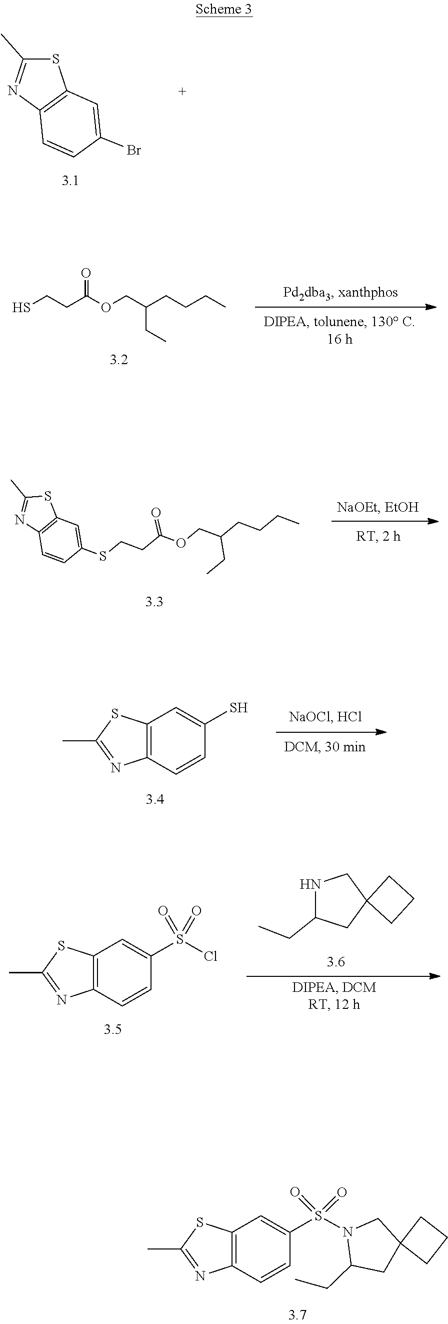

### 2-Ethylhexyl 3-((2-methylbenzo[d]thiazol-6-yl)thio)propanoate 3.3

A solution of 6-bromo-2-methylbenzo[d]thiazole 3.1 (400 mg, 1.754 mmol) and 2-ethylhexyl 3-mercaptopropanoate 3.2 (383 mg, 1.754 mmol) in toluene (4 ml) was degasified by argon and DIPEA (0.61 mL, 3.507 mmol) was added to it. Xantphos (51 mg, 0.088 mmol) was added to it followed by Pd₂(dba)₃ (30 mg, 0.053 mmol) and the reaction mixture was then stirred at 130° C. for 16 h in a sealed tube. The reaction mixture was filtered, and the volatiles were evaporated under reduced pressure. The crude was then purified by column chromatography over silica gel using 1:9 ethyl acetate-hexane provided intermediate 3.3 as colourless oil (551 mg, 86%). ¹H NMR (DMSO-d₆, 400 MHz): δ 8.05 (s, 1H), 7.83 (d, 1H), 7.42 (d, 1H), 3.93 (d, 2H), 3.21 (t, 2H), 2.79 (s, 3H), 2.63 (t, 2H), 1.50 (s, 1H), 1.37-1.20 (m, 8H), 0.90-0.77 (m, 6H).

### 2-Methylbenzo[d]thiazole-6-thiol 3.4

Intermediate 3.3 (200 mg, 0.547 mmol) was dissolved in EtOH (3 mL) and NaOEt (0.7 mL, 1.647 mmol) was added to the reaction mixture at 0° C. under nitrogen atmosphere and was stirred for 2 h. The volatiles were evaporated under reduced pressure and the crude was extracted with DCM (2×10 mL). The organic part was washed with water (10 mL) and brine (5 mL), dried over MgSO₄. The solvent was evaporated under reduced pressure. The crude intermediate 3.4 (150 mg) was used in the next step without further purification.

### 2-Methylbenzo[d]thiazole-6-sulfonyl chloride 3.5

Intermediate 3.4 (150 mg, 0.784 mmol) was dissolved in DCM (5 mL) and 7.5 M HCl (2 mL) was added to the reaction mixture at −10° C. under nitrogen temperature. Then, NaOCl (225 mg, 3.921 mmol) was added drop wise to the reaction mixture at the same temperature and was stirred for 30 min. The reaction mass was then diluted with water (5 mL) and extracted with DCM (2×10 mL), the organic part was washed with water (5 mL) and brine (5 mL), dried over MgSO₄. The solvent was evaporated under reduced pressure. The crude intermediate 3.5 was used in the forwarding step without further purification.

### 6-((7-Ethyl-6-azaspiro[3.4]octan-6-yl)sulfonyl)-2-methylbenzo[d]thiazole (Example 1)

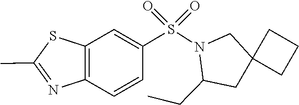 [SMILES: CCC1CC2(CCC2)CN1S(=O)(=O)c1ccc2nc(C)sc2c1]

Crude Intermediate 3.5 (110 mg, 0.444 mmol) was dissolved in DCM (5 mL) and DIPEA (0.27 mL, 1.568 mmol) was added to the reaction mixture at room temperature. Then the 7-ethyl-6-azaspiro[3.4]octane (109 mg, 0.784 mmol) in DCM (5 mL) was added dropwise to the reaction mixture at the same temperature and stirred it for 12 h. The reaction mass was then diluted with water and extracted with in DCM, the organic part was washed with water and brine, dried over MgSO₄. The solvent was evaporated under reduced pressure. The crude was purified by flash column chromatography to obtain 3.7 (Example 1) as colourless oil (54 mg, 19.6% yield, 98.9% purity). LC-MS: m/z found 351.15 [M+H]⁺, rt=2.14 min (Method 5) [Waters Xbridge C18 column (3.5 μm, 50×3 mm)]; ¹H NMR (DMSO-d₆, 400 MHz): δ 8.65 (d, 1H), 8.07 (d, 1H), 7.88 (dd, 1H), 3.54-3.42 (m, 1H), 3.32-3.22 (m, 2H), 2.86 (s, 3H), 2.00-1.72 (m, 4H), 1.71-1.44 (m, 4H), 1.19-0.99 (m, 2H), 0.86 (t, 3H).

### Example-2

### 6-(Benzylsulfonyl)-7-methyl-6-azaspiro[3.4]octane

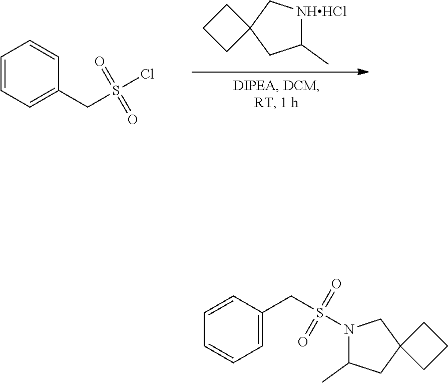

### General Method for Sulfonamide (Method A)

To a solution of 7-methyl-6-azaspiro[3.4]octane hydrochloride 1.6 (30 mg, 0.186 mmol) in DCM (1 ml) was added DIPEA (0.2 mL, 0.928 mmol) and the solution was cooled at 0° C. A solution of phenylmethanesulfonyl chloride (53 mg, 0.278 mmol) in DCM (1 mL) was added to it. The reaction mixture was stirred at RT for 1 h. The volatiles were evaporated under reduced pressure and the crude compound was purified by Reverse Phase Prep-HPLC and isolated as colourless Sticky Gum (25 mg, 26% yield, 99.55% purity). Prep-HPLC Method: Preparative HPLC was done on Waters auto purification instrument. Column name: YMC-Hydrosphere C18 (250×20 mm, 5μ) operating at ambient temperature and flow rate of 16 ml/min. Mobile phase: A=10 mM Ammonium Acetate in water, B=Acetonitrile; Gradient Profile: Mobile phase initial composition of 60% A and 40% B, then to 40% A and 60% B in 3 min, then to 26% A and 74% B in 18 min., then to 5% A and 95% B in 19 min., held this composition up to 23 min. for column washing, then returned to initial composition in 24 min. and held till 26 min.

LCMS: m/z found 280.2 [M+H]⁺, rt=8.33 min (Method 16) [Luna Omega Polar C18 column (3 μm, 100×4.6 mm)]; ¹H NMR (400 MHz, DMSO-d₆): δ 7.44-7.30 (m, 5H), 4.38 (s, 2H), 3.79-3.66 (m, 1H), 3.30-3.19 (m, 2H), 2.05 (dd, 1H), 2.02-1.77 (m, 6H), 1.59 (dd, 1H), 1.08 (d, 3H).

### Example-3

### 7-Methyl-6-((1-methyl-1H-imidazol-2-yl)sulfonyl)-6-azaspiro[3.4]octane

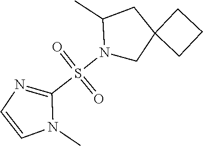 [SMILES: CC1CC2(CCC2)CN1S(=O)(=O)c1nccn1C]

The synthesis was performed as described in Method A using 7-methyl-6-azaspiro[3.4]octane hydrochloride 1.6 and 1-methyl-1H-imidazole-2-sulfonyl chloride. The compound was purified by Reverse Phase Prep-HPLC and isolated as white solid (11 mg, 26% yield, 99.77% purity). Prep-HPLC Method: Preparative HPLC was done on Waters auto purification instrument. Column name: YMC-Actus C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 80% A and 20% B, then to 65% A and 35% B in 3 min, then to 30% A and 70% B in 22 min., then to 5% A and 95% B in 23 min., held this composition up to 25 min. for column washing, then returned to initial composition in 26 min. and held till 28 min. LCMS: m/z found 270.0[M+H]⁺ rt=5.22 min (Method 10) [Xbridge C18 column (5 μm, 50×4.6 mm)]; ¹H NMR (400 MHz, DMSO-d₆): δ 7.42 (s, 1H), 7.06 (s, 1H), 4.01-3.88 (m, 1H), 3.87 (s, 3H), 3.44 (d, 1H), 3.38 (d, 1H), 2.10 (dd, 1H), 2.05-1.89 (m, 2H), 1.83-1.68 (m, 2H), 1.72-1.60 (m, 2H), 1.60-1.48 (m, 1H), 1.25 (d, 3H).

### Example 4

### 6-((3,4-Dihydro-2H-benzo[b][1,4]dioxepin-7-yl)sulfonyl)-7-methyl-6-azaspiro[3.4]octane

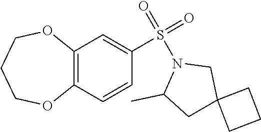 [SMILES: CC1CC2(CCC2)CN1S(=O)(=O)c1ccc2c(c1)OCCCO2]

The synthesis was performed essentially as described in Method A using 7-methyl-6-azaspiro[3.4]octane hydrochloride 1.6 and 3,4-dihydro-2H-benzo[b][1,4]dioxepine-7-sulfonyl chloride. The compound was purified by Reverse Phase Prep-HPLC and isolated as a light brown solid (19 mg, 18% yield, 99.74% purity). Prep-HPLC Method: Preparative HPLC was done on Waters auto purification instrument. Column name: YMC-Actus C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 60% A and 40% B, then to 40% A and 60% B in 3 min, then to 20% A and 80% B in 19 min., then to 5% A and 95% B in 20 min., held this composition up to 24 min. for column washing, then returned to initial composition in 25 min. and held till 28 min. LCMS: m/z found 338.15 [M+H]⁺, rt=2.00 min (Method 2) [YMC Triart C18 column (3 μm, 33×2.1 mm)]; ¹H NMR (400 MHz, DMSO-d₆): δ 7.38 (dd, 1H), 7.31 (d, 1H), 7.14 (d, 1H), 4.27-4.17 (m, 4H), 3.50 (q, 1H), 3.27 (d, 1H), 3.15 (d, 1H), 2.21-2.21 (m, 2H), 2.00-1.88 (m, 2H), 1.88-1.76 (m, 1H), 1.76-1.60 (m, 2H), 1.51 (dd, 1H), 1.41-1.30 (m, 1H), 1.27 (d, 4H).

### Example-5

### 6-((2,3-Dihydrobenzo[b][1,4]dioxin-6-yl)sulfonyl)-7-methyl-6-azaspiro[3.4]octane

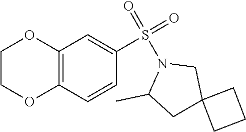 [SMILES: CC1CC2(CCC2)CN1S(=O)(=O)c1ccc2c(c1)OCCO2]

The synthesis was preformed essentially as described in Method A using 7-methyl-6-azaspiro[3.4]octane hydrochloride 1.6 and 2,3-dihydrobenzo[b][1,4]dioxine-6-sulfonyl chloride. The compound was purified by flash chromatography over silica gel using 3:7 ethyl acetate:hexane and isolated as white solid (50 mg, 32% yield, 99.05% purity). LCMS: m/z found 324.2 [M+H]+, rt=3.57 min (Method 3) [Zorbax C18 column (5 μm, 50×4.6 mm)]; 1H NMR (400 MHz, DMSO-d6): δ 7.28 (dd, 1H), 7.23 (d, 1H), 7.05 (d, 1H), 4.36-4.26 (m, 4H), 3.50 (q, 1H), 3.27 (d, 2H), 3.12 (d, 1H), 2.01-1.77 (m, 2H), 1.77-1.63 (m, 2H), 1.52 (dd, 1H), 1.45-1.29 (m, 2H), 1.27 (d, 3H).

### Example-6 and Example-7

### 6-((2,3-Dihydrobenzo[b][1,4]dioxin-6-yl)sulfonyl)-7-methyl-6-azaspiro[3.4]octane [EN-1] and 6-((2,3-dihydrobenzo[b][1,4]dioxin-6-yl)sulfonyl)-7-methyl-6-azaspiro[3.4]octane [EN-2]]

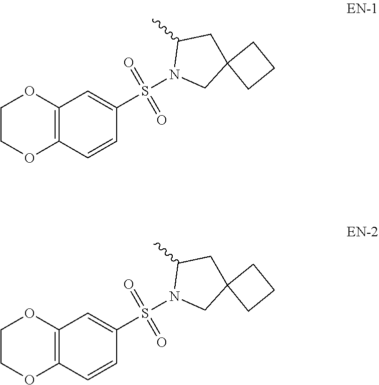

Chiral separation of 6-((2,3-dihydrobenzo[b][1,4]dioxin-6-yl)sulfonyl)-7-methyl-6-azaspiro[3.4]octane provided ˜25 mg each of both enantiomers as mentioned below. Chiral Separation Method: Chiral separation was done on Agilent 1200 series instrument. Column name: Chiralpak AS-H (250×21 mm), 5μ, operating at ambient temperature and flow rate of 21.0 mL/min. Mobile phase: the mixture of 70% Hexane and 30% Ethanol, held this isocratic mixture up to 25 min with wavelength of 245 nm.

Example 6 [EN-1]: White solid (25 mg, 18% yield, 99.61% purity). LCMS: m/z found 324.1 [M+H]⁺, rt=2.98 min (Method 9) [Xbridge C18 column (5 μm, 50×4.6 mm)]; ¹H NMR (400 MHz, DMSO-d₆): δ 7.32-7.20 (m, 2H), 7.05 (d, 1H), 4.38-4.25 (m, 4H), 3.50 (q, 1H), 3.27 (d, 1H), 3.12 (d, 1H), 1.98-1.77 (m, 3H), 1.74-1.62 (m, 2H), 1.52 (dd, 1H), 1.45-1.27 (m, 2H), 1.27 (d, 3H).

Example 7 [EN-2]: White solid (25 mg, 18% yield, 99.31%). LCMS: m/z found 324.1 [M+H]⁺, rt=2.98 min (Method 9) [Xbridge C18 column (5 μm, 50×4.6 mm)]; ¹H NMR (400 MHz, DMSO-d₆): δ 7.28 (dd, 1H), 7.23 (d, 1H), 7.05 (d, 1H), 4.36-4.26 (m, 4H), 3.57-3.43 (m, 1H), 3.28 (d, 1H), 3.13 (d, 1H), 2.01-1.78 (m, 3H), 1.77-1.62 (m, 2H), 1.52 (dd, 1H), 1.46-1.29 (m, 2H), 1.27 (d, 3H).

### Example-8

### 5-((7-Methyl-6-azaspiro[3.4]octan-6-yl)sulfonyl)thiophene-2-carbonitrile

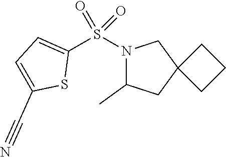 [SMILES: CC1CC2(CCC2)CN1S(=O)(=O)c1ccc(C#N)s1]

The synthesis was performed essentially as described in Method A using 7-methyl-6-azaspiro[3.4]octane hydrochloride 1.6 and 5-cyanothiophene-2-sulfonyl chloride. The compound was purified by Reverse Phase Prep-HPLC, and the compound (Example 8) was obtained as colourless sticky oil (42 mg, 59% yield, 99.68% purity). Preparative HPLC method: Preparative HPLC was done on Waters auto purification instrument. Column name: YMC-Actus C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 70% A and 30% B, then to 35% A and 65% B in 3 min, then to 10% A and 90% B in 22 min., then to 5% A and 95% B in 23 min., held this composition up to 25 min. for column washing, then returned to initial composition in 26 min. and held till 28 min. LCMS: m/z found 297.1 [M+H]⁺, rt=2.35 min (Method 14) [Acquity BEH C8 column (1.7 μm, 50×2.1 mm)]; ¹H NMR (400 MHz, DMSO-d₆): δ 8.11 (d, 1H), 7.86 (d, 1H), 3.65 (q, 1H), 3.40 (d, 1H), 3.25 (d, 1H), 2.04-1.94 (m, 2H), 1.39-1.25 (m, 4H), 1.91-1.81 (m, 1H), 1.79-1.66 (m, 2H), 1.60 (dd, 1H), 1.51-1.40 (m, 1H), 1.31 (d, 4H).

### Example-9 and Example-10

### 5-((7-Methyl-6-azaspiro[3.4]octan-6-yl)sulfonyl)thiophene-2-carbonitrile and 5-((7-methyl-6-azaspiro[3.4]octan-6-yl)sulfonyl)thiophene-2-carbonitrile

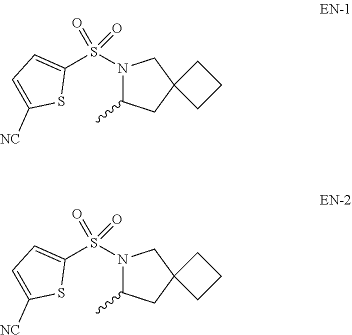

Chiral separation of 4-((7-methyl-6-azaspiro[3.4]octan-6-yl)sulfonyl)thiophene-2-carbonitrile provided ˜25 mg each of both enantiomers as mentioned below. Chiral Separation Method: Chiral separation was done on Agilent 1200 series instrument. Column name: Chiralpak IA (250×21 mm), 5μ, operating at ambient temperature and flow rate of 21.0 mL/min. Mobile phase: 0.1% Isopropylamine in the mixture of 80% Hexane, 10% Ethyl Acetate and 10% Ethanol, held this isocratic mixture up to 15 min with wavelength of 268 nm.

Example 9 [EN-1]: White solid (20 mg, 13% yield, 99.31%). LCMS: m/z found 297.12 [M+H]⁺, rt=2.06 min (Method 1) [Acquity BEH C8 column (1.7 μm, 50×2.1 mm)]; ¹H NMR (400 MHz, DMSO-d₆): δ 8.11 (d, 1H), 7.87 (d, 1H), 3.64 (q, 1H), 3.40 (d, 1H), 3.25 (d, 1H), 2.07-1.93 (m, 2H), 1.92-1.80 (m, 1H), 1.79-1.66 (m, 2H), 1.60 (dd, 1H), 1.53-1.39 (m, 1H), 1.30 (d, 4H).

Example 10 [EN-2]: White solid (20 mg, 13% yield, 99.78%). LCMS: m/z found 297.12 [M+H]⁺, rt=2.37 min (Method 1) [Acquity BEH C8 column (1.7 μm, 50×2.1 mm)]; ¹H NMR (400 MHz, DMSO-d₆): δ 8.10 (d, 1H), 7.86 (d, 1H), 3.64 (q, 1H), 3.40 (d, 1H), 3.25 (d, 1H), 2.06-1.93 (m, 2H), 1.90-1.80 (m, 1H), 1.77-1.65 (m, 2H), 1.60 (dd, 1H), 1.53-1.39 (m, 1H), 1.30 (d, 4H).

### Example-11

### 7-Methyl-6-(pyridin-4-ylsulfonyl)-6-azaspiro[3.4]octane

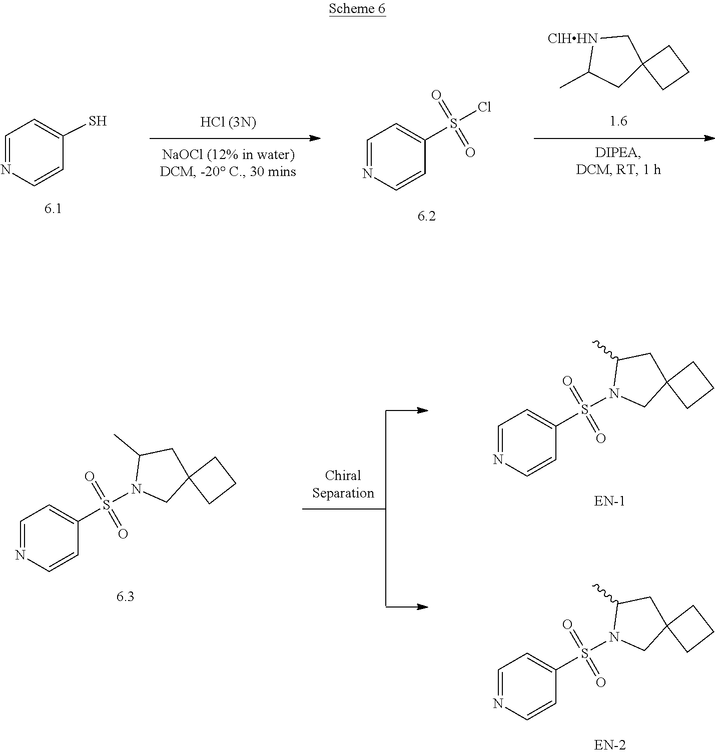

### Pyridine-4-sulfonyl chloride 6.2 (Method C)

Pyridine-4-thiol 6.1 (100 mg, 0.9 mmol) was dissolved in DCM (5 mL) and cooled at −20° C. Argon was bubbled to degasify and (3 N) HCl (3 mL) was added to it. The reaction mixture was stirred and the solution of NaOCl (12% in water) (3.5 mL) was added to it keeping the temperature below −10° C. It was stirred vigorously at −10° C. for 30 mins. The organic part was separated and dried. The organic part was cooled at 0° C. and DIPEA (1 mL) was added to it. 7-methyl-6-azaspiro[3.4]octane hydrochloride 1.6 (50 mg, 0.309 mmol) was added to it and the reaction mixture was stirred at RT for 1 h. The volatiles were then evaporated under reduced pressure and purified by column chromatography over silica gel using 3:7 ethyl acetate:hexane. The compound was isolated as brown sticky gum (40 mg, 49% yield, 97.30% purity). LCMS: m/z found 267.17 [M+H]⁺, rt=1.86 min (Method 1) [Acquity BEH C8 column (1.7 μm, 50×2.1 mm)]; ¹H NMR (400 MHz, DMSO-d₆): δ 8.90-8.83 (m, 2H), 7.81-7.75 (m, 2H), 3.61 (q, 1H), 3.34 (d, 1H), 3.23 (d, 1H), 2.02-1.90 (m, 2H), 1.89-1.77 (m, 1H), 1.78-1.60 (m, 2H), 1.54 (dd, 1H), 1.40-1.30 (m, 1H), 1.29 (d, 3H), 1.29-1.16 (m, 1H).

### Example-12 and Example-13

### 7-Methyl-6-(pyridin-4-ylsulfonyl)-6-azaspiro[3.4]octane and 7-methyl-6-(pyridin-4-ylsulfonyl)-6-azaspiro[3.4]octane

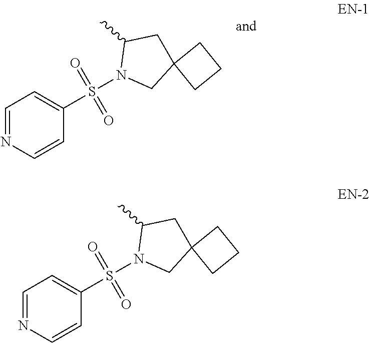

Chiral separation of 7-methyl-6-(pyridin-4-ylsulfonyl)-6-azaspiro[3.4]octane provided both enantiomers as mentioned below. Chiral Separation Method: Chiral separation was done on Agilent 1200 series instrument. Column name: Chiralpak IA (250×21 mm), 5μ, operating at ambient temperature and flow rate of 21.0 mL/min. Mobile phase: 0.1% Isopropylamine in the mixture of 80% Hexane and 20% Ethanol, held this isocratic mixture up to 15 min with wavelength of 260 nm.

Example 12 [EN-1]: Off white solid (7 mg, 21% yield, 96.81% purity). LCMS: m/z found 267.14 [M+H]⁺, rt=1.87 min (Method 1) [Acquity BEH C8 column (1.7 μm, 50×2.1 mm)]; ¹H NMR (400 MHz, DMSO-d₆): δ 8.87 (s, 2H), 7.78 (s, 2H), 3.61 (q, 1H), 3.49-3.33 (m, 2H), 3.23 (d, 2H), 1.94 (t, 2H), 1.88-1.78 (m, 1H), 1.68 (q, 2H), 1.54 (dd, 1H), 1.29 (d, 3H).

Example 13 [EN-2]: Off white solid (7 mg, 21% yield, 97.96% purity). LCMS: m/z found 267.14 [M+H]⁺, rt=1.87 min (Method 1) [Acquity BEH C8 column (1.7 μm, 50×2.1 mm)]; ¹H NMR (400 MHz, DMSO-d₆): δ 8.87 (d, 2H), 7.78 (d, 2H), 3.61 (q, 1H), 3.34 (d, 1H), 3.23 (d, 1H), 2.02-1.90 (m, 2H), 1.89-1.77 (m, 1H), 1.74-1.62 (m, 2H), 1.54 (dd, 1H), 1.40-1.30 (m, 1H), 1.29 (d, 3H), 1.27-1.17 (m, 1H).

### Example-14

### 7-Methyl-6-(pyrimidin-2-ylsulfonyl)-6-azaspiro[3.4]octane

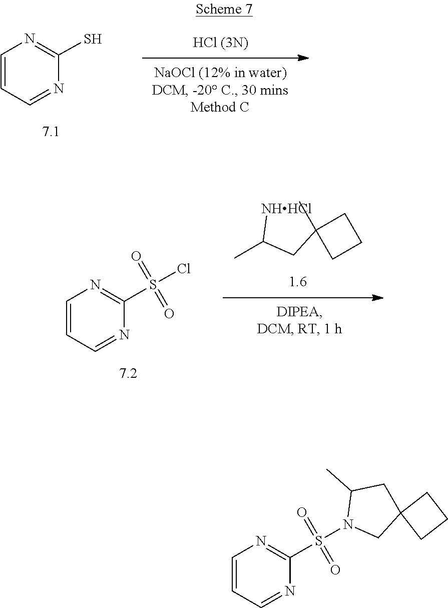

### Pyrimidine-2-sulfonyl chloride 7.2

Title compound was synthesized following Method C using pyrimidine-2-thiol 7.1 as a starting material

Example 14 was synthesized following Method A using 7-methyl-6-azaspiro[3.4]octane hydrochloride 1.6 and sulfonyl chloride 7.2 and was isolated as off white solid (56 mg, 85% yield, 99.32% purity). LCMS: m/z found 268.15 [M+H]⁺, rt=1.80 min (Method 1) [Acquity BEH C8 column (1.7 μm, 50×2.1 mm)]; ¹H NMR (400 MHz, DMSO-d₆): δ 9.04 (d, 2H), 7.80 (t, 1H), 3.99 (d, 1H), 3.50-3.38 (m, 2H), 2.20-2.07 (m, 1H), 2.06-1.96 (m, 1H), 1.96-1.84 (m, 1H), 1.84-1.68 (m, 2H), 1.67-1.54 (m, 2H), 1.52-1.38 (m, 1H), 1.25 (d, 3H).

### Example-15

### 6-(Benzofuran-6-ylsulfonyl)-7-methyl-6-azaspiro[3.4]octane

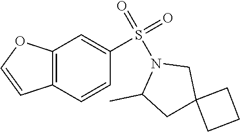 [SMILES: CC1CC2(CCC2)CN1S(=O)(=O)c1ccc2ccoc2c1]

The compound was synthesised following the procedure as described in Method A using 7-methyl-6-azaspiro[3.4]octane hydrochloride 1.6 and benzofuran-6-sulfonyl chloride. The compound was purified by flash chromatography over silica gel using 2:3 ethyl acetate:hexane and isolated as white solid (25 mg, 40% yield, 99.93% purity). LCMS: m/z found 306.18 [M+H]⁺, rt=2.10 min (Method 1) [Acquity BEH C8 column (1.7 μm, 50×2.1 mm)]; ¹H NMR (400 MHz, DMSO-d₆): δ 8.25 (d, 1H), 8.07 (s, 1H), 7.89 (d, 1H), 7.71 (dd, 1H), 7.12 (d, 1H), 3.58 (q, 1H), 3.21 (d, 1H), 2.01-1.74 (m, 4H), 1.69-1.56 (m, 2H), 1.51 (dd, 1H), 1.31 (d, 3H), 1.27-1.17 (m, 1H), 1.17-1.06 (m, 1H).

### Example-16 and Example-17

### 6-(Benzofuran-6-ylsulfonyl)-7-methyl-6-azaspiro[3.4]octane and 6-(benzofuran-6-ylsulfonyl)-7-methyl-6-azaspiro[3.4]octane

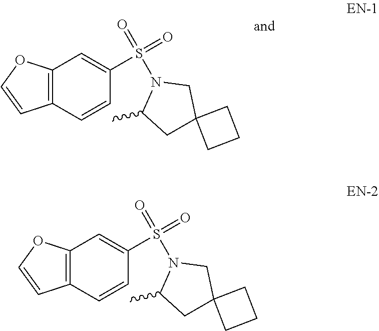

Chiral separation of racemic 6-(benzofuran-6-ylsulfonyl)-7-methyl-6-azaspiro[3.4]octane provided ˜40 mg each of both enantiomers as mentioned below. Chiral Separation Method: Chiral separation was done on Agilent 1200 series instrument. Column name: Chiralpak IG (250×21 mm), 5μ, operating at ambient temperature and flow rate of 21.0 mL/min. Mobile phase: 0.1% Isopropylamine in the mixture of 80% Hexane and 20% Ethanol, held this isocratic mixture up to 30 min with wavelength of 262 nm.

Example 16 [EN-1]: Light yellow sticky gum (40 mg, 28% yield, 97.67% purity). LCMS: m/z found 306.22 [M+H]⁺, rt=2.09 min (Method 1) [Acquity BEH C8 column (1.7 μm, 50×2.1 mm)]; ¹H NMR (400 MHz, DMSO-d₆): δ 8.26 (d, 1H), 8.07 (s, 1H), 7.89 (d, 1H), 7.71 (d, 1H), 7.12 (s, 1H), 3.66-3.50 (m, 1H), 3.21 (d, 1H), 2.02-1.72 (m, 3H), 1.70-1.42 (m, 3H), 1.31 (d, 3H), 1.27-1.15 (m, 3H).

Example 17 [EN-2]: Light yellow sticky gum (40 mg, 28% yield, 97.50% purity). LCMS: m/z found 306.22 [M+H]⁺, rt=2.09 min (Method 1) [Acquity BEH C8 column (1.7 μm, 50×2.1 mm)]; ¹H NMR (400 MHz, DMSO-d₆): δ 8.26 (d, 1H), 8.07 (s, 1H), 7.89 (d, 1H), 7.71 (dd, 1H), 7.13 (dd, 1H), 3.58 (q, 1H), 3.20 (d, 1H), 1.98-1.76 (m, 3H), 1.69-1.56 (m, 2H), 1.51 (dd, 1H), 1.31 (d, 3H), 1.27-1.05 (m, 3H).

### Example-18 to Example-21

### 2,2,2-Trifluoro-1-(7-((7-methyl-6-azaspiro[3.4]octan-6-yl)sulfonyl)-3,4-dihydroisoquinolin-2(1H)-yl)ethan-1-one (Example 18) 2,2,2-trifluoro-1-(7-((7-methyl-6-azaspiro[3.4]octan-6-yl)sulfonyl)-3,4-dihydroisoquinolin-2(1H)-yl)ethan-1-one (Example 19 [EN-1]), 2,2,2-trifluoro-1-(7-((7-methyl-6-azaspiro[3.4]octan-6-yl)sulfonyl)-3,4-dihydroisoquinolin-2(1H)-yl)ethan-1-one (Example 20 [EN-2]) and 1-(7-((7-methyl-6-azaspiro[3.4]octan-6-yl)sulfonyl)-3,4-dihydroisoquinolin-2(1H)-yl)-2-phenylethan-1-one (Example 21)

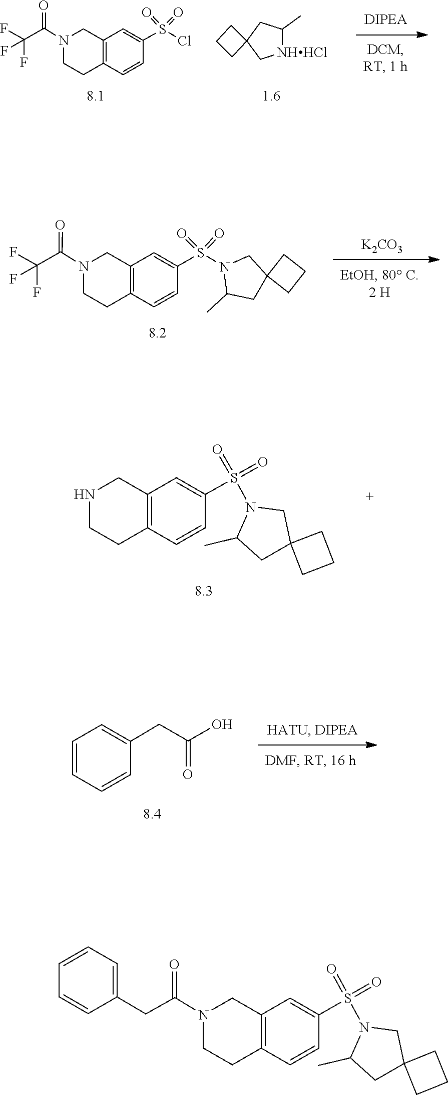

### 2,2,2-Trifluoro-1-(7-((7-methyl-6-azaspiro[3.4]octan-6-yl)sulfonyl)-3,4-dihydroisoquinolin-2(1H)-yl)ethan-1-one 8.2 (Example-18)

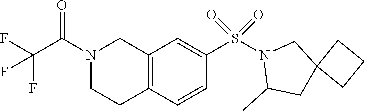 [SMILES: CC1CC2(CCC2)CN1S(=O)(=O)c1ccc2c(c1)CN(C(=O)C(F)(F)F)CC2]

Compound 8.2 was synthesised following the procedure described in Method A using 7-methyl-6-azaspiro[3.4]octane hydrochloride 1.6 and 2-(2,2,2-trifluoroacetyl)-1,2,3,4-tetrahydroisoquinoline-7-sulfonyl chloride. Compound 8.2 was isolated as colourless sticky gum (40 mg, 57% yield, 99.79% purity). LCMS: m/z found 417.2 [M+H]⁺, rt=2.15 min, (Method 13) [YMC Triart C18 column (3 μm, 33×2.1 mm)]; ¹H NMR (400 MHz, DMSO-d₆): δ 7.81 (d, 1H), 7.64 (d, 1H), 7.43 (d, 1H), 4.88 (d, 2H), 3.92-3.73 (m, 2H), 3.60-3.52 (m, 1H), 3.17 (t, 1H), 3.07-2.95 (m, 2H), 2.04-1.77 (m, 3H), 1.77-1.61 (m, 2H), 1.59-1.45 (m, 1H), 1.44-1.17 (m, 6H).

### 2,2,2-Trifluoro-1-(7-((7-methyl-6-azaspiro[3.4]octan-6-yl)sulfonyl)-3,4-dihydroisoquinolin-2(1H)-yl)ethan-1-one (Example-19; EN-1) and 2,2,2-trifluoro-1-(7-((7-methyl-6-azaspiro[3.4]octan-6-yl)sulfonyl)-3,4-dihydroisoquinolin-2(1H)-yl)ethan-1-one (Example-20; EN-2)

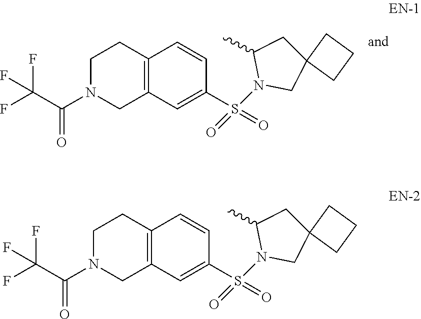

Chiral separation of racemic compound (Example 18) provided ˜5 mg each of both enantiomers as mentioned below. Chiral Separation Method: Chiral separation was done on Agilent 1200 series instrument. Column name: Chiralpak IA (250×20 mm), 5μ, operating at ambient temperature and flow rate of 18.0 mL/min. Mobile phase: 0.1% iso propylamine in the mixture of 80% hexane and 20% ethanol, held this isocratic mixture up to 25 min with wavelength of 212 nm.

Example 19 [EN-1]: Light brown sticky solid (15 mg, 29% yield, 99.02% purity). LCMS: m/z found 417.26 [M+H]⁺, rt=2.15 min, (Method 1) [Acquity BEH C8 column (1.7 μm, 50×2.1 mm)]; ¹H NMR (400 MHz, DMSO-d₆): δ 7.81 (d, 1H), 7.64 (d, 1H), 7.43 (d, 1H), 4.88 (d, 2H), 3.91-3.75 (m, 2H), 3.62-3.50 (m, 1H), 3.17 (t, 1H), 3.05-2.96 (m, 2H), 2.05-1.77 (m, 3H), 1.74-1.61 (m, 2H), 1.58-1.45 (m, 1H), 1.42-1.06 (m, 6H).

Example 20 [EN-2]: Light brown sticky solid (15 mg, 29% yield, 99.02% purity). LCMS: m/z found 417.26 [M+H]⁺, rt=2.15 min, (Method 1) [Acquity BEH C8 column (1.7 μm, 50×2.1 mm)]; ¹H NMR (400 MHz, DMSO-d₆): δ 7.81 (d, 1H), 7.63 (d, 1H), 7.43 (d, 1H), 4.88 (d, 2H), 3.82 (s, 2H), 3.60-3.52 (m, 1H), 3.17 (t, 1H), 3.08-2.93 (m, 2H), 1.97-1.82 (m, 3H), 1.77-1.60 (m, 2H), 1.58-1.48 (m, 1H), 1.43-1.30 (m, 2H), 1.28 (d, 3H), 1.26-1.10 (m, 1H).

### 7-((7-Methyl-6-azaspiro[3.4]octan-6-yl)sulfonyl)-1,2,3,4-tetrahydroisoquinoline 8.3

### Cleavage of —COCF₃ Group (Method D):

Intermediate 8.2 (170 mg, 0.408 mmol) was dissolved in EtOH (10 mL) and the solution of K₂CO₃ (225 mg, 1.633 mmol) in water (2.5 mL) was added to it. The RM was then heated at 80° C. for 2 h. The solvent was evaporated under reduced pressure and the crude was diluted with water. It was then extracted with 10% MeOH in DCM (2×15 mL), the organic part was washed with water (1×5 mL) and brine (1×5 mL), dried over MgSO₄ and the solvent was evaporated under reduced pressure. The crude intermediate 8.3 (102 mg) was used in the forwarding step without further purification.

### 1-(7-((7-Methyl-6-azaspiro[3.4]octan-6-yl)sulfonyl)-3,4-dihydroisoquinolin-2(1H)-yl)-2-phenylethan-1-one (Example-21)

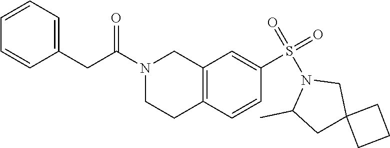 [SMILES: CC1CC2(CCC2)CN1S(=O)(=O)c1ccc2c(c1)CN(C(=O)Cc1ccccc1)CC2]

Amidation (Method E): Intermediate 8.3 (20 mg, 0.062 mmol) was dissolved in DMF (1 ml) and 2-phenylacetic acid 8.4 (17 mg, 0.125 mmol) was added to it. The reaction mixture was cooled at 5° C. and DIPEA (0.06 ml, 0.312 mmol) was added to it, followed by HATU (60 mg, 0.156 mmol). The RM was then stirred overnight at RT. The crude was directly purified by Reverse Phase Prep-HPLC provided desired compound (Example 21) as colourless sticky gum (12 mg, 44% yield, 92.07% purity). LCMS: m/z found 439.31 [M+H]⁺, rt=2.11 min (Method 1) [Acquity BEH C8 column (1.7 μm, 50×2.1 mm)]; ¹H NMR (400 MHz, DMSO-d₆): δ 7.63 (s, 1H), 7.59 (d, 1H), 7.37 (d, 1H), 7.33-7.14 (m, 5H), 4.78 (s, 2H), 3.81 (s, 2H), 3.74 (t, 2H), 3.61 (q, 1H), 3.32 (d, 1H), 3.21 (d, 1H), 2.86 (t, 2H), 2.04-1.86 (m, 3H), 1.79-1.70 (m, 2H), 1.62-1.43 (m, 3H), 1.28 (d, 3H).

### Example-22

### Ethyl 7-((7-methyl-6-azaspiro[3.4]octan-6-yl)sulfonyl)-3,4-dihydroisoquinoline-2(1H)-carboxylate

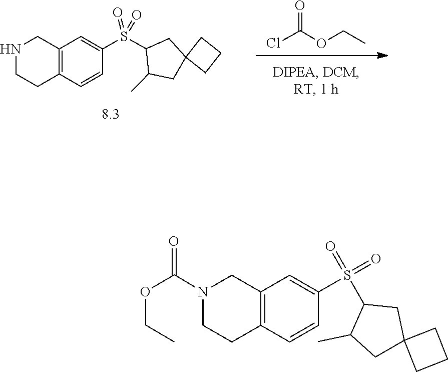

### General Amidation Protocol (Method B):

To a solution of isoquinoline 8.3 (25 mg, 0.078 mmol) in DCM (2 mL) was added DIPEA (0.07 mL, 0.39 mmol) at 0° C. The reaction mixture was stirred and ethyl chloroformate (13 mg, 0.117 mmol) was added to it. It was then stirred at RT for 1 h. The volatiles were evaporated under reduced pressure and the crude was purified by Column Chromatography over silica gel using 3:7 ethyl acetate:hexane and isolated as colourless sticky gum (15 mg, 49% yield, 96.40% purity). LCMS: m/z found 393.28 [M+H]⁺, rt=2.16 min (Method 1) [Acquity BEH C8 column (1.7 μm, 50×2.1 mm)]; ¹H NMR (400 MHz, DMSO-d₆): δ 7.68 (s, 1H), 7.60 (d, 1H), 7.39 (d, 1H), 4.64 (s, 2H), 4.09 (q, 2H), 3.69-3.47 (m, 3H), 3.31-3.26 (m, 1H), 3.16 (d, 1H), 2.87 (t, 2H), 2.03-1.76 (m, 3H), 1.73-1.64 (m, 2H), 1.52 (dd, 1H), 1.42-1.25 (m, 5H), 1.21 (t, 3H).

### Ethyl 7-((7-methyl-6-azaspiro[3.4]octan-6-yl)sulfonyl)-3,4-dihydroisoquinoline-2(1H)-carboxylate (Example 23) and Ethyl 7-((7-methyl-6-azaspiro[3.4]octan-6-yl)sulfonyl)-3,4-dihydroisoquinoline-2(1H)-carboxylate (Example 24)

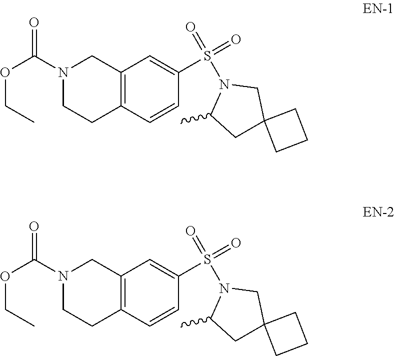

Chiral separation of 7-((7-methyl-6-azaspiro[3.4]octan-6-yl)sulfonyl)-3,4-dihydroisoquinoline-2(1H)-carboxylate (Example 22) provided both enantiomers as mentioned below. Chiral Separation Method: Chiral separation was done on Agilent 1200 series instrument. Column name: Chiralpak IG (250×21 mm), 5μ, operating at ambient temperature and flow rate of 21.0 mL/min. Mobile phase: 0.1% Isopropylamine in the mixture of 70% Hexane, 15% Dichloromethane and 15% Ethanol, held this isocratic mixture up to 25 min with wavelength of 234 nm.

Example 23, [EN-1]: Colourless sticky gum (20 mg, 14% yield, 95.13% purity). LCMS: m/z found 393.32 [M+H]⁺, rt=2.19 min (Method 1) [Acquity BEH C8 column (1.7 μm, 50×2.1 mm)]; ¹H NMR (400 MHz, DMSO-d₆): δ 7.68 (s, 1H), 7.60 (d, 1H), 7.39 (d, 1H), 4.64 (s, 2H), 4.09 (q, 2H), 3.64-3.50 (m, 3H), 3.16 (d, 1H), 2.87 (t, 2H), 2.01-1.78 (m, 5H), 1.75-1.61 (m, 2H), 1.52 (dd, 1H), 1.41-1.30 (m, 1H), 1.28 (d, 3H), 1.20 (t, 3H).

Example 24, [EN-2]: Colourless sticky gum (20 mg, 14% yield, 95.92% purity). LCMS: m/z found 393.31 [M+H]⁺, rt=2.19 min (Method 1) [Acquity BEH C8 column (1.7 μm, 50×2.1 mm)]; ¹H NMR (400 MHz, DMSO-d₆): δ 7.68 (s, 1H), 7.60 (d, 1H), 7.39 (d, 1H), 4.64 (s, 2H), 4.09 (q, 2H), 3.60 (t, 2H), 3.30-3.18 (m, 1H), 3.16 (d, 1H), 2.87 (t, 2H), 2.02-1.76 (m, 3H), 1.74-1.62 (m, 2H), 1.52 (dd, 1H), 1.41-1.31 (m, 1H), 1.28 (d, 4H), 1.20 (t, 4H).

### Example-25

### 2-Benzyl-6-((7-methyl-6-azaspiro[3.4]octan-6-yl)sulfonyl)-1,2,3,4-tetrahydroisoquinoline

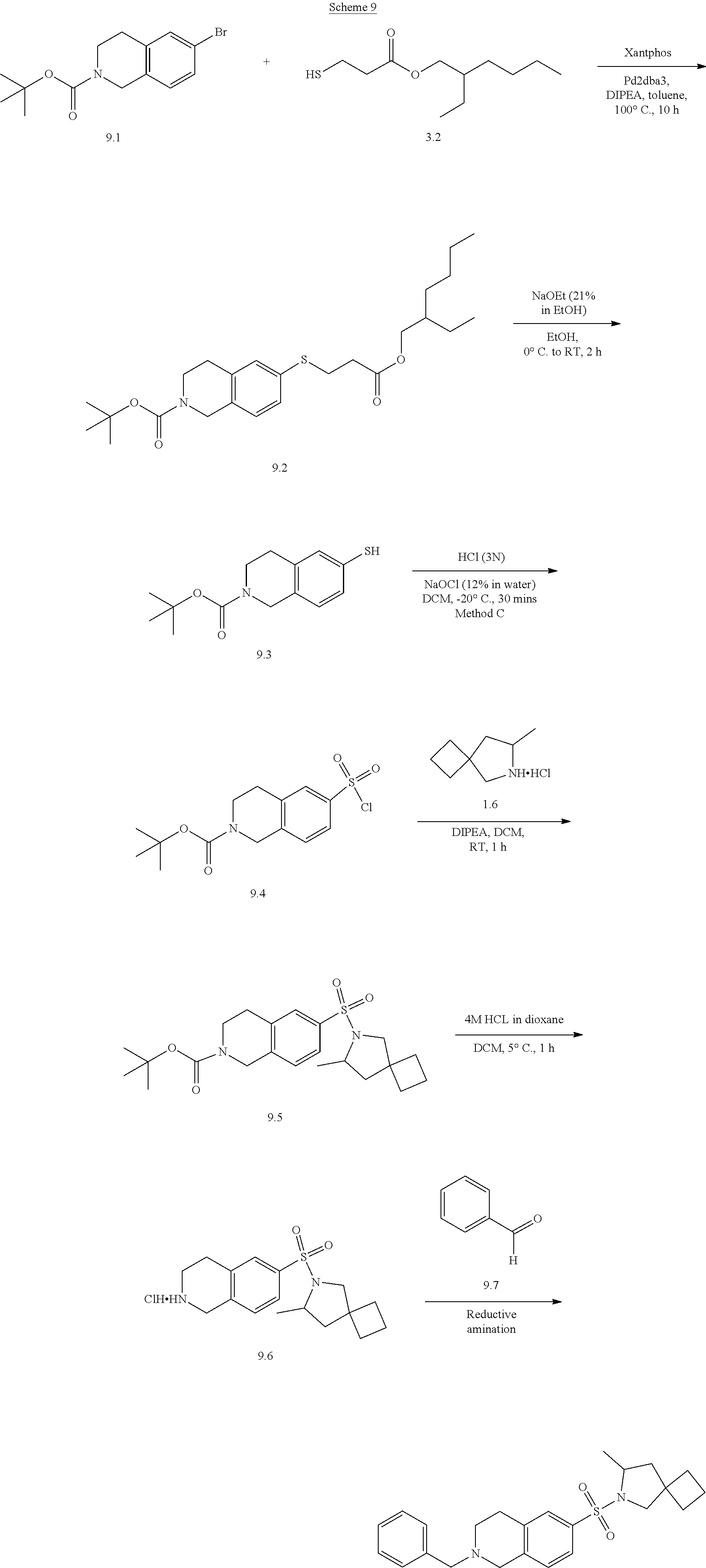

### tert-Butyl 6-((3-((2-ethylhexyl)oxy)-3-oxopropyl)thio)-3,4-dihydroisoquinoline-2(1H)-carboxylate 9.2

Buchwald reaction (Method F): A mixture of 3,4-dihydroisoquinoline 9.1 (2 g, 6.41 mmol) and 2-ethylhexyl 3-mercaptopropanoate 3.2 (1.537 mg, 6.41 mmol) was dissolved in toluene (20 ml) and DIPEA (22.3 ml, 12.821 mmol) was added to it. It was then degassed with argon and Xantphos (185 mg, 0.321 mmol) was added to it, followed by Pd₂dba₃ (176 mg, 0.192 mmol) under argon atm. The RM was then heated at 90° C. under argon atmosphere for 16 h. It was then filtered, and the solvent was evaporated under reduced pressure. The crude was further purified by column chromatography over silica gel using 30% ethyl acetate in hexane. Intermediate 9.2 was isolated as yellow oil (2.5 g, 87% yield). ¹H NMR (400 MHz, DMSO-d₆): δ 7.27-7.03 (m, 3H), 4.45 (s, 2H), 3.93 (d, 2H), 3.52 (t, 2H), 3.13 (t, 2H), 2.74 (t, 2H), 2.59 (t, 2H), 1.60-1.46 (m, 1H), 1.42 (s, 9H), 1.37-1.14 (m, 8H), 0.89-0.77 (m, 6H).

### Ethyl 6-mercapto-3,4-dihydroisoquinoline-2(1H)-carboxylate 9.3

Retro-aldol (Method G): To a stirred solution of intermediate 9.2 (200 mg, 0.445 mmol) in EtOH (10 ml), was added (21%) NaOEt in EtOH (0.3 ml) in ice cold condition and the reaction mixture was stirred at RT for 2 h. The volatile was evaporated, and the residue was diluted with water. It was then acidified with aqueous citric acid and then extracted with DCM (2×15 mL). The organic part was washed with brine (2×5 mL), dried over anhydrous MgSO4 and the solvent was evaporated under reduced pressure. The crude intermediate 9.3 (180 mg) was used directly in the forwarding step without further purification.

### tert-Butyl 6-(chlorosulfonyl)-3,4-dihydroisoquinoline-2(1H)-carboxylate 9.4

Sulfonyl chloride 9.4 was synthesized from intermediate 9.3 following the Method C.

### tert-Butyl 6-((7-methyl-6-azaspiro[3.4]octan-6-yl)sulfonyl)-3,4-dihydroisoquinoline-2(1H)-carboxylate 9.5

Sulfonamide 9.5 was synthesized following Method A using amine 1.6 and sulfonyl chloride 9.4. Intermediate 9.5 was isolated as light brown oil (50 mg, 36% yield).

### 6-((7-Methyl-6-azaspiro[3.4]octan-6-yl)sulfonyl)-1,2,3,4-tetrahydroisoquinoline hydrochloride 9.6

N-Boc deprotection (Method H): Intermediate 9.5 (30 mg, 0.071 mmol) was dissolved in DCM (1 mL) and (4 M) HCl in dioxane (0.5 mL) was added to it in cold condition. It was then stirred at 5° C. for 1 h. The volatiles were evaporated under reduced pressure and the crude intermediate 9.6 (˜30 mg) was used in the forwarding step.

### 2-Benzyl-6-((7-methyl-6-azaspiro[3.4]octan-6-yl)sulfonyl)-1,2,3,4-tetrahydroisoquinoline (Example 25)

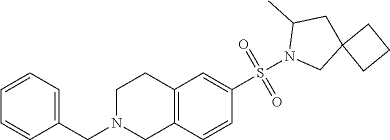 [SMILES: CC1CC2(CCC2)CN1S(=O)(=O)c1ccc2c(c1)CCN(Cc1ccccc1)C2]

Reductive amination (Method I): Intermediate 9.6 (20 mg, 0.056 mmol) was dissolved in DCE (1 mL) and benzaldehyde (11 mg, mmol) was added to it along with NaOAc (5 mg, 0.056 mmol). It was stirred for 6 h at RT and NaBH₃CN (15 mg, 0.224 mmol) was added to it. It was further stirred overnight at RT. The reaction mass was purified by Reverse Phase Prep-HPLC and the compound (Example-25) was isolated yellow sticky gum (18 mg, 78% yield, 99.41% purity). Prep-HPLC Method: Preparative HPLC was done on Waters auto purification instrument. Column name: YMC-Actus C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 70% A and 30% B, then to 25% A and 75% B in 3 min, then to 5% A and 95% B in 22 min., then to 5% A and 95% B in 23 min., held this composition up to 25 min. for column washing, then returned to initial composition in 26 min. and held till 28 min. LCMS: m/z found 411.31 [M+H]⁺, rt=1.65 min (Method 1) [Acquity BEH C8 column (1.7 μm, 50×2.1 mm)]; ¹H NMR (400 MHz, DMSO-d₆): δ 7.60-7.42 (m, 2H), 7.41-7.19 (m, 6H), 3.72-3.66 (m, 4H), 3.65-3.54 (m, 1H), 3.31 (dd, 1H), 3.20 (d, 1H), 2.90 (t, 2H), 2.75 (t, 2H), 2.06-1.85 (m, 3H), 1.82-1.68 (m, 2H), 1.63-1.42 (m, 3H), 1.33-1.22 (m, 3H).

### Example-26

### (7-((7-Methyl-6-azaspiro[3.4]octan-6-yl)sulfonyl)-3,4-dihydroisoquinolin-2(1H)-yl)(phenyl)methanone

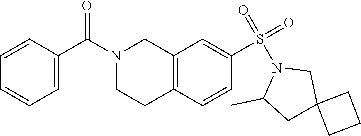 [SMILES: CC1CC2(CCC2)CN1S(=O)(=O)c1ccc2c(c1)CN(C(=O)c1ccccc1)CC2]

The compound was synthesised following the procedure as described in Method B using isoquinoline 8.3 and benzoyl chloride. The crude was purified by Reverse Phase Prep-HPLC and the compound (Example-26) was isolated as white solid (18 mg, 34% yield, 99.88% purity). Prep-HPLC Method: Preparative HPLC was done on Waters auto purification instrument. Column name: YMC-Actus Triart C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 70% A and 30% B, then to 40% A and 60% B in 3 min, then to 10% A and 90% B in 22 min., then to 5% A and 95% B in 23 min., held this composition up to 25 min. for column washing, then returned to initial composition in 26 min. and held till 28 min. LCMS: m/z found 425.27 [M+H]⁺, rt=2.11 min (Method 1) [Acquity BEH C8 column (1.7 μm, 50×2.1 mm)], ¹H NMR (400 MHz, DMSO-d₆) δ 4.79 (s, 2H), 3.04-2.90 (m, 2H), 2.08-1.85 (m, 3H), 1.83-1.68 (m, 2H), 1.64-1.42 (m, 3H), 3.06-2.97 (m, 1H), 7.70-7.34 (m, 8H), 3.72 (t, 2H), 3.64 (q, 1H), 3.32 (d, 1H), 3.22 (d, 1H), 1.28 (d, 3H).

### Example-27 and Example 28

### (7-((7-Methyl-6-azaspiro[3.4]octan-6-yl)sulfonyl)-3,4-dihydroisoquinolin-2(1H)-yl)(phenyl)methanone and

### (7-((7-methyl-6-azaspiro[3.4]octan-6-yl)sulfonyl)-3,4-dihydroisoquinolin-2(1H)-yl)(phenyl)methanone

Chiral separation of (7-((7-methyl-6-azaspiro[3.4]octan-6-yl)sulfonyl)-3,4-dihydroisoquinolin-2(1H)-yl)(phenyl)methanone (Example 26) provided ˜25 mg each of both enantiomers as mentioned below. Chiral Separation Method: Chiral separation was done on Agilent 1200 series instrument. Column name: Chiralpak IG (250×21 mm), 5μ, operating at ambient temperature and flow rate of 21.0 mL/min. Mobile phase: 0.1% Isopropylamine in the mixture of 50% Hexane, 25% Dichloromethane and 25% Ethanol, held this isocratic mixture up to 25 min with wavelength of 226 nm.

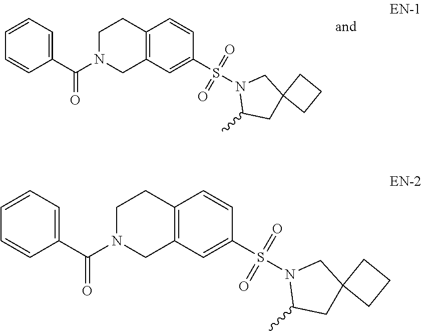

Example 27, [EN-1]: Off white solid (25 mg, 24% yield, 98.14% purity). LCMS: m/z found 425.34 [M+H]⁺, rt=2.12 min (Method 1) [Acquity BEH C8 column (1.7 μm, 50×2.1 mm)]; ¹H NMR (400 MHz, DMSO-d₆): δ 7.83-7.68 (m, 1H), 7.61 (d, 1H), 7.53-7.38 (m, 6H), 5.18-4.43 (m, 2H), 3.97-3.71 (m, 1H), 3.56 (s, 2H), 3.17 (s, 1H), 2.94 (s, 2H), 2.06-1.78 (m, 3H), 1.76-1.60 (m, 2H), 1.59-1.46 (m, 1H), 1.44-1.15 (m, 6H).

Example 28, [EN-2]: Off white solid (25 mg, 24% yield, 98.41% purity). LCMS: m/z found 425.34 [M+H]⁺, rt=2.11 min (Method 1) [Acquity BEH C8 column (1.7 μm, 50×2.1 mm)]; ¹H NMR (400 MHz, DMSO-d₆): δ 7.78-7.73 (m, 1H), 7.59 (d, 1H), 7.53-7.38 (m, 6H), 5.10-4.56 (m, 2H), 4.05-3.70 (m, 1H), 3.57 (s, 2H), 3.17 (s, 1H), 2.94 (s, 2H), 2.06-1.78 (m, 3H), 1.77-1.62 (m, 2H), 1.60-1.46 (m, 1H), 1.45-1.17 (m, 6H).

### Example 29

### 7-((7-Methyl-6-azaspiro[3.4]octan-6-yl)sulfonyl)-2-phenyl-1,2,3,4-tetrahydroisoquinoline

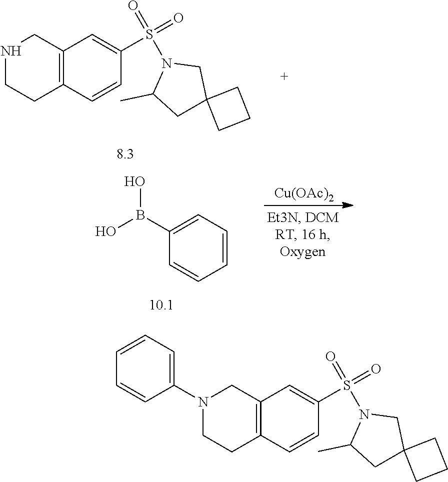 [SMILES: CC1CC2(CCC2)CN1S(=O)(=O)c1ccc2c(c1)CNCC2 ; OB(O)c1ccccc1 ; CC1CC2(CCC2)CN1S(=O)(=O)c1ccc2c(c1)CN(c1ccccc1)CC2 ; "Cu(OAc)2"]

Chan-Lam Coupling (Method J): Intermediate 8.3 (120 mg, 0.336 mmol) was dissolved in DCM (5 ml) and phenyl boronic acid 10.1 (123 mg, 1.009 mmol) was added to it. Et₃N (0.2 ml, 1.681 mmol) was added to the solution and oxygen was bubbled through it. Copper (II) acetate (153 mg, 0.841 mmol) was added to solution along with molecular sieve. The reaction mixture was then stirred at RT overnight under oxygen atmosphere. It was filtered and the filtrate was evaporated under reduced pressure. The crude was purified by column chromatography over silica gel using 30% ethyl acetate in hexane provided brown sticky gum (55 mg, 41% yield, 86.78% purity). LCMS: m/z found 397.0 [M+H]⁺, rt=8.16 min (Method 12) [Zorbax Ext C18 column (5 μm, 100×4.6 mm)]. ¹H NMR (400 MHz, DMSO-d₆): δ 7.72 (s, 1H), 7.58 (dd, 1H), 7.38 (d, 1H), 7.27-7.11 (m, 2H), 7.02 (d, 2H), 6.75 (t, 1H), 4.50 (s, 2H), 3.61-3.51 (m, 3H), 3.17 (d, 1H), 2.97 (t, 2H), 2.00-1.79 (m, 3H), 1.74-1.59 (m, 2H), 1.52 (dd, 1H), 1.40-1.17 (m, 6H).

### Example 30

### 2-Methyl-1-(7-((7-methyl-6-azaspiro[3.4]octan-6-yl)sulfonyl)-3,4-dihydroisoquinolin-2(1H)-yl)propan-1-one

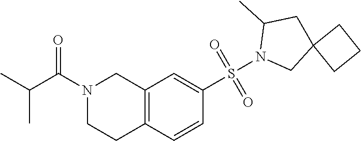 [SMILES: CC(C)C(=O)N1CCc2ccc(S(=O)(=O)N3CC4(CCC4)CC3C)cc2C1]

Compound was synthesized following amidation Method B using intermediate 8.3 and isobutyryl chloride. The compound was purified by column chromatography over silica gel using 30% ethyl acetate in hexane and isolated as light-yellow sticky gum (25 mg, 30% yield, 99.08% purity) LCMS: m/z found 391.30 [M+H]⁺, rt=2.02 min (Method 1) [Acquity BEH C8 column (1.7 μm, 50×2.1 mm)]. ¹H NMR (400 MHz, DMSO-d₆): δ 7.78-7.55 (m, 2H), 7.47-7.35 (m, 1H), 4.86-4.77 (m, 1H), 4.69 (d, 1H), 3.83-3.61 (m, 2H), 3.55 (d, 1H), 3.16 (d, 1H), 3.06-2.90 (m, 2H), 2.90-2.77 (m, 1H), 2.02-1.78 (m, 3H), 1.75-1.61 (m, 2H), 1.52 (dd, 1H), 1.43-1.17 (m, 6H), 1.06-0.95 (m, 6H).

### Example-31 and Example-32

### 2-Methyl-1-(7-((7-methyl-6-azaspiro[3.4]octan-6-yl)sulfonyl)-3,4-dihydroisoquinolin-2(1H)-yl)propan-1-one and

### 2-methyl-1-(7-((7-methyl-6-azaspiro[3.4]octan-6-yl)sulfonyl)-3,4-dihydroisoquinolin-2(1H)-yl)propan-1-one

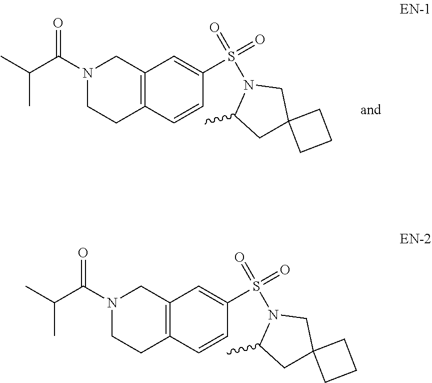

Chiral separation racemic compound (Example 30) provided ˜10 mg each of both isomers as mentioned below. Chiral Separation Method: CHIRALPAK IA (250×20 mm) 5u, Flow rate −18 ml/min, Mobile phase −HEXANE/ETOH −80/20, Solubility -MEOH, Wavelength −230 nm, Run time −20 min.

Example 31, [EN-1]: Colourless gum (10 mg, 51% yield, 85.39% purity). LCMS: m/z found 391.31 [M+H]⁺, rt=2.05 min (Method 1) [Acquity BEH C8 column (1.7 μm, 50×2.1 mm)]; 1H NMR (400 MHz, DMSO-d6): δ 7.70 (d, 1H), 7.60 (d, 1H), 7.40 (d, 1H), 4.83 (d, 1H), 4.69 (s, 1H), 3.84-3.47 (m, 3H), 3.20-3.16 (m, 1H), 3.06-2.90 (m, 2H), 2.88-2.78 (m, 1H), 2.01-1.78 (m, 3H), 1.74-1.60 (m, 2H), 1.52 (dd, 1H), 1.38-1.20 (m, 6H), 1.06-0.94 (m, 6H).

Example 32, [EN-2]: Colourless gum (9 mg, 46% yield, 90.41% purity). LCMS: m/z found 391.35 [M+H]⁺, rt=2.05 min (Method 1) [Acquity BEH C8 column (1.7 μm, 50×2.1 mm)]1H NMR (400 MHz, DMSO-d6): δ 7.69 (d, 1H), 7.60 (d, 1H), 7.39 (t, 1H), 4.83 (d, 1H), 4.69 (s, 1H), 3.82-3.60 (m, 2H), 3.59-3.49 (m, 1H), 3.16 (d, 1H), 3.04-2.91 (m, 2H), 2.82 (s, 1H), 2.02-1.76 (m, 3H), 1.74-1.60 (m, 2H), 1.57-1.47 (m, 1H), 1.39-1.21 (m, 6H), 1.11-0.92 (m, 6H).

### Example-33 and Example-34

### 1-(7-((7-Ethyl-6-azaspiro[3.4]octan-6-yl)sulfonyl)-3,4-dihydroisoquinolin-2(1H)-yl)-2,2,2-trifluoroethan-1-one and

### 1-(7-((7-ethyl-6-azaspiro[3.4]octan-6-yl)sulfonyl)-3,4-dihydroisoquinolin-2(1H)-yl)-2-methylpropan-1-one

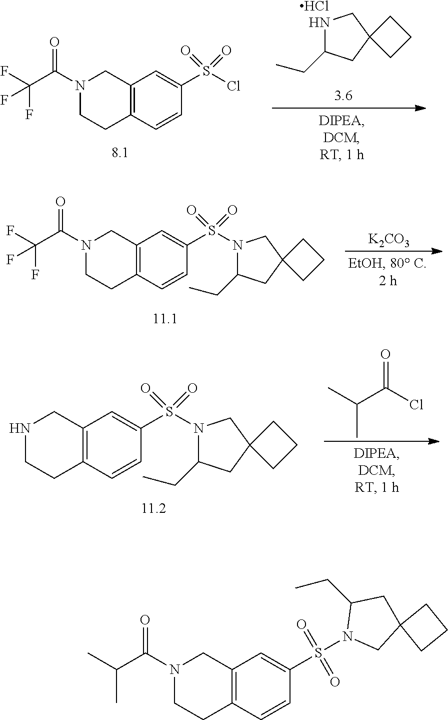

### 1-(7-((7-Ethyl-6-azaspiro[3.4]octan-6-yl)sulfonyl)-3,4-dihydroisoquinolin-2(1H)-yl)-2,2,2-trifluoroethan-1-one 11.1

Intermediate 11.1 was synthesised following the procedure described in Method A using 2-(2,2,2-trifluoroacetyl)-1,2,3,4-tetrahydroisoquinoline-7-sulfonyl chloride 8.1 and 7-ethyl-6-azaspiro[3.4]octane hydrochloride 3.6. The compound was purified by column chromatography over silica gel using 30% ethyl acetate in hexane and compound 11.1 (Example 33) was isolated as colourless sticky solid (28 mg, 13% yield, 99.43% purity). LCMS: m/z found 431.25 [M+H]⁺, rt=2.22 min (Method 1) [Acquity BEH C8 column (1.7 μm, 50×2.1 mm)]; ¹H NMR (400 MHz, DMSO-d₆) δ 7.74 (s, 1H), 7.65 (d, 1H), 7.43 (d, 1H), 4.86 (s, 2H), 3.85 (t, 2H), 3.60-3.46 (m, 1H), 3.35-3.23 (m, 2H), 3.02 (t, 2H), 2.03-1.80 (m, 4H), 1.79-1.60 (m, 3H), 1.58-1.38 (m, 3H), 0.86 (t, 3H).

7-((7-Ethyl-6-azaspiro[3.4]octan-6-yl)sulfonyl)-1,2,3,4-tetrahydroisoquinoline 11.2: Intermediate 11.2 was synthesised following the procedure described in Method D. The crude was used in the forwarding step without further purification.

### 1-(7-((7-Ethyl-6-azaspiro[3.4]octan-6-yl)sulfonyl)-3,4-dihydroisoquinolin-2(1H)-yl)-2-methylpropan-1-one

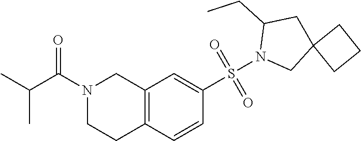 [SMILES: CCC1CC2(CCC2)CN1S(=O)(=O)c1ccc2c(c1)CN(C(=O)C(C)C)CC2]

The compound (Example 34) was synthesised following the procedure described in Method B using intermediate 11.2 and isobutyryl chloride. The compound was purified by column chromatography over silica gel using 30% ethyl acetate in hexane and obtained as off-white sticky solid (92 mg, 80% yield, 98.41% purity). LCMS: m/z found 405.37 [M+H]⁺, RT=2.10 min (Method 1) [Acquity BEH C8 column (1.7 μm, 50×2.1 mm)]; ¹H NMR (400 MHz, DMSO-d₆): δ 7.71 (d, 1H), 7.60 (d, 1H), 3.80-3.57 (m, 2H), 7.39 (d, 1H), 4.83 (d, 1H), 4.70 (s, 1H), 3.73 (s, 1H), 3.50-3.36 (m, 1H), 3.24 (s, 2H), 3.08-2.89 (m, 2H), 2.88-2.77 (m, 1H), 2.00-1.75 (m, 3H), 1.75-1.53 (m, 2H), 1.54-1.41 (m, 1H), 1.39-1.14 (m, 2H), 1.08-0.92 (m, 6H), 0.85 (t, 3H).

### Example-35

### 6-(Benzo[b]thiophen-6-ylsulfonyl)-7-ethyl-6-azaspiro[3.4]octane

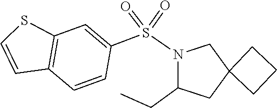 [SMILES: CCC1CC2(CCC2)CN1S(=O)(=O)c1ccc2ccsc2c1]

Compound was prepared for Method A using 7-ethyl-6-azaspiro[3.4]octane and benzo[b]thiophene-6-sulfonyl chloride. Purification was conducted by Reverse Phase prep-HPLC. Desired compound (Example 35) was isolated as yellow sticky gum (10 mg, 9% yield, 99.56% purity). Preparative HPLC was done on Waters auto purification instrument. Column name: YMC-Actus Triart C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 50% A and 50% B, then to 35% A and 65% B in 3 min, then to 5% A and 95% B in 22 min., then to 5% A and 95% B in 23 min., held this composition up to 25 min. for column washing, then returned to initial composition in 26 min. and held till 28 min. LCMS; m/z found 336.22 [M+H]⁺, rt=2.32 min, (Method 1) [Acquity BEH C8 column (1.7 μm, 50×2.1 mm)]; ¹H NMR (400 MHz, DMSO-d₆): δ 8.60 (s, 1H), 8.11-8.03 (m, 2H), 7.78 (dd, 1H), 7.60 (d, 1H), 3.57-3.42 (m, 1H), 3.27 (d, 2H), 1.98-1.85 (m, 2H), 1.85-1.73 (m, 2H), 1.70-1.45 (m, 4H), 1.21-1.02 (m, 2H), 0.86 (t, 3H).

### Example-36

### 6-(Furan-2-ylsulfonyl)-7-methyl-6-azaspiro[3.4]octane

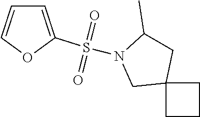 [SMILES: CC1CC2(CCC2)CN1S(=O)(=O)c1ccco1]

The synthesis was performed essentially as described in Method A using 7-methyl-6-azaspiro[3.4]octane hydrochloride 1.6 and furan-2-sulfonyl chloride. The compound was purified by Chromatography over silica gel using 3:7 ethyl acetate:hexane and isolated as light brown oil (35 mg, 20% yield, 99.56% purity). LCMS: m/z found 256.0 [M+H]⁺, rt=3.39 min (Method 3) [Zorbax C18 column (5 μm, 50×4.6 mm)]; ¹H NMR (400 MHz, Chloroform-d): δ 7.52 (dd, 1H), 7.00 (dd, 1H), 6.49 (dd, 1H), 3.81 (q, 1H), 3.42 (d, 1H), 3.33 (d, 1H), 2.08-1.86 (m, 3H), 1.89-1.69 (m, 2H), 1.66-1.54 (m, 2H), 1.52-1.39 (m, 1H), 1.37 (d, 3H).

### Example-37 and Example-38

### 6-(Furan-2-ylsulfonyl)-7-methyl-6-azaspiro[3.4]octane (EN-1) and

### 6-(furan-2-ylsulfonyl)-7-methyl-6-azaspiro[3.4]octane (EN-2)

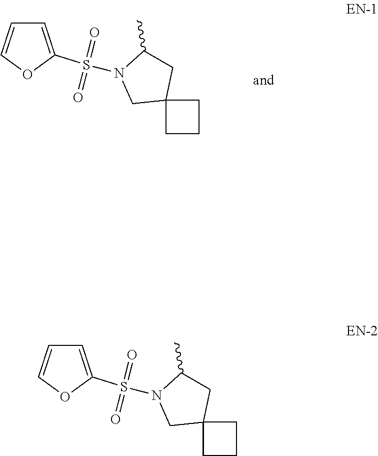

Chiral separation of Example 36 6-(furan-2-ylsulfonyl)-7-methyl-6-azaspiro[3.4]octane provided both enantiomers as mentioned below. Chiral Separation Prep-SFC Method: Column: CHIRALPAK IG (21×250 mm), 5μ; Flow: 20 g/min; Mobile Phase: 80% CO₂+20% (Methanol); ABPR: 100 bar; Temp: 35° C.; UV: 220 nm; DILUENT: Methanol.

Example 37, [EN-1]: Off White Solid (20 mg, 25% yield, 99.74% purity). LCMS: m/z found 256.1 [M+H]⁺, rt=5.55 min (Method 6) [Acquity BEH C8 column (1.7 μm, 50×2.1 mm)]; ¹H NMR (400 MHz, DMSO-d₆): δ 7.99 (s, 1H), 7.19 (d, 1H), 6.71 (dd, 1H), 3.66 (q, 1H), 3.33-3.20 (m, 2H), 2.08-1.91 (m, 2H), 1.89-1.80 (m, 1H), 1.78-1.62 (m, 2H), 1.57 (dd, 1H), 1.51-1.40 (m, 1H), 1.28 (d, 4H).

Example 38, [EN-2]: Off White Solid (20 mg, 25% yield, 99.84% purity). LCMS: m/z found 256.1 [M+H]⁺, rt=5.55 min (Method 6) [Acquity BEH C8 column (1.7 μm, 50×2.1 mm)]; ¹H NMR (400 MHz, DMSO-d₆): δ 7.99 (d, 1H), 7.19 (d, 1H), 6.71 (dd, 1H), 3.67 (q, 1H), 3.35-3.21 (m, 2H), 2.08-1.91 (m, 2H), 1.90-1.79 (m, 1H), 1.80-1.62 (m, 2H), 1.57 (dd, 1H), 1.50-1.40 (m, 1H), 1.28 (d, 4H).

### Example-39

### 7-Methyl-6-(pyrazin-2-ylsulfonyl)-6-azaspiro[3.4]octane

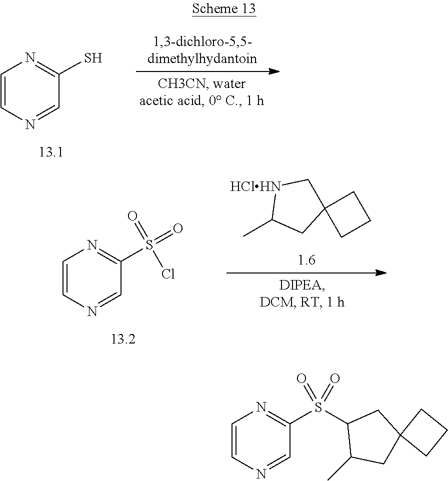

### Pyrazine-2-sulfonyl chloride 13.2

Sulfonyl Chloride (Method M): To a stirred solution of Pyrazine-2-thiol 13.1 (50 mg, 0.446 mmol) in CH3CN (1 mL) was added water (0.1 mL) and catalytic amount of acetic acid. It was then cooled at 0° C. and 1,3-dichloro-5,5-dimethylhydantoin (105.5 mg, 0.536 mmol) was added portion wise. The reaction mixture was then stirred at 0° C. for 1 h. The reaction mixture was diluted with water (2 mL) and extracted with DCM (2×2 mL). The organic part was separated and dried over anhydrous Na₂SO₄. It was then filtered, and the filtrate was used in the forwarding step without evaporation.

### 7-methyl-6-(pyrazin-2-ylsulfonyl)-6-azaspiro[3.4]octane (Example 39)

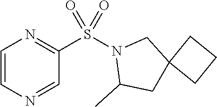 [SMILES: CC1CC2(CCC2)CN1S(=O)(=O)c1cnccn1]

The titled compound was synthesised using sulfonyl chloride 13.2 and 7-methyl-6-azaspiro[3.4]octane hydrochloride 1.6 following the Method A and triethylamine as base. The compound (Example-39) was obtained as light brown gum (30 mg, 25% yield, 99.36% purity). LCMS: m/z found 268.15 [M+H]⁺, rt=1.90 min (Method 1) [Acquity BEH C8 column (1.7 μm, 50×2.1 mm)]; ¹H NMR (400 MHz, DMSO-d₆): δ 9.14 (d, 1H), 8.97 (d, 1H), 8.89-8.83 (m, 1H), 3.93 (q, 1H), 3.39 (s, 2H), 2.09-1.91 (m, 2H), 1.90-1.80 (m, 1H), 1.79-1.63 (m, 2H), 1.57 (dd, 1H), 1.47-1.36 (m, 1H), 1.28 (d, 3H), 1.27-1.20 (m, 1H).

### Example-40

### N-(5-((7-Methyl-6-azaspiro[3.4]octan-6-yl)sulfonyl)-1,3,4-thiadiazol-2-yl)acetamide

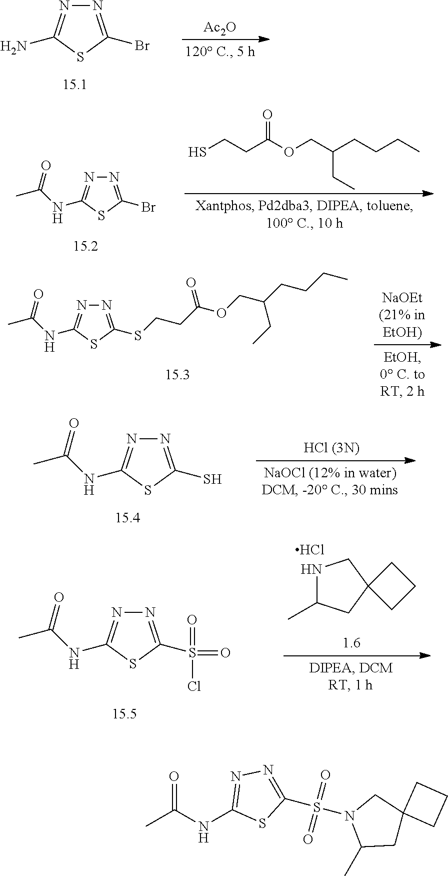

### N-(5-bromo-1,3,4-thiadiazol-2-yl)acetamide 15.2

5-bromo-1,3,4-thiadiazol-2-amine 15.1 (500 mg, 2.809 mmol) was heated with acetic anhydride (10 mL) at 120° C. for 5 h. The reaction mixture was concentrated under reduced pressure and the crude was triturated with pentane and dried under reduced pressure. The crude intermediate 15.2 was obtained as light brown solid (330 mg) and it was used in the forwarding step without further purification.

### N-(5-((7-methyl-6-azaspiro[3.4]octan-6-yl)sulfonyl)-1,3,4-thiadiazol-2-yl)acetamide (Example 40)

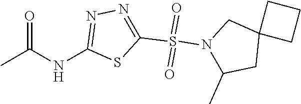 [SMILES: CC(=O)Nc1nnc(S(=O)(=O)N2CC3(CCC3)CC2C)s1]

Desired compound (Example 40) was synthesized from intermediate 15.2 following the procedure described in Example 25. It was obtained as white solid (13 mg, 17% yield, 98.13% purity). LCMS: m/z found 331.16 [M+H]⁺, rt=1.89 min (Method 1) [Acquity BEH C8 column (1.7 μm, 50×2.1 mm)]; ¹H NMR (400 MHz, DMSO-d₆): δ 13.11 (s, 1H), 3.95-3.79 (m, 1H), 3.46 (d, 1H), 3.39 (d, 1H), 2.24 (s, 3H), 2.12-1.98 (m, 2H), 1.88 (dd, 1H), 1.80-1.67 (m, 2H), 1.62 (dd, 1H), 1.55-1.44 (m, 1H), 1.43-1.34 (m, 1H), 1.31 (d, 3H).

### Example-41

### 6-((2,2-Difluorobenzo[d][1,3]dioxol-4-yl)sulfonyl)-7-methyl-6-azaspiro[3.4]octane

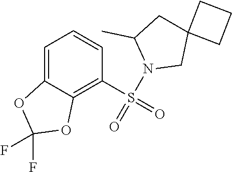 [SMILES: CC1CC2(CCC2)CN1S(=O)(=O)c1cccc2c1OC(F)(F)O2]

The synthesis was performed essentially as described in Method A using amine 1.6 and 2,2-difluorobenzo[d][1,3]dioxole-4-sulfonyl chloride. The compound was purified by Reverse Phase Prep-HPLC and isolated as yellow sticky gum (5 mg, 4% yield, 97.35% purity). Prep-HPLC Method: Preparative HPLC was done on Waters auto purification instrument. Column name: YMC-Actus C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 70% A and 30% B, then to 35% A and 65% B in 3 min, then to 10% A and 90% B in 22 min., then to 5% A and 95% B in 23 min., held this composition up to 25 min. for column washing, then returned to initial composition in 26 min. and held till 28 min. LCMS: m/z found 346.24 [M+H]⁺, rt=1.95 min (Method 2) [YMC Triart C18 column (3 μm, 33×2.1 mm)]; ¹H NMR (400 MHz, DMSO-d₆): δ 7.75 (d, 1H), 7.56 (d, 1H), 7.42 (t, 1H), 3.70 (q, 1H), 3.37 (d, 1H), 3.28 (d, 2H), 2.08-1.91 (m, 2H), 1.92-1.80 (m, 1H), 1.81-1.61 (m, 1H), 1.57 (dd, 1H), 1.48-1.36 (m, 1H), 1.29 (d, 3H), 1.27-1.19 (m, 1H).

### Example-42 and Example 43

### (6-(Thiophen-2-ylsulfonyl)-6-azaspiro[3.4]octan-7-yl)methanol and

### 7-(Methoxymethyl)-6-(thiophen-2-ylsulfonyl)-6-azaspiro[3.4]octane

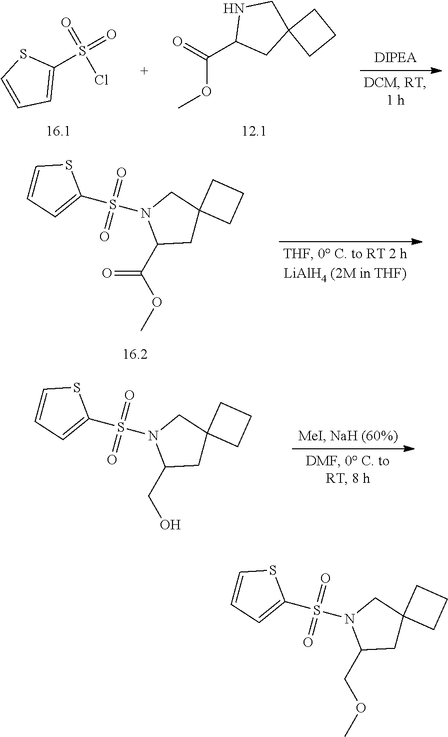 [SMILES: O=S(=O)(Cl)c1cccs1 ; COC(=O)C1CC2(CCC2)CN1 ; COC(=O)C1CC2(CCC2)CN1S(=O)(=O)c1cccs1 ; O=S(=O)(c1cccs1)N1CC2(CCC2)CC1CO ; COCC1CC2(CCC2)CN1S(=O)(=O)c1cccs1]

### Methyl 6-(thiophen-2-ylsulfonyl)-6-azaspiro[3.4]octane-7-carboxylate 16.2

The synthesis was performed essentially as described in Method A using methyl 6-azaspiro[3.4]octane-7-carboxylate 12.1 and thiophene-2-sulfonyl chloride 16.1. The compound was purified by column chromatography over silica gel using 3:7 ethyl acetate: hexane and isolated as light-yellow gum (380 mg, 81% yield). ¹H NMR (400 MHz, DMSO-d₆): δ 8.02 (d, 1H), 7.74 (d, 1H), 7.27 (t, 1H), 4.10 (t, 1H), 3.68 (s, 3H), 3.40 (d, 1H), 3.29 (d, 2H), 2.17 (dd, 1H), 1.99-1.87 (m, 2H), 1.84-1.60 (m, 2H), 1.48 (s, 1H), 1.33-1.23 (m, 1H).

### (6-(Thiophen-2-ylsulfonyl)-6-azaspiro[3.4]octan-7-yl)methanol (Example 42)

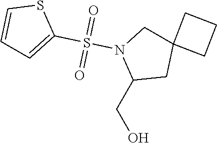 [SMILES: O=S(=O)(c1cccs1)N1CC2(CCC2)CC1CO]

The synthesis was performed essentially as described in Method N using intermediate 16.2. The compound was purified by column chromatography over silica gel using 70% ethyl acetate in hexane and isolated as light brown oil (193 mg, 55% yield, 97.05% purity). LCMS: m/z found 288.16 [M+H]⁺, rt=1.74 min (Method 1) [Acquity BEH C8 column (1.7 μm, 50×2.1 mm)]; ¹H NMR (400 MHz, DMSO-d₆): δ 8.00 (dd, 1H), 7.70 (dd, 1H), 7.26 (dd, 1H), 4.86 (t, 1H), 3.72-3.62 (m, 1H), 3.57-3.46 (m, 1H), 3.44-3.33 (m, 1H), 3.16 (d, 1H), 2.06-1.81 (m, 4H), 1.80-1.65 (m, 3H), 1.51-1.25 (m, 2H).

### 7-(Methoxymethyl)-6-(thiophen-2-ylsulfonyl)-6-azaspiro[3.4]octane (Example 43)

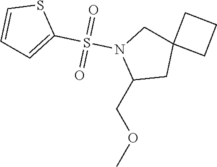 [SMILES: COCC1CC2(CCC2)CN1S(=O)(=O)c1cccs1]

To a stirred solution of Example 42 (80.0 mg, 0.279 mmol) in DMF (5 mL) was added NaH (10 mg, 0.418 mmol) at 0° C. and the reaction mixture was stirred for 30 mins. Mel (0.02 mL, 0.279 mmol) was added to it at 0° C. and the reaction mixture was warmed to RT and stirred for 8 h. Crude reaction mass was quenched with aqueous NH₄Cl (5 mL) and extracted with ethyl acetate (2×10 mL). The combined organic part was washed with water (5 mL) and brine (5 mL), dried over anhydrous Na₂SO₄ and the solvent was evaporated under reduced pressure. The crude was purified by Reverse Phase Prep-HPLC. The compound (Example 43) was obtained as white solid (20 mg, 24% yield, 98.74% purity). Prep-HPLC Method: Preparative HPLC was done on Waters auto purification instrument. Column name: YMC-Actus C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 70% A and 30% B, then to 40% A and 60% B in 3 min, then to 10% A and 90% B in 22 min., then to 5% A and 95% B in 23 min., held this composition up to 25 min. for column washing, then returned to initial composition in 26 min. and held till 28 min. LCMS: m/z found 302.17 [M+H]⁺, rt=5.50 min (Method 6) [Acquity BEH C8 column (1.7 μm, 50×2.1 mm)]; ¹H NMR (400 MHz, DMSO-d₆): δ 8.01 (d, 1H), 7.73 (d, 1H), 7.28-7.24 (m, 1H), 3.68-3.54 (m, 2H), 3.37 (d, 1H), 3.29 (d, 4H), 3.20 (d, 1H), 2.01-1.91 (m, 1H), 1.90-1.77 (m, 3H), 1.76-1.61 (m, 2H), 1.48-1.35 (m, 1H), 1.33-1.22 (m, 1H).

### Example-44

### 7-Methyl-6-((4-phenylthiophen-2-yl)sulfonyl)-6-azaspiro[3.4]octane

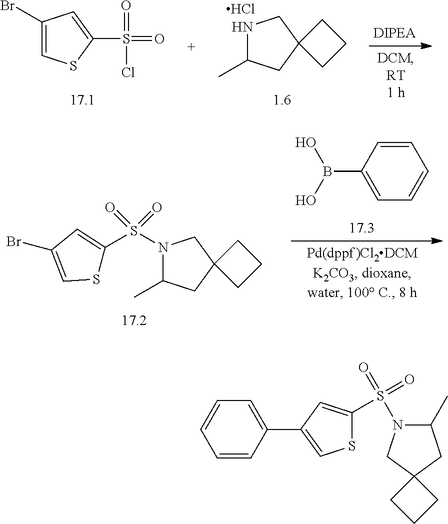

6-((4-Bromothiophen-2-yl)sulfonyl)-7-methy-6-azaspiro[3.4]octane 17.2: The synthesis was performed essentially as described in Method A using amine 1.6 and 4-bromothiophene-2-sulfonyl chloride 17.1. The compound was purified by column chromatography over silica gel using 3:7 ethyl acetate:hexane and isolated as light-yellow gum (80 mg, 59% yield). ¹H NMR (400 MHz, DMSO-d₆): δ 8.10 (d, 1H), 7.79 (d, 1H), 3.61 (q, 1H), 3.35 (d, 1H), 3.24 (d, 1H), 2.04-1.94 (m, 2H), 1.91-1.79 (m, 1H), 1.78-1.64 (m, 2H), 1.58 (dd, 1H), 1.50-1.37 (m, 1H), 1.37-1.23 (m, 4H).

### 7-Methyl-6-((4-phenylthiophen-2-yl)sulfonyl)-6-azaspiro[3.4]octane (Example 44)

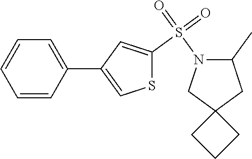 [SMILES: CC1CC2(CCC2)CN1S(=O)(=O)c1cc(-c2ccccc2)cs1]

Suzuki Coupling (Method P): To a stirred solution of 17.2 (60 mg, 0.403 mmol) in 1,4-dioxane (1 mL) and water (1 mL) was added 17.3 (59 mg, 0.483 mmol) and K₂CO₃ (167 mg, 1.208 mmol). The reaction mixture was degasified with argon and Pd(dppf)Cl₂·DCM (6.6 mg, 0.0080 mmol) was added to it. It was then stirred at 100° C. for 8 h. The reaction mass was filtered, and the filtrate was concentrated under reduced pressure. It was diluted with ethyl acetate (10 mL) and the organic part was washed with water (5 ml) followed by brine (5 mL). It was dried over anhydrous Na₂SO₄ and concentrated under reduced pressure. The crude product was purified by Reverse Phase Prep-HPLC. The compound (Example 44) was isolated as colourless sticky gum (30 mg, 21% yield, 99.80% purity). Prep-HPLC Method: Preparative HPLC was done on Waters auto purification instrument. Column name: YMC-Actus Triart C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 70% A and 30% B, then to 35% A and 65% B in 3 min, then to 5% A and 95% B in 22 min., then to 5% A and 95% B in 23 min., held this composition up to 25 min. for column washing, then returned to initial composition in 26 min. and held till 28 min. LCMS: m/z found 348.19 [M+H]⁺, rt=2.26 min (Method 1) [Acquity BEH C8 column (1.7 μm, 50×2.1 mm)]; ¹H NMR (400 MHz, DMSO-d₆): δ 8.29 (d, 1H), 8.14 (d, 1H), 7.84-7.76 (m, 2H), 7.44 (t, 2H), 7.35 (t, 1H), 3.69 (q, 1H), 3.37 (d, 1H), 2.04-1.92 (m, 2H), 1.90-1.78 (m, 1H), 1.76-1.59 (m, 2H), 1.57 (dd, 1H), 1.46-1.34 (m, 1H), 1.33 (d, 3H), 1.31-1.22 (m, 1H).

### Example-45

### 7-Methyl-6-(phenylsulfonyl)-6-azaspiro[3.4]octane

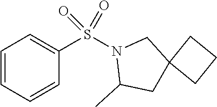 [SMILES: CC1CC2(CCC2)CN1S(=O)(=O)c1ccccc1]

The synthesis was performed essentially as described in Scheme 3 using amine 1.6 and benzenesulfonyl chloride. The compound was purified by Reverse Phase Prep-HPLC and the compound (Example 45) was obtained as colourless sticky oil (5.2 mg, 8% yield, 99.97% purity). Preparative HPLC method: Preparative HPLC was done on Waters auto purification instrument. Column name: YMC-Actus C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 70% A and 30% B, then to 40% A and 60% B in 3 min, then to 10% A and 90% B in 22 min., then to 5% A and 95% B in 23 min., held this composition up to 25 min. for column washing, then returned to initial composition in 26 min. and held till 28 min. LCMS: m/z found 266.3 [M+H]⁺, rt=1.86 min (Method 7) [YMC Triart C18 column (3 μm, 33×2.1 mm)]; ¹H NMR (400 MHz, DMSO-d₆): δ 7.85-7.78 (m, 2H), 7.69 (t, 1H), 7.62 (t, 2H), 3.53 (q, 1H), 3.29 (d, 1H), 3.16 (d, 1H), 2.00-1.76 (m, 3H), 1.75-1.57 (m, 2H), 1.51 (dd, 1H), 1.37-1.20 (m, 4H), 1.14 (q, 1H).

### Example-46 and Example-47

### 7-Methyl-6-(phenylsulfonyl)-6-azaspiro[3.4]octane ([EN-1]); and

### 7-methyl-6-(phenylsulfonyl)-6-azaspiro[3.4]octane ([EN-2])

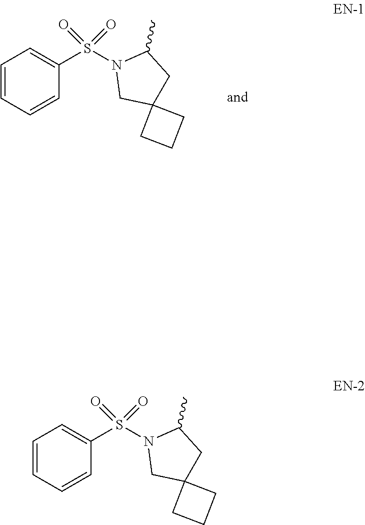

Chiral separation by Normal Phase of racemic 7-methyl-6-(phenylsulfonyl)-6-azaspiro[3.4]octane (Example 45) provided both isomers as mentioned below.

Chiral Separation Method: Chiral separation was done on Agilent 1200 series instrument. Column name: Chiralpak IA (250×20 mm), 5μ, operating at ambient temperature and flow rate of 18.0 mL/min. Mobile phase: 0.1% Isopropylamine in the mixture of 85% Hexane, and 15% Ethanol, held this isocratic mixture up to 15 min with wavelength of 230 nm.

Example 46 [EN-1]: White solid (69 mg, 23% yield, 90.08% purity). LCMS: m/z found 266.04 [M+H]⁺, rt=1.99 min (Method 2) [YMC Triart C18 column (3 μm, 33×2.1 mm)]; ¹H NMR (400 MHz, DMSO-d₆) δ 7.86-7.76 (m, 2H), 7.74-7.57 (m, 3H), 3.53 (q, 1H), 3.33-3.22 (m, 1H), 3.16 (d, 1H), 1.99-1.75 (m, 3H), 1.75-1.56 (m, 2H), 1.51 (dd, 1H), 1.30 (d, 3H), 1.28-1.21 (m, 1H), 1.19-1.04 (m, 1H).

Example 47 [EN-2]: White solid (89 mg, 30% yield, 97.94% purity). LCMS: m/z found 266.04 [M+H]⁺, rt=1.99 min (Method 2) [YMC Triart C18 column (3 μm, 33×2.1 mm)]; ¹H NMR (400 MHz, DMSO-d₆) δ 7.86-7.78 (m, 2H), 7.73-7.66 (m, 1H), 7.66-7.57 (m, 2H), 3.53 (q, 1H), 3.31-3.24 (m, 1H), 3.16 (d, 1H), 1.97-1.75 (m, 3H), 1.75-1.56 (m, 2H), 1.51 (dd, 1H), 1.30 (d, 3H), 1.29-1.19 (m, 1H), 1.21-1.07 (m, 1H).

### Example-48

### 7-Methyl-6-(pyridin-2-ylsulfonyl)-6-azaspiro[3.4]octane

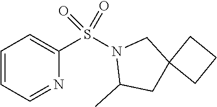 [SMILES: CC1CC2(CCC2)CN1S(=O)(=O)c1ccccn1]

The synthesis was performed essentially as described in Method A using amine 1.6 and pyridine-2-sulfonyl chloride. The compound was purified by Reverse Phase Prep-HPLC and the compound (Example 48) was obtained as colourless sticky oil (69 mg, 92% yield, 99.41% purity). Preparative HPLC method: Preparative HPLC was done on Waters auto purification instrument. Column name: YMC-Actus C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 70% A and 30% B, then to 40% A and 60% B in 3 min, then to 10% A and 90% B in 22 min., then to 5% A and 95% B in 23 min., held this composition up to 25 min. for column washing, then returned to initial composition in 26 min. and held till 28 min. LCMS: m/z found 267.2[M+H]⁺, rt=2.25 min (Method 14) [Acquity BEH C8 column (1.7 μm, 50×2.1 mm)]; ¹H NMR (400 MHz, DMSO-d₆): δ 8.74 (d, 1H), 8.14-8.04 (m, 1H), 7.94 (d, 1H), 7.73-7.65 (m, 1H), 3.94 (q, 1H), 3.35 (d, 2H), 2.06-1.90 (m, 2H), 1.89-1.79 (m, 1H), 1.79-1.60 (m, 2H), 1.55 (dd, 1H), 1.48-1.35 (m, 1H), 1.27 (d, 3H), 1.25-1.17 (m, 1H).

### Example-49

### 7,7-Dimethyl-6-(thiophen-3-ylsulfonyl)-6-azaspiro[3.4]octane

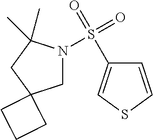 [SMILES: CC1(C)CC2(CCC2)CN1S(=O)(=O)c1ccsc1]

The synthesis was performed essentially as described in Method A using 7,7-dimethyl-6-azaspiro[3.4]octane hydrochloride and thiophene-3-sulfonyl chloride. The compound was purified by Reverse Phase Prep-HPLC and the compound (Example 49) was obtained as colourless sticky oil (5 mg, 8.0% yield, 99.94% purity). Preparative HPLC method: Preparative HPLC was done on Waters auto purification instrument. Column name: Sunfire C18 (150×19 mm, 10μ) operating at ambient temperature and flow rate of 16 ml/min. Mobile phase: A=10 mM Ammonium Acetate in water, B=Acetonitrile; Gradient Profile: Mobile phase initial composition of 60% A and 40% B, then to 40% A and 60% B in 4 min, then to 28% A and 72% B in 16 min., then to 5% A and 95% B in 17 min., held this composition up to 20 min. for column washing, then returned to initial composition in 21 min. and held till 23 min. LCMS: m/z found 286.3 [M+H]⁺, rt=5.92 min (Method 4) [YMC Triart C18 column (3 μm, 33×2.1 mm)]; ¹H NMR (400 MHz, DMSO-d₆): δ 7.92 (dd, 1H), 7.63 (dd, 1H), 7.18 (t, 1H), 3.41 (s, 2H), 2.02-1.91 (m, 2H), 1.89-1.77 (m, 6H), 1.34 (s, 6H).

### Example-50

### 6-((2,2-Difluorobenzo[d][1,3]dioxol-5-yl)sulfonyl)-7-ethyl-6-azaspiro[3.4]octane

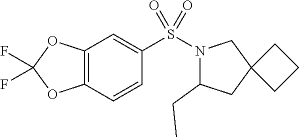 [SMILES: CCC1CC2(CCC2)CN1S(=O)(=O)c1ccc2c(c1)OC(F)(F)O2]

The synthesis was performed essentially as described in Method A using 7-ethyl-6-azaspiro[3.4]octane hydrochloride and 2,2-difluorobenzo[d][1,3]dioxole-5-sulfonyl chloride. The compound was purified by column chromatography over silica gel using 20-30% Ethyl acetate in hexane as eluent and the compound (Example 50) was obtained as colourless sticky oil (27 mg, 39% yield, 99.96% purity). LCMS: m/z found 360.21 [M+H]⁺, rt=2.36 min (Method 1) [Acquity BEH C8 column (1.7 μm, 50×2.1 mm)]; ¹H NMR (400 MHz, DMSO-d₆): δ 7.94 (s, 1H), 7.73 (d, 1H), 7.63 (d, 1H), 3.49-3.39 (m, 1H), 3.26 (s, 2H), 2.01-1.75 (m, 4H), 1.76-1.42 (m, 4H), 1.39-1.17 (m, 2H), 0.84 (t, 3H).

### Example-51

### 2-Ethyl-5-((7-ethyl-6-azaspiro[3.4]octan-6-yl)sulfonyl)benzo[d]oxazole

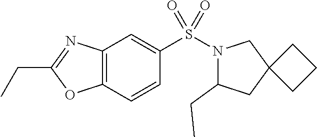 [SMILES: CCc1nc2cc(S(=O)(=O)N3CC4(CCC4)CC3CC)ccc2o1]

The synthesis was performed essentially as described in Method A using 7-ethyl-6-azaspiro[3.4]octane hydrochloride and 2-ethylbenzo[d]oxazole-5-sulfonyl chloride. The compound was purified by column chromatography over silica gel using 20-30% Ethyl acetate in hexane as eluent and the compound (Example 51) was obtained as colourless sticky oil (61 mg, 90% yield, 99.89% purity). LCMS: m/z found 349.32 [M+H]⁺, rt=2.20 min (Method 1) [Acquity BEH C8 column (1.7 μm, 50×2.1 mm)]; ¹H NMR (400 MHz, DMSO-d₆): δ 8.11 (s, 1H), 7.90 (d, 1H), 7.84 (dd, 1H), 3.52-3.38 (m, 1H), 3.26 (s, 2H), 3.03 (q, 2H), 1.96-1.86 (m, 2H), 1.85-1.74 (m, 2H), 1.71-1.44 (m, 4H), 1.36 (t, 3H), 1.29-1.03 (m, 2H), 0.85 (t, 3H).

### Example-52

### 2-Methyl-1-(5-((7-methyl-6-azaspiro[3.4]octan-6-yl)sulfonyl)isoindolin-2-yl)propan-1-one

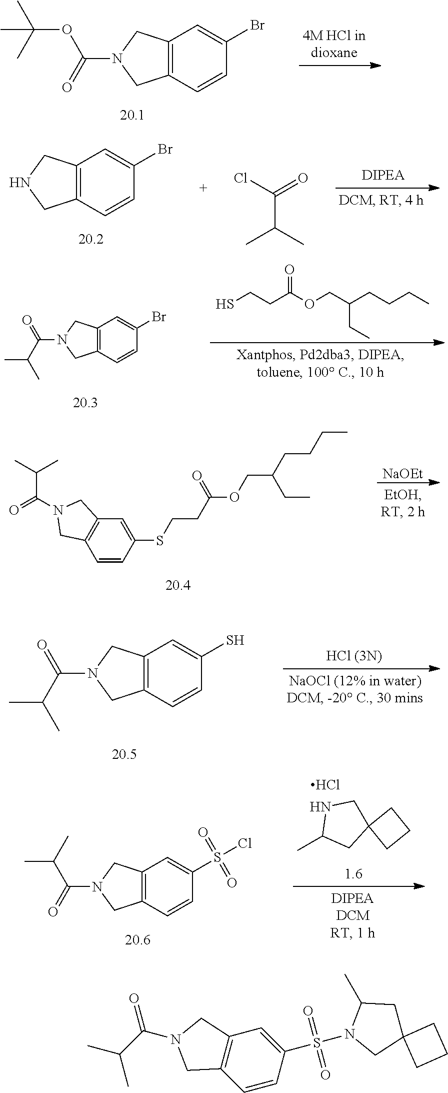

5-Bromoisoindoline 20.2: Intermediate 20.2 was synthesised from tert-butyl 5-bromoisoindoline-2-carboxylate 20.1 following produced described in Method H. The crude intermediate 20.2 was used in the forwarding step without further purification. ¹H NMR (400 MHz, DMSO-d₆) δ 9.72 (s, 2H), 7.64 (s, 1H), 7.56 (dd, 1H), 7.37 (d, 1H), 5.77 (s, 4H), 4.53-4.42 (m, 4H).

1-(5-Bromoisoindolin-2-yl)-2-methylpropan-1-one 20.3: Intermediate 20.3 was synthesised following the procedure described in Method B using intermediate 20.2 and isobutyryl chloride. The crude was purified by column chromatography over silica gel using 20-30% Ethyl acetate in hexane as eluent. ¹H NMR (400 MHz, DMSO-d₆) δ 7.57 (d, 1H), 7.48 (d, 1H), 7.30 (t, 1H), 4.87 (d, 2H), 4.60 (d, 2H), 2.81-2.68 (m, 1H), 1.05 (d, 6H).

### 2-methyl-1-(5-((7-methyl-6-azaspiro[3.4]octan-6-yl)sulfonyl)isoindolin-2-yl)propan-1-one (Example 52)

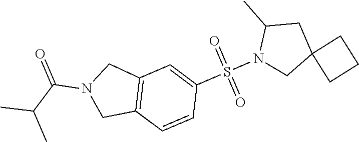 [SMILES: CC(C)C(=O)N1Cc2ccc(S(=O)(=O)N3CC4(CCC4)CC3C)cc2C1]

Desired compound (Example 52) was synthesized from intermediate 20.3 following the procedure described in Example 25. It was obtained as white solid (12 mg, 10% yield, 99.56% purity). LC-MS: m/z found 377.31 [M+H]⁺, rt=1.96 min (Method 1) [Acquity BEH C8 column (1.7 μm, 50×2.1 mm)]; ¹H NMR (400 MHz, DMSO-d₆): δ 7.81 (d, 1H), 7.75 (d, 1H), 7.57 (dd, 1H), 4.98 (s, 2H), 4.70 (s, 2H), 3.55 (q, 1H), 3.33 (d, 1H), 3.15 (dd, 1H), 2.83-2.70 (m, 1H), 2.00-1.91 (m, 1H), 1.90-1.79 (m, 2H), 1.75-1.63 (m, 2H), 1.58-1.50 (m, 1H), 1.43-1.32 (m, 1H), 1.29 (d, 4H), 1.07 (dd, 6H).

### Example-53

### 7-Methyl-6-((5-methylfuran-2-yl)sulfonyl)-6-azaspiro[3.4]octane

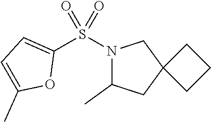 [SMILES: Cc1ccc(S(=O)(=O)N2CC3(CCC3)CC2C)o1]

The synthesis was performed essentially as described in Method A using 5-methylfuran-2-sulfonyl chloride and amine 1.6. It was purified by column chromatography over silica gel using 20-30% Ethyl acetate in hexane as eluent and the compound (Example 53) was obtained as colour less sticky gum (80 mg, 88% yield, 99.35% purity). LCMS: m/z found 270.0 [M+H]⁺, rt=3.57 min, (Method 3) [Zorbax Ext C18 column (5 μm, 50×4.6 mm)]; ¹H NMR (400 MHz, DMSO-d₆): δ 7.06 (d, 1H), 6.33 (s, 1H), 3.67 (q, 1H), 3.29-3.17 (m, 2H), 2.33 (s, 3H), 2.09-1.93 (m, 2H), 1.90-1.63 (m, 3H), 1.60-1.43 (m, 2H), 1.37-1.20 (m, 4H).

### Example-54 and Example-55

### 7-Methyl-6-((5-methylfuran-2-yl)sulfonyl)-6-azaspiro[3.4]octane (EN-1) and

### 7-methyl-6-((5-methylfuran-2-yl)sulfonyl)-6-azaspiro[3.4]octane (EN-2)

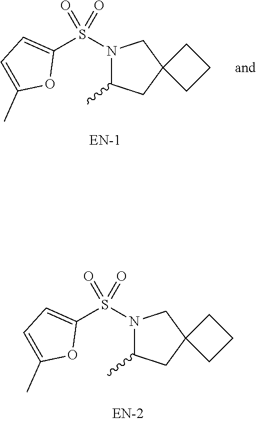

Chiral separation of racemic compound (Example 53) provided ˜18 mg each of both isomers as mentioned below. Chiral separation method: chiral separation was done on an Agilent 1200 series instrument. Column name: Chiralpak IG (250×21 mm), 5μ, operating at ambient temperature and flow rate of 21.0 mL/min. Mobile phase: 0.1% isopropylamine in a mixture of 90% Hexane and 10% Ethanol, held this isocratic mixture up to 20 min with wavelength of 238 nm.

(Example 54 [EN-1]): Light yellow oil (18 mg, 17% yield, 99.74% purity). LCMS: m/z found 270.33 [M+H]⁺, rt=5.14 min, (Method 4) [YMC Triart C18 column (3 μm, 33×2.1 mm)]; ¹H NMR (400 MHz, DMSO-d₆): δ 7.04 (d, 1H), 6.32 (d, 1H), 3.67 (q, 1H), 3.30-3.19 (m, 2H), 2.33 (s, 3H), 2.04 (dd, 1H), 2.01-1.91 (m, 1H), 1.90-1.74 (m, 1H), 1.76-1.64 (m, 2H), 1.56 (dd, 1H), 1.54-1.43 (m, 1H), 1.37-1.28 (m, 1H), 1.28 (d, 3H).

(Example 55 [EN-2]): Light yellow oil (18 mg, 17% yield, 99.73% purity). LCMS: m/z found 270.33 [M+H]⁺, rt=1.86 min, (Method 2) [YMC Triart C18 column (3 μm, 33×2.1 mm)]; ¹H NMR (400 MHz, DMSO-d₆): δ 7.05 (d, 1H), 6.33 (d, 1H), 3.67 (q, 1H), 3.30-3.19 (m, 2H), 2.33 (s, 3H), 2.09-1.91 (m, 2H), 1.90-1.74 (m, 1H), 1.76-1.64 (m, 2H), 1.56 (dd, 1H), 1.52-1.43 (m, 1H), 1.37-1.28 (m, 1H), 1.28 (d, 3H).

### Example-56

### 5-Chloro-6-((7-methyl-6-azaspiro[3.4]octan-6-yl)sulfonyl)benzo[d]oxazol-2(3H)-one

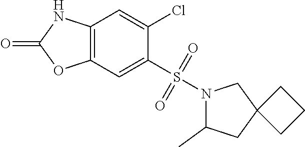 [SMILES: CC1CC2(CCC2)CN1S(=O)(=O)c1cc2oc(=O)[nH]c2cc1Cl]

The synthesis was performed essentially as described in Method A using 5-chloro-2-oxo-2,3-dihydrobenzo[d]oxazole-6-sulfonyl chloride and 7-methyl-6-azaspiro[3.4]octane hydrochloride 1.6. It was purified by column chromatography over silica gel using 20-30% Ethyl acetate in hexane as eluent and the compound (Example 56) was obtained as colourless sticky solid (43 mg, 64% yield, 99.25% purity). LCMS: m/z found 357.23 [M+H]⁺, rt=1.77 min (Method 2) [YMC Triart C18 column (3 μm, 33×2.1 mm)]; ¹H NMR (400 MHz, DMSO-d₆): δ 12.29 (s, 1H), 7.84 (s, 1H), 7.36 (s, 1H), 3.91 (q, 1H), 3.42-3.33 (m, 2H), 2.10 (dd, 1H), 2.05-1.86 (m, 2H), 1.84-1.62 (m, 4H), 1.59 (dd, 1H), 1.13 (d, 3H).

### Example-57 and Example-58

### 5-Chloro-6-((7-methyl-6-azaspiro[3.4]octan-6-yl)sulfonyl)benzo[d]oxazol-2(3H)-one (EN-1); and

### 5-chloro-6-((7-methyl-6-azaspiro[3.4]octan-6-yl)sulfonyl)benzo[d]oxazol-2(3H)-one (EN-2)

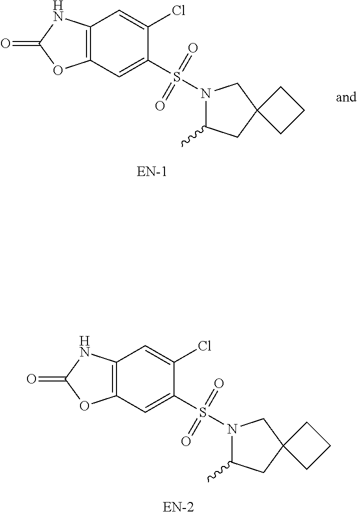

Chiral separation of racemic compound Example 56 provided ˜5 mg each of both enantiomers as mentioned below. Chiral Separation Method: Column name: i-Cellulose-C (21.1 mm×250 mm). 5μ, Flow: 25 g/min, Mobile phase: 70% CO2+30% (0.5% isopropylamine in isopropanol), ABPR:120 bar, Temp.: 35° C., UV: 220 nm, diluents: Methanol.

Example 57 [EN-1]: Colourless sticky solid (4.35 mg, 3% yield, 97.07% purity) LCMS: m/z found 357.14 [M+H]⁺, rt=1.93 min, (Method 1) [Acquity BEH C8 column (1.7 μm, 50×2.1 mm)]; ¹H NMR (400 MHz, DMSO-d₆): δ 7.74-7.49 (m, 2H), 7.46 (s, 1H), 7.00 (s, 1H), 3.86 (q, 1H), 2.14-1.85 (m, 3H), 1.82-1.51 (m, 5H), 1.20-1.12 (m, 5H).

Example 58 [EN-2]: Colourless sticky solid (4.35 mg, 3% yield, 96.29% purity). LCMS: m/z found 357.14 [M+H]⁺, rt=1.93 min, (Method 1) [Acquity BEH C8 column (1.7 μm, 50×2.1 mm)]; ¹H NMR (400 MHz, DMSO-d₆): δ 7.83-7.45 (m, 2H), 7.40 (s, 1H), 6.93 (s, 1H), 3.84 (q, 1H), 2.05 (dd, 1H), 2.03-1.84 (m, 2H), 1.82-1.51 (m, 5H), 1.20-1.03 (m, 5H).

### Example-59

### 5-((7-Methyl-6-azaspiro[3.4]octan-6-yl)sulfonyl)-1,3-dihydro-2H-benzo[d]imidazol-2-one

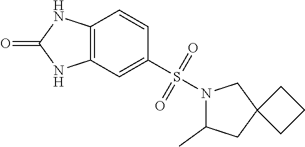 [SMILES: CC1CC2(CCC2)CN1S(=O)(=O)c1ccc2[nH]c(=O)[nH]c2c1]

The synthesis was performed essentially as described in Method A using 2-oxo-2,3-dihydro-1H-benzo[d]imidazole-5-sulfonyl chloride and 7-methyl-6-azaspiro[3.4]octane hydrochloride 1.6. It was purified by column chromatography over silica gel using 30% Ethyl acetate in hexane as eluent and the compound (Example 59) was obtained as white solid (40 mg, 57% yield, 94.89% purity)

LCMS: m/z found 322.34 [M+H]⁺, rt=1.51 min, (Method 2) [YMC Triart C18 column (3 μm, 33×2.1 mm)]; ¹H NMR (400 MHz, DMSO-d₆): δ 11.12 (s, 1H), 10.94 (s, 1H), 7.42 (dd, 1H), 7.26 (s, 1H), 7.10 (d, 1H), 3.70-3.56 (m, 1H), 3.48 (q, 1H), 3.22-3.04 (m, 2H), 2.06-1.76 (m, 3H), 1.75-1.59 (m, 2H), 1.51 (dd, 1H), 1.38-1.15 (m, 4H)

### Example-60

### 7-Methyl-6-(thiophen-2-ylsulfonyl)-6-azaspiro[3.5]nonane

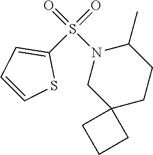 [SMILES: CC1CCC2(CCC2)CN1S(=O)(=O)c1cccs1]

The synthesis was performed essentially as described in Method A using 7-methyl-6-azaspiro[3.5]nonane and thiophene-2-sulfonyl chloride. The compound was purified by column chromatography over silica gel using 30% EtOAc in hexane and was obtained as white solid (20 mg, 50% yield, 99.38% purity). LCMS: m/z found 286.20 [M+H]⁺, rt=1.94 min, (Method 1) [Acquity BEH C8 column (1.7 μm, 50×2.1 mm)]; ¹H NMR (400 MHz, DMSO-d₆): δ 7.95 (dd, 1H), 7.63 (dd, 1H), 7.19 (dd, 1H), 4.03 (t, 1H), 3.52 (d, 1H), 2.80 (d, 1H), 1.89-1.76 (m, 2H), 1.78-1.43 (m, 7H), 1.40-1.30 (m, 1H), 0.90 (d, 3H).

### Example-61

### 6-((7-Ethyl-6-azaspiro[3.4]octan-6-yl)sulfonyl)-2,3-dihydrobenzo[b]thiophene 1,1-dioxide

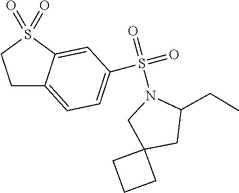 [SMILES: CCC1CC2(CCC2)CN1S(=O)(=O)c1ccc2c(c1)S(=O)(=O)CC2]

The synthesis was performed essentially as described in Method A using amine 3.6 and 2,3-dihydrobenzo[b]thiophene-6-sulfonyl chloride 1,1-dioxide. The compound was purified by column chromatography over silica gel using 40% EtOAc in hexane and was obtained as colourless sticky solid (27 mg, 65% yield, 96.75% purity). LCMS: m/z found 370.18 [M+H]⁺, rt=2.02 min, (Method 1) [Acquity BEH C8 column (1.7 μm, 50×2.1 mm)]; ¹H NMR (400 MHz, DMSO-d₆): δ 8.15-8.04 (m, 2H), 7.79 (d, 1H), 3.70 (t, 2H), 3.46 (t, 3H), 2.05-1.41 (m, 10H), 1.28-1.19 (m, 2H), 0.85 (t, 3H).

### Example-62 and Example-63

### 6-((7-Ethyl-6-azaspiro[3.4]octan-6-yl)sulfonyl)-2,3-dihydrobenzo[b]thiophene 1,1-dioxide (A [EN-1]); and

### 6-((7-ethyl-6-azaspiro[3.4]octan-6-yl)sulfonyl)-2,3-dihydrobenzo[b]thiophene 1,1-dioxide ([EN-2])

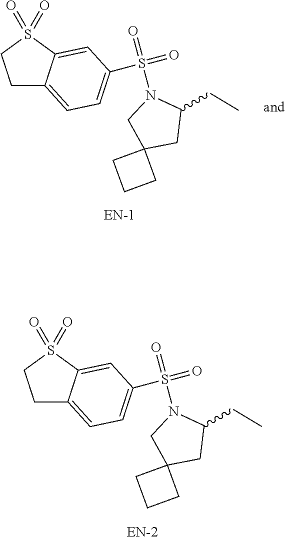 [SMILES: CCC1CC2(CCC2)CN1S(=O)(=O)c1ccc2c(c1)S(=O)(=O)CC2 ; CCC1CC2(CCC2)CN1S(=O)(=O)c1ccc2c(c1)S(=O)(=O)CC2]

Chiral separation of racemic compound (Example 61) provided ˜15 mg each of both isomers as mentioned below. The enantiomers were separated by Normal Phase Chiral Preparative HPLC: Chiral separation was done on Agilent 1200 series instrument. Column name: Chiralpak IC (250×21 mm), 5μ, operating at ambient temperature and flow rate of 21.0 mL/min. Mobile phase: 0.1% Iso Propylamine in the mixture of 70% Hexane, 15% Dichloromethane and 15% Ethanol, held this isocratic mixture up to 25 min with wavelength of 236 nm.

Example 62 [EN-1]: Light brown sticky solid (15 mg, 29% yield, 99.02% purity) LCMS: m/z found 370.22 [M+H]⁺, rt=2.00 min, (Method 1) [Acquity BEH C8 column (1.7 μm, 50×2.1 mm)]; ¹H NMR (400 MHz, DMSO-d₆): δ 8.11 (dd, 1H), 8.06 (s, 1H), 7.79 (d, 1H), 3.70 (t, 2H), 3.46 (t, 3H), 2.01-1.75 (m, 5H), 1.76-1.56 (m, 4H), 1.55-1.44 (m, 1H), 1.32-1.18 (m, 2H), 0.85 (t, 3H).

Example 63 [EN-2]: Light brown sticky solid (15 mg, 29% yield, 97.40% purity) LCMS: m/z found 370.22 [M+H]⁺, rt=2.00 min, (Method 1) [Acquity BEH C8 column (1.7 μm, 50×2.1 mm)]; ¹H NMR (400 MHz, DMSO-d₆): δ 8.11 (dd, 1H), 8.07 (s, 1H), 7.79 (d, 1H), 3.70 (t, 2H), 3.46 (t, 3H), 2.01-1.73 (m, 5H), 1.76-1.55 (m, 4H), 1.56-1.44 (m, 1H), 1.33-1.17 (m, 2H), 0.85 (t, 3H)

### Example-64

### 4-Ethyl-5-((7-methyl-6-azaspiro[3.4]octan-6-yl)sulfonyl)thiazole

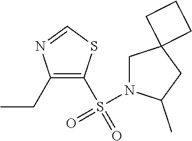 [SMILES: CCc1ncsc1S(=O)(=O)N1CC2(CCC2)CC1C]

Desired compound (Example 64) was synthesized from 5-bromo-4-ethylthiazole following the procedure described in Example 25. It was purified by Column Chromatography over silica gel using 3:7 ethyl acetate:hexane and obtained as colourless sticky gum (7 mg, 10% yield, 90.44% purity). LCMS: m/z found 301.24 [M+H]⁺, rt=2.17 min, (Method 1) [Acquity BEH C8 column (1.7 μm, 50×2.1 mm)]; ¹H NMR (400 MHz, DMSO-d₆): δ 9.30 (s, 1H), 3.63 (q, 1H), 3.39 (d, 1H), 3.24 (s, OH), 3.17 (d, 1H), 3.05 (q, 2H), 2.06-1.95 (m, 2H), 1.93-1.84 (m, 1H), 1.80-1.68 (m, 2H), 1.66-1.48 (m, 2H), 1.44-1.34 (m, 1H), 1.29 (d, 3H), 1.25 (t, 3H).

### Example-65

### 6-((1,3-Dihydroisobenzofuran-5-yl)sulfonyl)-7-methyl-6-azaspiro[3.4]octane

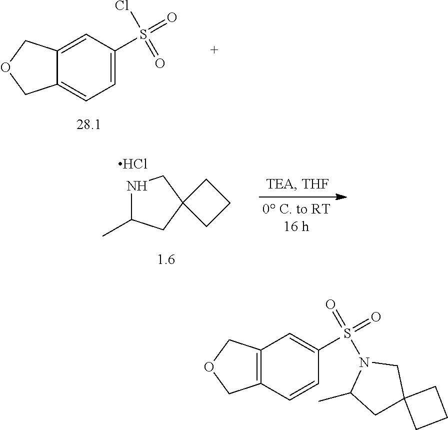

Synthesis of sulfonamide (Method U): To a solution of 7-methyl-6-azaspiro[3.4]octane hydrochloride 1.6 (40.8 mg, 0.252 mmol) in THF (2 mL) was added TEA (0.12 mL, 0.915 mmol) followed by 3-dihydroisobenzofuran-5-sulfonyl chloride 28.1 (50 mg, 0.229 mmol) at 0° C. The reaction was warmed to RT and stirred for 16 h. The reaction mixture was concentrated under reduced pressure to afford crude compound which was purified by column chromatography over silica gel using 20% ethyl acetate in hexane to afford desired compound (Example 65) as off-white solid (50 mg, 71% yield, 95.9% purity). LCMS: m/z found 308.32 [M+H]⁺, rt=1.81 min (Method 2) [YMC Triart C18 column (3 μm, 33×2.1 mm)]; ¹H NMR (400 MHz, DMSO-d₆): δ 7.81-7.69 (m, 2H), 7.53 (d, 1H), 5.06 (s, 4H), 3.54 (q, 1H), 3.15 (d, 1H), 2.02-1.76 (m, 3H), 1.73-1.62 (m, 2H), 1.53 (dd, 1H), 1.43-1.18 (m, 6H).

### Example-66

### 6-(Benzofuran-2-ylsulfonyl)-7-methyl-6-azaspiro[3.4]octane

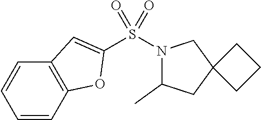 [SMILES: CC1CC2(CCC2)CN1S(=O)(=O)c1cc2ccccc2o1]

The synthesis was performed essentially as described in Method U using benzofuran-2-sulfonyl chloride and 7-methyl-6-azaspiro[3.4]octane hydrochloride 1.6. The crude was purified by column chromatography over silica gel using 20% ethyl acetate in hexane and desired compound (Example 66) was isolated as colorless sticky gum (42 mg, 60% yield, 99.95% purity). LCMS: m/z found 306.2 [M+H]⁺, rt=2.47 min (Method 14) [Acquity BEH C8 column (1.7 μm, 50×2.1 mm)]; ¹H NMR (400 MHz, DMSO-d₆): δ 7.81 (d, 1H), 7.74 (d, 1H), 7.67 (s, 1H), 7.58-7.49 (m, 1H), 7.40 (t, 1H), 3.82 (q, 1H), 3.38 (s, 2H), 2.07 (dd, 1H), 2.04-1.92 (m, 1H), 1.88-1.76 (m, 1H), 1.76-1.54 (m, 3H), 1.42-1.33 (m, 1H), 1.33 (d, 3H), 1.31-1.22 (m, 1H).

### Example-67

### 2-((7-Methyl-6-azaspiro[3.4]octan-6-yl)sulfonyl)benzo[d]thiazole

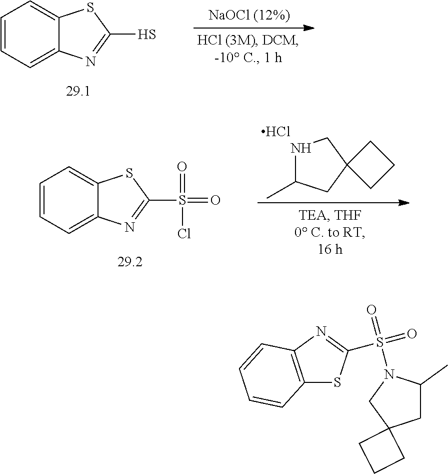

### Benzo[d]thiazole-2-sulfonyl chloride 29.1

The intermediate 29.2 was synthesised from benzo[d]thiazole-2-thiol 29.1 following the protocol described in Method C. The volatiles were evaporated under reduced pressure at 20° C. and resulting crude was used in the forwarding step.

### 2-((7-Methyl-6-azaspiro [3.4]octan-6-yl)sulfonyl)benzo[d]thiazole (Example 67)

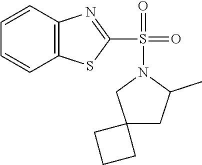 [SMILES: CC1CC2(CCC2)CN1S(=O)(=O)c1nc2ccccc2s1]

The synthesis was performed essentially as described in Method U using intermediate 29.2 and 7-methyl-6-azaspiro[3.4]octane hydrochloride 1.6. The crude was purified by column chromatography over silica gel using 20% ethyl acetate in hexane and desired compound (Example 67) was isolated as colorless sticky gum (32 mg, 69% yield, 97.26% purity). LCMS: m/z found 323.2 [M+H]⁺, rt=2.45 min (Method 14) [Acquity BEH C8 column (1.7 μm, 50×2.1 mm)]; ¹H NMR (400 MHz, DMSO-d₆): δ 8.33-8.20 (m, 2H), 7.74-7.61 (m, 2H), 4.01 (q, 1H), 3.64-3.56 (m, 1H), 3.55-3.46 (m, 2H), 2.08 (dd, 1H), 1.90-1.80 (m, 1H), 1.73-1.57 (m, 3H), 1.43-1.32 (m, 4H), 1.31-1.21 (m, 1H).

### Example-68

### 5-((7-Methyl-6-azaspiro[3.4]octan-6-yl)sulfonyl)benzo[d]isoxazole

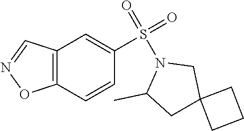 [SMILES: CC1CC2(CCC2)CN1S(=O)(=O)c1ccc2oncc2c1]

The synthesis was performed essentially as described in Method U using benzo[d]isoxazole-5-sulfonyl chloride and 7-methyl-6-azaspiro[3.4]octane hydrochloride 1.6. The crude was purified by column chromatography over silica gel using 20% ethyl acetate in hexane and desired compound (Example 68) was isolated as light brown sticky gum (42 mg, 70% yield, 99.62% purity). LCMS: m/z found 304.93 [M+H]⁺, rt=2.53 min (Method 9) [Xbridge C18 column (5 μm, 50×4.6 mm)]; ¹H NMR (400 MHz, DMSO-d₆): δ 12.21 (s, 1H), 8.06 (d, 1H), 7.91 (dd, 1H), 7.16 (d, 1H), 3.54 (q, 1H), 3.18 (d, 2H), 2.01-1.88 (m, 2H), 1.87-1.75 (m, 1H), 1.79-1.59 (m, 2H), 1.51 (dd, 1H), 1.40-1.13 (m, 5H).

### Example-69

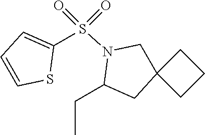 [SMILES: CCC1CC2(CCC2)CN1S(=O)(=O)c1cccs1]

### 7-Ethyl-6-(thiophen-2-ylsulfonyl)-6-azaspiro[3.4]octane

The synthesis was performed essentially as described in Method U using thiophene-2-sulfonyl chloride and 7-ethyl-6-azaspiro[3.4]octane 3.6. The crude was purified by column chromatography over silica gel using 30% ethyl acetate in hexane and desired compound (Example 69) was isolated as off white solid (38.5 mg, 78% yield, 99.9% purity). LCMS: m/z found 286.15 [M+H]⁺, rt=2.14 min (Method 1) [Acquity BEH C8 column (1.7 μm, 50×2.1 mm)]; ¹H NMR (400 MHz, DMSO-d₆): δ 7.99 (dd, 1H), 7.70 (dd, 1H), 7.24 (t, 1H), 3.49-3.37 (m, 1H), 3.30-3.22 (m, 2H), 2.05-1.77 (m, 4H), 1.77-1.58 (m, 3H), 1.57-1.45 (m, 1H), 1.40-1.32 (m, 1H), 1.26-1.16 (m, 1H), 0.86 (t, 3H).

### Example-70 and Example-71

### 7-Ethyl-6-(thiophen-2-ylsulfonyl)-6-azaspiro[3.4]octane [EN-1]) and 7-ethyl-6-(thiophen-2-ylsulfonyl)-6-azaspiro[3.4]octane [EN-2]

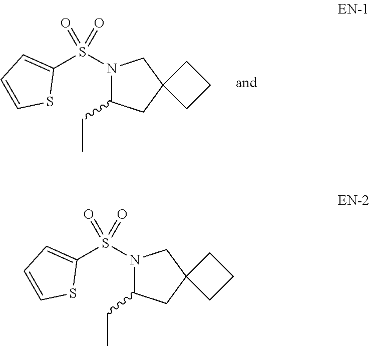 [SMILES: CCC1CC2(CCC2)CN1S(=O)(=O)c1cccs1 ; CCC1CC2(CCC2)CN1S(=O)(=O)c1cccs1]

Chiral separation racemic compound (Example 69) provided ˜30 mg each of both isomers as mentioned below. Chiral Separation Method: Chiral separation was done on Agilent 1200 series instrument. Column name: Chiralpak IG (250×21 mm), 5μ, operating at ambient temperature and flow rate of 21.0 mL/min. Mobile phase: 0.1% Isopropylamine in the mixture of 80% Hexane and 20% Ethanol, held this isocratic mixture up to 15 min with wavelength of 244 nm.

Example 70 [EN-1]: White solid (28 mg, 10% yield, 99.61% purity).

¹H NMR (400 MHz, DMSO-d₆) δ 7.98 (d, 1H), 7.70 (d, 1H), 7.25 (t, 1H), 3.49-3.38 (m, 1H), 3.25 (d, 1H), 2.03-1.77 (m, 4H), 1.75-1.57 (m, 4H), 1.58-1.45 (m, 1H), 1.41-1.31 (m, 1H), 1.26-1.17 (m, 1H), 0.86 (t, 3H).

Example 71 [EN-2]: White solid (31 mg, 11% yield, 99.78% purity). ¹H NMR (400 MHz, DMSO-d₆): δ 7.98 (d, 1H), 7.80-7.62 (m, 1H), 7.24 (t, 1H), 3.49-3.37 (m, 1H), 3.25 (d, 1H), 2.04-1.78 (m, 4H), 1.76-1.57 (m, 4H), 1.57-1.45 (m, 1H), 1.42-1.30 (m, 1H), 1.27-1.14 (m, 1H), 0.87 (t, 3H).

### Example-72

### 7-Ethyl-6-(furan-2-ylsulfonyl)-6-azaspiro[3.4]octane

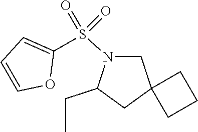 [SMILES: CCC1CC2(CCC2)CN1S(=O)(=O)c1ccco1]

The synthesis was performed essentially as described in Method U using furan-2-sulfonyl chloride and 7-ethyl-6-azaspiro[3.4]octane 3.6. The crude was purified by column chromatography over silica gel using 30% ethyl acetate in hexane and desired compound (Example 72) was isolated as pale-yellow solid (35 mg, 80% yield, 99.78% purity). LCMS: m/z found 270.00 [M+H]⁺, rt=1.74 min (Method 8) [Acquity BEH C18 column (1.7 μm, 30×2.1 mm)]; ¹H NMR (400 MHz, DMSO-d₆): δ 8.01-7.96 (m, 1H), 7.19 (d, 1H), 6.71 (dd, 1H), 3.60-3.48 (m, 1H), 3.31-3.23 (m, 2H), 2.08-1.79 (m, 4H), 1.80-1.57 (m, 3H), 1.58-1.36 (m, 2H), 1.33-1.21 (m, 1H), 0.84 (t, 3H).

### Example-73

### 3-Methyl-2-(thiophen-2-ylsulfonyl)-2-azaspiro[4.4]nonane

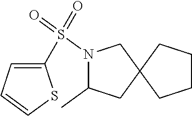 [SMILES: CC1CC2(CCCC2)CN1S(=O)(=O)c1cccs1]

The synthesis was performed essentially as described in Method U using thiophene-2-sulfonyl chloride and 3-methyl-2-azaspiro[4.4]nonane. The crude was purified by column chromatography over silica gel using 30% ethyl acetate in hexane and desired compound (Example 73) was isolated as pale-yellow sticky gum (25 mg, 78% yield, 98.34% purity). LCMS: m/z found 286.1 [M+H]⁺, rt=3.13 min (Method 9) [Xbridge C18 column (5 μm, 50×4.6 mm)]; ¹H NMR (400 MHz, DMSO-d₆): δ 8.00 (d, 1H), 7.69 (d, 1H), 7.28-7.25 (m, 1H), 3.62-3.48 (m, 1H), 3.25 (d, 1H), 3.10 (d, 1H), 1.83 (dd, 1H), 1.60-1.38 (m, 6H), 1.35 (d, 4H), 0.93-0.82 (m, 1H), 0.67-0.56 (m, 1H).

### Example-74

### 5-Methyl-6-(thiophen-2-ylsulfonyl)-6-azaspiro[2.5]octane

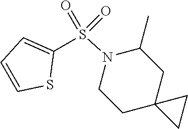 [SMILES: CC1CC2(CCN1S(=O)(=O)c1cccs1)CC2]

The synthesis was performed essentially as described in Method U using thiophene-2-sulfonyl chloride and 5-methyl-6-azaspiro[2.5]octane. The crude was purified by column chromatography over silica gel using 30% ethyl acetate in hexane and desired compound (Example 74) was isolated as colorless oil (46 mg, 31% yield, 99.7% purity). LCMS: m/z found 272.0 [M+H]⁺, rt=2.97 min (Method 9) [Xbridge C18 column (5 μm, 50×4.6 mm)]; ¹H NMR (400 MHz, DMSO-d₆): δ 7.96 (dd, 1H), 7.66 (dd, 1H), 7.21 (dd, 1H), 4.20-4.12 (m, 1H), 3.71-3.61 (m, 1H), 3.21-3.08 (m, 1H), 1.87 (dd, 1H), 1.74-1.61 (m, 1H), 1.11 (d, 3H), 0.88-0.77 (m, 2H), 0.49-0.39 (m, 1H), 0.39-0.31 (m, 1H), 0.13 (t, 2H).

### Example-75

### 3-Cyclohexyl-2-(thiophen-2-ylsulfonyl)-2-azaspiro[4.4]nonane

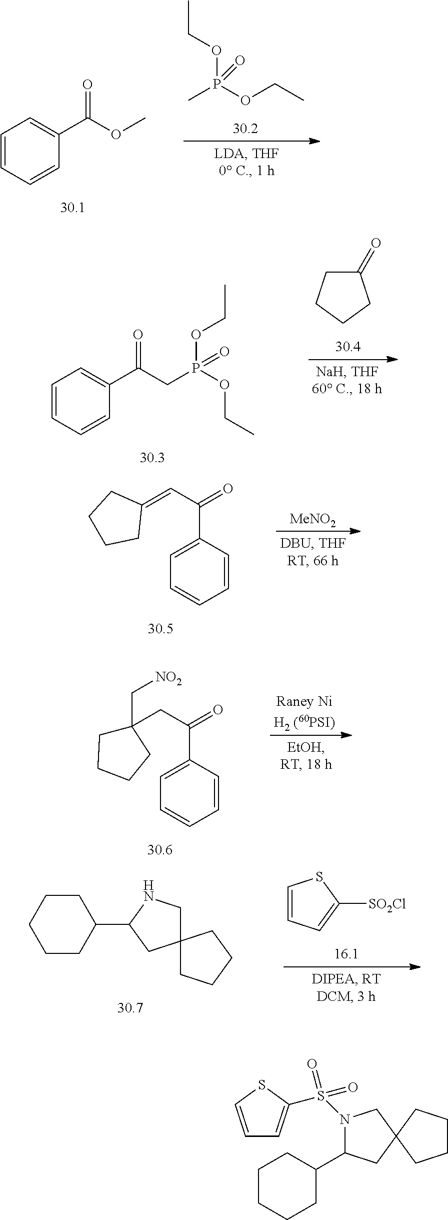

Diethyl (2-oxo-2-phenylethyl)phosphonate 30.3: In a 3-neck flask, methyl benzoate 30.1 (3 g, 22.03 mmol) and methyl diethylphosphonate 30.2 (4.3 g, 28.64 mmol) were dissolved in THF (35 mL) and cooled to 0° C. LDA (23.2 mL, 0.5 mol in THF) was added drop wise to the solution and stirred for 30 minutes at 0° C. The reaction was quenched with saturated aqueous NH₄Cl solution (40 mL) and extracted with diethyl ether (3×75 mL). The combined organic layer was washed with cold water (15 mL) and brine (15 mL), dried over anhydrous Na₂SO₄ and the solvent was evaporated under reduced pressure. Flash chromatography on silica gel afforded intermediate 30.3 (2.8 g, 50% yield). ¹H NMR (400 MHz, DMSO-d₆): δ 8.02 (d, 2H), 7.66 (t, 1H), 7.53 (t, 2H), 4.08-3.89 (m, 4H), 1.41 (d, 3H), 1.27-1.13 (m, 6H).

2-Cyclopentylidene-1-phenylethan-1-one 30.5: A 50 mL flame-dried round-bottomed flask was charged with NaH (60% dispersion in mineral oil, 170 mg, 7.13 mmol) and dry THF (5 mL). The flask was sealed with a rubber septum and purged with N₂ (gas). To the mixture was added the solution of diethyl (2-oxo-2-phenylethyl)phosphonate 30.3 (1.6 g, 6.24 mmol) in THF (5 mL) dropwise and the reaction mixture was stirred for 15 min at RT. Cyclopentanone 30.4 (500 mg, 5.94 mmol) was added to the reaction mixture and it was refluxed overnight. The reaction mixture was quenched with saturated aqueous NH₄Cl solution and extracted with diethyl ether. (3×75 mL). The combined organic layer was washed with cold water (15 mL) and brine (15 mL), dried over anhydrous Na₂SO₄ and the solvent was evaporated under reduced pressure to obtain the crude product as dark yellow oil. It was further purified by flash column chromatography over silica gel using 20% EA in hexane and intermediate 30.5 was isolated as pale-yellow oil (280 mg, 26% yield). ¹H NMR (400 MHz, DMSO-d₆): δ 8.02 (d, 2H), 7.66 (t, 1H), 7.53 (t, 2H), 4.08-3.89 (m, 4H), 1.41 (d, 3H), 1.27-1.13 (m, 6H).

2-(1-(Nitromethyl)cyclopentyl)-1-phenylethan-1-one 30.6: To a stirred solution of intermediate 30.5 (280 mg, 1.53 mmol) in THF (3 mL) was added DBU (700 mg, 4.59 mmol) followed by nitromethane (110 mg, 1.83 mmol) at 0° C. and the reaction mixture was stirred at RT for 18 h. DBU (700 mg, 4.59 mmol) was added to it and stirred for another 48 h. Cold water (50 mL) was added to the reaction mass and it was extracted with ethyl acetate (3×25 mL). The combined organic part was washed with brine (2×10 mL) and dried over anhydrous MgSO₄. The solvent was evaporated under reduced pressure and the crude was purified by column chromatography over silica gel using 20% EA in hexane to afford intermediate 30.6 as yellow oil (60 mg, 16% yield). ¹H NMR (400 MHz, Chloroform-d) δ 7.93 (d, 2H), 7.56 (t, 1H), 7.46 (t, 2H), 4.74 (s, 2H), 3.26 (s, 2H), 1.90-1.79 (m, 2H), 1.78-1.60 (m, 6H).

3-Cyclohexyl-2-azaspiro[4.4]nonane 30.7: Intermediate 30.6 (60 mg, 0.24 mmol) was dissolved in EtOH (10 mL) and Raney Ni (wet) was added to it. The reaction mixture was then allowed to shake under hydrogen atmosphere under 60 PSI pressure at RT for 18 h. It was then filtered through celite, and the filtrate was concentrated under reduced pressure to afford crude intermediate 30.7 (40 mg, 82% yield). It was used in the forwarding step without further purification.

### 3-Cyclohexyl-2-(thiophen-2-ylsulfonyl)-2-azaspiro[4.4]nonane (Example 75)

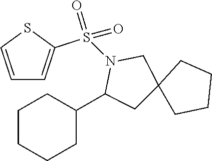 [SMILES: O=S(=O)(c1cccs1)N1CC2(CCCC2)CC1C1CCCCC1]

The synthesis was performed essentially as described in Method A using thiophene-2-sulfonyl chloride and intermediate 30.7. The crude was purified by column chromatography over silica gel using 30% ethyl acetate in hexane and desired compound (Example 75) was isolated as colorless sticky solid (10 mg, 14% yield, 97.94% purity). LCMS: m/z found 354.27 [M+H]⁺, rt=2.47 min (Method 1) [Acquity BEH C8 column (1.7 μm, 50×2.1 mm)]; ¹H NMR (400 MHz, Chloroform-d): δ 7.59-7.51 (m, 2H), 7.10 (dd, 1H), 3.66-3.55 (m, 1H), 3.34 (d, 1H), 3.15 (d, 1H), 2.22-2.07 (m, 1H), 1.87-1.63 (m, 1H), 1.64-1.55 (m, 1H), 1.51-1.38 (m, 1H), 1.35-1.27 (m, 2H), 1.23-1.16 (m, 2H), 0.90-0.78 (m, 2H)

### Example-76

### 7-Methyl-6-((5-methylthiophen-2-yl)sulfonyl)-6-azaspiro[3.4]octane

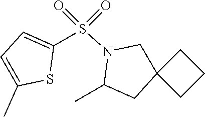 [SMILES: Cc1ccc(S(=O)(=O)N2CC3(CCC3)CC2C)s1]

The synthesis was performed essentially as described in Scheme 3 using 7-methyl-6-azaspiro[3.4]octane hydrochloride 1.6 and thiophene-2-sulfonyl chloride. The compound was purified by Chromatography over silica gel using 3:7 ethyl acetate:hexane and isolated as colourless gum (140 mg, 68% yield, 99.40% purity). LCMS: m/z found 286.34 [M+H]⁺, rt=1.91 min (Method 2) [YMC Triart C18 column (3 μm, 33×2.1 mm)]; ¹H NMR (400 MHz, Chloroform-d): δ 7.37 (d, 1H), 6.79-6.73 (m, 1H), 3.71-3.58 (m, 1H), 3.46 (d, 1H), 3.18 (d, 1H), 2.51 (s, 3H), 2.10-1.98 (m, 1H), 2.03-1.85 (m, 2H), 1.86-1.71 (m, 2H), 1.67-1.54 (m, 2H), 1.54-1.42 (m, 1H), 1.37 (d, 3H).

### Example-77 and Example-78

### 7-Methyl-6-((5-methylthiophen-2-yl)sulfonyl)-6-azaspiro[3.4]octane [EN-1]; and

### 7-methyl-6-((5-methylthiophen-2-yl)sulfonyl)-6-azaspiro[3.4]octane [EN-2]

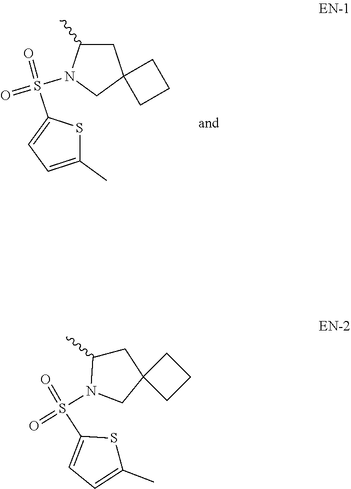

Chiral separation of 7-methyl-6-((5-methylthiophen-2-yl)sulfonyl)-6-azaspiro[3.4]octane (Example 76) provided both enantiomers as mentioned below. Chiral Separation Method: Chiral separation was done on Agilent 1200 series instrument. Column name: Chiralpak IG (250×21 mm), 5μ, operating at ambient temperature and flow rate of 21.0 mL/min. Mobile phase: 0.1% Iso propylamine in the mixture of 90% hexane, 5% dichloromethane and 5% ethanol, held this isocratic mixture up to 25 min with wavelength of 260 nm.

Example 77 [EN-1]: Off white solid (45 mg, 20% yield, 99.91% purity). LCMS: m/z found 286.0 [M+H]⁺, rt=6.70 min (Method 10) [Xbridge C18 column (5 μm, 50×4.6 mm)]; ¹H NMR (400 MHz, DMSO-d₆): δ 7.49 (d, 1H), 7.00-6.93 (m, 1H), 3.57 (q, 1H), 3.42-3.31 (m, 1H), 3.16 (d, 1H), 2.05-1.80 (m, 3H), 1.77-1.65 (m, 2H), 1.57 (dd, 1H), 1.53-1.42 (m, 1H), 1.40-1.30 (m, 1H), 1.29 (d, 3H).

Example 78 [EN-2]: Off white solid (48 mg, 21% yield, 99.86% purity). LCMS: m/z found 286.0 [M+H]⁺, rt=1.91 min (Method 2) [YMC Triart C18 column (3 μm, 33×2.1 mm)]; ¹H NMR (400 MHz, DMSO-d₆): δ 7.49 (d, 1H), 6.96 (d, 1H), 3.57 (q, 1H), 3.42-3.33 (m, 1H), 3.16 (d, 1H), 2.03-1.80 (m, 3H), 1.78-1.64 (m, 2H), 1.57 (dd, 1H), 1.53-1.42 (m, 1H), 1.40-1.32 (m, 1H), 1.29 (d, 3H).

### Example-79

### 2-((7-Methyl-6-azaspiro[3.4]octan-6-yl)sulfonyl)quinoline

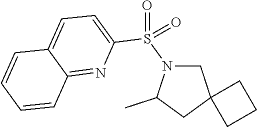 [SMILES: CC1CC2(CCC2)CN1S(=O)(=O)c1ccc2ccccc2n1]

The synthesis was performed essentially as described in Method A using 7-methyl-6-azaspiro[3.4]octane hydrochloride 1.6 and quinoline-2-sulfonyl chloride. The compound was purified by Reverse Phase Preparative HPLC and isolated as brown sticky gum (11 mg, 9% yield, 99.89% purity). Prep-HPLC Method: Preparative HPLC was done on Waters auto purification instrument. Column name: YMC-Actus C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 70% A and 30% B, then to 35% A and 65% B in 3 min, then to 10% A and 90% B in 22 min., then to 5% A and 95% B in 23 min., held this composition up to 25 min. for column washing, then returned to initial composition in 26 min. and held till 28 min. LCMS: m/z found 317.2 [M+H]⁺, rt=2.40 min (Method 14) [Acquity BEH C8 column (1.7 μm, 50×2.1 mm)]; ¹H NMR (400 MHz, DMSO-d₆): δ 8.70 (d, 1H), 8.15 (d, 2H), 8.02 (d, 1H), 7.93 (t, 1H), 7.79 (t, 1H), 4.08 (q, 1H), 3.48 (q, 2H), 2.06 (dd, 1H), 2.01-1.92 (m, 1H), 1.89-1.82 (m, 1H), 1.74-1.54 (m, 3H), 1.44-1.32 (m, 1H), 1.31 (d, 3H), 1.28-1.17 (m, 1H).

### Example-80

### 4-Methyl-7-((7-methyl-6-azaspiro[3.4]octan-6-yl)sulfonyl)quinoline

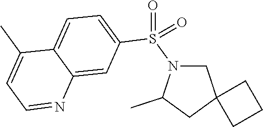 [SMILES: Cc1ccnc2cc(S(=O)(=O)N3CC4(CCC4)CC3C)ccc12]

The synthesis was performed essentially as described in Method A using 7-methyl-6-azaspiro[3.4]octane hydrochloride 1.6 and 4-methylquinoline-7-sulfonyl chloride. The compound was purified by Reverse Phase Preparative HPLC and isolated as off white solid (22 mg, 28% yield, 99.72% purity). Prep-HPLC Method: Preparative HPLC was done on Waters auto purification instrument. Column name: YMC-Actus C18 (250×20 mm, 5μ) operating at ambient temperature and flow rate of 16 ml/min. Mobile phase: A=20 mM Ammonium Bicarbonate in water, B=Acetonitrile; Gradient Profile: Mobile phase initial composition of 70% A and 30% B, then to 35% A and 65% B in 3 min, then to 10% A and 90% B in 22 min., then to 5% A and 95% B in 23 min., held this composition up to 25 min. for column washing, then returned to initial composition in 26 min. and held till 28 min. LCMS: m/z found 331.2 [M+H]⁺, rt=2.29 min (Method 14) [Acquity BEH C8 column (1.7 μm, 50×2.1 mm)]; ¹H NMR (400 MHz, DMSO-d₆): δ 8.90 (d, 1H), 8.40 (s, 1H), 8.32 (d, 1H), 7.95 (d, 1H), 7.52 (d, 1H), 3.74 (q, 1H), 3.41 (d, 1H), 3.32 (d, 1H), 2.75 (s, 3H), 2.06-1.88 (m, 3H), 1.78-1.65 (m, 2H), 1.60 (dd, 1H), 1.45 (d, 2H), 1.32 (d, 3H).

### Example-81 and Example-82

### 4-Methyl-7-((7-methyl-6-azaspiro[3.4]octan-6-yl)sulfonyl)quinoline [EN-1]; and

### 4-methyl-7-((7-methyl-6-azaspiro[3.4]octan-6-yl)sulfonyl)quinoline [EN-2]

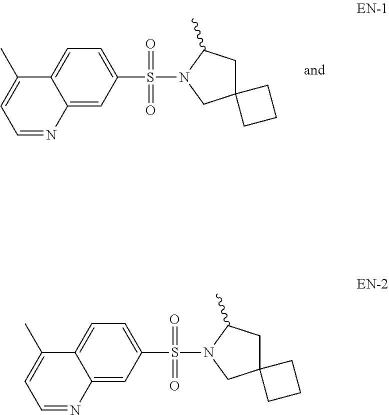

Chiral separation of 4-methyl-7-((7-methyl-6-azaspiro[3.4]octan-6-yl)sulfonyl)quinoline (Example 80) provided both enantiomers as mentioned below. Chiral Separation Method: Chiral separation was done on Agilent 1200 series instrument. Column name: Chiralpak IC (250×21 mm), 5μ, operating at ambient temperature and flow rate of 21.0 mL/min. Mobile phase: the mixture of 50% Hexane, 25% Dichloromethane and 25% Ethanol, held this isocratic mixture up to 20 min with wavelength of 232 nm.

Example 81 [EN-1]: White solid (60 mg, 16% yield, 99.19% purity). LCMS: m/z found 331.24 [M+H]⁺, rt=1.94 min (Method 1) [Acquity BEH C8 column (1.7 μm, 50×2.1 mm)]; ¹H NMR (400 MHz, DMSO-d₆): δ 8.91 (d, 1H), 8.41-8.29 (m, 2H), 7.98 (dd, 1H), 7.57 (d, 1H), 3.66 (q, 1H), 3.39 (d, 1H), 3.26 (d, 1H), 2.73 (s, 3H), 2.03-1.73 (m, 3H), 1.71-1.47 (m, 3H), 1.32 (d, 3H), 1.29-1.15 (m, 2H).

Example 82 [EN-2]: White solid (60 mg, 16% yield, 98.72% purity). LCMS: m/z found 331.24[M+H]⁺, rt=1.94 min (Method 1) [Acquity BEH C8 column (1.7 μm, 50×2.1 mm)]; ¹H NMR (400 MHz, DMSO-d₆): δ 8.91 (d, 1H), 8.41-8.29 (m, 2H), 7.98 (d, 1H), 7.56 (d, 1H), 3.64 (q, 1H), 3.39 (d, 1H), 3.26 (d, 1H), 2.73 (s, 3H), 2.02-1.76 (m, 3H), 1.72-1.46 (m, 3H), 1.33 (d, 3H), 1.30-1.10 (m, 2H).

### Example-83

### 7-Methyl-6-(pyrimidin-5-ylsulfonyl)-6-azaspiro[3.4]octane

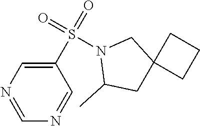 [SMILES: CC1CC2(CCC2)CN1S(=O)(=O)c1cncnc1]

The synthesis was performed essentially as described in Method A using 7-methyl-6-azaspiro[3.4]octane hydrochloride 1.6 and pyrimidine-5-sulfonyl chloride. The compound was purified by Reverse Phase Preparative HPLC and isolated as off white solid (65 mg, 70% yield, 97.73% purity). Prep-HPLC Method: Preparative HPLC was done on Waters auto purification instrument. Column name: Sunfire C18 (150×19 mm, 10μ) operating at ambient temperature and flow rate of 16 ml/min. Mobile phase: A=0.1% Formic acid in water, B=Acetonitrile; Gradient Profile: Mobile phase initial composition of 70% A and 30% B, then to 60% A and 40% B in 3 min, then to 30% A and 70% B in 22 min., then to 5% A and 95% B in 23 min., held this composition up to 25 min. for column washing, then returned to initial composition in 26 min. and held till 28 min. LCMS: m/z found 268.15 [M+H]⁺, rt=1.80 min (Method 1) [Acquity BEH C8 column (1.7 μm, 50×2.1 mm)]; ¹H NMR (400 MHz, DMSO-d₆): δ 9.48 (s, 1H), 9.26 (s, 2H), 3.67 (q, 1H), 3.46-3.33 (m, 1H), 2.08-1.89 (m, 2H), 1.89-1.76 (m, 1H), 1.76-1.61 (m, 2H), 1.55 (dd, 1H), 1.30 (d, 4H), 1.26-1.14 (m, 2H).

### Example-84

### 6-((2-Isopropoxypyridin-4-yl)sulfonyl)-7-methyl-6-azaspiro[3.4]octane

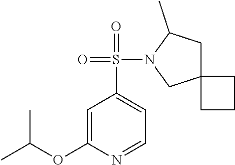 [SMILES: CC(C)Oc1cc(S(=O)(=O)N2CC3(CCC3)CC2C)ccn1]

The synthesis was performed as described in Method A, using 7-methyl-6-azaspiro[3.4]octane hydrochloride 1.6 and 2-isopropoxypyridine-4-sulfonyl chloride. The compound was purified by column chromatography over silica gel using 1:3 ethyl acetate:hexane and isolated as off white solid (30 mg, 36% yield, 98.66% purity). LCMS: m/z found 325.21 [M+H]⁺, rt=2.29 min (Method 1) [Acquity BEH C8 column (1.7 μm, 50×2.1 mm)]; ¹H NMR (400 MHz, DMSO-d₆): δ 8.39 (d, 1H), 7.30 (dd, 1H), 7.00 (s, 1H), 5.30 (q, 1H), 3.62 (q, 1H), 3.28-3.22 (m, 2H), 2.03-1.93 (m, 2H), 1.87-1.78 (m, 1H), 1.75-1.65 (m, 2H), 1.54 (dd, 1H), 1.46-1.17 (m, 11H).

### Example-85

### 2,2,2-Trifluoro-1-(8-((7-methyl-6-azaspiro[3.4]octan-6-yl)sulfonyl)-3,4-dihydroisoquinolin-2(1H)-yl)ethan-1-one

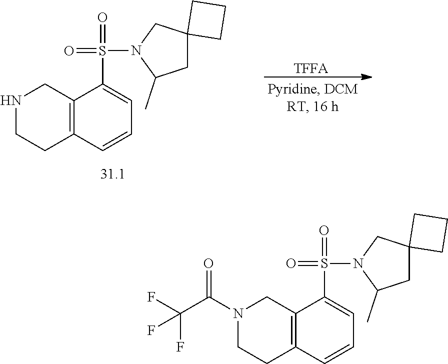 [SMILES: CC1CC2(CCC2)CN1S(=O)(=O)c1cccc2c1CNCC2 ; "TFFA" ; CC1CC2(CCC2)CN1S(=O)(=O)c1cccc2c1CN(C(=O)C(F)(F)F)CC2]

The starting compound 8-((7-methyl-6-azaspiro[3.4]octan-6-yl)sulfonyl)-1,2,3,4-tetrahydroisoquinoline 31.1 was synthesised from tert-butyl 8-bromo-3,4-dihydroisoquinoline-2(1H)-carboxylate in a similar manner to that described in Example 25. Compound 31.1 (50 mg, 0.156 mmol) was dissolved in DCM (5 mL) and cooled in ice bath. Pyridine (0.04 mL, 0.469 mmol) was added to the solution followed by TFAA (0.05 mL, 0.344 mmol). The reaction mixture was stirred at RT for 16 h. The volatiles were evaporated under reduced pressure and the crude was purified by column chromatography over silica gel using 1:3 ethyl acetate:hexane and isolated as off white solid (15 mg, 23% yield, 96.39% purity). LCMS: m/z found 417.42 [M+H]⁺, rt=2.05 min (Method 2) [YMC Triart C18 column (3 μm, 33×2.1 mm)]; ¹H NMR (400 MHz, DMSO-d₆): δ 7.81 (d, 1H), 7.56-7.43 (m, 2H), 5.20 (s, 2H), 3.86 (s, 3H), 3.35 (d, 1H), 3.10-3.02 (m, 1H), 2.18-2.08 (m, 1H), 2.04-1.96 (m, 1H), 1.87-1.73 (m, 4H), 1.66 (dd, 1H), 1.28 (s, 3H), 1.16 (d, 3H).

### Example-86

### 6-((2-Methoxypyridin-4-yl)sulfonyl)-7-methyl-6-azaspiro[3.4]octane

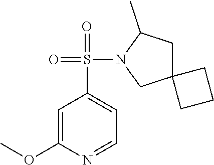 [SMILES: COc1cc(S(=O)(=O)N2CC3(CCC3)CC2C)ccn1]

The synthesis was performed as described in Method A using 7-methyl-6-azaspiro[3.4]octane hydrochloride 1.6 and 2-methoxypyridine-4-sulfonyl chloride. 2-methoxypyridine-4-sulfonyl chloride was synthesised from 2-methoxypyridine-4-thiol in a similar manner to that described in Example 11. The crude was purified by column chromatography over silica gel using 1:3 ethyl acetate:hexane and isolated as off white solid (35 mg, 46% yield, 93.63% purity). LCMS: m/z found 297.41 [M+H]⁺, rt=1.95 min (Method 2) [YMC Triart C18 column (3 μm, 33×2.1 mm)]; ¹H NMR (400 MHz, DMSO-d₆): δ 8.41 (d, 1H), 7.30 (d, 1H), 7.08 (s, 1H), 3.96 (s, 3H), 3.72 (q, 1H), 3.37 (d, 1H), 3.29 (d, 1H), 2.06-1.87 (m, 3H), 1.85-1.70 (m, 2H), 1.66-1.48 (m, 3H), 1.28 (d, 3H).

### Example-87

### 7-Ethyl-6-((2-methoxypyridin-4-yl)sulfonyl)-6-azaspiro[3.4]octane

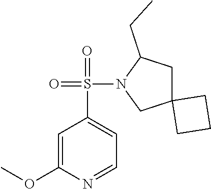 [SMILES: CCC1CC2(CCC2)CN1S(=O)(=O)c1ccnc(OC)c1]

The synthesis was performed using a Scheme analogous to that described in Scheme 3 using amine 3.6 and 2-methoxypyridine-4-sulfonyl chloride. The crude was purified by column chromatography over silica gel using 1:3 ethyl acetate:hexane and isolated as off white solid (38 mg, 48% yield, 99.13% purity). LCMS: m/z found 311.25 [M+H]⁺, rt=2.22 min (Method 1) [Acquity BEH C8 column (1.7 μm, 50×2.1 mm)]; ¹H NMR (400 MHz, DMSO-d₆): δ 8.41 (d, 1H), 7.31 (d, 1H), 7.08 (s, 1H), 3.96 (s, 3H), 3.65-3.51 (m, 1H), 3.34 (q, 2H), 2.07-1.62 (m, 7H), 1.61-1.43 (m, 3H), 0.86 (t, 3H).

### Example-88

### 6-((2-Ethoxypyridin-4-yl)sulfonyl)-7-methyl-6-azaspiro[3.4]octane

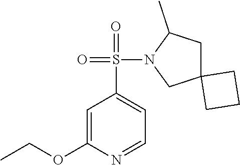 [SMILES: CCOc1cc(S(=O)(=O)N2CC3(CCC3)CC2C)ccn1]

The synthesis was performed as described in Method A using 7-methyl-6-azaspiro[3.4]octane hydrochloride 1.6 and 2-ethoxypyridine-4-sulfonyl chloride. 2-ethoxypyridine-4-sulfonyl chloride was synthesised from 4-bromo-2-ethoxypyridine in a similar manner to that described in Example 25. The crude was purified by column chromatography over silica gel using 1:3 ethyl acetate:hexane and isolated as off white solid (35 mg, 44% yield, 98.99% purity). LCMS: m/z found 311.25 [M+H]⁺, rt=2.19 min (Method 1) [Acquity BEH C8 column (1.7 μm, 50×2.1 mm)]; ¹H NMR (400 MHz, DMSO-d₆): δ 8.40 (d, 1H), 7.33 (dd, 1H), 7.07 (s, 1H), 4.37 (q, 2H), 3.62 (q, 1H), 3.24 (d, 1H), 2.01-1.91 (m, 2H), 1.89-1.77 (m, 2H), 1.77-1.62 (m, 2H), 1.54 (dd, 1H), 1.45-1.36 (m, 1H), 1.33 (t, 4H), 1.28 (d, 3H).

### Example-89

### 4-(Methoxymethyl)-5-((7-methyl-6-azaspiro[3.4]octan-6-yl)sulfonyl)thiazole

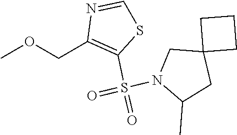 [SMILES: COCc1ncsc1S(=O)(=O)N1CC2(CCC2)CC1C]

The synthesis was performed as described in Method A using 7-methyl-6-azaspiro[3.4]octane hydrochloride 1.6 and 4-(methoxymethyl)thiazole-5-sulfonyl chloride. 4-(methoxymethyl)thiazole-5-sulfonyl chloride was synthesised from 5-bromo-4-(methoxymethyl)thiazole in a similar manner to that described in Example 25. The crude was purified by column chromatography over silica gel using 1:3 ethyl acetate:hexane and isolated as off white solid (45 mg, 50% yield, 94.14% purity). LCMS: m/z found 317.19 [M+H]⁺, rt=2.00 min (Method 1) [Acquity BEH C8 column (1.7 μm, 50×2.1 mm)]; ¹H NMR (400 MHz, DMSO-d₆): δ 8.43 (s, 1H), 4.73 (s, 2H), 3.85 (q, 1H), 3.41 (s, 3H), 2.05-1.91 (m, 2H), 1.89-1.78 (m, 2H), 1.78-1.62 (m, 2H), 1.55 (dd, 1H), 1.49-1.38 (m, 1H), 1.34-1.20 (m, 5H).

### Example-90

### (5-((7-Methyl-6-azaspiro[3.4]octan-6-yl)sulfonyl)thiazol-4-yl)methanol

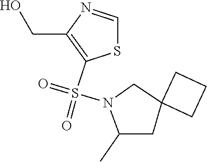 [SMILES: CC1CC2(CCC2)CN1S(=O)(=O)c1scnc1CO]

The synthesis was performed as described in Method A using 7-methyl-6-azaspiro[3.4]octane hydrochloride 1.6 and 4-(hydroxymethyl)thiazole-5-sulfonyl chloride. 4-(hydroxymethyl)thiazole-5-sulfonyl chloride was synthesised from (5-bromothiazol-4-yl)methanol in a similar manner to that described in Example 25. The crude was purified by column chromatography over silica gel using 50% ethyl acetate in hexane and isolated as off white solid (40 mg, 43% yield, 98.40% purity). LCMS: m/z found 303.17 [M+H]⁺, rt=1.79 min (Method 1) [Acquity BEH C8 column (1.7 μm, 50×2.1 mm)]; ¹H NMR (400 MHz, DMSO-d₆): δ 6.31 (t, 1H), 4.73 (d, 2H), 3.85 (q, 1H), 3.26 (d, 1H), 2.04-1.91 (m, 3H), 1.88-1.60 (m, 3H), 1.54 (dd, 1H), 1.48-1.36 (m, 1H), 1.31-1.13 (m, 5H).

### Example-91

### 6-(Benzofuran-6-ylsulfonyl)-7-ethyl-6-azaspiro[3.4]octane

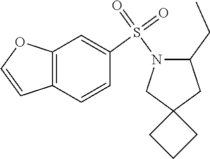 [SMILES: CCC1CC2(CCC2)CN1S(=O)(=O)c1ccc2ccoc2c1]

The synthesis was performed as described in Method A using 7-methyl-6-azaspiro[3.4]octane hydrochloride 1.6 and benzofuran-6-sulfonyl chloride. The crude was purified by column chromatography over silica gel using 30% ethyl acetate in hexane and isolated as white solid (15 mg, 25% yield, 99.04% purity). LCMS: m/z found 320.27 [M+H]⁺, rt=2.23 min (Method 1) [Acquity BEH C8 column (1.7 μm, 50×2.1 mm)]; ¹H NMR (400 MHz, DMSO-d₆): δ 8.25 (d, 1H), 8.08 (s, 1H), 7.88 (d, 1H), 7.72 (dd, 1H), 7.15-7.10 (m, 1H), 3.55-3.39 (m, 1H), 3.27 (s, 2H), 1.99-1.73 (m, 4H), 1.70-1.44 (m, 4H), 1.21-1.02 (m, 2H), 0.85 (t, 3H).

### Example-92

### 7-Ethyl-6-((2-methylpyridin-4-yl)sulfonyl)-6-azaspiro[3.4]octane

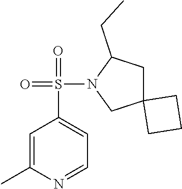 [SMILES: CCC1CC2(CCC2)CN1S(=O)(=O)c1ccnc(C)c1]

The synthesis was performed as described in Method A using amine 3.6 and 2-methylpyridine-4-sulfonyl chloride. 2-methylpyridine-4-sulfonyl chloride was synthesised from 2-methylpyridine-4-thiol in a similar manner to that described in Example 11. The crude was purified by column chromatography over silica gel using 1:3 ethyl acetate:hexane and isolated as off white solid (35 mg, 46% yield, 93.63% purity). LCMS: m/z found 295.30 [M+H]1, rt=2.08 min (Method 1) [Acquity BEH C8 column (1.7 μm, 50×2.1 mm)]; ¹H NMR (400 MHz, DMSO-d₆): δ 8.70 (d, 1H), 7.66 (s, 1H), 7.57 (d, 1H), 3.55-3.43 (m, 1H), 3.32-3.22 (m, 2H), 2.59 (s, 3H), 2.01-1.75 (m, 4H), 1.76-1.43 (m, 3H), 1.36-1.13 (m, 3H), 0.85 (t, 3H).

### Example-93

### (3,5-Dimethylisoxazol-4-yl)(7-((7-methyl-6-azaspiro[3.4]octan-6-yl)sulfonyl)-3,4-dihydroisoquinolin-2(1H)-yl)methanone

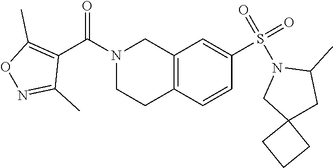 [SMILES: Cc1noc(C)c1C(=O)N1CCc2ccc(S(=O)(=O)N3CC4(CCC4)CC3C)cc2C1]

The synthesis was performed essentially as described in Method B using 3,5-dimethylisoxazole-4-carbonyl chloride and intermediate 8.3. The crude was purified by column chromatography over silica gel using 30% ethyl acetate in hexane and desired compound (Example 93) was isolated as white solid (15 mg, 43% yield, 94.85% purity). LCMS: m/z found 444.26 [M+H]⁺, rt=2.02 min (Method 1) [Acquity BEH C8 column (1.7 μm, 50×2.1 mm)]; 1H NMR (400 MHz, DMSO-d6): δ 7.74 (s, 1H), 7.60 (d, 1H), 7.41 (d, 1H), 5.03-4.56 (m, 2H), 4.01-3.43 (m, 3H), 3.15 (d, 1H), 2.92 (s, 2H), 2.37 (s, 3H), 2.15 (s, 3H), 2.02-1.78 (m, 3H), 1.75-1.45 (m, 3H), 1.42-1.13 (m, 6H).

### Example-94

### 1-(7-((7-Methyl-6-azaspiro[3.4]octan-6-yl)sulfonyl)-3,4-dihydroisoquinolin-2(1H)-yl)ethan-1-one

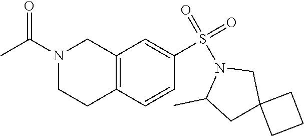 [SMILES: CC(=O)N1CCc2ccc(S(=O)(=O)N3CC4(CCC4)CC3C)cc2C1]

The synthesis was performed essentially as described in Method B using acetyl chloride and intermediate 8.3. The crude was purified by column chromatography over silica gel using 30% ethyl acetate in hexane and desired compound (Example 94) was isolated as white solid (12 mg, 42% yield, 99.85% purity). LCMS: m/z found 363.29 [M+H]⁺, rt=1.92 min (Method 1) [Acquity BEH C8 column (1.7 μm, 50×2.1 mm)]; 1H NMR (400 MHz, DMSO-d6): δ 7.68 (d, 1H), 7.60 (t, 1H), 7.40 (d, 1H), 4.76 (s, 1H), 4.69 (s, 1H), 3.66 (q, 2H), 3.55 (q, 1H), 3.31-3.23 (m, 1H), 3.15 (d, 1H), 2.94 (t, 1H), 2.83 (t, 1H), 2.09 (s, 3H), 2.01-1.78 (m, 3H), 1.75-1.61 (m, 2H), 1.58-1.47 (m, 1H), 1.41-1.29 (m, 1H), 1.28 (t, 4H).

### Example-95

### 5-Methyl-2-((7-methyl-6-azaspiro[3.4]octan-6-yl)sulfonyl)thiazole

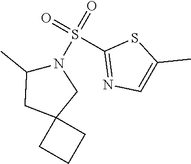

The synthesis was performed essentially as described in Method A using 5-methylthiazole-2-sulfonyl chloride and 7-methyl-6-azaspiro[3.4]octane hydrochloride 1.6. The crude was purified by Reverse Phase Preparative HPLC and desired compound (Example 95) was isolated as colourless gum (50 mg, 44% yield, 97.64% purity). LCMS: m/z found 287.21 [M+H]⁺, rt=2.07 min (Method 1) [Acquity BEH C8 column (1.7 μm, 50×2.1 mm)]1H NMR (400 MHz, DMSO-d6): δ 7.83 (s, 1H), 3.86 (q, 1H), 3.45-3.34 (m, 2H), 2.54 (s, 3H), 2.06-1.93 (m, 2H), 1.93-1.81 (m, 1H), 1.82-1.63 (m, 2H), 1.60 (dd, 1H), 1.54-1.42 (m, 1H), 1.39-1.16 (m, 4H).

### Example-96

### 6-Ethoxy-2-((7-methyl-6-azaspiro[3.4]octan-6-yl)sulfonyl)benzo[d]thiazole

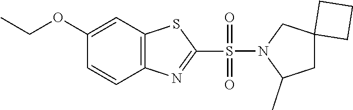 [SMILES: CCOc1ccc2nc(S(=O)(=O)N3CC4(CCC4)CC3C)sc2c1]

The synthesis was performed essentially as described in Method A using 6-ethoxybenzo[d]thiazole-2-sulfonyl chloride and 7-methyl-6-azaspiro[3.4]octane hydrochloride

1.6. The crude was purified by flash chromatography and desired compound (Example 96) was isolated as colourless gum (30 mg, 20% yield, 96.25% purity). LCMS: m/z found 367.25 [M+H]⁺, rt=2.32 min (Method 1) [Acquity BEH C8 column (1.7 μm, 50×2.1 mm)]; 1H NMR (400 MHz, DMSO-d6): δ 8.09 (d, 1H), 7.81 (d, 1H), 7.25 (dd, 1H), 4.14 (q, 2H), 3.98 (q, 1H), 3.53-3.42 (m, 2H), 2.12-1.93 (m, 2H), 1.91-1.79 (m, 1H), 1.77-1.55 (m, 3H), 1.42-1.31 (m, 6H), 1.31-1.19 (m, 2H).

### Example-97

### 3-Ethyl-2-(thiophen-2-ylsulfonyl)-2-azaspiro[4.4]nonane

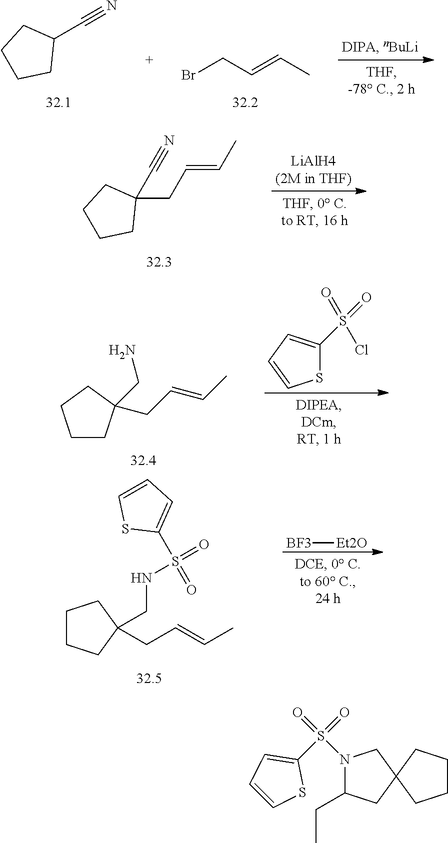

(E)-1-(But-2-en-1-yl)cyclopentane-1-carbonitrile 32.3: To a solution of DIPA (1.8 mL, 12.612 mmol) in THF (15 mL) was added n-BuLi (2.5M in hexane) (4.41 mL, 11.035 mmol) at −78° C. It was stirred at −78° C. for 20 min and the solution of cyclopentanecarbonitrile 32.1 (1 g, 10.51 mmol) in THF (10 mL) was added to it drop wise keeping the temperature of the reaction below −70° C. It was then stirred at that temperature for ˜30 min. A solution of crotylbromide 32.2 (1.2 mL, 11.561 mmol) in THF (2 mL) was added to it drop-wise and was stirred at −70° C. for 90 min. The reaction mixture was quenched with saturated aqueous ammonium chloride solution (20 mL) and was extracted with EtOAc (3×20 mL). Combined organic layer was washed with brine (2×15 mL), dried over anhydrous Na₂SO₄ and concentrated under reduced pressure to afford intermediate 32.3 as light brown oil (1.520 g, 96%). The crude compound was used in the forwarding step without further purification.

(E)-(1-(But-2-en-1-yl)cyclopentyl)methanamine 32.4: The reduction of intermediate 32.3 was performed essentially as described in Method N. The crude intermediate amine 32.4 was isolated as light brown oil (510 mg) and it was used in the forwarding step without further purification.

(E)-N-((1-(but-2-en-1-yl)cyclopentyl)methyl)thiophene-2-sulfonamide 32.5: The synthesis was performed using a method analogous to that described in Scheme 3 using intermediate 32.4 and thiophene-2-sulfonyl chloride. The crude (1.2 g) was used in the forwarding step without further purification.

### 3-Ethyl-2-(thiophen-2-ylsulfonyl)-2-azaspiro[4.4]nonane

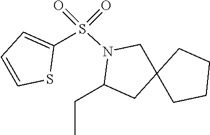 [SMILES: CCC1CC2(CCCC2)CN1S(=O)(=O)c1cccs1]

Cyclisation Method V: To a solution of intermediate 32.5 (500 mg, 1.67 mmol) in DCE (10 mL) was added BF₃·Et₂O (0.6 mL, 5.009 mmol) at 0° C. It was stirred at 10° C. for 1 h and then gradually warmed to RT and heated at 60° C. for 24 h. It was neutralized with saturated aqueous NaHCO₃ solution (2 mL) and diluted with water (10 mL). It was extracted with EtOAc (2×15 mL) and combined organic layer was washed with brine (10 mL), dried over anhydrous Na₂SO₄ and concentrated under reduced pressure. The crude was purified by column chromatography over silica gel using 30% ethyl acetate in hexane and desired compound (Example-97) was isolated as white sticky solid (50 mg, 10% yield, 99.79% purity). LCMS: m/z found 300.19 [M+H]⁺, rt=2.20 min (Method 1) [Acquity BEH C8 column (1.7 μm, 50×2.1 mm)]; ¹H NMR (400 MHz, DMSO-d₆): δ 7.99 (dd, 1H), 7.70 (dd, 1H), 7.26 (dd, 1H), 3.49-3.37 (m, 1H), 3.21 (d, 1H), 3.15 (d, 1H), 2.06-1.91 (m, 1H), 1.83-1.73 (m, 1H), 1.65-1.28 (m, 8H), 0.89-0.77 (m, 4H), 0.65-0.53 (m, 1H).

### Example-98 and Example-99

### 3-Ethyl-2-(thiophen-2-ylsulfonyl)-2-azaspiro[4.4]nonane [EN-1]; and 3-ethyl-2-(thiophen-2-ylsulfonyl)-2-azaspiro[4.4]nonane [EN-2]

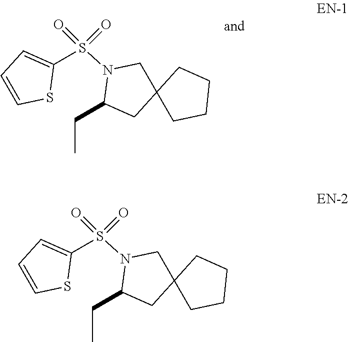

Chiral separation of 3-Ethyl-2-(thiophen-2-ylsulfonyl)-2-azaspiro[4.4]nonane (Example 97) provided both enantiomers as mentioned below. Chiral Separation Method: CHIRALPAK IG (250×21 mm) 5u, Flow rate—21 ml/min, Mobile phase—HEXANE/ETOH-90/10, Solubility—DCM, Wave length −244 nm, Run time-30 min.

Example 98 [EN-1]: Colourless gum (7 mg, 29% yield, 99.50% purity). LCMS: m/z found 300.19 [M+H]⁺, rt=2.24 min (Method 1) [Acquity BEH C8 column (1.7 μm, 50×2.1 mm)]; 1H NMR (400 MHz, DMSO-d6): δ 7.97 (d, 1H), 7.68 (d, 1H), 7.23 (t, 1H), 3.45-3.33 (m, 1H), 3.22-3.09 (m, 2H), 2.03-1.89 (m, 1H), 1.75 (dd, 1H), 1.63-1.28 (m, 7H), 1.26-1.02 (m, 3H), 0.82 (t, 3H).

Example 99 [EN-2]: Colourless gum (6 mg, 25% yield, 99.19% purity). LCMS: m/z found 300.19 [M+H]⁺, rt=2.24 min (Method 1) [Acquity BEH C8 column (1.7 μm, 50×2.1 mm)]; 1H NMR (400 MHz, DMSO-d6): δ 7.97 (d, 1H), 7.68 (d, 1H), 7.23 (t, 1H), 3.45-3.33 (m, 1H), 3.22-3.10 (m, 2H), 2.04-1.89 (m, 1H), 1.75 (dd, 1H), 1.64-1.27 (m, 7H), 1.25-1.05 (m, 3H), 0.82 (t, 3H).

### Example-100

### 2-Methyl-1-(7-((3-methyl-2-azaspiro[4.4]nonan-2-yl)sulfonyl)-3,4-dihydroisoquinolin-2(1H)-yl)propan-1-one

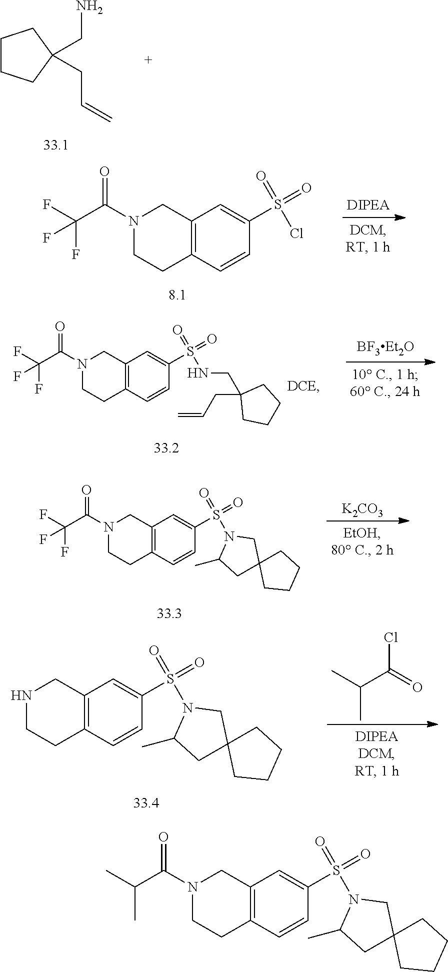

### N-((1-Allylcyclopentyl)methyl)-2-(2,2,2-trifluoroacetyl)-1,2,3,4-tetrahydroisoquinoline-7-sulfonamide 33.2

The synthesis was performed essentially as described in Method A using (1-allylcyclopentyl)-methanamine 33.1 and 2-(2,2,2-trifluoroacetyl)-1,2,3,4-tetrahydro-isoquinoline-7-sulfonyl chloride 8.1. (1-allylcyclopentyl)-methanamine 33.1 was synthesised from cyclopentanecarbonitrile 32.1 in a similar manner to that described in Example 97. The crude was purified by column chromatography over silica gel using 30% ethyl acetate in hexane and intermediate 33.2 was isolated as colourless gum. ¹H NMR (400 MHz, DMSO-d₆) δ 7.72 (d, 1H), 7.64 (s, 1H), 7.52 (s, 1H), 7.42 (s, 1H), 5.91-5.62 (m, 1H), 5.02 (d, 2H), 4.84 (d, 2H), 3.83 (s, 2H), 3.00 (s, 2H), 2.57 (s, 2H), 2.04 (d, 2H), 1.50 (s, 4H), 1.33 (d, 4H).

### 2,2,2-Trifluoro-1-(7-((3-methyl-2-azaspiro[4.4]nonan-2-yl)sulfonyl)-3,4-dihydroisoquinolin-2(1H)-yl)ethan-1-one 33.3

Intermediate 33.2 was cyclised to form intermediate 33.3 following the procedure described in Method V. The Crude was purified by flash chromatography to afford 33.3 as colourless gum. ¹H NMR (400 MHz, DMSO-d₆): δ 7.65 (t, 1H), 7.45 (d, 1H), 6.51 (s, 1H), 4.88 (d, 2H), 3.92-3.73 (m, 1H), 3.57-3.48 (m, 1H), 3.19 (d, 1H), 3.13-3.05 (m, 1H), 3.01 (q, 2H), 1.79 (dd, 1H), 1.59-1.34 (m, 10H), 1.32 (d, 3H).

### 7-((3-Methyl-2-azaspiro[4.4]nonan-2-yl)sulfonyl)-1,2,3,4-tetrahydroisoquinoline 33.4

The synthesis was performed essentially as described in Method D starting form intermediate 33.3. The crude intermediate 33.4 was used in the forwarding step without further purification.

### 2-Methyl-1-(7-((3-methyl-2-azaspiro[4.4]nonan-2-yl)sulfonyl)-3,4-dihydroisoquinolin-2(1H)-yl)propan-1-one (Example 100)

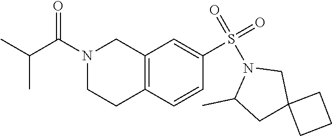 [SMILES: CC(C)C(=O)N1CCc2ccc(S(=O)(=O)N3CC4(CCC4)CC3C)cc2C1]

The synthesis was performed essentially as described in Method B using intermediate 32.4 and isobutyryl chloride. The crude was purified by column chromatography over silica gel using 25% ethyl acetate in hexane and the desired compound (Example 100) was isolated as colourless gum (35 mg, 31% yield, 93.30% purity). LCMS: m/z found 405.31 [M+H]⁺, rt=2.09 min (Method 1) [Acquity BEH C8 column (1.7 μm, 50×2.1 mm)]; 1H NMR (400 MHz, DMSO-d6): δ 7.71 (d, 1H), 7.60 (d, 1H), 7.41 (d, 1H), 4.82 (s, 1H), 4.71 (d, 1H), 3.76-3.65 (m, 2H), 3.52 (q, 1H), 3.18 (d, 1H), 3.08 (d, 1H), 3.01-2.92 (m, 2H), 2.88-2.80 (m, 1H), 1.88-1.69 (m, 1H), 1.63-1.28 (m, 10H), 1.17-0.95 (m, 6H), 0.94-0.54 (m, 2H).

### Example-101

### 1-(7-((3-Ethyl-2-azaspiro[4.4]nonan-2-yl)sulfonyl)-3,4-dihydroisoquinolin-2(1H)-yl)-2,2,2-trifluoroethan-1-one

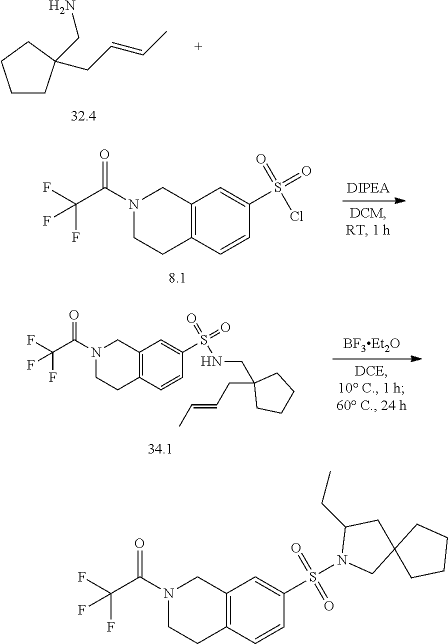 [SMILES: C/C=C/CC1(CN)CCCC1 ; O=C(N1CCc2ccc(S(=O)(=O)Cl)cc2C1)C(F)(F)F ; C/C=C/CC1(CNS(=O)(=O)c2ccc3c(c2)CN(C(=O)C(F)(F)F)CC3)CCCC1 ; CCC1CC2(CCCC2)CN1S(=O)(=O)c1ccc2c(c1)CN(C(=O)C(F)(F)F)CC2]

(E)-N-((1-(but-2-en-1-yl)cyclopentyl)methyl)-2-(2,2,2-trifluoroacetyl)-1,2,3,4-tetrahydroisoquinoline-7-sulfonamide 34.1: The synthesis was performed essentially as described in Method A using intermediate 32.4 and sulfonyl chloride 34.1. The crude was used in the forwarding step without further purification.

### 1-(7-((3-Ethyl-2-azaspiro[4.4]nonan-2-yl)sulfonyl)-3,4-dihydroisoquinolin-2(1H)-yl)-2,2,2-trifluoroethan-1-one

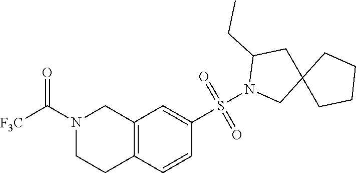 [SMILES: CCC1CC2(CCCC2)CN1S(=O)(=O)c1ccc2c(c1)CN(C(=O)C(F)(F)F)CC2]

Intermediate 34.1 was cyclised to form intermediate desired compound (Example 101) following the procedure described in Method V. The crude was purified by flash chromatography to afford Example 101 as colourless gum (35 mg, 30% yield, 99.24% purity). LCMS: m/z found 445.13 [M+H]⁺, RT=2.15 min (Method 2) [Acquity YMC Triart C18 column (3 μm, 33×2.1 mm)]; 1H NMR (400 MHz, DMSO-d6): δ 7.88-7.78 (m, 1H), 7.65 (t, 1H), 7.44 (d, 1H), 4.87 (d, 2H), 3.96-3.69 (m, 2H), 3.45-3.36 (m, 1H), 3.20-3.09 (m, 2H), 3.01 (q, 2H), 2.06-1.90 (m, 1H), 1.82-1.66 (m, 1H), 1.62-1.28 (m, 8H), 0.83 (t, 4H), 0.81-0.55 (m, 1H).

### Example-102

### 7-Methyl-6-(thiophen-2-ylsulfonyl)-6-azaspiro[3.4]octane

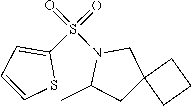 [SMILES: CC1CC2(CCC2)CN1S(=O)(=O)c1cccs1]

The synthesis was performed using a method analogous to that described in Method A using 7-methyl-6-azaspiro[3.4]octane hydrochloride 1.6 and thiophene-2-sulfonyl chloride. The crude was purified by column chromatography over silica gel using 20% ethyl acetate in hexane and isolated as light brown oil (133 mg, 88% yield, 99.85% purity). LCMS: m/z found 272.3 [M+H]⁺, rt=1.85 min (Method 2) [YMC Triart C18 column (3 μm, 33×2.1 mm)]; ¹H NMR (400 MHz, DMSO-d₆) δ 7.99 (dd, 1H), 7.69 (d, 1H), 7.24 (t, 1H), 3.58 (q, 1H), 3.43-3.34 (m, 1H), 3.19 (d, 1H), 2.03-1.79 (m, 3H), 1.76-1.62 (m, 2H), 1.56 (dd, 1H), 1.46-1.34 (m, 1H), 1.31 (d, 3H), 1.29-1.17 (m, 1H).

### Example-103 and Example-104

### 7-Methyl-6-(thiophen-2-ylsulfonyl)-6-azaspiro[3.4]octane [EN-1] and 7-methyl-6-(thiophen-2-ylsulfonyl)-6-azaspiro[3.4]octane [EN-2]

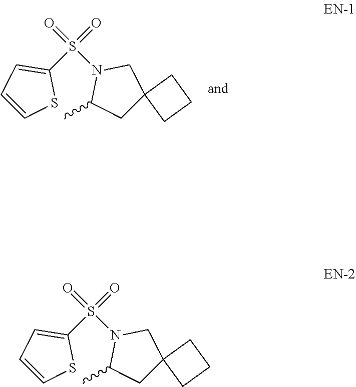

Chiral separation of racemic compound Example 102 provided ˜15 mg each of both isomers as mentioned below. The enantiomers were separated by Normal Phase Chiral Preparative HPLC. Chiral Preparative HPLC Method: Chiral separation was done on Agilent 1200 series instrument. Column name: Chiralpak AY-H (250×21 mm), 5μ, operating at ambient temperature and flow rate of 21.0 mL/min. Mobile phase: 0.1% Isopropylamine in the mixture of 80% Hexane and 20% Ethanol, held this isocratic mixture up to 20 min with wavelength of 244 nm.

[EN-1](Example-103): White solid (15 mg, 15% yield, 99.81% purity). LCMS: m/z found 272.27 [M+H]⁺, rt=1.85 min (Method 2) [YMC Triart C18 column (3 μm, 33×2.1 mm)]; ¹H NMR (400 MHz, DMSO-d₆): δ 7.99 (d, 1H), 7.69 (d, 1H), 7.25 (t, 1H), 3.59 (q, 1H), 3.44-3.27 (m, 1H), 3.19 (d, 1H), 2.04-1.79 (m, 3H), 1.77-1.62 (m, 2H), 1.56 (dd, 1H), 1.43-1.34 (m, 1H), 1.31 (d, 3H), 1.28-1.17 (m, 1H).

[EN-2](Example-104): White solid (15 mg, 15% yield, 99.72% purity). LCMS: m/z found 272.27 [M+H]⁺, rt=1.85 min (Method 2) [YMC Triart C18 column (3 μm, 33×2.1 mm)]; ¹H NMR (400 MHz, DMSO-d₆): δ 7.99 (d, 1H), 7.69 (d, 1H), 7.25 (t, 1H), 3.58 (q, 1H), 3.41-3.27 (m, 1H), 3.19 (d, 1H), 2.03-1.79 (m, 3H), 1.75-1.62 (m, 2H), 1.56 (dd, 1H), 1.43-1.37 (m, 1H), 1.30 (d, 3H), 1.26-1.17 (m, 1H).

### Example-105

### 2,4-Dimethyl-5-((7-methyl-6-azaspiro[3.4]octan-6-yl)sulfonyl)thiazole

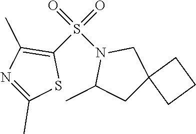 [SMILES: Cc1nc(C)c(S(=O)(=O)N2CC3(CCC3)CC2C)s1]

The synthesis was performed using a Scheme analogous to that described in Method A using 7-methyl-6-azaspiro[3.4]octane hydrochloride 1.6 and 2,4-dimethylthiazole-5-sulfonyl chloride. The crude was purified by column chromatography over silica gel using 20% ethyl acetate in hexane and isolated as light brown oil (106 mg, 95% yield, 99.68% purity). LCMS: m/z found 301.32 [M+H]⁺, rt=1.82 min (Method 2) [YMC Triart C18 column (3 μm, 33×2.1 mm)]; ¹H NMR (400 MHz, DMSO-d₆): δ 3.63 (q, 1H), 3.38 (d, 1H), 3.15 (d, 1H), 2.65 (s, 3H), 2.57 (s, 3H), 2.07-1.94 (m, 2H), 1.93-1.84 (m, 1H), 1.82-1.69 (m, 2H), 1.66-1.54 (m, 2H), 1.49 (t, 1H), 1.28 (d, 3H).

### Example-106 and Example-107

### 2,4-Dimethyl-5-((7-methyl-6-azaspiro[3.4]octan-6-yl)sulfonyl)thiazole [EN-1] and 2,4-dimethyl-5-((7-methyl-6-azaspiro[3.4]octan-6-yl)sulfonyl)thiazole ([EN-2])

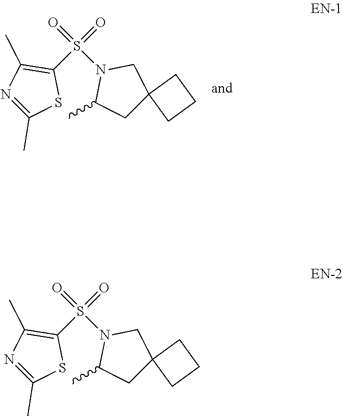

Chiral separation of racemic compound Example 105 provided both isomers as mentioned below. The enantiomers were separated by Normal Phase Chiral Preparative HPLC. Chiral Preparative HPLC Method: Chiral separation was done on Agilent 1200 series instrument. Column name: Chiralpak IC (250×20 mm), 5μ, operating at ambient temperature and flow rate of 18.0 mL/min. Mobile phase: 0.1% Isopropylamine in the mixture of 90% Hexane and 10% Ethanol, held this isocratic mixture up to 25 min with wavelength of 254 nm.

(Example-106 [EN-1]): Light brown oil (58 mg, 30% yield, 99.75% purity). LCMS: m/z found 301.2 [M+H]⁺, rt=1.82 min (Method 14) [Acquity BEH C8 column (1.7 μm, 50×2.1 mm)]; ¹H NMR (400 MHz, DMSO-d₆) δ 3.68-3.58 (m, 1H), 3.38 (d, 1H), 3.16 (d, 1H), 2.66 (s, 3H), 2.58 (s, 3H), 2.07-1.95 (m, 2H), 1.95-1.85 (m, 1H), 1.83-1.70 (m, 2H), 1.66-1.55 (m, 2H), 1.53-1.43 (m, 1H), 1.29 (d, 3H).

(Example-107) [EN-2]: Light brown oil (42 mg, 22% yield, 99.85% purity). LCMS: m/z found 301.2 [M+H]⁺, rt=1.82 min (Method 14) [Acquity BEH C8 column (1.7 μm, 50×2.1 mm)]; ¹H NMR (400 MHz, DMSO-d₆) δ 3.64 (q, 1H), 3.38 (d, 1H), 3.16 (d, 1H), 2.66 (s, 3H), 2.58 (s, 3H), 2.06-1.95 (m, 2H), 1.95-1.84 (m, 1H), 1.81-1.71 (m, 2H), 1.66-1.54 (m, 2H), 1.53-1.43 (m, 1H), 1.29 (d, 3H).

### Example-108

### 6-((5-Chlorothiophen-2-yl)sulfonyl)-7-methyl-6-azaspiro[3.4]octane

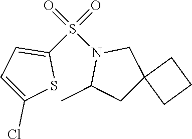 [SMILES: CC1CC2(CCC2)CN1S(=O)(=O)c1ccc(Cl)s1]

The synthesis was performed using a method analogous to that described in Method A using 7-methyl-6-azaspiro[3.4]octane hydrochloride 1.6 and 5-chlorothiophene-2-sulfonyl chloride. The crude was purified by column chromatography over silica gel using 20% ethyl acetate in hexane and isolated as colourless oil (90 mg, 95% yield, 99.89% purity). LCMS: m/z found 306.1 [M+H]⁺, rt=2.48 min (Method 14) [Acquity BEH C8 column (1.7 μm, 50×2.1 mm)]; ¹H NMR (400 MHz, DMSO-d₆): δ 7.62 (d, 1H), 7.33 (d, 1H), 3.60 (q, 1H), 3.36 (d, 1H), 3.21 (d, 1H), 2.06-1.93 (m, 2H), 1.87 (t, 1H), 1.79-1.66 (m, 2H), 1.65-1.54 (m, 1H), 1.55-1.43 (m, 1H), 1.43-1.33 (m, 1H), 1.29 (d, 3H).

### Example-109

### 5-((7-Methyl-6-azaspiro[3.4]octan-6-yl)sulfonyl)thiazole

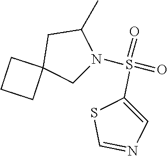 [SMILES: CC1CC2(CCC2)CN1S(=O)(=O)c1cncs1]

The synthesis was performed using a Scheme analogous to that described in Method A using 7-methyl-6-azaspiro[3.4]octane hydrochloride 1.6 and thiazole-5-sulfonyl chloride. The crude was purified by column chromatography over silica gel using 30% ethyl acetate in hexane and isolated as light brown oil (14 mg, 92% yield, 99.08% purity). LCMS: m/z found 273.0 [M+H]⁺, rt=3.46 min (Method 3) [Zorbax C18 column (5 μm, 50×4.6 mm)]; ¹H NMR (400 MHz, DMSO-d₆): δ 9.43 (s, 1H), 8.47 (s, 1H), 3.59 (q, 1H), 3.39 (d, 1H), 3.20 (d, 1H), 2.05-1.92 (m, 2H), 1.92-1.80 (m, 1H), 1.78-1.64 (m, 2H), 1.59 (dd, 1H), 1.49-1.37 (m, 1H), 1.31 (d, 3H), 1.30-1.22 (m, 1H).

### Example-110

### 6-((5-fluorothiophen-2-yl)sulfonyl)-7-methyl-6-azaspiro[3.4]octane

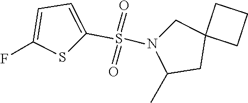 [SMILES: CC1CC2(CCC2)CN1S(=O)(=O)c1ccc(F)s1]

The synthesis was preformed essentially as described in Method A using 5-fluorothiophene-2-sulfonyl chloride and 7-methyl-6-azaspiro[3.4]octane hydrochloride using pyridine as base in THF. The compound was purified by flash chromatography over silica gel using 3:7 ethyl acetate:hexane and isolated as colourless oil (24 mg, 17% yield, 94.61% purity). ¹H NMR (400 MHz, DMSO-d₆): δ 7.50 (t, J=4.0 Hz, 1H), 6.99 (d, J=4.4 Hz, 1H), 3.64-3.54 (m, 1H), 3.42-3.32 (m, 1H), 3.20 (d, J=10.7 Hz, 1H), 2.04-1.94 (m, 2H), 1.87 (dd, J=1.8, 5.9 Hz, 1H), 1.80-1.65 (m, 2H), 1.62-1.47 (m, 2H), 1.46-1.34 (m, 1H), 1.29 (d, J=6.2 Hz, 3H).

### Example-111

### 5-((7-methyl-6-azaspiro[3.4]octan-6-yl)sulfonyl)nicotinonitrile

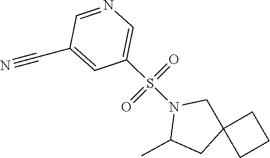 [SMILES: CC1CC2(CCC2)CN1S(=O)(=O)c1cncc(C#N)c1]

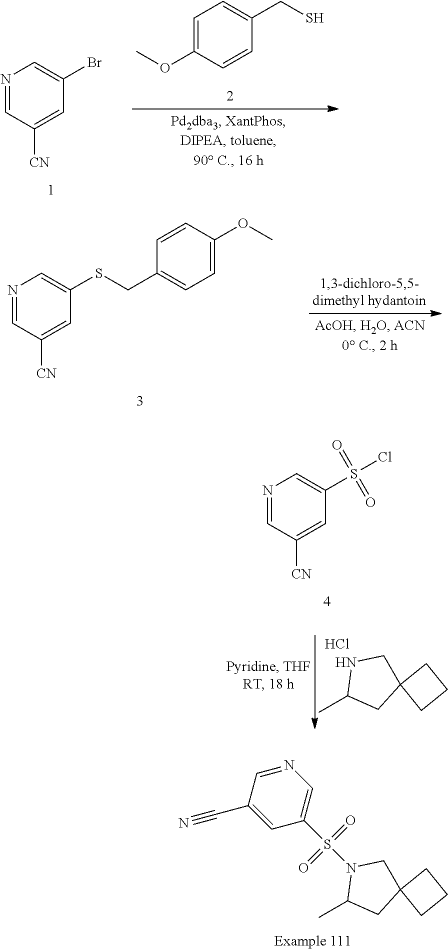

5-((4-methoxybenzyl)thio)nicotinonitrile (3): Buchwald Coupling (Method I): A mixture of 5-bromonicotinonitrile (1) (10 g, 54.64 mmol), and (4-methoxyphenyl)methanethiol (7.6 mL, 54.64 mmol) was dissolved in toluene (50 mL) and DIPEA (19 mL, 109.3 mmol) was added to it. It was then degassed with argon and Xantphos (1.6 g, 2.73 mmol) was added to the reaction mixture, followed by Pd₂dba₃ (1.5 g, 1.64 mmol) under argon atmosphere. The reaction mixture was heated at 90° C. for 16 h. The crude reaction mass was then filtered through a celite bed and the filtrate was evaporated. The crude residue was diluted with water (100 mL) and extracted with ethyl acetate (3×100 mL). The combined organic part was washed with water (100 mL) and brine (100 mL), dried (anhydrous Na₂SO₄), and the solvent was evaporated under reduced pressure. The crude was further purified by column chromatography over silica gel using 50% ethyl acetate in hexane and desired intermediate 3 was isolated as light yellow solid (12.0 g, 86% yield). 1H NMR (400 MHz, DMSO-d6): δ 8.78 (d, J=1.7 Hz, 1H), 8.73 (d, J=2.2 Hz, 1H), 8.32 (t, J=2 Hz, 1H), 7.29 (d, J=8.6 Hz, 2H), 6.87 (d, J=8.6 Hz, 2H), 4.34 (s, 2H), 3.71 (s, 3H).

5-cyanopyridine-3-sulfonyl chloride (4): Intermediate 3 (5.0 g, 19.53 mmol) was dissolved in CH₃CN (20 mL) and to the solution a mixture of water (5 mL) and AcOH (1 mL) was added. The resulting mixture was then cooled to −5° C. and 1,3-dichloro-5,5-dimethyl hydantoin (5.7 g, 29.28 mmol) was added to it. The reaction was stirred for 2 h at −5° C. The RM was then quenched with water and extracted with DCM (3×50 mL). The organic part was washed with cold water (50 mL), dried and the solvent was evaporated under reduced pressure at RT. The crude residue was further purified by column chromatography over silica gel using 30% ethyl acetate in hexane and the desired intermediate 4 was isolated as light yellow solid (1.5 g, 38% yield). ¹H NMR (400 MHz, DMSO-d6): δ 8.95-9.02 (m, 2H), 8.32-8.5 (bs, 1H).

5-((7-methyl-6-azaspiro[3.4]octan-6-yl)sulfonyl)nicotinonitrile (Example 111): To a stirred solution of 7-methyl-6-azaspiro[3.4]octane hydrochloride salt (159 mg, 0.99 mmol) in pyridine (0.8 mL, 9.87 mmol) 5-cyanopyridine-3-sulfonyl chloride (200 mg, 0.987 mmol) in THF (2 mL) was added at RT and the reaction mixture was stirred overnight. The volatiles were evaporated under reduced pressure and the crude obtained was purified by combi-flash chromatography using 30% EA-Hexane to afford desired compound Example 111 as an off-white solid (80 mg, 28% yield, and 99.98% HPLC purity). LCMS: m/z found 292.2 [M+H]⁺; rt=2.75 min (Method 4) [Waters Xbridge C18 column (3.5 μm, 50×3 mm)]; ¹H NMR (400 MHz, DMSO-d6): δ 9.31 (d, J=1.7 Hz, 1H), 9.25 (d, J=2.1 Hz, 1H), 8.82 (s, 1H), 3.75-3.61 (m, 1H), 2.03-1.90 (m, 3H), 1.87-1.76 (m, 1H), 1.80-1.58 (m, 3H), 1.53 (dd, J=7.1, 12.5 Hz, 1H), 1.30 (d, J=6.2 Hz, 3H), 1.29-1.08 (m, 2H).

### PHARMACOLOGICAL EXAMPLES

### Example 112: Human Cav2.3 Channel Calcium-Influx Assay

### Cell Line

HEK-293 T-Rex cells were transfected with pcDNA3.1-Kir2.1 to generate a stable cell line. After antibiotic selection, the obtained stable pools were analysed using FLIPR^{TETRA} (membrane potential assay), and a limiting dilution was performed and a HEK-293 T-Rex/Kir2.1 clone was selected. The clone was transfected with pcDNA3.1-Cav2.3e and pBud-Cav-β4-α2δ1 to generate a stable cell line where the Cav2.3e expression is inducible. After antibiotic selection, functional clone pool analysis and two successive limiting dilutions, the final clone underwent a qPCR analysis and a biophysical and pharmacological validation using patch-clamp.

### Assay

A compound plate, containing 10 test compounds in eight-points dose response with n=4, was prepared. Test compounds dose response curves were prepared in automated fashion in 100% DMSO at CyBio Felix and starting from a stock solution in 100% DMSO; a defined volume was serially moved into destination wells pre-filled with the desired DMSO volume; stock solution concentration volumes were dependent on the final compounds concentrations to be tested and the final DMSO content used in the assay. For the assay test compounds dose response curves ranged from 30 μM and half log dilution with 0.3% final DMSO. In order to obtain the 8-points dose response curve all the concentrations were created by starting from a 10 mM DMSO stock solution and then moving 9.5 μL into a destination well pre-filled with 20.5 μL of 100% DMSO and repeating this step seven times. This initial step was done into 96 MTP plates, 10 compounds/plate. One 384 MTP was then reformatted to contain all the 10 compounds at 8 concentrations, quadruplicate data points. This so obtained 384 MTP compound plate served as source plate in a “mother to child” process with a CyBi®-Well dispenser in which 0.7 μL of compounds were moved into a destination plate pre-filled with 57.6 μL of Tyrode's buffer 0 mM K+, thus obtaining 4× concentrated compounds solution. In columns 1-2 and 23-24 the control wells were added. Both “source” compound plate and “destination” compound plate were barcoded and a relationship between the two plates was thus generated.

The day before the experiment, cells were detached by gentle wash with DPBS, followed by 5 min incubation at 37° C. with Trypsin solution. Detached cells were diluted with OptiMEM+Doxycycline at 0.2 μg/mL, counted and plated in Poly-D-Lysine coated black/clear bottom (15.000 c/well in 20 μl/well) by the use of a MATRIX WellMate dispenser. Plates were placed into a humidified incubator at 37° C. with 5% CO₂ until the experimental day. 24 h after seeding 10 μL/well of 1.5× Fluo8 NW dye solution prepared in Tyrode's buffer 0 mM K⁺ were added on top of the seeding medium. Cell plates were incubated for 40-60 min at RT in the dark. We then Injected off-line 10 μL/well of test compounds and controls 4× concentrated in Tyrode's Buffer 0 mM K+ with the CyBi®-Well instrument. Cell plates were incubated 3 minutes at RT. Finally, we injected 20 μL/well of 3× concentrated activator solution (K20-Na130-Ca2 buffer: 20 mM KCl, 130 mM NaCl, 2 mM CaCl2, 10 mM HEPES, 10 mM Glucose, final concentrations, prepared starting from “KO-Na150-Ca2” and “K150-Na0-Ca2” buffers) at FDSS7000EX instrument and read emitted fluorescence for 130 seconds.

Data analysis was performed with Genedata Screener® software and reported compounds activity as % effect in relation to the normalization standards. The Kinetic Response Value (KRV) is calculated as follows:

KRV=Maximal fluorescence recorded from second 5 to second 130 minus baseline fluorescence, computed as average from second 1 to second 2, of the kinetic trace. The KRV was normalized versus Neutral Controls and Inhibitor Controls in order to obtain the Activity[%] for each well. The normalization places the compound activity values on an equivalent scale and makes them comparable across plates or different compound batches. Therefore, the compound activity values were scaled (based on the two references) to a common range (two-point normalization). The following equation was used by the software to normalize the signal values to the desired signal range:

$$N ( x ) = CR + [ ( ( x - < cr > ) / ( < s r > - < c r > ) ) . ( SR - CR ) ]$$

where: x is the calculated signal value of a well (KRV); <cr> is the median of the calculated signal values (KRV) for the Central Reference wells of a plate (median of Neutral Controls); <sr> is the median of the calculated signal values (KRV) for the Scale Reference wells of a plate (median of Inhibitor Controls); CR is the desired median normalized value for the Central Reference (0) and SR is the desired median normalized value for the Scale Reference (−100).

The final equation to calculate the Activity % can be simplified as follow:

% Activity=−100·(x−<NeutralControls>)/(<NeutralControls>−<InhibitorControls>) where full inhibition corresponds to % Activity=−100

The fitting of the dose-response curve of each test compound is performed in the Analyzer module of the Screener software on the normalized values and applying the “smart fit” strategy. This strategy allowed an automatic selection between the “Constant Fit” and the “Hill Fit” model calculating which fit model best matched the experimental data. The Constant Fit was applied when no change of activity was detected across the measured concentrations, and the corresponding compounds were further classified as inactive. The Hill Fit was applied when the observed activity significantly changed with the compound concentration. In case of Hill Fit, Hill equation was used to determine the concentration at which activity reaches 50% of maximum level, i.e., AC₅₀.

Y=S₀+((S_{inf}−S₀)/(1+(10^{LogAC50}/10^{X})ⁿ))

where X is Log 10 of compound concentration. The equation has four parameters:

Zero Activity (S0)—Activity level at zero concentration of test compound; Infinite Activity (S_{inf})—Activity level at infinite concentration of test compound; AC₅₀-Concentration at which activity reaches 50% of maximum level. This term corresponds to IC₅₀ in this assay; AND Hill coefficient (n)—Measure of the slope at AC₅₀.

The pIC₅₀ values measured in this assay for the exemplified compounds is set out in the table below:

| | Cav2.3 | | Cav2.3 | | Cav2.3 |
|---|---|---|---|---|---|
| Example | (pIC₅₀) | Example | (pIC₅₀) | Example | (pIC₅₀) |
| 1 | 5.85 | 2 | 5.37 | 3 | 4.52 |
| 4 | 5.38 | 5 | 5.53 | 6 | 5.14 |
| 7 | 5.66 | 8 | 5.59 | 9 | 5.10 |
| 10 | 5.57 | 11 | 5.49 | 12 | 5.18 |
| 13 | 5.78 | 14 | 4.33 | 15 | 5.73 |
| 16 | 5.32 | 17 | 5.88 | 18 | 5.61 |
| 19 | 5.75 | 20 | 5.37 | 21 | 6.02 |
| 22 | 6.13 | 23 | 6.30 | 24 | 5.79 |
| 25 | 5.53 | 26 | 5.96 | 27 | 6.07 |
| 28 | 5.82 | 29 | 5.32 | 30 | 6.04 |
| 31 | 6.19 | 32 | 5.80 | 33 | 5.73 |
| 34 | 5.96 | 35 | 5.42 | 36 | 5.28 |
| 37 | 5.10 | 38 | 5.47 | 39 | 4.97 |
| 40 | 5.08 | 41 | 5.08 | 42 | 4.61 |
| 43 | 4.99 | 44 | 5.17 | 45 | 5.47 |
| 46 | 5.11 | 47 | 5.80 | 48 | 4.80 |
| 49 | 5.41 | 50 | 5.03 | 51 | 5.56 |
| 52 | 5.70 | 53 | 5.31 | 54 | 5.09 |
| 55 | 5.39 | 56 | 5.08 | 57 | 5.29 |
| 58 | 4.94 | 59 | 4.95 | 60 | 5.32 |
| 61 | 5.75 | 62 | 6.17 | 63 | 5.25 |
| 64 | 5.43 | 65 | 5.40 | 66 | 5.04 |
| 67 | 5.01 | 68 | 4.40 | 69 | 5.60 |
| 70 | 5.02 | 71 | 5.93 | 72 | 5.36 |
| 73 | 5.77 | 74 | 5.22 | 75 | 4.43 |
| 76 | 5.10 | 77 | 4.86 | 78 | 5.44 |
| 79 | 5.20 | 80 | 5.55 | 81 | 5.34 |
| 82 | 4.97 | 83 | 5.05 | 84 | 5.35 |
| 85 | 4.72 | 86 | 5.87 | 87 | 5.70 |
| 88 | 5.60 | 89 | 5.00 | 90 | 4.95 |
| 91 | 5.62 | 92 | 5.61 | 93 | 5.69 |
| 94 | 5.70 | 95 | 5.11 | 96 | 5.03 |
| 97 | 5.60 | 98 | 4.84 | 99 | 5.72 |
| 100 | 6.15 | 101 | 5.61 | 102 | 5.57 |
| 103 | 5.08 | 104 | 5.85 | 105 | 5.34 |
| 106 | 5.66 | 107 | 5.17 | 108 | 4.94 |
| 109 | 5.40 | 110 | 5.30 | 111 | 5.71 |

The inventors have found that for some high affinity compounds, the addition of pluronic acid (0.05%) aided solubility and in doing so increased the measured pIC₅₀ values by approximately 0.5 units. However, all of the reported pIC₅₀ values presented above did not include pluronic acid.

### Example 113: Whole-Cell Patch Clamp Assay

HEK-293 T-Rex/Kir2.1/Cav2.3e-β4-α2δ1 cells were seeded 96 hours before experiment and doxycycline at 0.2 μg/mL was added 24 hours before experiment. Just before the experiments cells were washed twice with D-PBS w/o Ca2+/Mg2+ and gently detached from the flask with Detachin. Cells were then re-suspended in the suspension solution (25 mL EX-CELL ACF CHO medium; 0.25 mL of 100× Penicillin/Streptomycin) and placed on the Automated Patch-clamp platform (QPatch 16X).

Standard whole-cell voltage clamp experiments were performed at room temperature using the single hole technology and the following intracellular and extracellular solutions (ICS and ECS, respectively) were used. ICS (mM): CsF 60, CsCl 50, NaCl 10, EGTA 20, BAPTA 5, HEPES 10, NaGTP 0.3, MgATP 5 (pH 7.2; ECS (mM): NMDG-Cl 120, BaCl2 20, HEPES 10, EGTA 7 (pH 7.4). After establishment of the seal and the passage in the whole-cell configuration, the Cav2.3 dependent current was evoked by challenging the cells with the following voltage protocol: the plasma membrane was held at −80 mV then a 50 ms-long depolarizing pulse at 0 mV, followed by a 50 ms-long hyperpolarizing pulse at −100 mV were applied and finally the plasma membrane was held back to −80 mV. This voltage protocol was applied every 10 seconds. Cav2.3 dependent current was first measured in control condition (vehicle, 0.1% DMSO), in the presence of increasing concentration of the compound under investigation and finally SNX 482 was added to fully block any R-type Cav2.3 dependent current.

For data collection, the Sophion proprietary software was used while the analysis was performed off-line using Excel and GraphPad Prism. The inward current (Area Under Curve) measured during each depolarizing pulse was normalized to cell membrane capacitance (Cm) to obtain the average inward current density in each condition (control, compound under investigation and SNX 482). Then, the inhibitory effect of the compound under investigation was evaluated as % of the remaining inward current, normalized to the internal control. When the percentage of inhibition exerted by the highest concentration tested was superior to 50%, the dose-response curves data was fitted with the following equation: Y=100/(1+10\hat{ }((Log IC₅₀—X)*Hillslope)); where X: log of concentration; Y: normalized response, 100% down to 0%, decreasing as X increases; Log IC₅₀: same log units as X; Hillslope: slope factor or hill slope, unit less. All data were expressed as mean±S.E

The pIC₅₀ values for some of the exemplified compounds of the invention are set out in the table below:

| | Cav2.3 |
|---|---|
| Example | (pIC₅₀) |
| 1 | 5.55 |
| 21 | 6.03 |
| 22 | 6.12 |
| 23 | 6.08 |
| 26 | 6.28 |
| 27 | 6.08 |
| 28 | 5.77 |
| 30 | 5.77 |
| 31 | 5.39 |
| 32 | 5.63 |
| 33 | 5.55 |
| 71 | 5.80 |
| 100 | 5.93 |
| 104 | 5.43 |

### Example 114: Ex Vivo Activity in Substantia Nigra Dopamine Neurons in Mouse Brain Slice Experiments

Compounds of the invention may be tested on R-type calcium current with whole cell patch clamp electrophysiology in substantia nigra dopamine neurons in an ex. vivo brain slice preparation according to the methods described in Siller et al., Elife, 11:e67464 (2022) https://doi.org/10.7554/eLife. 67464

### Example 115: In Vivo Activity in the Maximal Electroshock Stimulation Model (MES) in Mouse

Compounds of the invention may be tested in an in vivo model of epilepsy, the MES model in mouse, according to the method described in Kehne et al, Neurochemistry Research 42:1894-1903 (2017) I https://doi.org/10.1007/s11064-017-2275-z.

1. A compound of the formula (I), or a pharmaceutically acceptable salt thereof: 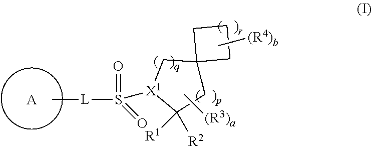
wherein:
X¹ is CH or N;
R¹ is selected from: halo, C₁₋₆ alkyl, C₁₋₆ haloalkyl, C₃₋₆ cycloalkyl, and C₃₋₆ cycloalkyl-C₁₋₆ alkyl-,
wherein said C₁₋₆ alkyl, C₃₋₆ cycloalkyl and C₃₋₆ cycloalkyl-C₁₋₆ alkyl-is each optionally substituted by one or more substituents independently selected from: halo, C₁₄ alkyl, ═O, —CN, —OR^{1A}, —S(O)ₓR^{1A} and —NR^{1A}R^{1B};
R² is selected from: H, halo, C₁₋₆ alkyl and C₁₋₆ haloalkyl; or
R¹ and R² together with the carbon atom to which they are attached form a C₃₋₆ cycloalkyl,
wherein said C₃₋₆ cycloalkyl is optionally substituted by one or more substituent selected from: halo, C₁₋₄ alkyl, ═O, —CN, —OR^{2A}, —S(O)ₓR^{2A} and —NR^{2A}R^{2B};
each R³ and R⁴ is independently selected from: halo, C₁₋₆ alkyl and C₁₋₆ haloalkyl;
L is independently selected from: a bond, and C₁₋₃ alkylene;
Ring A is selected from 5- to 12-membered heteroaryl and C₆₋₁₀ aryl; wherein Ring A is optionally substituted by one or more R⁵;
each R⁵ is independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
wherein said C₁₋₆ alkyl, C₂₋₆ alkenyl and C₂₋₆ alkynyl is optionally substituted by one or more R⁸;
R⁶ and R⁷ are each independently selected from: H, C₁₋₆ alkyl, C₁₋₆ haloalkyl and Q¹, wherein said C₁₋₆ alkyl is optionally substituted by one or more R⁹;
each R⁸ and R⁹ is independently selected from: halo, —CN, —OR^{8A}, —S(O)ₓR^{8A}, —R^{8A}R^{8B} and Q²;
each Q¹ and Q² is independently selected from: C₃₋₆ cycloalkyl, 4- to 7-membered heterocyclyl, phenyl and 5- or 6-membered heteroaryl,
wherein said C₃₋₆ cycloalkyl and 4- to 7-membered heterocyclyl is optionally substituted by one or more R¹⁰,
wherein said phenyl and 5- or 6-membered heteroaryl is optionally substituted by one or more R¹¹;
each R¹⁰ is independently selected from: halo, ═O, —CN, —NO₂, C₁₋₄ alkyl, C₁₋₄ haloalkyl, —OR^{10A}, —S(O)₂R^{10A}, —NR^{10A}R^{10B}, —C(O)R^{10A}, —OC(O)R^{10A}, —C(O)OR^{10A}, —NR^{10B}C(O)R^{10A}, —C(O)NR^{10A}R^{10B}, —NR^{10B}C(O)OR^{10A}, —OC(O)NR^{10A}R^{10B}, —NR^{10B}SO₂R^{10A} and —SO₂NR^{10A}R^{10B},
wherein said C₁₋₄ alkyl is optionally substituted by 1 or 2 substituents selected from: halo, —CN, —OR^{10C}, —NR^{10C}R^{10D} and —SO₂R^{10C};
each R¹¹ is independently selected from: halo, —CN, —NO₂, C₁₋₄ alkyl, C₁₋₄ haloalkyl, —OR^{7A}, —S(O)₂R^{11A}, —NR^{11A}R^{11B}, —C(O)R^{11A}, —OC(O)R^{11A}, —C(O)OR^{11A}, —NR^{11B}C(O)R^{11A}, —C(O)NR^{11A}R^{11B}, —NR^{11B}C(O)OR^{11A}, —OC(O)NR^{11A}R^{11B}, —NR^{11B}SO₂R^{11A} and —SO₂NR^{11A}R^{11B},
wherein said C₁₋₄ alkyl is optionally substituted by 1 or 2 substituents selected from: halo, —CN, —OR^{11C}, —NR^{11C}R^{11D} and —SO₂R^{11C};
R^{1A}, R^{1B}, R^{2A}, R^{2B}, R^{8A}, R^{8B}, R^{10A}, R^{10B}, R^{10C}, R^{10D}, R^{11A}, R^{11B}, R^{11C}, R^{11D} are at each occurrence independently selected from: H, C₁₋₄ alkyl and C₁₋₄ haloalkyl; and wherein any —NR^{1A}R^{1B} NR^{2A}R^{2B}, —NR⁶R⁷, —NR^{8A}R^{8B}, —NR^{10A}R^{10B}, —NR^{10C}R^{10D}, —NR^{11A}R^{11B} and —NR^{11A}R^{11B} within a substituent may form a 4- to 6-membered heterocyclyl, wherein said 4- to 6-membered heterocyclyl is optionally substituted by one or more substituents selected from: halo, ═O, C₁₋₄ alkyl and C₁₋₄ haloalkyl;
a and b are each independently an integer from 0 to 4;
p is 1 or 2;
q is 1 or 2;
r is 0, 1 or 2; and
each x is independently 0, 1, or 2;
with the proviso that the compounds in List 1 are excluded:

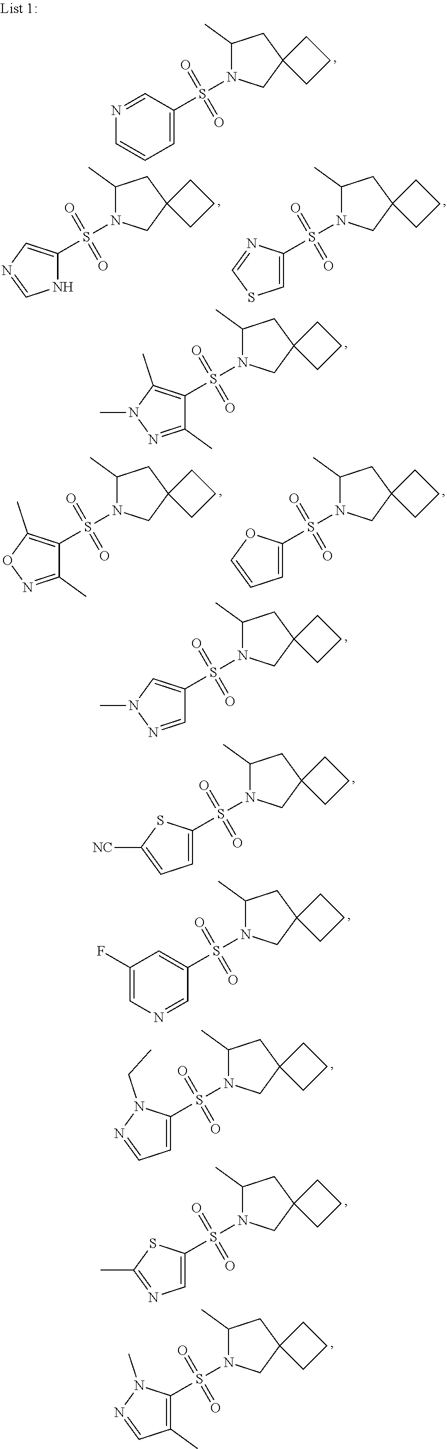

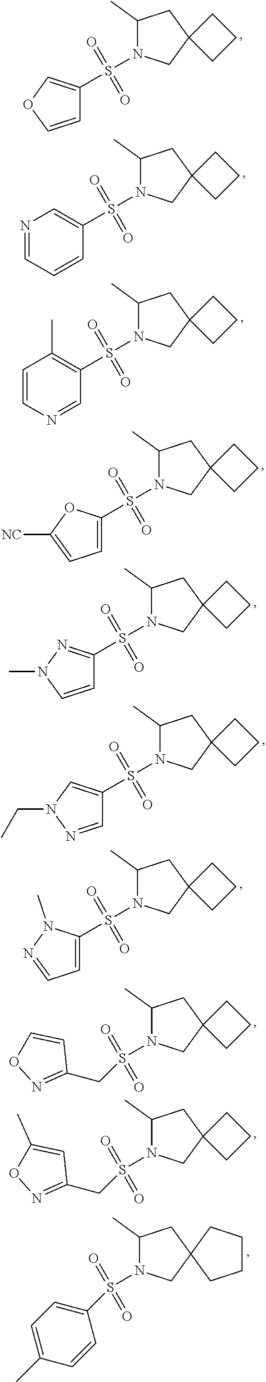

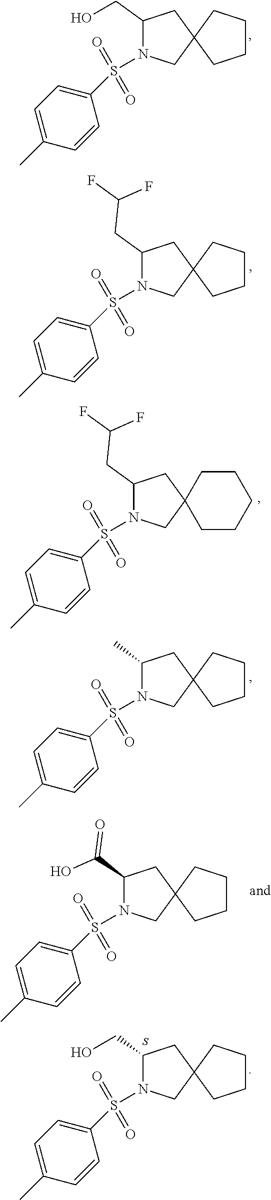

2. The compound according to claim 1, wherein Ring A is selected from: furanyl, thienyl, imidazolyl, pyrazolyl, oxazolyl, isoxazolyl, thiazolyl, isothiazolyl, thiadiazolyl, phenyl, pyridyl, pyrimidinyl, pyrazinyl, 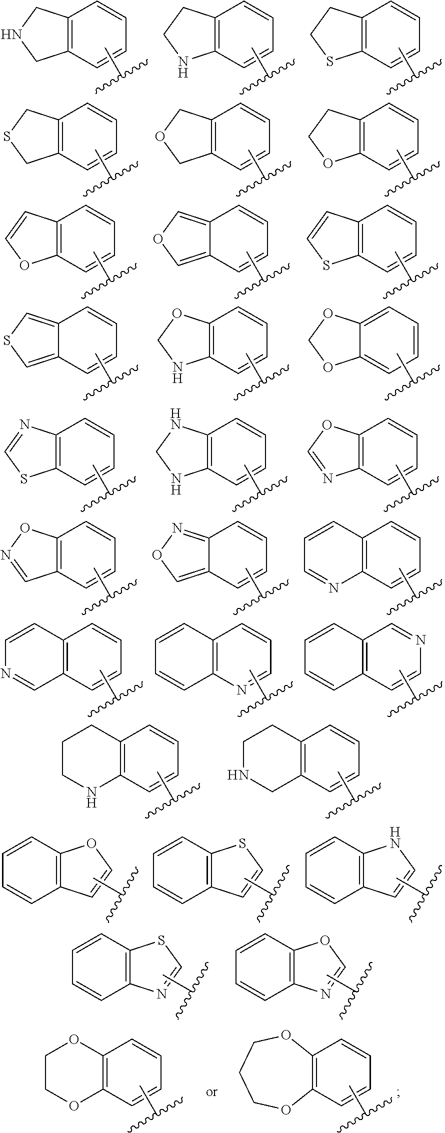
wherein Ring A is optionally substituted with one or more R⁵.

3. The compound according to claim 1, wherein each R⁵ is independently selected from: halo, —CN, —NO₂, ═O, C₁₋₆ alkyl, C₁₋₆ haloalkyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶SO₂R⁷, and —SO₂NR⁶R⁷.

4. The compound according to claim 1, wherein the compound is of the formula (XIII), or a pharmaceutically acceptable salt thereof: 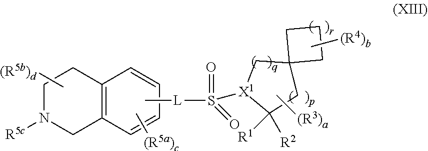
wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,
wherein said C₁₋₆ alkyl, C₂₋₆ alkenyl and C₂₋₆ alkynyl is optionally substituted by one or more R⁸;
c is an integer from 0 to 3;
d is an integer from 0 to 4.

5. The compound according to claim 1, wherein the compound is of the formula (XIX), or a pharmaceutically acceptable salt thereof: 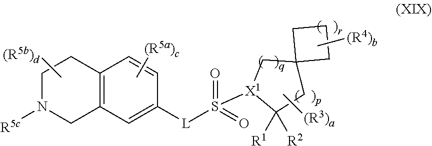
wherein:
R^{5a}, and R^{5b} are each independently selected from: halo, —CN, —NO₂, ═O, C₁₋₆ alkyl, C₁₋₆ haloalkyl, C₂₋₆ alkenyl, C₂₋₆ alkynyl, Q¹, —OR⁶, —S(O)ₓR⁶, —NR⁶R⁷, —C(O)R⁶, —OC(O)R⁶, —C(O)OR⁶, —NR⁶C(O)R⁷, —C(O)NR⁶R⁷, —NR⁶C(O)OR⁷, —OC(O)NR⁶R⁷, —NR⁶SO₂R⁷, and —SO₂NR⁶R⁷,
R^{5c} is independently selected from: C₁₋₆ alkyl, C₁₋₆ haloalkyl, C₂₋₆ alkenyl, C₂₋₆ alkynyl, Q¹, —S(O)ₓR⁶, —C(O)R⁶, —C(O)OR⁶, —C(O)NR⁶R⁷, and —SO₂NR⁶R⁷,
wherein said C₁₋₆ alkyl, C₂₋₆ alkenyl and C₂₋₆ alkynyl is optionally substituted by one or more R⁸;
c is an integer from 0 to 3;
d is an integer from 0 to 4.

6. The compound according to claim 1, wherein X¹ is N.

7. The compound according to claim 1, wherein L is a bond or methylene.

8. The compound according to claim 1, wherein L is a bond.

9. The compound according to claim 1, wherein R¹ is selected from: methyl, ethyl, cyclohexyl, —CH₂OH, and CH₂OMe; and R² is selected from H and methyl.

10. The compound according to claim 1, wherein R¹ is selected from methyl and ethyl; and R² is H.

11. The compound according to claim 1, wherein the
group of the formula 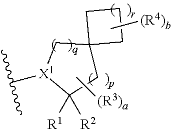
is selected from:

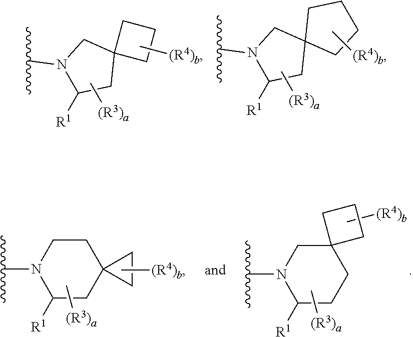

12. The compound according to claim 1, wherein the
group of the formula 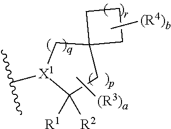
is selected from:

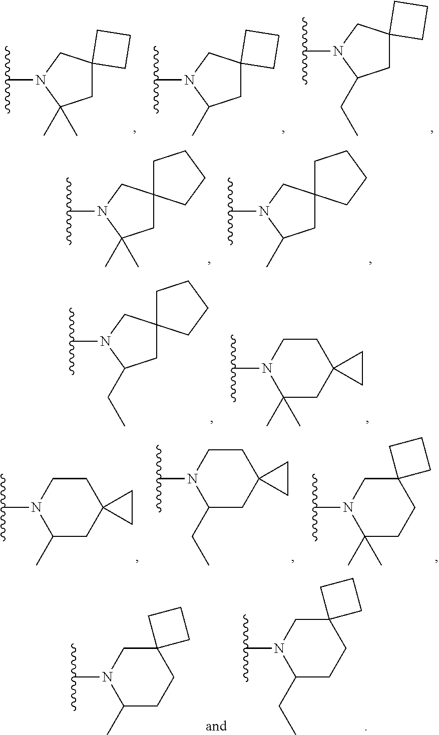

13. The compound according to claim 1, wherein the
group of the formula 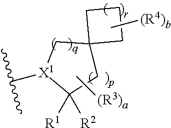
is

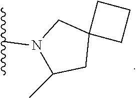 [SMILES: *N1CC2(CCC2)CC1C]

14. The compound according to claim 1, wherein the compound is selected from Compound List A in the description, or a pharmaceutically acceptable salt thereof.

15. A pharmaceutical composition comprising a compound according to claim 1, except the compounds from List 1 are not excluded, or a pharmaceutically acceptable salt thereof, and a pharmaceutically acceptable excipient.

16. (canceled)

17. (canceled)

18. A method of treating a disease or medical disorder mediated by Cav2.3 in a subject in need thereof, the method comprising administering to the subject an effective amount of a compound according to claim 1, except the compounds from List 1 are not excluded, or a pharmaceutically acceptable salt thereof.

19.-24. (canceled)
